(19) United States
(12) Patent Application Publication
(10) Pub. No.: US 2003/0092601 A1
(43) Pub. Date: May 15, 2003

(54) Microcompetition and human disease

(76) Inventor: Hanan Polansky, Rochester, NY (US)

Correspondence Address: Brown, Pinnisi and Michaels, P.C., 400 M&T Bank Building-118 North Tioga Street, Ithaca, NY, 14850-4343, US

(21) Appl. No.: 09/732,360

(22) Filed: Dec. 7, 2000

Related U.S. Application Data
(60) Provisional application No. 60/169,518, filed on Dec. 7, 1999.
(60) Provisional application No. 60/183,184, filed on Feb. 17, 2000.

(51) Int. Cl. A61K 31/00

(52) U.S. Cl. 514/1

(57) ABSTRACT

Cellular microcompetition for the transcription factor human GA binding protein (GABP) is a risk factor associated with obesity and obesity-related diseases such as osteoarthritis, atherosclerosis, obstructive sleep apnea, various cancers, and periodontitis. The invention uses this novel discovery to develop assays which determine the level of microcompetition in a cell. Other assays developed from the knowledge that microcompetition is occurring in cells are also disclosed. This novel discovery led to the development of assays which can determine the level of microcompetition in a cell and to select compounds to target this microcompetition syndrome. In addition, methods to treat a patient for microcompetition based disease are taught.

### BACKGROUND OF THE INVENTION

1. Field of the Invention

The invention pertains to the field of human diseases. More particularly, the invention pertains to how microcompetition for transcription factors contributes to obesity, cancer, atherosclerosis, osteoarthritis, hypertension and diabetes.

2. Description of Related Art

The cause of many cases of obesity, cancer, atherosclerosis, osteoarthritis, and diabetes is unknown. Therefore, treatment is focused on symptomolgy and effects of the diseases and has limited effectiveness. In many cases, known treatments are associated with serious negative side effects.

Recently, the National Cancer Institute (NIH Guide 2000) announced a program aimed to “reorganize the ‘front-end’ or gateway to drug discovery.” The new approach promotes a three stage discovery process. The first stage is discovery of the molecular mechanisms that underlie neoplastic transformations, cancer growth and metastasis. Next stage is selection of a novel molecular target within the discovered biochemical pathway known to have a unique difference between a healthy and a cancerous cell. The final stage is design of new drugs that modify the selected target. The program encourages moving away from screening agents by their clinical effects, such as shrinking tumor cells, in vivo or in vitro, to screening agents or designing drugs by their effects on a specific molecular mechanism. According to the NCI, screening by clinical effects identified drugs that demonstrated clear limitations in clinical efficacy, while screening by desired molecular effects should produce more efficaious and specific drugs.

The best drugs are those that are specifically designed to reverse the molecular events that cause disease. However, it is critical that the molecular mechanisms that underlie obesity, atherosclerosis, osteoarthritis, hypertension and diabetes be understood before drugs can be effectively designed to treat the disease.

### SUMMARY OF THE INVENTION

For the first time, this invention teaches the novel concept of microcompetition for GABP. There is a limited amount of GABP•p300 transcription complex available in the cell. The present invention teaches that cellular microcompetition for the transcription factor human GA binding protein (GABP) results in diseases such as include cancer, atherosclerosis, osteoarthritis and obesity. Moreover, other disruptions of the GABP pathway from microcompetition, diets, mutations, toxins, or drugs have a similar effect to microcompetition. This new understanding of the biochemical pathway surrounding GABP can be used to identify or design drugs to treat these diseases.

The invention includes methods of treating adverse affects associated with disruptions of the GABP pathway by microcompetition, diets, mutations, toxins, or drugs. Specifically, administering to a patient an effective amount of an agent that overcomes the disruption of the GABP pathway. The diseases that can be treated include cancer, atherosclerosis, osteoarthritis and obesity. The specific treatments can be accomplished in a variety of ways.

To most directly remove microcompetition, an agent that decreases foreign DNA N-boxes in cells can be used to eliminate the competitive binding sites. Examples of such agents are: Ganciclovir, ddI, ddC, and garlic and others described in the description. The present invention includes a method of identifying other compounds to treat the effects of microcompetition by assaying for compounds that eliminate competitive binding sites.

Other treatments of the effects of microcompetition or other disruptions of the GABP pathway are agents that stimulate phosphorylation of a GABP kinase, increases concentration of a GABP kinase, and increases affinity between GABP and GABP kinase. Examples of such agents are: dietary fiber (via sodium butyrate), sodium butyrate, acarbose, vanadate, vectors to knockout PTP1B, and others described in the description. The present invention includes a method of identifying other compounds to treat disruptions of the GABP pathway by assaying for compounds that have and affect on a GABP kinase.

Another treatment option is to use an agent that decreases the oxidative effect on GABP. Examples of such agents are: garlic and other anti-oxidants described in the description. The present invention includes a method of identifying other compounds to treat disruptions of the GABP pathway by assaying for compounds that decrease oxidative effect on GABP.

Agents that increase the concentration or effectiveness of a GABP stimulator can also be used to more effectively utilize GABP. In the converse, agents that decrease the concentration or effectiveness of a GABP suppressor can also be administered. In addition, treatment can employ an agent that increases concentration of GABPα, increases concentration of GABPβ, decreases concentration of GABPγ, increases phosphorylation of GABP, increases affinity between GABP and p300/CBP, or increases concentration of p300/CBP. Such agents would largely overlap with the agents above. However, they produce more specific effects. The present invention includes a method of identifying other compounds to treat disruptions of the GABP pathway by assaying for compounds that that have these specific effects.

### BRIEF DESCRIPTION OF THE DRAWINGS

FIG. 1 shows a schematic illustration of the extracellular signaling cascade and its effect on GABP.

FIG. 2 shows a schematic illustration of the activation of MAPK by MEK-1, a MAPKK, and deactivation of MAPK by PP2A, a serine/threonine phosphatase, PTP1B, a tyrosine-specific phosphatase, or MKP-1, a dual specificity phosphatase. A diamond represents a kinase, an ellipse, a phosphatase, an arrow, phosphorylation, and a T-headed line, dephosphorylation.

FIG. 3 shows a schematic illustration of the relationship between ERK signaling and microcompetition for available GABP.

FIG. 4 shows a schematic illustration of the how phosphorylated GABP stimulates the transcription of the sensitized receptor and how the new receptors increase the sensitivity of the pathway to the change in the concentration of the GABP kinase agent.

FIG. 5 shows a schematic illustration of an example of feedback inhibition involving GABP.

FIG. 6 shows a schematic illustration of the effects of downstream control relative to a sensitized receptor.

FIG. 7 shows a schematic illustration of the effect of LPS, RSVL and RA on NF-κB and on ETS.

FIG. 8 shows a graphic illustration of the change in TF activity as a function of time for a control cell and a cell harboring a GABP viral genome.

FIG. 9 shows a graphic illustration of how microcompetition reduces lipolysis per adipocyte.

FIGS. 10a-10c show graphic illustrations of the measured relationship between epinephrine infusion and glycerol release.

FIG. 11 shows a graphic illustration of how microcompetition reduces Rb transcription.

FIG. 12 shows a schematic illustration of arachidonic acid metabolism.

FIG. 13 shows a schematic illustration of the difference between the microcompetition equilibrium and the healthy system equilibrium.

FIG. 14 shows a schematic illustration of how aberrant GABP expression and function can be restored.

### DETAILED DESCRIPTION OF THE INVENTION

The present invention starts from the discovery that microcompetition is involved in a variety of human diseases. It is only by looking through the lens of the present invention that a discernable pattern of disease progression and symptomology is understood. From this understanding the inventor was able to develop new assays, screening regiments and treatments.

Once microcompetition was discovered to play a role in human disease, the present inventor looked back at previous work to see if it was possible to find published observations consistent with microcompetition. Having made the original discovery, the inventor has been able to piece together and relate a mosaic of individual studies and information that heretofore seemed entirely unrelated.

The present invention started as a new theory of human disease and testing the hypothesis was also performed in a novel way. Once the theory was developed, a novel mechanism of action and relationship between biochemical agents was proposed, followed by a set of predictions of the effect of modification of one or more of those biochemical agents. However, it was unnecessary to perform thousands of experiments to test the hypothesis, because others had studied the biochemical agents and recorded the effects of modifying those agents. By looking at the results of thousands of studies on dozens of biochemical agents, the set of predicions was tested and supported. Close to 600 papers are referenced in this disclosure, each providing a piece of information that forms the totality of this invention.

Much of this disclosure is similar to a mosaic. In the same way, cermaic plates or colored glass are shattered and rearranged by the mosaic artist to form a new piece of art, the applicant has similarly used pieces of information evidence gleened from work of other researchers to understand the mechanism of human disease in an entirely new way.

The present invention teaches the relationship between microcompetition and human disease and this detailed description starts with a detailed explanation of microcompetition. It then progresses through the affected pathways and teaches the pieced together evidence supporting the microcompetition model. Based upon this model, a series of new assays, screening regiments and treatments are described. The full citation for each reference is provided at the end of the detailed disclosure and is cited in an abbreviated fashion within the text to make the disclosure more readable.

### A. Microcompetition

1. Definition

A situation where DNA sequences compete for the same transcription complex will be called microcompetition. Assume that the cellular availability of least one of the proteins constructing the transcription complex is limited. Assume that the complex binds DNA of two genes, and that binding stimulates the transcription of one of these genes. Then, microcompetition for the transcription complex reduces binding of the complex to the gene, resulting in reduced transcription.

2. Molecular Effect

The following studies demonstrate the effect of microcompetition on various cellular genes.

a) Human metallothionein-II_{A} (hMT-II_{A})

CV-1 cells were cotransfected with constant amount of a plasmid containing the hMT-II_{A} promoter (−286 nt relative to the start of transcription to +75 nt) fused to the bacterial gene coding chloramphenicol acetyltransferase (hMT-II_{A}-CAT) and increasing amounts of the plasmid containing the viral SV40 early promoter and enhancer fused to bacterial gene coding for aminoglycoside resistance (pSV2Neo). A 2.4-fold molar excess of the plasmid containing the viral enhancer reduced 90% of CAT activity. No microcompetition was observed with the viral plasmid after deletion of the SV40 enhancer.

The efficient inhibition of hMT-II_{A} promoter activity by the SV40 enhancer suggests that the enhancer has a high affinity for a limiting transcription complex which also binds the hMT-II_{A} promoter. Moreover, although both the hMT-II_{A} promoter and the SV40 enhancer bind the Sp1 transcription factor, further studies ruled out the idea that the two plasmids compete for Sp1 or factors which bind the TATA box (Scholer 1986¹)

b) Platelet Derived Growth Factor-B (PDGF-B)

JEG-3 choriocarcinoma cell line were transiently cotransfected with a constant amount of PDGF-B promoter/enhancer-driven CAT reporter gene (PDGF-B-CAT) and increasing amounts of a plasmid containing either the human cytomegalovirus promoter/enhancer fused to the βgal reporter gene (CMV-βgal) or the viral SV40 early promoter and enhancer elements fused to βgal (SV40-βgal).

Both CMV-βgal and SV40-βgal repressed the activity of PDGF-B-CAT in a concentration-dependent manner. Mutational studies of the SV40 promoter/enhancer element showed that the sequence in SV40-βgal which competes with PDGF-B is located within the SV40 enhancer region (Adam 1996²). However, no specific DNA box or transcription factors were identified.

c) Collagen type I α2 chain (COL1A2)

Skin fibroblasts were infected with temperature senstitive Rous Sarcoma Virus (ts-RSV). The amount of COL1A2 RNA was measured in cells grown at the permissive (T) or nonpermissive (N) temperature for transformation. In skin fibroblasts the amount of COL1A2 RNA was decreased 5-fold. A similar experiment showed a reduction of 3.3-fold in the amount of COL1A1 RNA (Allebach 1985³).

WI-38 human lung fibroblasts were transformed by a clone of SV40. The mRNA of the α2(I) chain was absent in the SV40 transformed WI-38 fibroblasts, whereas the mRNA of the α1(I) chain was detected on the same blot. The study eliminated a few possible reasons for the reduced expression of the α2(I) chain in the infected cells. The chromosomes which normally harbor the α2(I) and α1(I) genes appeared to be perfectly normal. Restriction mapping of the α2(I) gene in the transformed cells did not show any gross insertion of the viral genome within the gene or its promoter. Methylation analysis of the promoter and 3′ regions of the gene did not revel any detectable hypermethylation (Parker 1989⁴).

Normal cells synthesize the standard form of collagen type I consisting of two α1(I) chains and one α2(I) chain. Tumors caused by the polyomavirus, on the other hand, mainly synthesize a α1(I) trimer (Moro 1977⁵) A high concentration of trimers was also found in SV40 transformed WI-38 human lung fibroblasts (Parker 1992⁶). Microcompetition mainly decreases the expression of the α2(I) chain (see Allebach 1985 and Parker 1989 above). The relative shortage of the α2(I) chain in infected cells stimulates formation of the α1(I) trimers.

d) Integrin (β2 leukocyte, CD18)

Human monocytes were infected with human immunodeficiency virus type 1 (HIV-1). The surface expression of CD18, CD11a, CD11b, CD11c, CD58, CD62L, CD54, and CD44 was measured in HIV-1 infected cells and mock-infected cells. The extent and kinetics of CD11a, CD11b, CD11c CD58 and CD62L expression were similar in HIV-1 infected cells and mock-infected cells. CD18, CD54 and CD44 showed a significant decrease in expression in the HIV-1 infected cells. When monocytes were treated with a heat-inactivated HIV-1 virus, the expression of CD54 and CD44 was similar to the expression in mock-infected cells, however, the expression of CD18 was reduced.

According to Le Naour, et al., (1997⁷) “treatment with heat-inactivated virus shown that regulation of CD18 expression is dependent on early HIV-related regulatory mechanisms whereas regulation of CD44 and CD54 requires viral events taking place after retrotranscription of viral RNA.”

Adult T-cell leukemia (ATL) is etiologically associated with the human T-cell leukemia virus type 1 (HTLV-1). The mRNA of CD18 was measured in three human T-cell acute-lymphoblastic-leukemia cell lines, MOLT-4, Jurkat and CEM negative for HTLV-1, four T-cell lines, MT-2, TCL-Kan, C91/PL and C8166, which were established by transformation with HTLV-1, one T-cell line, TOM-1, derived from an HTLV-1 carrier and is positive for HTLV-1, and four cell lines, MT-1, TL-Om1, H582 and HuT102, which are ATL derived T-cell lines and are positive for HTLV-1. Overall, non ATL derived, HTLV-1 negative cell lines showed high levels of CD18 mRNA. The non ATL derived, HTLV-1 positive cell lines showed moderate levels of CD18 mRNA. The ATL derived, HTLV-1 positive cell lines showed a low levels of CD18 mRNA (Ibid, FIG. 7, Tanaka 1995⁸).

Southern-blotting analysis did not reveal any gross structural changes in the CD18 gene. To test the CD18 promoter activity in the ATL derived, HTLV-1 positive cell lines, TL-Om1, H582 and HuT102 were transfected with a CD18 promoter-driven CAT reporter gene. The same construct was transfected in the non ATL derived, HTLV-1 negative Jurkat cells. The results showed high CAT expression in the Jurkat cells and low CAT expression in the 3 ATL derived, HTLV-1 positive cell lines. Tanaka, et al., (1995) conclude that “the down regulation of the CD18 gene in these ATL cell lines was due to lack of transcription factor(s) necessary for CD18 gene expression.” The paper does not identify the transcription factor, neither does it provide an explanation for the reduced availability of the unknown factor(s).

The Epstein-Barr virus (EBV) selectively infects human B cells causing infectious mononucleosis (IM). Lymphoblastoid cell lines (LCLs) were derived from EBV-infected B cells obtained from normal individuals, IM patients, or by in vitro EBV transformation of normal B cells. LCLs grow as large cell clusters. In contrast, Burkitt lymphoma (BL) cells grow mostly as single cells or loose clusters. The CD18 surface expression was measured in 10 LCLs and 10 BL cell lines. Approximately one-third of the cell population in each LCL was CD18-negative. In comparison, the majority of the malignant cells in each BL cell were CD-18 negative (Patarroyo 1988⁹).

In all these studies, the competition between the viral and the cellular DNA reduces the transcription of the CD18 gene.

3. GABP Transcription Factor

a) N-box and GABP

The DNA motif (A/C)GGA(A/T)(G/A) (N-box) is the core binding sequence of the transcription factor known as GA Binding Protein (GABP), Nuclear Respiratory Factor 2 (NRF-2)¹⁰, E4 Transcription factor 1 (E4TF1)¹¹ and Enhancer Factor 1A (EF-1A)¹². In this report we refer to the transcription factor as GABP and to the motif as the N-box.

b) GABPα, β and γ(GABP as activator, repressor)

Five subunits of GABP are known. GABPα, GABPβ1, GABPβ2 (together called GABPβ, GABPγ1 and GABPγ2 (together called GABPγ). None of the GABP subunits can stimulate transcription alone, either in vivo or in vitro.

GABPα is an ets-related DNA-binding protein. GABPα binds the N-box.

GABPα forms a heterocomplex with GABPβ. GABPβ has an amino acid sequence in the amino-terminal which includes a four tandem repeat. The four tandem repeat is responsible for heterodimerization. GABPβ also contains a leucine zipper-like motif in the carboxyl terminal. This motif allows it to homodimerize. Through the hetrodimerization and homodimerization domains, GABPα and GABPβ form a α₂β₂heterotetrameric complex, which stimulates transcription efficiently in vitro and in vivo.

GABPα also forms a heterocomplex with GABPγ. An idenctical four tandem repeat in GABPγ is responsible for heterodimerization between GABPα and GABPγ. However, GABPγ is lacking the leucine zipper-like motif and, therefore, does not homodimerize. The heterodimer does not stimulate transactivation.

The degree of transactivation by GABP appears to be a result of the relative concentrations of GABPβ and GABPγ in the cell (Suzuki 1998¹³). An increase in GABPβ relative to GABPγ increases transcription, while an increase of GABPγ relative to GABβ represses transcription. A log regression on Suzuki, et al., results, produced the following formula for the fold increase in transcription as a function of relative concentrations of GABPβ and GABPγ (brackets indicate concentration).

Fold increase in transcription=2.785×([GABPβ]/[GABPγ])^{0.06}

The degree of transactivation by GABP is a function of the ratio between GABPβ and GABPγ. By controling this ratio the cell regulates the transcription of genes with binding sites for GABP (Suzuki 1998).

c) GABP Binds p300

GABP binds the p300 acetyltranferase. p300 belongs to the p300/CBP family of proteins. GABPα binds directly to the C-terminal of p300 and much more weakly to the N-terminal. GABPβ does not bind directly to p300 (Bannert 1999¹⁴).

d) Cellular Availability of p300 is Limited

Although p300/CBP is widely expressed, its cellular availability is limited. A few studies demostrated that competitive binding of cellular proteins to p300/CBP had an inhibitory effect on the activation by certain transcription factors. Competitive binding of p300 or CBP to the glucocorticoid receptor (GR) or retinoic acid receptor (RAR) inhibited activation of a promoter dependent on the AP-1 transcription factor (Kamei 1996¹⁵). Competitive binding of CBP to STAT1α inhibited activation of a promoter dependent on both the AP-1 and ets transcription factors (Horvai 1997¹⁶). Competitive binding of p300 to STAT2 inhibited activation of a promoter dependent on the NF-κB RelA transcription factor (Hottiger 1998¹⁷).

e) Viral GABP Enhancers

The (A/C)GGA(A/T)(G/A) motif is the core binding sequence of many viral enhancers. Some of the viral enhancers include the Polyomavirus Enhancer Area 3 (PEA3) (5108/5113, and 5202/5207) (Asano 1990¹⁸), E1A enhancer (−300/−295, −200/−195) (Higashino 1993¹⁹), (GABP binds to the promoter of the adenovirus early-region 4, or E4, hence the name E4TF1), Rous Sarcoma Virus (RSV) enhancer (189/194) (Laimins 1984²⁰), Herpes Simplex Virus 1 (HSV-1) (in the promoter of the immediate early gene ICP4) (LaMarco 1989²¹), (Douville 1995²²), Cytomegalovirus (CMV)(IE-1 enhancer/promoter region) (Boshart 1985²³), Moloney Murine Leukemia Virus (Mo-MuLV) enhancer (8024/8048) (Gunther 1994²⁴), Human Immunodeficiency Virus (HIV) (the two NF-κB binding motifs in the HIV LTR) (Flory 1996²⁵), Epstein-Barr virus (EBV) (20 copies in the +7421/+8042 oriP/enhancer) (Rawlins 1985²⁶), and Human T-cell lymphotropic virus (HTLV) (8 sites in the enhancer (Mauclere 1995²⁷), one site in the LTR) (Kormfeld 1987²⁸). Moreover, some viral enhancers, for example SV40, lack a precise N-box, but still bind the GABP transcription factor (Bannert 1999²⁹).

Some papers present evidence supporting binding of GABP to the N-boxes of these viral enhancers. Flory, et al., 1996³⁰ show binding of GABP to the HIV LTR, Douville, et al., 1995³¹ show binding of GABP to the promoter of ICP4 of HSV-1. Bruder, et al. 1991³² and Bruder, et al., 1989³³ show binding of GABP to the adenovirus E1A enhancer element I. Ostapchuk, et al., 1986³⁴ show binding of GABP (called EF-1A in this paper) to the polyomavirus enhancer. Gunther, et al., 1994³⁵ show binding of GABP to Mo-MuLV.

Other papers show evidence of competition between these viral enhancers and enhancers of other viruses. Shcoler and Gruss, 1984³⁶ show competition between the Moloney Sarcoma Virus (ISV) enhancer and SV40 enhancer and also competition between the RSV enhancer and the BK virus enhancer.

4. Mirocompetition for GABP•p300

GABPα binds p300 (Bannert 1999³⁷). Therefore, microcompetition for GABP is also microcompetition for GABP•p300. Since cellular availability of p300 is limited, cellular availability of GABP•p300 is also limited.

A virus which binds the GABP complex will be called a GABP virus. Microcompetition for GABP•p300 between a GABP virus and a cellular GABP gene reduces cellular availability of the GABP•p300 complex to the cellular gene. If the cellular gene is stimulated by the complex, the cellular gene shows reduced transcription. If the cellular gene is repressed by the complex, the cellular gene shows increased transcription.

5. GABP•p300 Binding Regulation

a) Extracellular Signal-regulated Kinase (ERK, MAPK) Pathway

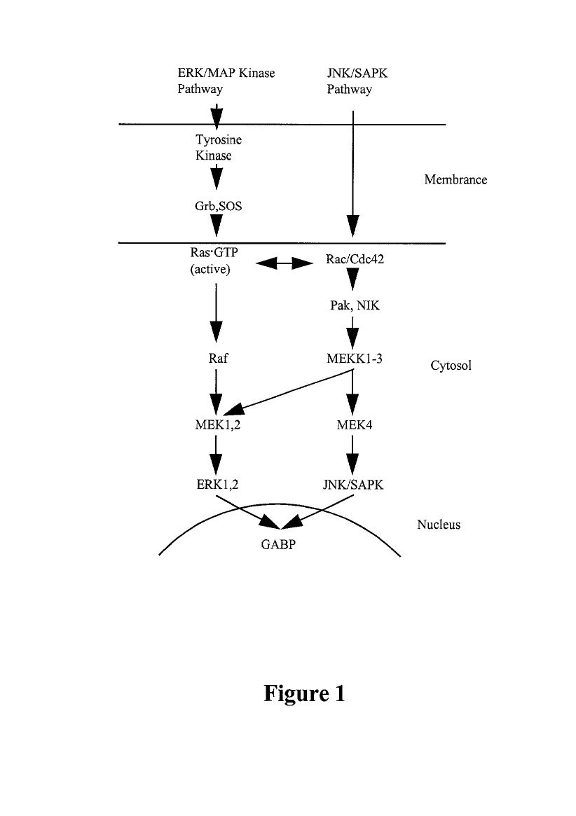

Extracellular signals are transmitted to the nucleus in many ways. Often signal transduction occurs through activation of a kinase found in the cytoplasm. Once activated, the kinase translocates to the nucleus where it phosphorylates certain transcription factors. Phosphorylation modifies a factor capacity to regulate gene expression. The extracellular signal-regulated kinase (ERK, previously called MAP kinase) pathway, see FIG. 1, is an example of such a signaling cascade.

Growth factors and other extracellular agents that support proliferation activate ERK. The signal is propagated through sequential activation of multiple kinases. These kinases amplify a weak signal into large changes in output. The cascade can be regulated positively or negatively at each level. All of the MAP kinases are activated by dual phosphorylation on a Thr-Xaa-Tyr motif, after which they function as proline-directed Ser/Thr kinases with a minimal target sequence of Ser/Thr-Pro (Hipskind, 1998³⁸).

Raf (MAPKKK) is activated by an unclear mechanism usually dependent upon Ras. By interacting with Ras, Raf is relocalized to the membrane, which appears to be important for activation. The Raf family has three known members, c-Raf (or Raf-1), B-Raf and A-Raf. Each of these proteins can function as MAPKKK depending upon cell type. Other kinases can also fimction in this capacity (i.e.—MEKKs 1 and 3) and the possibility remains open for other specific activators of the ERK cascade.

Raf activates the MAPKK MEK (MEK1 and MEK2), a kinase that phosphorylates both the Thr and Tyr residues in the activation motif in ERK. There are five members of the ERK family to date, ERK1 (p44), ERK2 (p42), ERK3, ERK4, and ERK5/BMK1 (for Big MAP Kinase). Activation results in translocation of ERK to the nucleus, where it targets transcription factors and the basal transcription comple. X

Dephosphorylation of either Thy or Tyr residue inactivates ERK. There are three classes of ERK inactivators: Type 1/2 serine/threonine phosphatases, such as PP2A, tyrosine-specific phosphatases (also called protein-tyrosine phosphatase, denoted PTP), such as PTP1B, and dual specificity phosphatases, such as MKP-1. For a recent reviews on the role of these classes of phosphatases in the regulation the MAP kinase activity, see Camps 2000³⁹, Saxena 2000⁴⁰ and Keyse 1998⁴¹. Let “ERK phosphatase” be any phosphatase which inactivates ERK. The class of all ERK phosphatases is a super class of the above three classes of ERK inactivators.

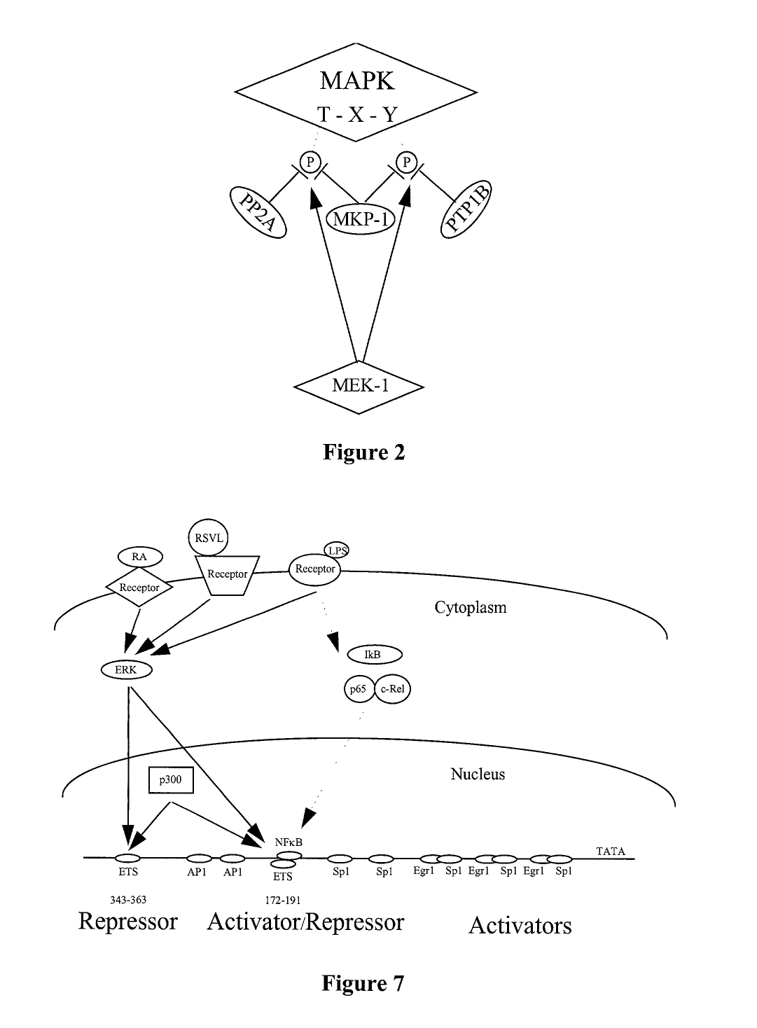

FIG. 2 illustrates activation of MAPK by MEK-1, a MAPKK, and deactivation of MAPK by either PP2A, a serine/threonine phosphatase, PTP1B, a tyrosine-specific phosphatase, or MKP-1, a dual specificity phosphatase. A diamond represents a kinase, an ellipse, a phosphatase, an arrow, phosphorylation, and a T-headed line, dephosphorylation.

The JNK/SAPK pathway is discussed below.

b) GABP Kinase Agent

A molecule which stimulates the phosphorylation of ERK will be called “GABP kinase agent.” Some GABP kinase agent are sodium butyrate (SB), trichostatin A (TSA), trapoxin, phorbol ester (phorbol 12-myristate 13-acetate, PMA, TPA), retinoic acid (RA, vitamin A), zinc and copper, interferon-γ (IFNγ), new differentiation factor (NDF, or heregulin), estron, etradiol (E2), interleukin 1 β (IL-1 β), interleukin 6 (IL-6), tumor necrosis factor α (TNFα), transforming growth factor β (TGFβ), oxytocin (OT).

c) Sodium Butyrate (SB), Trichostatin A (TSA) and Trapoxin

The GABP kinase agent sodium butyrate (SB), trichostatin A (TSA) and trapoxin were tested for their effects on the major promoter (M) of human choline acetyltransferase (ChAT). The human choline acetyltransferase (ChAT) gene was activated by sodium butyrate, trichostatin A, and trapoxin A, in transient and stable transfection studies (Espinos 1999⁴²). These agents also stimulated ERK1 and ERK2 phosphorylation. Treatemtn with the specific MAP kinase kinase (MEK) inhibitor, PD98059, or overexpression of dominant-negative mutants of Ras and ERK2, suppressed the sodium butyrate activation of the ChAT promoter (Espinos 1999).

Transcriptional activation of cellular and transfected genes by histone deacetylase (HDAC) inhibitors is blocked by H7, an inhibitor of serine/threonine protein kinases. In transient transfections with the human ChAT gene, the cells were treated for 1 hour with H7, and then sodium butyrate or trapoxin were added in the continued presence of H7. Under these conditions, H7 inhibited the activation by both trapoxin and sodium butyrate (Espinos 1999). Similar experiments were performed using the RSV LTR and the SV40 enhancer. Activation of these enhancer regions by sodium butyrate or trapoxin was suppressed by H7. In addition, activation of RSV LTR by sodium butyrate was blocked by the MEK inhibitor PD98059, while activation of the SV40 promoter was depressed about three-fold (Espinos 1999).

Transcription of the nicotinic acetylcholine receptor (AChR) in adult muscle is restricted to the nuclei located at the neuromuscular junction. The N-box, a promoter element, contributes to this specialized synaptic expression of the AChR δ- and ε-subunits. GABP binds to the N-box in vitro. GABP subunits contain phosphorylation sites by MAP kinases, and these kinases mediate the heregulin-elicited stimulation of transcription of AChR genes in cultured chick myotubes. Phosphorylation studies in chick primary myotubes showed that heregulin stimulated GABPα and GABPβ phosphorylation. Both subunits of GABP are phosphorylated in vivo by MAP kinases and heregulin enhances their phosphorylation (Schaeffer 1998⁴³).

d) Phorbol Ester (Phorbol 12-myristate 13-acetate, PMA, TPA), Thapsigargin

The murine macrophage cell line RAW 264.7 was stimulated with thapsigargin, an endomembrane Ca(2+)-ATPase inhibitor, and TPA, the protein kinase C activator. Both thapsigargin (30 nM) and TPA (30 nM) induced phosphorylation of p44/p42 MAP kinase and production of histamine in a time- and concentration-dependent manner. The specific MEK1 inhibitor, PD98059 strongly suppressed both thapsigargin and TPA induced histamine production. Another MEK1 inhibitor, U-0126, also inhibited both thapsigargin and TPA-induced histamine production in a concentration-dependent manner (Shiraishi 2000⁴⁴).

TPA induced in vitro differentiation of pluripotent K562 human leukemia cells. Treatment of K562 cells with TPA resulted in growth arrest, polyploidy, morphological changes, and increased cell-cell and cell-substrate adhesion. These PMA-induced changes were preceded by a rapid rise in MEK1 activity that resulted in a sustained ERK2 activation. The MEK1 inhibitor, PD098059, reversed both growth arrest and the morphological changes induced by TPA treatment. These results demonstrate that the TPA-induced signaling cascade initiated by protein kinase C activation requires activity of the MEK/ERK signaling complex in regulating cell cycle arrest (Herrera 1998⁴⁵).

TPA was used in inhibition of apoptosis in HL-60 cells stimulated with the JNK/SAPK activator anisomycin. An increase in ERK activity was associated with an anti-apoptotic effect. The MEK1 inhibitor, PD98059, inhibited the TPA-mediated ERK activity and abrogated the anti-apoptotic effects of TPA. Moreover, inhibition of apoptosis was attenuated by pretreatment with PKC inhibitors (Stadheim 1998⁴⁶).

e) Retinoic Acid (RA, Vitamin A)

Consider Yen 1999⁴⁷ “Among the three major mitogen-activated protein kinase (MAPK) cascades—the extracellular signal regulated kinase (ERK) pathway, the c-JUN N-terminal/stress-activated protein kinase (JNK/SAPK) pathway, and the reactivating kinase (p38) pathway—retinoic acid selectively utilizes ERK but not JNK/SAPK or p38 when inducing myeloid differentiation of HL-60 human myeloblastic leukemia cells. Retinoic acid is known to activate ERK2. The present data show that the activation is selective for this MAPK pathway. JNK/SAPK or p38 are not activated by retinoic acid.”

f) Interferon-γ (IFNγ)

IFNγ activated both ERK and PKC in human peripheral blood monocytes (Liu 1994⁴⁸) IFNγ also induced ERK activation in rat C6 glioma cells. The MEK1 specific inhibitor, PD98059, blocked this activation. Transfection of the dominant-negative form of c-Ha-Ras (Asn-17) in the C6 glioma cells abrogated IFNγ-induced ERK1 and ERK2 activation. These results indicate that IFNγ-induced ERK1 and ERK2 activation and that p21ras and MEK1 are required for this activation (Nishiya 1997⁴⁹).

g) Heregulin (HRG, or New Differentiation Factor, NDF)

Heregulinβ1 (HRGβ1) induced ERK activation and cell differentiation in AU565 breast carcinoma cells. ERK activation remained elevated for 2 h following high doses of HRG. The MEK specific inhibitor, PD98059, inhibited activation of ERK and completely blocked HRG-induced differentiation reversing cell growth arrest. A transient transfection of a mutant constitutively active MEK1 construct into AU565 cells induced differentiation in the absence of HRG. Treatment with HRG potentiated this response. This study indicates that HRG induces a sustained activation of the MEK/ERK pathway and that this activation is essential for inducing differentiation of AU565 cells (Lessor 1998⁵⁰).

HRG activated the MAP kinase isoforms p44ERK1 and p42ERK2 and the p70/p85 S6 kinase in AU565, T47D and HC11 cells. HRG stimulation caused growth arrest of the AU565 cells and proliferation of the T47D or HCl 1 cells. HRG also stimulated tyrosine phosphorylation and in vitro kinase activity of ErbB-2. When PKC was activated by TPA, another GABP kinase agent, HRG was no longer able to activate ErbB-2 in T47D cells, blocking cell proliferation. Activation of ErbB-2 by point mutation or monoclonal antibodies, also stimulated the MAPK and the p70/p85 S6 kinase pathways. The same monoclonal antibodies also induced AU565 cell differentiation (Marte 1995⁵¹).

HRGβ2 stimulation of MDA MB-453 cells resulted in tyrosine phosphorylation of p185c-erbB2 and p180erbB4 receptors in a time- and dose-dependent fashion. Activation of ERK (>30-fold over untreated controls) was observed upon receptor(s) activation, as was the induction of the immediate early gene c-fos (>200-fold) (Sepp-Lorenzino 1996⁵²). In another study, HRGβ2, the ligand for erbB3 and erbB4, caused ERK activation and mitogensis of growth arrested T-47D human breast cancer cells. The MEK1 specific inhibitor, PD98059, completely blocked the HRG-induced entry into S-phase (Fiddes 1998⁵³).

h) Zinc (Zn) and Copper (Cu)

Egr1, an immediate early transcription factor, is induced after brain insults by an unknown mechanism. Short exposure to zinc led to sustained ERK activation (Park 1999⁵⁴). The MEK1 inhibitor, PD098059, inhibited ERK1/2 activation, Egr1 induction, and neuronal death by zinc. The study concluded that zinc activates ERK1/2 (Park 1999). In another study, zinc enhanced ERK activity in serum-starved Swiss 3T3 cells treated with insulin and phosphocholine (Kiss 1997⁵⁵).

The human bronchial epithelial cell line BEAS was exposed to noncytotoxic levels of metals including Cu and Zn. Kinase activity assays and Western blots (with phospho-specific MEK1 antibody) showed that MEK1 is activated by Cu or Zn treatment. Additional Western blots using phospho-specific ERK1/2 antibody showed that PD98059, the selective MEK1 inhibitor, blocked the metal induced phosphorylation of ERK1/2 (Wu 1999⁵⁶). Activity assays of another study showed a dramatic activation of ERK, JNK and p38 in BEAS cells exposed to Zn, while Cu exposure led to a relatively small activation of ERK (Samet 1998⁵⁷).

i) Estron, Estradiol

Treatment of human mammary cancer MCF-7 cells with estradiol stimulates rapid and transient activation of ERK1/2. Estradiol activates the tyrosine kinase/p21ras/ERK pathway in MCF-7 cells (Migliaccio 1996⁵⁸).

Uterine smooth muscle from rats pretreated with estradiol-17 β alone or with estradiol-17 β and progesterone were tested for ERK expression and activity by immunoblotting with ERK1/2 antibodies and phosphorylation assays. Estrogen and progesterone both enhanced ERK activity (Ruzycky 1996⁵⁹).

In another study, immunoblot analysis and phosphorylation assays showed that estradiol-17 β (E2) stimulated ERK1/2 in rat cardiomyocytes. Specifically, the activation of ERK1/2 was rapid and transient, while a rapid but sustained increase of JNK phosphorylation was observed (Nuedling 1999⁶⁰).

j) Interleukin 1 β (IL-1β)

Treatment with IL-1β in cultured human airway smooth muscle cells increased levels of phosphorylated ERK (p42 and p44) 8.3- and 13-fold, respectively. Pretreatment of the cells with the MEK1 inhibitor PD98059 decreased ERK phosphorylation (Laporte 1999⁶¹).

IL-1β treatment of HepG2 cells activated three ERK cascades, p46/54(JNK), p38, and ERK1/2. There was maximal induction of 20-, 25-, and 3-fold, respectively, in these three cascades (Kumar 1998⁶²). In another study, Western blotting and kinase assays showed that IL-1β activates ERK1/2 and p38 in islets and rat insulinoma cells (Larsen 1998⁶³).

k) Interleukin 6 (IL-6)

The cytokine IL-6 utilizes 80-kDa ligand-binding and 130-kDa signal-transducing subunits to trigger cellular responses. Treatment of the human B cell line, AF-10, with rIL-6 activated ERK. Activation of ERK in AF-10 cells occurred at the same time as the appearance of 42- and 44-kDa tyrosine phosphoproteins (p42 and p44) (Daeipoou 1993⁶⁴). Presence of the tyrosine protein kinase inhibitors, genistein and geldanomycin, reduced activation of ERK induced by rIL-6. These results indicate that IL-6 activates ERK1/2.

l) Tumor Necrosis Factor α (TNFα)

TNFα stimulates IL-6 production in renal cells in culture. Human mesangial cells (HMCs) and human proximal tubular (HPT) cells were treated for 24 hours with TNFα in the presence and absence of the specific p38 and ERK1/2 inhibitors SB203580 and PD98059, respectively, either alone or in combination. TNFα activated p38 and ERK1/2. The inhibitors SB203580 and PD98059 inhibited basal and TNFα-stimulated IL-6 production in both cell types (Leonard 1999⁶⁵).

m) Transforming Growth Factor β (TGFβ)

TFGβ inhibits many epithelial cell types. Both TFGβ1 and TFGβ2 trigger rapid activation of p44MAPK in two proliferating epithelial cell lines, IEC4-1 and CCL64. Results for a third TFGβ resistant cell line, IEC4-6 showed no activation of p44MAPK after TFGβ stimulation. Resting cultures of IEC4-1 cells treated with TFGβ2 led to no significant change in either DNA synthesis or p44MAPK activity. However, addition of the growth-stimulatory combination of factors (epidermal growth factor, insulin, and transferrin (EIT)) to quiescent and proliferating IEC4-1 cells stimulated DNA synthesis and led to activation of p44MAPK. The specificity for the cellular effects of growth factors may not actually occur at the level of MAPK activation, but instead at downstream events including phosphorylation of transcriptional complexes and gene activation (Hartsough 1995⁶⁶).

TFGβ1 also stimulates articular chondrocyte cell growth and the formation of the extracellular matri. XIn vitro kinase assays showed a rapid activation of ERK induced by TFGβ1 (Yonekura 1999⁶⁷). The stimulation peaked at 5 min, and dropped back to basal levels within 240 min after TFGβ1 stimulation. After 240 minutes of stimulation, the c-jun N-terminal kinase activity increased only about 2.5-fold, while there was no significant change in p38MAPK activity. PD98059 decreased TFGβ1 induced Elk1 phosphorylation in a dose-dependent manner (Yonekura 1999).

n) Oxytocin (OT)

Oxytocin (OT) treatment triggers the rapid phosphorylation of ERK2 in Chinese hamster ovary (CHO) cells (Strakova 1998⁶⁸). The MEK1 specific inhibitor, PD98059, significantly reduced OT-stimulated prostaglandin (PGE) synthesis (Strakova 1998). Oxytocin receptors (OTRs) are found in a number of human breast tumors and tumor cells. In a study of breast cancer cells (Hs578T cells), OT stimulated ERK2 phosphorylation and PGE2 synthesis in Hs578T cells (Copland 1999⁶⁹).

The rat oxytocin receptor was transfected into Chinese hamster ovary cells. Oxytocin stimulated ERK2 phosphorylation and PGE synthesis through protein kinase C activity (Hoare 1999⁷⁰). Deletion of 51 amino acid residues from the carboxyl terminus of the oxytocin receptor resulted in decreased affinity for oxytocin. Cells expressing the truncated receptor showed no oxytocin-stimulated ERK2 phosphorylation and PGE synthesis (Hoare 1999).

6. Phosphorylation of GABP

ERK phosphorylates GABPα and GABPβ. Phosphorylation does not change the binding of GABP to DNA (Flory, 1996⁷¹, Avots, 1997⁷², Hoffmleyer, 1998⁷³, Tomaras, 1999⁷⁴).

Phosphorylation is known to increase binding or stabilize the complex of p300 and other transcription factors, such as NF-κB unit p65 and Bbf (Zhong 1998⁷⁵, Bevilacqua 1997⁷⁶). The following sections present evidence consistent with the discovery that ERK phosphorylation of GABP leads to increased binding of p300 to GABP, stabilize the GABP•p300 comple. X

a) ERK Phosphorylation of GABP Increases N-Box DNase-I Hypersensitivity

Histone acetylation occurs post-translationally, and reversibly, on the ε-NH₃+groups of lysine residues embedded in the N-terminal tails of core histones. Histone acetyltransferases (HATs) transfer the acetyl moiety from acetyl coenzyme A to the ε-NH3+groups of internal lysine residues. Introduction of the acetyl group to lysine neutralizes the positive charge, increases hydorphobicity and leads to unfolding of chromatin (Kuo 1998⁷⁷) Histone hyperacetylation correlates with sensitivity to digestion by deoxyribonuclease I (DNase-I) (Hebbes 1994⁷⁸). Moreover, binding of a transcription complex with HAT activity to DNA enhances DNase-I hypersensitivity around the DNA binding site. p300 shows HAT enzymatic activity. Hence, binding the GABP•p300 complex enhances DNase-I hypersensitivity around the N-box.

Porcine peripheral blood mononuclear cells (PBMC) were stimulated with the GABP kinase agent TPA. The treatment consistently enhanced the DNase-I hypersensitivity of the third intron enhancer of the TNFα gene (Kuhnert 1992⁷⁹) The major transcription factor that binds the enhancer site in the third intron of TNFα gene is GABP (Tomaras 1999⁸⁰). TPA treatment phosphorylated ERK, which phosphorylated GABP. Phosphorylation of GABP increased binding of p300. The HAT activity of p300 acetylated the histones and enhanced DNase-I hypersensitivity of the third intron enhancer.

b) ERK Phosphorylation of GABP Synergizes with p300 Stimulation

Human neuroepithelioma CHP126 cells were transfected with a construct containing the promoter of human choline acetyltransferase (ChAT) gene fused to the luciferase reporter gene (ChAT-luciferase). These cell were stimulated with the GABP kinase agent trapoxin. Trapoxin increased luciferase expression 8-fold. In a second experiment the cell were transfected with an expression vector for full-length p300. p300 increased luciferase expression 5- to 10-fold. In a third experiment the cells were transfected with p300 and stimulated with trapoxin. The combined treatment increased luciferase expression 94-fold (Espinos 1999⁸¹). Trapoxin phosphorylated ERK. ERK phosphorylated GABP. The combinded effect of GABP phosphorylation and p300 transfection on transcription was more than additive.

Greater than additive increase in transcription demonstrates that two stimulators act in the same pathway, or in pathways that merge, to increase trascription from a single promoter. If the stimulators were acting independently, the largest possible amount of transcription from the two together would be the sum of the two pathways, with each stimulator increasing transcription as if the other were not present (Herschlag 1993⁸²). A compeling interpretation of the “more than additive” results above is that phosphorylation of GABP increased binding of p300.

c) Inhibition of ERK Phosphorylation Blocks p300 Stimulation

H7 is an inhibior of serine/threonine protein kinases. ERK is serine/threonine protein kinase and, therefore, inhibited by H7. Activation of the ChAT promoter by either the GABP kinase agent trapoxin or the GABP kinase agent sodium butyrate was inhibited by 40 μM of H7. Activation of the ChAT promoter by p300 was also inhibited by H7 in a dose-dependent manner. H7 also suppressed the synergistic activation of the ChAT promoter triggered by trapoxin and p300 (Espinos 1999⁸³). Inhibition of GABP phosphorylation decreased binding of p300, which reduced trascription.

d) Inhibition of p300 Binding Blocks Stimulation by ERK Phosphorylation

GABP binds p300 in between amino acids 1572 and 2370 (Bannert 1999⁸⁴). Adenovirus E1A protein binds p300 between amino acids 1572 and 1818 (Eckner 1994⁸⁵). E1A and GABP share an overlapping binding site on p300. By displacing GABP from p300, E1A reduces the effectiveness of GABP phosphorylation. The activation of the SV40 minimal promoter and the ChAT promoter by the GABP kinase agent sodium butyrate and by p300 was suppressed by adenovirus E1A protein (Espinos 1999).

ERK phosphorylation of GABP increases transcription. Raf-1, a kinase involved in the ERK pathway, works with GABP to stimulate the HIV-1 promoter activity (Flory 1996⁸⁶). These results support the idea that Raf-1 activates GABPα-and GABPβ-mediated gene expression. Further tests showed that GABP is phosphorylated in vivo by Raf-1 kinase activators (serum and TPA) and constitutive versions of Raf-1 kinase. The basal phosphorylation level of GABPα and GABPβ increased 2- to 4-fold after stimulation with serum and TPA (Flory 1996).

To identify kinases of GABPα and β, bacterially expressed GABPα and β proteins were tested as substrates in in vitro kinase assays. Raf-1 did not phosphorylate GABP subunits in vitro, but phosphorylation of both GABPα and GABPβ was detected in the reaction mixture containing MEK1, ERK2, GABPα, and GABPβ. ERK1 yielded similar results. A kinase-inactive ERK1 did not phosphorylate GABPα and β (Flory 1996). These results suggest that ERK1 directly phosphorylates both GABPPα and GABPPβ.

A DNA segment in the upstream region of the human IL-2 gene contains a transcription enhancer (−502 to −413). The enhancer binds the transcription factor GABPα and GABPβ at −462 nt to −446 nt (designated ERE-B) and −440 to −424 nt (designated ERE-A) (Avots 1997⁸⁷). GABP is a target of the MAP signal transduction pathway in T cells. c-Raf enhances IL-2 induction through GABP factors. Co-transfection of CAT reporter genes controlled by the distal enhancer with GABPα and β expression vectors into cells showed an increase in CAT activities. Mutation of one of both ERE motifs wiped out the induction, underscoring the important function role of GABP binding for induction of the distal enhancer. These data indicate that the c-Raf mediated increase of IL-2 induction is, at least partially, mediated by the GABP factors binding to the two ERE motifs (Avots 1997). According to Avots, et al., there appears to be an important role for the MAP pathway in induction of GABP factors binding to and controlling the distal IL-2 ERE enhancer motifs in T cells.

7. GABP Kinase Agent and Microcompetition

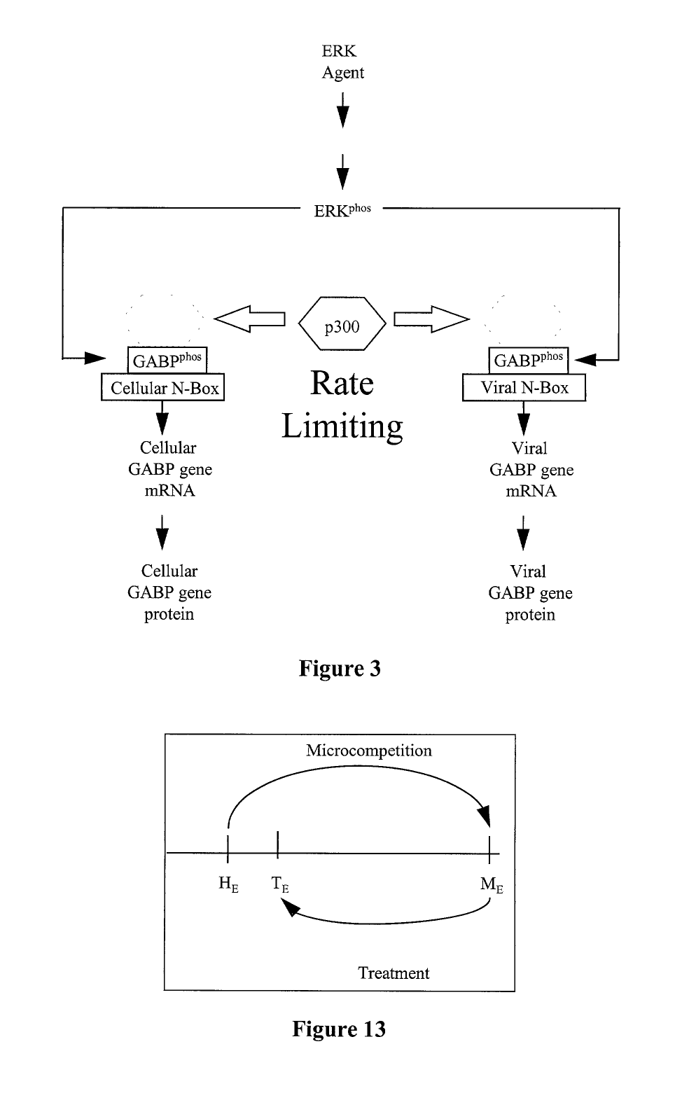

The relationship between ERK signaling and microcompetition is summarized in FIG. 3. Microcompetition between a GABP virus and cellular DNA reduces the availability of GABP to cellular genes. Let [N-box_{V}] denote the cellular concentration of viral N-boxes. Let [GABP_{C}] and [GABP_{V}] denote the concentration of GABP bound to cellular genes and viral DNA, respectively. [GABP_{V}] is a function of [N-box_{V}]. For every [N-box_{V}]>0, microcompetition reduces [GABP_{C}]. An GABP kinase agent phosphorylates GABP and stimulates p300 binding. If [N-box_{V}] is fixed, the GABP kinase agent stimulates the transcription of GABP stimulated genes and suppresses the transcription of GABP inhibited genes.

Fixed [N-box_{V}] seems to hold in cases of latent infection. In such cases, ERK phosphorylation of GABP_{V} stimulates the formation of N-box_{V}•GABP_{V}•p300 complexes. However, there is no increase in viral replication, which might have further reduced the availability of p300 to cellular genes and diminished or even canceled the ERK effect.

8. JNK/SAPK Pathway

a) Phosphorylation of GABP

Another signaling pathway which phosphorylates GABP is JNK/SAPK (see FIG. 1). Consider the following study.

To study the effect of JNK/SAPK phosphorylation on GABP the in vivo effects, HEK-293, human embryonic kidney cells were transfected with GABPα and GABPβ expression vectors alone, or in combination with SAPKβ expression vector and metabolically labeled with [³²P]orthophosphate. The cells were treated with anisomycin to strongly activate SAPK without affecting ERK activity. The results showed increased phosphorylation of both GABPα and GABPβ upon anisomycin stimulation. The phosphorylation was further increased with SAPKβ overexpression (Hofflmeyer 1998⁸⁸, FIGS. 5A and B). The study next tested the ability of these kinases to phosphorylate GABP in vitro, using ERK as a positive control. In vivo activated and immunopurified GST-tagged SAPKβ, but not Flag-tagged p38, phosphorylated both subunits of GABP (Ibid, FIG. 6B). Bacterially expressed, purified, and preactivated GST-SAPKαI also phosphorylated both GABP subunits in vitro like GST-c-Jun (Ibid, FIG. 6C). Both activated SEK and 3pK did not phosphorylate GABP. Next, the study tested another JNK/SAPK isozyme, JNK1/SAPKγ. In addition to ERK, untreated or TPA/ionomycin-stimulated A3.01 cells (a human T lymphoma cell line) phosphorylated both GABPα and GABPβ in vitro (Ibid, FIG. 6A). Based on these results, Hoffineyer, et al., concluded that “the ability of three different isoforms of JNK/SAPK (SAPKα, SAPKβ, and JNK1) to phosphorylate GABP in vitro, in combination with the in vivo phosphorylation of GABP upon SAPK activation by anisomycin, suggests that GABP is targeted by JNK/SAPK-activating pathways.”

Let a GABP kinase be any enzyme which phosphorylates GABP. Since GABP is a new concept, we sometimes revert to ERK instead of GABP kinase. However, in such cases ERK actually means GABP kinase.

9. N-box! GABP Binding Regulation

Oxidative stress decreases the binding of GABP to the N-box and reduces transcription of GABP stimulate genes and increases transcription of GABP suppressed genes. Consider the following study.

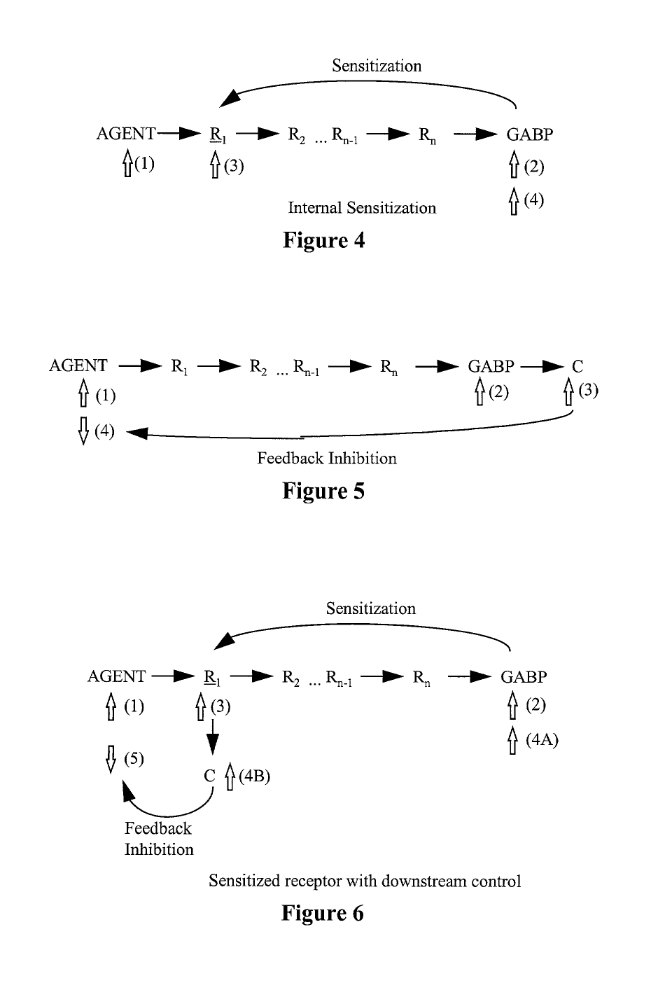

Mouse 3T3 cells were treated for 2 h with diethyl maleate (DEM), a glutathione (GSH)-depleting agent, in the presence or absence of N-acetylcysteine (NAC), an antioxidant and a precursor of GSH synthesis. Following treatment, the cells were harvested, and nuclear extracts were prepared in the absence of a reducing agent. GABP DNA binding activity was measured by EMSA analysis using oligonucleotide probes containing a single N-box (AGGAAG) or two tandem N-boxes (AGGAAGAGGAAG). Treatment of 3T3 cells with DEM resulted in a dramatic decrease in the formation of the GABP hetrodimer (GABPAGABPβ, (Martin 1996⁸⁹, FIG. 2A, lane 2) and hetrotetramer (GABPα₂GABPβ₂) (Ibid, FIG. 2A, lane 6) complexes on the single and double N-bo. XInhibition of GABP DNA binding activity by DEM treatment was prevented by simultaneous addition of NAC (Ibid, FIG. 2A, lane 4 and 8). The reduction of GABP DNA binding activity was not due to loss of GABP protein since the amount of GABPα and GABPβ1 was unaffected by DEM or NAC treatment. Dithiothreitol (DTT) is an antioxidant. DTT treatment of nuclear extracts prepared from DEM-treated 3T3 cells restored GABP binding activity. Treatment of 3T3 nuclear extracts with 5 mM GSSG nearly abolished GABP DNA binding. Based on these observations Martin et al., concluded that GABP DNA binding activity is inhibited by oxidative stress, i.e. GSH depletion. The study also measured the effect of DEM treatment on expression of transiently transfected luciferase reporter constructs containing a TATA box with either upstream double N-box or C/EBP binding site (Ibid, FIG. 4). DEM treatment had no effect on luciferase expression from C/EBP-TA-Luc after 6 or 8 h treatment (Ibid, FIG. 4). However, DEM treatment of cells transfected with double N-box-TATA-Luc, resulted in a 28% decrease in luciferase expression after 6 h and a 62% decrease after 8 h (Ibid, FIG. 4). Based on these results, Martin et al., concluded that glutathione depletion inhibits GABP DNA binding activity resulting in reduced expression of GABP-regulated genes.

Oxidative stress decreases GABP binding to the N-box, which decreases transcription of a GABP stimulated gene and increases transcription of a GABP repressed gene.

Microcompetition for GABP also decreases binding of GABP to the N-bo. XTake a GABP gene sensitive to oxidative stress through GABP only¹. The effect of microcompetition on the transcription of this gene is similar to the effect of oxidative stress. In other words, for this gene, microcompetition can be viewed as “excess oxidative stress.” ¹ Oxidative stress also modifies binding of other transcription factors, such as AP1, and NF-κB.

### B. Molecular Effect of Microcompetition

1. Signaling

a) Sensitization by GABP

The statement “A stimulates B” means that A stimulates the expression of B either directly or indirectly. Let “AGENT” be a GABP kinase agent which activates the transcription factor GABP. Let GABP stimulate the expression of a protein P. Let [AGENT]₁ and [AGENT]₂ be two concentrations of AGENT with corresponding concentrations [P]₁ and [P]₂. The intensity of signal [AGENT]₁ relative to [AGENT]₂ is equal [AGENT]₁/[AGENT]₂=[P]₁/[P]₂. The intensity of an ERK signal is measured by its effect on transcription of the protein P.

Let AGENT be a GABP kinase agent which activates the transcription factor GABP. Let (AGENT, GABP) denote the signaling pathway that leads from AGENT to GABP. Every protein R, such that R is an element of the signalling cascade (AGENT, GABP) will be called an “ERK receptor for AGENT.” In other words, AGENT activates the R protein, which in turn activates GABP. For example, the leptin long receptor is an ERK receptor for leptin, and metallothionein is an ERK receptor for zinc.

Let AGENT be a GABP kinase agent. If there is a protein R in the signalling cascade (AGENT, GABP), such that AGENT stimulates the expression of R, the (AGENT, GABP) pathway will be called “sensitized” and R will be called the “sensitized receptor,” denoted R. Sensitization increases the intensity of a given signal by increasing the number of receptors available to be activated by a given amount of GABP kinase agent.

Let R be a sensitized receptor in (AGENT, GABP). If the expression of R is stimulated by GABP, R will be called an “internally sensitized receptor.” FIG. 4 shows an increase in AGENT stimulates the phosphorylation of GABP (step 1 and 2 in the figure). The phosphorylated GABP stimulates the transcription of R₁, the sensitized receptor (step 3). The new R₁ receptors increase the sensitivity of the pathway to the change in the concentration of the GABP kinase agent, that is, increase the probablity of binding between the GABP kinase agent and R₁. The increased binding further increases the number of phosphorylated GABP molecules (step 4).

GABPβ and γ are similar proteins. The only difference between them is the homodimerization section in the C-terminal region. Antibodies which is not specific to the C-terminus bind both proteins. Such antibodies are not sensitive enough to identify a relative change in their expression. However, since GABPβ and GABPγ are almost always bound to GABPα, (Suzuki 1998⁹⁰), and since GABPβ is an activator and GABPγ is a suppresser (Suzuki 1998), an increase in GABPα with an increase in gene expression indicates an increase in the GABPβ concentration relative to γ.

In the pathway (OT, OTR, GABP), the receptor OTR is stimulated by GABP (Hoare 1999⁹¹). In (zinc or copper, hMT-II_{A}, GABP), hMT-II_{A} is a receptor stimulated by GABP (see discussion above). In the pathway (LPS, CD18, GABP), CD18 is a receptor stimulated by GABP (Rosmarin 1998⁹²). In the pathway, (IL-2, IL-2Rβ, γc, GABP), IL-2Rβ and γc are two receptors stimulated by GABP (Markiewicz 1996, Lin 1993).

According to the definition of an ERK receptor, GABP is also an ERK receptor. In addition, some GABP kinase agents increase the expression of GABP turning GABP from an ERK receptor into a sensitized receptor. Consider the following examples.

IFNγ: Evidence suggests that interferon-γ (IFNγ) regulates GABP DNA binding by increasing the amount of the GABP proteins present in bone marrow-derived macrophages (BMDM) nuclei. IFNγ treatment of BMDM leads to induction of the binding activity (Tomaras, 1999). Since the GABPβ and GABPγ are almost always bound to GABPα, (Suzuki 1998), an increase in β indicates an increase in GABPα.

The increase in DNA binding activity correlates with an increase in immunodetectable GABPα (Tomaras 1999⁹³). The essential sites for activity of GABP within the third intron map to a highly conserved tandem repeat of ets-transcription factor binding sites. Mutations in the ets site within the intron inhibited this activity. A dominant-negative ets plasmid also completely negated this cooperativity. It was determined that a GGAA sequence repeat is a transcriptionally active site which interacts with an ets transcription factor. Specifically, GABP binds to this region. GABP binding activity is increased by treatment with IFNγ in BMDM (Tomaras 1999).

Heregulin: Heregulin increases GABPα expression (Schaffer 1998). Western blot analysis of heregulin treated and non-treated cells showed that heregulin treatment leads to a 2-fold increase in the protein level of GABPα, while the GABPβ protein level was unaffected (Schaffer 1998).

PMA: Bottinger, et al., 1994⁹⁴ defined the minimal defined promoter for CD18 (β2 integrin) expression in myeloid and lymphoid cells by generating 5′ and 3′ deletion constructs of a segment ranging 785 bp upstream and 19 bp downstream of a major transcription start site. The region extending from nucleotides −302 to +19 supported cell-restricted and phorbol ester-inducible expression. Two adjacent promoter regions, from nt −81 to −68 (box A) and −55 to −41 (box B), were revealed by DNase I footprinting of this region. DNA-binding proteins that interact with box A and box B were identified through electrophoretic mobility shift assays. Using box A as a probe yielded a major complex, designated BA-1. This complex increased in intensity after phorbol ester-induced differentiation of the cells. The complex was also detected using the radiolabeled box B element. The complex is homologous to GABP. Antiserum specific to GABPα or GABPβ abrogated binding of BA-1, while antisera to other ets-transcription factors had no effect (Bottinger 1994).

Expression of CD18 corresponds to the DNase I protection profiles observed in vitro, suggesting that the complexes that bind over the protected elements mediate tissue specific expression of the CD18 gene. In T cells, the BA-1 complex forms over the box A and box B elements and is apparently responsible for the DNase I protection seen. Despite the formation of the same complex in the HeLa CD18 negative cell line, there is no observed DNase I protection (Bottinger 1994).

The different effects of PMA treatment observed in T cells and HeLa cells can be explained in the following way. In T cells the expression of GABPα and GABPβ increases. Since GABPαβ is an activator, Bottinger observed increased expression of CD18 and DNase-I protection on the CD18 promoter. In HeLa cells the expression of GABPα and GABPγ increases. Since the GABPαγ is a suppresser, Bottinger observes no expression of CD18 and little DNase-I protection on the CD18 promoter.

b) Resistance

Traditionally, there are two definition of resistance, cellular level resistance and patient level resistance:

Cellular level resistance: Let L denote a ligand and O a cell. Let L produce the effect Y in O. The cell O will be called “L resistant” if a given concentration of L produces a smaller Y effect in O relative to control.

Patient level resistance: Let L denote a ligand. A patient will be called “L resistant” if the patient shows elevated levels of L relative to controls. Patient level resistance is sometimes calle hyper-L-emia. Example: Insulin resistance and hyperinsulinemia.

FIG. 5 shows an example of feedback inhibition involving GABP. Let AGENT be a GABP kinase agent. Let C be a protein. If the expression of AGENT depends on the expression of C, C will be called a “control” for AGENT. If an increase in C represses the expression of AGENT, or increases its degradation, C will be called a “negative control” and the effect on AGENT “feedback inhibition.”

Let AGENT be a GABP kinase agent with the (AGENT, GABP) pathway. If GABP stimulates C, C will be called a “GABP stimulated” control. AGENT phosphorylaes GABP (step 1 and 2). GABP increases the transcription of C (stpe 3). C decreases the expression of the GABP kinase agent (step 4).

Cellular Level Resistance

Let AGENT be a GABP kinase agent with the (AGENT, GABP) pathway. Let AGENT produce the effect Y in the cell O. Let the Y effect be dependent on transcription of a GABP gene X in O. Under microcompetition in O, a given concentration of AGENT produces a smaller concentration of X and a smaller Y effect.

Patient Level Resistance

Let AGENT be a GABP kinase agent with the (AGENT, GABP) pathway. Let C be a negative control for AGENT which is also GABP stimulated. Microcompetition for GABP elevates the concentration of AGENT. As a GABP kinase agent, AGENT phosphorylates the pool of GABP molecules. Phosphorylation of GABP increases C. The added C represses AGENT. However, microcompetition reduce the size of the GABP pool, or the amount of GABP available to stimulate C. Therefore, microcompetition diminishes the increase in the control C, which lessens the repression effect on A. In the above figure, the size of the arrow in step 2 is smaller, hence the size of the arrow in step 3 is smaller and so is the size of the arrow in step 4.

Note that the control C in the above figure is down stream from GABP. What if the control is positioned between the GABP kinase agent and GABP? Would microcompetition cause patient level resistance in such a pathway?

FIG. 6 shows the effects of downstream control relative to a sensitized receptor. Let R be an internally sensitized receptor in (AGENT, GABP). Let C be a negative control for AGENT. If R stimulates C (C is downstream from R), microcompetition for GABP elevates the concentration of AGENT. AGENT phosphorylaes GABP (step 1 and 2). GABP increases the transcription of R₁ (stpe 3). R₁ increases the effect on GABP (step 4A) and increases the expression of the control C (step 4B), which decreases the expression of the GABP kinase agent (step 5). Microcompetition decreases the size of the arrows in step 2, 3, 4A, 4B and 5.

If the control is down stream from the sensitized receptor, microcompetition causes patient level resistance.

Consider the following two pathways (OT, OTR, GABP), (zinc or copper, hMT-II_{A}, GABP) as examples. In these pathways, the sensitized receptor directly binds the GABP kinase agent. Therefore, the control must be down stream from the sensitized receptor, and the pathways must show patient level resistance under microcompetition. This conclusion can be reached independent of any information about the control. The pathway (LPS, CD18, GABP) is similar. Elicitation of a bioequivalent reaction requires a higher concentration of LPS in a cell infected by a GABP virus compared to a non infected cell. The pathway (IL-2, IL-2Rβ, γc, GABP) is different (see below).

Let the set {(AGENTᵢ, GABP, C₁)} include all pathways with a GABP kinase agent AGENTᵢ and control C₁ downstream from GABP. For all AGENTᵢ, microcompetition for GABP reduces the expression of C₁, which, in steady state, increases the concentration of AGENTᵢ. Using the resistance terminology, it can be said that microcompetition for GABP causes cells infected with a GABP virus to show AGENTᵢ patient level resistance.

2. Transcription

a) Retinoblastoma Susceptible Gene (Rb)

The Rb promoter includes a N-box at (−198, −193). Several plasmids were produced. pXRP1 included the normal (−686, −4) segment ofthe Rb promoter. pXRP3 included the same segment with a mutated N-bo. XRBF-1×4 included 4 copies of the Rb N-box as promoter. All promoters controled the expression of the luciferase (luc) reporter gene. Cotransfection of hGABPα and hGABPβ₁ expression plasmids with pXRP1 into SL2 Drosophila cells showed a 10-fold increase in the reporter gene activity. Cotransfection with RBF-1×4 showed a 13-fold increase. Cotranfection with pXRP3, the mutated N-box, showed no increase (Sowa 1997⁹⁵). Based on these observations, and other results, Sowa, et al, concluded that hGABP has a strong transactivating effect on the Rb gene promoter, suggesting that hGABP is the main transcativator for the core promoter element of the Rb gene.

GABP viruses microcompete with the Rb promoter for GABP. Therefore, viral infection of cells decreases Rb expression. Moreover, the higher the concentration of viral DNA, the greater the decrease in Rb expression.

b) Breast Cancer Type 1 Gene (BRCA1)

The BRCA1 promoter includes three N-boxes at (−200, −178). Plasmids with point mutations in the central N-box, alone or in combination with mutations in the other N-boxes, were transfected in MCF-7, a human breast cell line. The mutated plasmids showed a 3-fold reduction in promoter activity (Atlas 2000⁹⁶, FIG. 2). Nuclear extracts from MCF-7 formed a specific complex with the N-boxes region. Through crosslinking, supershif assays and binding to recombinant GABPαβ (Ibid, FIGS. 4, 5) . . . GABPαβ was identified as the main transcription factor interacting with the N-boxes. An artificial promoter containing the mutlimerized N-boxes region was transactivated by cotransfection with GABPα and GABPβ₁ in both MCF-7 and T47D, another human breast cell line (Ibid, FIG. 6). These observations indicate that BRCA1 is a GABP stimulated gene.

GABP viruses microcompete with the BRCA1 promoter for GABP. Therefore, viral infection of cells decreases BRCA1 expression. Moreover, the higher the concentration of viral DNA, the greater the decrease in BRCA1 expression.

c) Fas gene (Fas, APO-1, CD95)

The Fas promoter includes two N-boxes at (−857, −852) and (−833, −828). Jurkat cells, a T cell line, were transiently transfected with luciferase reporter gene driven by different length of the Fas promoter. The cells were stimulated for 10 h with anti-CD3 mAb, PMA and PMA/ionomycin. Deletion of the two N-boxes reduced activation by 50-75% (Li 1999⁹⁷, FIG. 1). Mutation of the N-boxes also reduced stimulated luciferase activity (Ibid, FIG. 7). Cell stimulation resulted in formation of specific complexes on the N-boxes region. Mutation of the N-boxes reduced formation of these complexes (Ibid, FIG. 4). Antibodies against GABPα and β inhibited formation of these complexes (Ibid, FIG. 6A). Two or four copies of the Fas/GABP site (−863, −820) were inserted into reporter plasmid pGL3/promoter. Anti-CD3 mAb, PMA and PMA/ionomycin stimulated luciferase activity 8-20 fold in Jurkat transfected cells (Ibid, FIG. 9). Mutation of the N-boxes significantly reduced the induction of luciferase activity in response to stimulation. These observations indicate that Fas is a GABP stimulated gene.

GABP viruses microcompete with the Fas promoter for GABP. Therefore, viral infection of cells decreases Fas expression. Moreover, the higher the concentration of viral DNA, the greater the decrease in Fas expression.

d) Tissue Factor (TF) Gene

Transcription

ETS related factors repress TF transcription. Consider the following studies. The first studies show that ETS related factor(s) bind the (−363 to −343) and (−191 to −172) segments.

A study used DNase I footprinting to map the sites of protein-DNA interaction on the (−383 to +8) fragment of the TF promoter. The study used nuclear extracts prepared from uninduced and lipopolysaccharide-induced THP-1 monocytic cells. Six regions were identified. Region number 7 (−363 to −343) and region number 2 (−191 to −172) contain an N-bo. XTHP-1 extracts formed two complexes on a consensus N-bo. XBoth complexes were competed with excess unlabeled N-box and 200-fold excess of a (−363 to −343) probe. The (−191 to −172) probe, although not as effective as the (−363 to −343) probe, showed approximately 30% reduction in the N-box complex formation (Donovan-Peluso 1994⁹⁸, FIG. 9).

Another study used the (−231 to −145) fragment of the TF promoter as probe. Nuclear extracts prepared from uninduced and lipopolysaccharide-induced THP-1 monocytic cells formed two complexes on the (−231 to −145) probe. To characterize the proteins that interact with the DNA sequence, the study used the sc-112x antibody from Santa Cruz Biotechnology. According to the manufacturer literature, the antibody has broad cross-reactivity with members of the ETS family. Incubation of the antibody with the nuclear extracts abrogated the formation of the upper complex on the (−231 to −145) probe (Groupp 1996⁹⁹, FIG. 5).

The following study shows that (−191 to −172) segment also binds NF-κB. Monocytic THP-1 cells were stimulated with LPS for various times up to 24 h. TF mRNA increased by 30 min and reached a peak at 1 h. Levels dropped considerably by 2 h returning, eventually, to preinduction levels (Hall 1999¹⁰⁰, FIG. 1). The same study conducted EMSA studies using the (−213 to −172) fragment of the TF promoter. The results showed that two complexes, marked III and IV, appear at 30 min, with binding reaching a peak at 1-2 h. At 4 h and later, the complexes are no longer detected. A 100-fold molar excess of a (−213 to −172) probe, or a NF-κB consensus oligonucleotide, compete complexes III and IV (Ibid, FIG. 2B). An antibody against p65 and to a lesser extent, anti-c-Rel, supershifted complex III. These data demonstrate a transient binding of two NF-κB complexes to the (−213 to −172) fragment between 30 min and 2 h. However, the affinity of complexes on the NF-κB site was much lower than the affinity of the complexes on the adjacent proximal AP1 site.

This study also provides evidence indicating that LPS induces proteolysis of IκB and translocation of p65 and c-Rel from the cytoplasm to the nucleus. Western blot analyses showed that very little p65 was present in the nucleus in unstimulated cells. After 10 min of LPS induction, nuclear p65 begins to appear and peak at 1 h declining again by 2 h. A concomitant decrease in cytoplasmic p65 corresponds to the observed increase in nuclear p65 (Ibid, FIG. 4).

The following study shows that the (−363 to −343) factor(s) repress TF transcription. Holzmuller, et al., (1999¹⁰¹) call the (−363 to −343) fragment of the TF promoter the Py-bo. XA deletion of the 5′-half of the Py-box increased expression of a luciferase reporter gene (Ibid, FIGS. 3A and B). The relative increase was similar for LPS induced or nontreated cells and was independent of the existence of NF-κB site (Ibid, FIG. 3C). Mutation of the N-box part of the Py-box resulted in complete loss of binding activity to the Py-box.

The following study shows competition between ETS related factor(s) and NF-κB for (−191 to −172). Donovan-Peluso, et al., (1994¹⁰², see above) showed that the (−191 to −172) probe was less effective in competing with the consensus N-box compared to the (−363 to −343) probe. According to the authors, the data suggest that there might be competition for binding to the (−191 to −172) fragment by NF-κB and ETS related factors. In such a case, NF-κB binding to a (−191 to −172) probe reduces the concentration of the probe available to for ETS binding. This competition can explain the reduced ability of (−191 to −172) to compete for ETS binding relative to (−363 to −343). Moreover, the NF-κB site and the N-box in the (−191 to −172) fragment overlap. The presence of overlapping sites also suggests competition where occupancy by the either factor might preclude binding by the other.

Microcompetition between a GABP virus and the TF promoter decreases the availability of the ETS related complexes in the nucleus.

NF-κB binding to (−191 to −172) increases transcription. Competition between NF-κB and ETS related factors for (−191 to −172) suggests that the decrease in availability of the ETS related factors in the nucleus increases the binding of NF-κB to the (−191 to −172) fragment and increases TF expression.

Binding of ETS related factor(s) to the (−363 to −343) fragment represses transcription. The repression is similar in extracts from untreated, or LPS- or TNF-α-induced cells. Moreover, the repression is independent of NF-κB binding. This observation suggests that the ETS related factor(s) suppress transcription in quiescent cells and maintain the rates in activated cells at a moderate level (Holzmuller 1999¹⁰³). The decrease in availability of the ETS related factor(s) in the nucleus reduces the (−363 to −343) repression and increases TF expression.

The GABP virus microcompetes with the TF promoter for the ETS related factor(s), therefore, viral infection of monocytes/macrophages increases TF expression. Moreover, the higher the concentration of viral DNA, the greater the increase in TF expression.

The results in the following studies with GABP viruses are consistent with the proposed effect of microcompetition on TF transcription. A few studies measured the expression of TF relative to an internal control. The studies used two controls CMVβgal (Moll 1995¹⁰⁴, Nathwani 1994¹⁰⁵) and pRSVCAT (Mackman 1990¹⁰⁶). Although the studies used different transfection protocols; Moll, et al., (1995) used psoralen- and UV-inactivated biotinylated andenovirus and streptavidine-poly-L-lysine as vectors for DNA delivery, Nathwani, et al., (1994) used electoporation and Mackman, et al., (1990) used DEAT-dextran, they all report an increase in TF expression relative to a promoterless plasmid. According to Moll, et al., (1995), the cells “are being already partially activated following the transfection procedure.” The level of activation was similar in unstimulated and LPS stimulated cells. The internal controls include promoters of GABP viruses. The control promoters microcompete with the TF promoter for the ETS related factor(s). The reduced availability of the ETS related factor(s) increases the transcription of the reporter gene fused to the TF promoter.

Confluent monolayers of human umbilical vein endothelial cells (HUVEC) were exposed to 0.1 μg/ml LPS for 4 hours and HSV-1. At appropriate time intervals, TF procoagulant activity (PCA) was assessed by clotting assays. Maximal TF PCA activity was observable 4 hours after infection and was still detectable 20 hours post infection. Both the HSV infection and LPS exposure show a similar activity profile over time. However, the maximal activity induced by HSV is about a ⅓ of LPS. Further studies with specific blocking antibodies to human TF support the notion that the PCA is indeed due to TF.

HUVEC were also infected with HSV-1 inactivated by either ultra-violet-irradiation or heat. The cellular TF PCA was measured in lysates of control, LPS stimulated (0.1 mg/ml for 4 hours), or infected cells. Virally infected cells were maintained in culture for up to 48 hours and visually inspected for cytopathic effects as evidence from lytic infection. Obvious morphologic changes were evident in cells infected with competent virus after 18 to 24 hours. In comparison, no signs of infection were visible in cells infected with heat or UV-treated virus even after 48 hours. The TF PCA of the different treatments measured 4 hours post infection is summarized in the following table.

| | TF PCA |
|---|---|
| | (U/ml) |
| Control | 74 |
| LPS | 1753 |
| HSV-1 | 773 |
| Heated HSV-1 (80° × 30 min) | 691 |
| UV irradiated HSV-1 | 384 |

Virus inactivated by UV or heat is still capable of inducing TF activity (Key 1993¹⁰⁷). This study measures the effect of infection with an inactivated GABP virus on TF transcription. The reduced TF transcription is consistent with microcompetition between the viral DNA and the TF promoter for the ETS related factor(s).

Studies with GABP kinase agents are also consistent with the proposed effect of microcompetition on TF transcription. Many papers report the effects of c-Fos/c-Jun, c-Rel/p65, Sp1 and Egr-1 binding on TF transcription. LPS and PMA are GABP kinase agent and, therefore, phosphorylate the ETS related factors. However, LPS and PMA also stimulates the binding of NF-κB and Egr-1, respectively, to the TF promoter.

FIG. 7 shows the effect of LPS on NF-κB (dotted lines) and and on ERK (solid lines). As such, LPS and PMA are not useful in isolating the effect of ETS phosphorylation on TF transcription. The next section presents two GABP kinase agent, all-trans retinoic acid (ATRA) and resveratrol, which have no effect on NF-κB, Ap1 and Sp1. As GABP kinase agent, ATRA and resveratrol phosphorylate the ETS related factor(s), stimulate the binding of p300, and, therefore, should repress TF transcription.

Consider the effect of all-trans retinoic acid (ATRA). Monocytes were incubated for 30 minutes with various doses of ATRA before LPS stimulation. ATRA inhibited LPS induction of TF expression in a dose-dependent manner (Oeth 1998¹⁰⁸, FIG. 1A). The LPS induction of TF activity was also inhibited by ATRA in THP-1 monocytic cells (Ibid, FIG. 2A). Specifically ATRA reduced the basal levels of TF mRNA in unstimulated cells and abolished the LPS induction of TF mRNA (Ibid, FIG. 3A). However, ATRA did not affect DNA binding of the c-Fos/c-Jun, c-Rel/p65 or Sp1 transcription factor to the AP1, NF-κB and Sp1 sites.

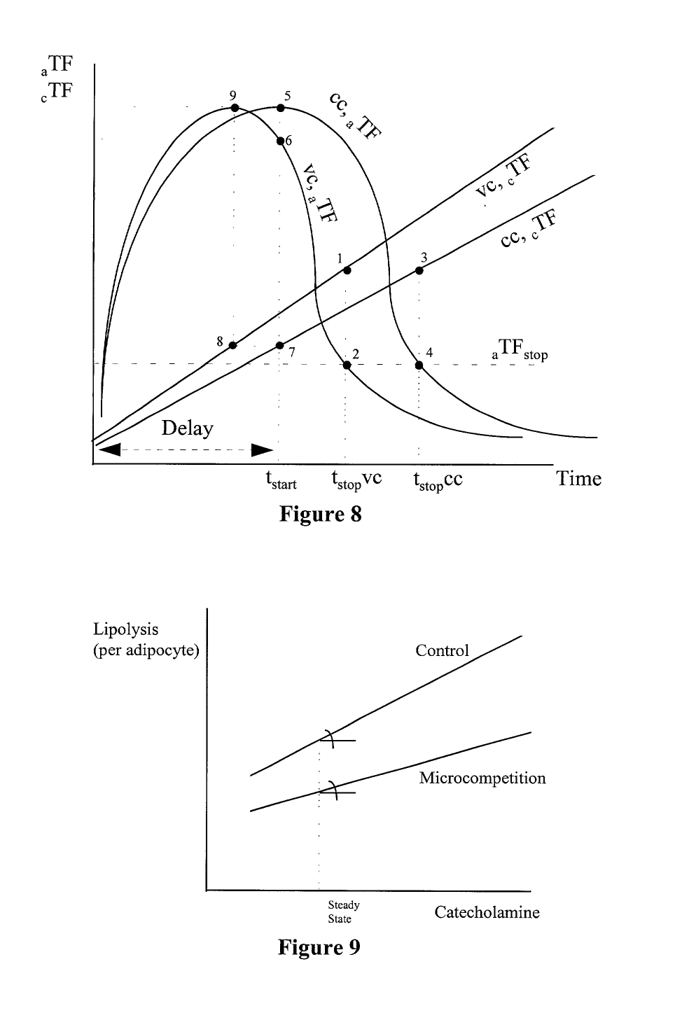

Consider the effect of resveratrol (RSVL). Confluent monolayers of human umbilical vein endothelial cells (HUVEC) were treated with resveratrol (100 μmol/L) for 2 hours. Following resveratrol treatment, the cells were stimulated for 6 hours with LPS, TNFα, IL-1β or PMA. The results showed that resveratrol markedly suppressed LPS-, TNFα-, IL-1β-, and PMA-induced TF activity (Pendurthi 1999¹⁰⁹, FIG. 1A). The inhibition varied from 60% to more than 90%. HUVEC monolayers were also treated with different concentrations of resveratrol (0 to 200 μmol/L) for 2 hours. Following resveratrol treatment, the cells were stimulated with TNFα, IL-1β, or PMA. The data showed that resveratrol inhibited the induction of TF expression in a dose-dependent manner. To test the effect of resveratrol in monocytes, mononuclear cell fractions were treated with various concentrations of resveratrol (0 to 100 μmol/L) for 2 hours, and then stimulated with LPS (100 ng/mL) for 5 hours. The results showed that resveratrol inhibited LPS-induced TF expression in monocytes in a dose-dependent manner (Ibid, FIG. 2). To test the effect of resveratrol on TF mRNA, HUVEC monolayers were treated with various concentrations of resveratrol (0, 5, 20, 100, and 200 μmol/L) for 2 hours, and then stimulated with LPS, TNFα, IL-1β, or PMA for 2 hours. Resveratrol treatment reduced TF transcription in a dose-dependent manner. However, the reduced transcription was not due to diminished binding of c-Fos/c-Jun or c-Rel/p65 to the TF promoter. Resveratrol did not significantly change the binding of c-Fos/c-Jun to the AP-1 sites. Resveratrol treatment had no significant effect on binding activity to the AP-1 site in either unstimulated or LPS-, TNFα-, IL-1β-, or PMA-stimulated endothelial cells (Ibid, FIG. 7). Resveratrol also did not significantly changes the binding of NF-κB to the TF promoter. Unstimulated cells showed little binding of NF-κB, whereas LPS, TNFα, IL-1β, or PMA induced formation of a prominent DNA-protein complex on the NF-κB site. Preincubation of cells with resveratrol (100 μmol/L) for 2 hours had no effect on formation of the NF-κB DNA-protein complex (Ibid, FIG. 8).

Both ATRA and resveratrol are GABP kinase agent and, therefore, phosphorylate the ETS related factor(s). In general, phosphorylation of ETS related factor(s) stimulates binding of p300. The ETS•p300 complex, when bound to the TF promoter, represses TF transcription. The repression is independent of NF-κB, Ap1 or Sp1.

Deactivation (“Encryption”) as a Function of Membrane Concentration

TF surface dimers are inactive. According to Bach, et al., (1997¹¹⁰), surface TF exists in two forms, monomers and dimers. Both monomers and dimers bind FVIIa. However, only monomers are active. Self-association of TF monomers prevents access to an essential macromolecular substrate binding site. The concept of inactive (cryptic) dimers is consistent with the crystal structures of the extracellular domain of TF. The structure suggest that TF dimerization does not block FVIIa binding but covers the macromolecular substrate binding site on the opposite face of TF.

Bach, et al., (1997) provide ample evidence consistent with this model. Consider the following experiments. HL-60 cells were exposed to 10⁻⁶ mol/L PMA for various times. The intact cells were assayed for TF procoagulant activity (PCA) either before or following a brief exposure to 10 μmol/L ionomycin. In comparison to a PMA treatment, a combined ionomycin and PMA testament resulted in a dramatic increase in expression of TF PCA (Ibid, FIG. 1). The rapid appearance of the activity shows that de novo protein synthesis was not involved (Ibid, FIG. 2). The calcium influx activated the latent TF PCA. Also, the inhibition by calmidzaolium (CMZ) implicates calmodulin (CaM) as an essential link in the process (Ibid, FIGS. 3, 4). Moreover, FVIIa bound to TF on untreated cells as well as ionophore-treated cells (Ibid, FIG. 5, experiment 1 and 2). Thus, restricted formation of TF-FVIIa does not account for inactive (cryptic) TF PCA. The TF-FVIIa complex readily bound the pseudosubstrate tissue factor pathway inhibitor-activated factor X (TFPI-FXa) on ionophore-treated cells, but was resistant to TFPI-FXA inhibition on untreated cells. Similar inhibition on ionophore-treated cells was demonstrated with XK1, another pseudosubstrate of TF-FVIIa. These results suggests that calcium influx exposes a TFPI-FXa/XK1 binding site on TF. Lastly, the HL-60 cells were treated with DTSSP, a monobifunctional amino-reactive protein shown to cross-link cell surface TF. Following the treatment, TF was immunopurified and visualized by Western blotting. The products of DTSSP cross-linking were TF dimers (Ibid, FIG. 7, lane 1, 2). When the cells were treated with ionomycin before cross-linking, almost no cross-linking was observed (Ibid, FIG. 7, lane 3). The decreased cross-linking suggests that TF does not self-associated on ionophore treated cells. Both the TF cross-linking and the encrypted TF PCA were preserved by treating the cells with CMZ before the addition of ionophore (Ibid, FIG. 7, lane 4).

Increase in surface concentration induces dimers, which reduces activity. Nemerson, et al., (1998¹¹¹) link the surface concentration of TF with its rate of catalytic activity. To establish such a link Nemerson and Giesen incorporated a recombinant TF (TF₁₋₂₄₃), which contained the transmembrane, but not the cytoplasmic domain, into appropriate phospholipid vesicles and measured their catalytic activity (k_{cat}). The results showed that the k_{cat}, or catalytic rate constant, which reflects the catalytic activity of each TF-FVIIa molecule, fell monotonically as a function of TF surface density. Moreover, following exposure of vesicles with high surface-density of TF (about 50 molecules of TF on the surface of a 100 nm vesicle) to a cross-linking reagent, Nemerson and Giesen were able to detect dimers and higher n-mers. Nemerson and Giesen suggested that these results are consistent with a model where clustered TF molecules have lower maximal catalytic activity compared to dispersed molecules.

Nemerson, et al., (1998) used a recombinant TF (TF₁₋₂₄₃), which contained the transmembrane, but not the cytoplasmic domain. To test the significance of the cytoplasmic domain in activation, Wolberg, et al., (2000¹¹²) transfected cells with either full length TF, or TF lacking its cytoplasmic domain. The results showed that TF activation by a calcium ionophore was independent of the cytoplasmic domain.

TF activity is regulated through formation of dimers. Schecter, et al., (1997¹¹³) show the effect of agonists stimulation on TF surface concentration and activity over time. TF mRNA was barely detectable in quiescent aortic smooth muscle cells (SMC) (Ibid, FIG. 1). FCS induced a marked rise in TF mRNA levels, beginning at ˜1 h and persisting for ˜8 h. Accumulation of TF mRNA in response to PDGF BB and α-thrombin was similar to that seen with 10% FCS (Ibid, FIG. 1). To test the effect of the rise in TF mRNA on protein synthesis over time, quiescent SMC were treated with growth agonist and examined by immunostaining every hour for the first 4 h, and every 2 h for additional 20 h. Untreated quiescent SMC showed minimal TF antigen. Cells stimulated with 10% FCS, PDGF AA, or BB, or thrombin receptor peptide, produced a pronounced perinuclear staining of TF antigen beginning at ˜2 h and peaking at 4-6 h. At 4-6 hours, TF antigen was also detected diffusely on the ruffled edges of the plasma membrane. Perinuclear staining persisted for ˜8-10 h after stimulation, and then gradually dissipated. At 16-24 h, a patchy distribution of antigen staining near or on the membrane was noted with diminished prinuclear staining. Schecter, et al., (1997¹¹⁴) measured the intensity of immunofluorescent staining along a line which traverses the nucleus and connects opposite sides of the cell membrane, and displayed the results graphically. At 4 h, the graph shows a bimodal distribution with two peaks, around the nucleus and along the membrane (Ibid, FIG. 5a, insert). At 16 h, the graph shows a much smaller peak around the nucleus and a much larger peak along the membrane (Ibid, FIG. 5b, insert).

Schecter, et al, (1997¹¹⁵) also measured the effect of PDGF simulation on TF activity. PDGF induced an approximately fivefold increase in surface TF activity (Ibid, FIG. 7) 4-6 h after treatment, with a return to baseline by 20 h.

The temporal events reported in this study show that the initial increase in TF membrane staining (4 h post stimulation) is associated with an increase in TF activity, while the subsequent increase in membrane staining (16 h post stimulation) is associated with a decrease in TF activity. The patches of TF staining on the cell surface are most prominent at a time (10-12 h after agonist stimulation) when surface TF activity is minimal. The study finds this relationship intriguing and proposes that the patches may represent inactive TF multimers.

e) P-selectin Gene

P-selectin (CD62P, GMP140, LECCAM-3, PADGEM) is expressed in megakaryocytes and endothelial cells. In endothelial cells P-selectin is stored in specialized granules known as Weibel-Palade (WP) bodies. After activation with inflammatory mediators, such as histamine, thrombin, or complement proteins, WP bodies fuse with the plasma membrane, resulting in increased P-selectin expression on the endothelial apical surface. One function of P-selectin is to mediate leukocyte adherence to activated endothelium.

Two conserved N-boxes were identified in the mouse and human P-selectin genes. The mouse distal N-box is positioned at (−327, −322) and the proximal at (−104, −99). The human distal N-box is positioned at (−314, −309) and the proximal at (−103, −108). A labeled probe encoding the murine proximal N-box formed two DNA-protein complexes with nuclear extracts from BAEC (Pan 1998¹¹⁶, FIG. 6B), bEnd.3, HEL and CHRF288 cells. Complex formation varied with different batches of nuclear extracts, characteristic of GABP binding. Competition with a HSV1 IE N-box probe, which binds GABP, prevented complex formation with BAEC nuclear extracts (Ibid, FIG. 6D). Based on these observations, Pan, et al., concluded that the proximal N-box most likely binds the ubiquitously expressed GABP.

Mutation of the AGGAAG proximal N-box to AGCTAAG eliminated the DNA-protein complex formation (Pan 1998, FIG. 6C). BAEC transfected with a reporter gene directed by the murine P-selectin promoter with the mutated N-box showed a 2-10-fold increased expression compared to the wild-type promoter (Ibid, FIG. 6F). The increased transcription indicates that binding of the Ets related factor to the proximal N-box represses the P-selectin gene. Deletion of the distal N-box had no effect on the reporter gene expression. The increase transcription of the mutated gene indicates that GABP is a repressor of P-selectin.

GABP viruses microcompete with the P-selectin promoter for GABP. Therefore, viral infection of endothelial cells increases P-selectin expression. Moreover, the higher the concentration of viral DNA, the greater the increase in P-selectin expression.

f) β₂ Integrin Gene

β₂ integrin (CD18) is a leukocyte-specific adhesion molecule. GABP binds three N-boxes in the CD18 promoter and transactivates the gene (Rosmarin 1995¹¹⁷, Rosmarin 1998¹¹⁸).

Latent infection by a GABP virus results in microcompetition between the viral DNA and CD18 promoter which decreases the expression of CD18 (see Le Naour 1997¹¹⁹, Tanaka 1995¹²⁰, Patarroyo 1988¹²¹ above). Moreover, the higher the concentration of viral DNA, the greater the decrease in CD18 expression.

g) α₄ Integrin Gene

α₄ integrin (CD49d) is expressed in B cells, thymocytes, monocytes/macrophages, granulocytes and dendritic cells. α₄ binds β₁ integrin to form α₄β₁ (CD49d/CD29, VLA-4). α₄β₁ binds vascular cell adhesion molelcule-1 (VCAM-1), which appears on the surface of activated endotheilal cells, and fibronectin (Fn), a major component of the extra-cellular matrix (ECM).

Rosen, et al., (1994¹²²) show that GABP binds the (−51, −46) N-box in the α₄ promoter. The binding of GABP activated the transcription of the α₄ integrin gene in Jurkat cells, a T-cell line. They also show that microcompetition with an Ets binding site from the Moloney sarcoma virus long terminal repeat inhibited binding of GABP to the α₄ integrin promoter. GABP viruses microcompete with the α₄ promoter for GABP. Therefore, viral infection of macrophages decreases α₄ expression. Moreover, the higher the concentration of viral DNA, the greater the decrease in α₄ expression.

h) Hormone Sensitive Lipase (HSL) Gene

Hormone sensitive lipase (HSL, Lipe, EC 3.1.1.3) is an intracellular neutral lipase highly expressed in adipose tissue. HSL is the rate limiting enzyme in triacylglycerol and diacylglycerol hydrolysis. HSL also mediates cholesterol esters hydrolysis generating free cholesterol in steroidogenic tissues and macrophages.

Consider infromation about the N-bo. XThe region −780 bp 5′ of exon B to the start of exon 1 was suggested to include potential regulatory sites of the human HSL gene in adipocytes (Talmud 1998¹²³, Grober 1997¹²⁴). This region includes 15 N-boxes. Moreover, three pairs are located at short distances of each other. The distance between the pair at (+268, +272), (+279, +285) is 5 bp or 1.0 helical turn (HT), at (+936, +942), (+964, +970) is 22 bp or 2.5 HT, and at (+1,253, +1259), (+1270, +1276) is 11 bp or 1.5 HT.

Of the dozens of known ETS factors, only GABP, as a tetrameric complex, binds two N-boxes. Typically, the N-boxes are separated by multiples of 0.5 helical turns. Consider the following table (based on Yu 1997¹²⁵, FIG. 1):

| Gene | Sequence | Dist.* |
|---|---|---|
| Murine | CTTCCTCCTGGGCGCGCTCTCGAGTGCGCGCTCGGAAG | 26 bp |
| Laminin B2 | | 3.0 HT |
| Human | TTTCCGCTGCATCCAGACTTCCT | 11 bp |
| type IV | | 1.5 HT |
| collagenase | | |
| Human CD4 | AGGAGCCTTGCCATCGGGCTTCCT | 12 bp |
| | | 1.5 HT |
| Murine CD4 | AGGAGCCTCACGACCAGGCTTCCT | 12 bp |
| | | 1.5 HT |
| Murine | CGGAAGTCCCGCCCATCTTGCTCAGCCTGTTCCCGGAAG | 27 bp |
| COX Vb | | 3.0 |
| Murine | CTTCCGGTTGCGGGCCCCGTTCTTCCG | 15 bp |
| COX IV | | 2.0 HT |
| Ad2-ML | CGTCCTCACTCTCTTCCG | 6 bp |
| | | 1.0 HT |
| Helical turns | \| \| \| \| \| \| \| | |
| | 0 0.5 1.0 1.5 2.0 2.5 3.0 | |

*Distance measure in bp (base pair) or HT (helical turns).

The 1.0, 2.5 and 1.5 helical turns separating the HSL N-boxes pairs is consistent with characteristic GABP heterotetramer binding.

It is interesting to note that the HSL testis-specific promoter also includes two N-boxes separated by 11 bp or 1.5 helical turns (Blaise 1999¹²⁶)

Many “TATA-less” promoters bind GABP to an N-box in their initiator element. Specifically, HSL is a TATA-less gene.

Three N-boxes on the HSL gene, (+35, +41) in exon B and (+964, +970), (+1110, +1116) in intron B are conserved in the mouse HSL gene (see sequence U69543 in Talmud 1998¹²⁷).

The Swiss mouse embryo 3T3-L1 fibroblasts can differentiate into adipocyte-like cells. The undifferentiated cells contain a very low level of HSL activity. Differentiated adipocyte-like cells show a 19-fold increase in HSL activity (Kawamura 1981¹²⁸)

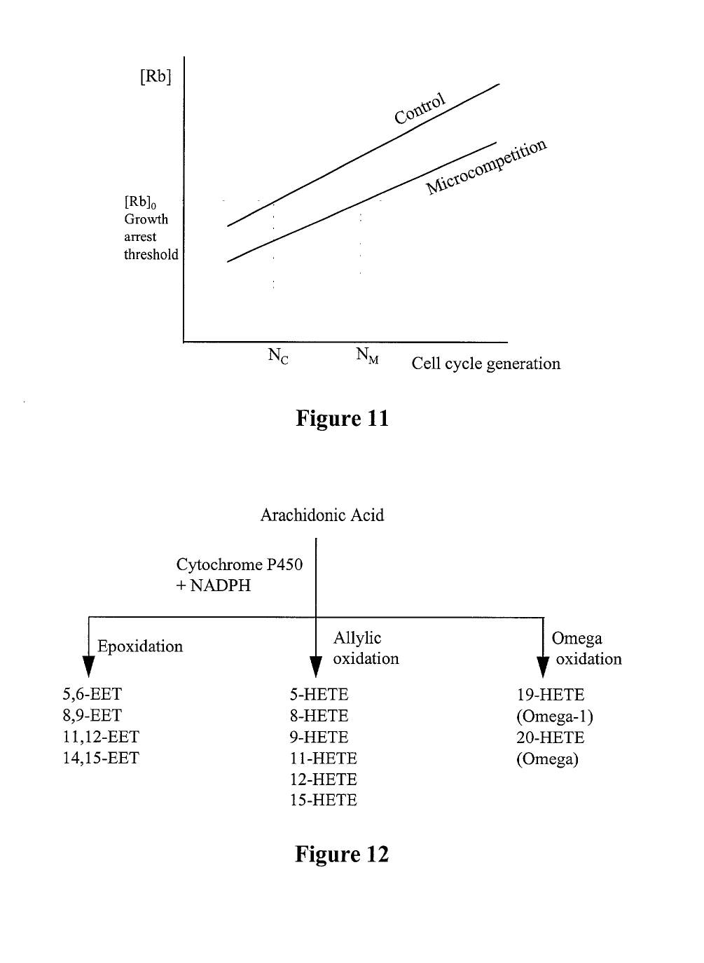

Consider transfection studies. 3T3-L1 preadipocytes were induced to differentiate by incubation with insulin (10 μg/ml), dexamethasone (10 nM), and iBuMeXan (0.5 mM) for 8 consecutive days following cell confluency. HSL mRNA was measured in undifferentiated confluent controls and differentiated 3T3-L1 cells transfected with the ZIPNeo vector. Although differentiated 3T3-L1 cells usually show significance HSL activity, the 3T3-L1 differentiated cells transfected with ZIPNeo showed decreased HSL mRNA (Gordeladze 1997¹²⁹, FIG. 11 left). ZIPNeo carries the Moloney murine leukemia virus LTR which binds GABP. Microcompetition between the viral LTR and HSL promoter leads to reduced expression of the HSL gene.

### C. Clinical Effects of Microcompetition—Cancer

1. Effect of Microcompetition on Cell Proliferation and Differentiation

The current paradigm holds that, in vivo, viral proteins are the mediators of host cell manipulation. Consider, as examples, the extensive research published on the SV40 large T antigen, Epstein-Barr virus BRLF1 protein, papilomavirus type 16 E6 or E7 oncoproteins or adenovirus E1A. The possibility of host cell manipulation independent of viral protein is ignored.

This paradigm is so ingrained that even when protein-independent manipulation presents itself in the lab, the investigators disregard its significance. Consider the following studies as examples. Each study uses two types of plasmids. One plasmid includes a gene of interest, cellular Rb or viral T antigen. The other plasmid includes the neomycin-resistant (Neo) gene only under the control of a viral promoter. This plasmid is regarded “empty,” and is, therefore, used as control. All three studies report results showing a significant effect of the “empty” plasmid on cell cycle, increased proliferation and reduced differentiation. However, none of these studies includes any reference to these results. The results are completely ignored.

A possible exception might be integration of viral DNA into cellular genome. Such integration results in mutations, deletions or methylation of in host cell DNA. However, even this manipulation of cellular function is mediated, in many cases, by viral proteins. Consider, as examples, the HIV-1 IN protein, or the retrovirus integrase which mediate viral integration.

2. Microcompetition Stimulates Proliferation

HuH-7 human hepatoma cells were transfected with pBARB, a plasmid in which the β-actin promoter regulates the expression of Rb gene and the simian virus (SV40) promoter regulates the expression of the neomycin-resistant (neo) gene. The cells were also transfected with the pSV-neo plasmid, which only includes the SV40 promoter and the neo gene. Since pSV-neo does not include the β-actin promoter and the Rb gene, it was regarded “empty” and was used as control. The cells were incubated in the chemical defined medium IS-RPMI with 5% FBS or serum free IS-RPMI. The number of viable cells were counted at various times. Rb transfection resulted in reduced cell proliferation at day 6 relative to non transfected “wild” type HuH-7 cells. Transfection of the “empty” vector resulted in increased proliferation. The “empty” vector includes the SV40 promoter. The SV40 promoter binds GABP. Microcompetition between the viral promoter and cellular genes leads to increased proliferation (see the identity of the cellular genes in the pathogenesis section below).

3. Microcompetition Inhibits Differentiation

HSV-neo is a plasmid that expresses the neomycin-resistant gene under the control of murine Harvey sarcoma virus long terminal repeat (LTR) (Armelin 1984¹³⁰). ZIPNeo expresses the neomycin-resistant gene under the control of the Moloney murine leukemia virus long terminal (Cepko 1984¹³¹). PVU0 carries an intact early region of the SV40 genome which expresses the SV40 large tumor antigen and SV40 small tumor antigen (Higgins 1996¹³²). The murine 3T3-L1 preadipocytes were transfected with PVU0. The cells were also transfected with HSV-neo and ZIPNeo as “empty” controls. Following transfection, the cells were cultured under differrentiation inducing conditions. Glycerophosphate dehydrogenase (GPD) activity was measured as a marker of differentiation. The results are presented in the following table (Higgins 1996¹³³, Table 1, first four lines).

| | | GPD activity |
|---|---|---|
| Vector | Cell line | (U/mg of protein) |
| None | L1 | 2,063 1,599 |
| HSV-neo | L1-HNeo | 1,519 1,133 |
| ZIPNeo | L1-ZNeo | 1,155 1,123 |
| PVU0 | L1-PVU0 | 47,25 |

Transfection of PVU0 and expression of the large and small T antigens resulted in a statistical significant decrease in GPD activity. However, transfection of the “empty” vectors, HSV-neo and ZIPNeo, although less than PVU0, also reduced GPD activity. In a t-test, assuming unequal variances, the p-value for the difference between the HSV-neo vector and no vector is 0.118, and the p-value for the difference between ZIPNeo and no vector is 0.103. Given that the sample includes only two observations, a p-value around 10% for vectors carring two different LTRs indicates a trend. Both the murine Harvey sarcoma virus LTR and the Moloney murine leukemia virus LTR bind GABP. Microcompetition between the viral LTR and the 3T3-L1 preadipocyte GABP genes regulating cell cycle leads to the reduced differentiation, indicated by the reduced GPD activity.

The wild-type early region of SV40 was inserted into the “empty” ZIPNeo plasmid (same plasmid as in Higgins 1996, see above). The new plasmid is called the “wild-type” (WT). WT expresses the SV40 large T antigen. 3T3-F442A preadipocytes were transfected with either WT or ZIPNeo. Accumulation of triglyceride, assayed by oil red staining, was used as a marker of differentiation. Seven days postconfluence, the staining of cells was recorded. Transfection with WT, the vector expressing SV40 large T antigen, reduced differentiation. However, transfection with the “empty” vector, although less than WT, also reduced differentiation (Cherington 1988¹³⁴, FIGS. 4 A, B and C).

ZIPNeo utilizes the Moloney murine leukemia virus long terminal (LTR). The LTR binds GABP. Microcompetition between the viral LTR and the cellular genes regulating cell cycle leads to the reduced differentiation, indicated by reduced accumulation of triglyceride.

4. Pathogenesis

a) Rb

Cell cycle starts with a growth period (G1). Prior to a time in late G1, called R-point, the cell “decides” whether to divide or exit cell cycle. An exit results in growth arrest, differentiation, senescence or apoptosis. A decision to divide leads to a series of orderly processes starting with DNA synthsis (S), a second growth period (G2), mitosis and cell division (M), and a return to G1. As cells progress through the cell cycle, pRb undergoes a series of phosphorylation events. In G0 and early G1, pRb is primarily unphosphorylated. As cells approach the G1/S boundary, pRb becomes phosphorylated by cyclin D/CDK4 and cyclin D/CDK6 kinases represented by a higher-molecular-weight species of pRb. Further phosphorylation by cyclin E/CDK2 kinase occurs in late G1. Phosphorylation is progressive and continuous throughout the S phase and into G2/M. Phosphopeptide analysis demonstrated that pRb is phosphorylated on more than a dozen distinct serine or threonine residues throughout the cell cycle (Sellers 1997¹³⁵).

Let un-pRb denote the unphosphorylated form of pRb, hypo-pRb, the hypo or under phosphorylated form of pRb and hyper-pRb, the hyperphosphorylated form of pRb. Un/hypo-pRb denotes the set of all pRb either un or hypophosphorylated.

Accumulation of un/hypo-pRb leads to G1 arrest. This hypothesis is supported by many observations. For instance, E2F is a transcription factor associated with cell proliferation. Un/hypo-, but not hyper-pRb, binds and inactivates E2F. Cellular introduction of viral oncogenes such as HPV16 E7, adenovirus E1A, and simian virus 40 (SV40) large T antigen result in cell proliferation. These viral oncogenes bind un/hypo-, but not hyper-pRb and disable its suppresive capacity. The human osteogenic sarcoma cell line SAOS-2 lacks full length nuclear pRb protein. Transfection of the Rb gene in these cells result in G0/G1 growth arrest. Co-transfection of cyclin D2, E or A resulted in pRb phosphorylation and a release from G0/G1 arrest (Dou 1998¹³⁶)

The following studies show increased Rb transcription in arrested or differentiated cells.

Murine erythroleukemia (MEL) cells are virus-transformed erythroid precursor cells which can be induced to differentiate by a variety of chemicals. MEL cells were induced to differentiate with dimethyl sulfoxide (DMSO) or hexamethylene bisacetamide (HMBA). Expression of globin was used as a marker of differentiation. The cells showed a 11- and 7-fold increase in Rb mRNA following DMSO and HMBA treatment, respectively, with maxim induction on day three of induction (Coppola 1990¹³⁷, FIG. 1). This increase preceded the accumulation of globin mRNA, the marker of differentiation. The peak in Rb mRNA occurred simultaneously with growth arrest and terminal differentiation. Another cell line, S2 myoblasts derived from C3H10T1/2 mouse embryonic by 5-azacytidine treatment, was induced to differentiate by depletion of mitogens from the medium. Expression of α-actin, a muscle specific gene, was used as a marker of differentiation. Seven to twelve hours following feeding with 2% hours serum (low mitogen conditions), the cells showed an increase in pRb mRNA. The increased continued over the next 48 hours (Ibid, FIG. 2). The study estimates a 10-fold Rb mRNA induction. The increase was accompanied by an increase in α-actin expression. In a B cell line, A20 and a pre-B cell line 300-18, Rb gene is expressed at very low levels compared to actin. In three plasmacytoma lines, representing very late stages of B cell differentiation, Rb mRNA was 8-fold higher. The results are consistent with the MEL and S2 cells. All cell lines showed an increase in steady-state Rb mRNA in late stages of differentiation, which is maintained in dividing cells. Based on these observations, Coppola, et al., concluded that in all three lineages (erythroid, muscle, and B-cell) differentiation is associated with increased Rb mRNA.

An enriched epithelial cell population from 20-day fetal rat lungs was immortalized with a replication-defective retrovirus encoding a temperature-sensitive SV40 T antigen (T Ag). One cell line, designated 20-3, maintained a tight epithelial-like morphology. At the permissive temperature (33° C.), 20-3 cells grow with a doubling time of 21 h. At the non-permissive temperature (40° C.), doubling time increased to more than 80 h (Levine 1998¹³⁸, FIG. 4a). 20-3 cells, incubated at the permissive temperature (33° C.) show almost no Rb mRNA. At the non-permissive temperature (40° C.) the cells show more than 100-fold increase in Rb mRNA (Ibid, FIG. 6b). The increase is significant at 24 h after temperature shift-up and peaks at 48-72 h (Ibid, FIG. 7a). Terminally differentiated and growth arrested alveolar type 1 cells are first observed at day 20-21 of gestation. Prior to this time the lung shows active growth and cell proliferation. Total RNA was isolated from 17- and 21-day fetal lungs and assayed for Rb mRNA. The results show a 2.5-fold increase in Rb mRNA during this period relative to control gene EFTu.

P19 embryonal carcinoma cells were induced to differentiate into neuroectoderm with retinoic acid (RA). Undifferentiated cells show very low levels of Rb mRNA and protein. Twenty four hours following RA exposure, the cells showed a marked increase in Rb expression with mRNA levels increasing 15-fold by 4-6 days (Slack 1993¹³⁹, FIG. 2). RAC65 is a mutant clone of P19 cells that fails to differentiate. The cells contain a truncated RARα receptor. Following RA exposure, the cells showed no increase in Rb mRNA (Ibid, FIG. 3). P19 cells transfected with RB-CAT, a reporter gene driven by the Rb promoter, expressed CAT with kinetics similar to the Rb gene (Ibid, FIGS. 5b, 6). The post-mitotic neurons developed in RA-treated cultures contained only the hypophosphorylated form of pRb (Ibid, FIGS. 7, 8). Based on these observations, Slack, et al., concluded that the increased Rb expression associated with cell differentiation appears to result from enhanced transcription.

DS 19/Sc9 is a MEL cell line. HMBA treatment of DS 19/Sc9 cells in G1 prolonged the next G1 (Richon 1992¹⁴⁰, FIG. 2A). The cells emereged from the prolonged G1, progressed through cell cycle for at least another two to five generation (cycle time of 10 to 12 h), and permanently arrested in G1/G0 expressing characteristic of terminal erythroid differentiation. Over 90% of the DS19/Sc9 cells became irreversibly committed to differentiate by 48 h of culture with HMBA. Protein extracts prepared from asynchronous cultures induced with HMBA demonstrated a 2-to 3-fold increase in total amount of pRb. There was no change in proportions of hypo- to hyper-pRb (Ibid, FIG. 4A). An increase in the level of total pRb was detected as early as 24 h after onset of culture with HMBA, and pRb increased as the number of cells recruited to terminal differentiation increased through 100 h of cultured (Ibid, FIG. 4A). HMBA-induced an increase in pRb in all phases of the cell cycle. No change in Rb protein level was detected in DS19/Sc9 cultured without HMBA. The increase in pRb in cells cultured with HMBA was accompanied by an increase in the level of Rb mRNA. An 3.6-fold increase in Rb transcription was observed. There was no change in mRNA stability. DS 19/VCR-C is a vincristine-resistant variant of the parental DS19/Sc9 with accelerated rate of differentiation. HMBA treatment of DS19/VCR-C showed a more prolonged G1 arrest and a higher percentage of cell committed to terminal differentiation compared to DS19/Sc9. During G1 arrest, DS19/VCR-C also showed more hypo-pRb compared to DS19/Sc9. In HMBA-induced MEL cells, every cell division increased the absolute amount of pRb protein, whereas the degree of phosphorylation continues to fluctuate through cell cycle progression. This increase was accompanied by an increase in mRNA resulting from increased rate of transcription. Based on these observations, Richon, et al., proposes the following model. An inducer increases Rb transcription resulting in higher hypo- and total-pRb concentration. The increase in hypo-pRb prolongs G1. However, the initial increase in hypo-pRb is most likely not sufficient for permanent G1 arrest. Therefore, cells reenter cell cycle for a few more generations. While cells continue to divide, the increased rate of transcription results in hypo-pRb accumulation. When a critical hypo-pRb concentration is reached, the cell irreversibly commits to terminal differentiation. This model describe the determination of the commitment to differentiate as a stochastic process with progressive increases in the probability of G1/G0 arrest and differentiation established through successive cell divisions.

Many studies report a relationship between Rb phosphorylation, cell cycle arrest and differentiation. These studies use the reduced gel mobility of hyper-pRb relative to un/hypo-pRb to show protein phosphorylation or dephosphorylation. Since these studies are interested in the transition between the two states, they do not report changes in total concentration of each kind of pRb. Specifically, they do not use densitometry measures. However, in some cases, eye inspection of the blots can provide valuable information. Consider the following study. Actively growing LS174T colon cancer cells, which constitutively express Rb protein, were induced to differentiate with sodium butyrate. Three days following exposure, a lower molecular weight, or unphosphorylated pRb molecules became visible. After the fourth day of treatment, when a significant growth inhibition was observed, the unphosphorylated species were predominant (Schwartz 1998¹⁴¹, FIG. 5). A careful inspection of the blots in FIG. 5 suggests that the concentration of hypo-pRb at day 4 (lane 6) is higher than the initial concentration of hyper-pRb (lane 1 and 2). Even if we assume that dephosphorylation of hyper-pRb produces a hypo-pRb species associated with growth arrest (and not protein degradation), the difference in total concentrations at day 0 and day 4 indicates a potential need for increased transcription (an increase in mRNA stability, or rate of translation is also possible).

The transcription of the Rb gene increases with growth arrest and differentiation. Rb is a GABP stimulated gene. Microcompetition decreases Rb transcription. Decreased Rb expression increases the probability of developing cancer.

b) BRCA1

Transcription or translation inactivation of the BRCA1 gene increases cell proliferation.

Normal mammary ephithelial cells and MCF-7 breast cancer cells were treated with unmodified 18 base deoxyribonucleotides complementary to the BRCA1 translational initition site. The anti-BRCAI oligonucleotides decreased BRCA1 mRNA by 70-90% compared to control oligonucleotides (Thompson 1995¹⁴² , FIG. 6). The anti-BRCA1 treated cells showed accelerated proliferation rate (Ibid, FIG. 4a,c).

NIH3T3 cells were transfected with a vector expressing BRCA1 antisense RNA resulting in reduced expression of endogenous BRCA1 protein. The transfected cells, unlike parental and sense transfectants, showed accelerated growth rate, anchorage independent growth and tumorigenicity in nude mice (Rao 1996¹⁴³, FIG. 4).

Retroviral transfer of wild-type BRCA1 gene to breast and ovarian cancer cell lines inhibited growth in vitro. Transfection of wild-type BRCA1 also inhibited development of MCF-7 tumors in nude mice. Peritoneal treatement with retroviral vector expressing wild-type BRCA1 inhibited tumor growth and increased survival among mice with established MCF-7 tumors (Hold 1996¹⁴⁴). A phase I clinical study with gene transfer of BRCA1 to 12 patients with extensive metastatic cancer showed stable disease for 4-16 weeks in eight patients, tumor reduction in three patients and radiographic shrinkage of measurable disease in one patient (Tait 1997¹⁴⁵).

Reduced expression of BRCA1 resulted in increased cell proliferation. Increased expression of BRCA1 resulted in reduced tumor development.

The majority of familial breast cancer and ovarian cancer cases result from germline mutations in the BRCA1 gene. However, sporadic breast cancer is different. Many studies showed decreased BRCA1 transcription in sporadic breast tumors (Russel 2000¹⁴⁶, Rio 1999¹⁴⁷, Rice 1998¹⁴⁸, Magdinier 1998¹⁴⁹, Ozcelik 1998¹⁵⁰, Thompson 1995¹⁵¹). The decrease intensifies with tumor progression. The cause of the decreased transcription is unknown. Two possible causes, somatic mutations and promoter methylation, do not seem to provide an explanation. Somatic mutations of the BRCA1 gene are rare in sporadic breast and ovarian tumors (Russel 2000¹⁵², Rio 1999¹⁵³, Futreal 1994¹⁵⁴, Merajver 1995¹⁵⁵), and methylation of the BRCA1 promoter was demonstrated in only a small percentage of sporadic breast cancer samples (Catteau 1999¹⁵⁶, Magdinier 1998¹⁵⁷, Rice 1998¹⁵⁸, Dobrovic 1997¹⁵⁹). The majority of breast and ovarian tumors show neither somatic mutations nor promoter methylation.

BRCA1 is a GABP stimulated gene. Microcompetition decreases BRCA1 transcription. Decreased BRCA1 expression increases the probability of developing breast and ovarian cancer.

c) Fas

Cell population is determined by balancing cell growth and cell death. Programmed cell death, or apoptosis, is the final step in a series of morphological and biochemical events. Fas antigen is a 48-kDA cell surface receptor homologous to the tumor necrosis factor (TNF) family of transmembrane proteins. Fas ligation by the Fas ligand, or by antibodies, triggers rapid cell apoptosis.

The Fas induced apoptosis was initially identified in the immune system. Ligation of Fas induced apoptosis in activated T cells, B cells, and natural killer cells. In addition, Fas was identified in many epithelial cells. Although the role of Fas in non-lymphoid tissues is not completely understood, maintenance of normal cell turnover and removal of potentially oncogenic cells have been suggested. Consider, as example, the epithelial layer of colonic mucosa. These cells show rapid rate of cell turnover and high expression of Fas. It is conceivable that the high rate of colonocytes removal is Fas induced.

Germline mutations in Fas gene is associated with spontaneous development of plasmcytoid tumor in lpr mice (Davidson 1998¹⁶⁰) and neoplasms in two autoimmune lymphoproliferative syndorme (ALPS) patients (Drappa 1996¹⁶¹).

Many studies showed progressive reduction in Fas expression in many cancers. Consider, Keane, et al., (1996¹⁶²) results in breast carcinomas, Gratas, et al., (1998¹⁶³) results in esophageal carcinomas, Strand, et al, (1996¹⁶⁴) results in hepatocellular carcinomas, Moller, et al., (1994¹⁶⁵) results in colon carcinomas and Leithauser, et al., (1993¹⁶⁶) results in lung carcinomas. The reduce Fas expression results from reduced transcription of the Fas gene. Consider the observations in Das, et al., (2000¹⁶⁷) showing reduced Fas transcription in ovarian, cervical and endometrical carcinoma tissues and in four ovarian and three cervical carcinoma cell lines. Also consider the results in Butler, et al., (1998¹⁶⁸) demonstrating reduced Fas transcription in colon tumors, and in Keane, et al., (1996¹⁶⁹) showing reduced Fas mRNA levels in six out of seven breast cancer cell lines. As in the case of BRCA1 gene, the cause of the decreased transcription is unknown. The same two possible causes, somatic mutations and promoter methylation, also fail to exmplain the Fas transcription reduction. Allelic loss or somatic mutations of the Fas gene are rare (Bertoni 2000¹⁷⁰, Lee 1999A¹⁷¹, Lee 1999B ¹⁷², Shin 1999¹⁷³, Butler 1998¹⁷⁴), and no methylation was found in the Fas promoter (Butler 2000¹⁷⁵). The majority of carcinomas show no somatic mutations or promoter methylation in the Fas gene.

Fas is a GABP stimulated gene. Microcompetition decreases Fas transcription. Decreased Fas expression increases the probability of developing cancer.

5. Signaling

GABP kinase agents phosphorylate GABP, increase Rb, BRAC1 and Fas transcription and induce cell cycle arrest and differentiation.

Consider a study with a constitutive active MAP kinase kinase 1 (MEK1). AU565 breast carcinoma cells were transiently transfected with a constitutively active MEK1 mutant or a control vector. Expression of the consitiutively active MEK1 resulted in a significant increase in ERK activity as determined by use of an antibody against phosphorylated ERK (Lessor 1998¹⁷⁶, FIGS. 6A, B). Oil Red O staining was used as a measure of cell differentiation. 53.6% of cells trasfected with the consititutively activated MEK1 vector were Oil Red O positive. In contrast, only 20.8% of the cells transfected with the control vector were 20.8% Oil Red O positive. Based on these observations, Lessor, et al., concluded that constitute activation of the MEK/ERK pathway in AU565 cells is sufficient to mediate differentiation.

Consider a study with heregulinβ1 (HRGβ1). AU565 breast carcinoma cells were treated with 10 ng/ml HRGβ1 for 7 days. The treatment increased ERK activity 4-fold after 10 min. The initial increase dropped to control levels by 15 min. Following the drop, a second sustained increase in activity was observed for 105 min (Lessor 1998¹⁷⁷, FIG. 1). HRGβ1 treatment decreased cell number by 56% as compared to the non treated controls (Ibid, FIG. 4). Addition of 0-10 μM PD98059, a specific MEK inhibitor (see above) resulted in a dose-dependent reversal of HRGβ1-induced cell growth arrest (Ibid, FIG. 4). Pretreatment with PD98059 also inhibited HRGβ1-induced differentiation in a dose-dependent manner (Ibid, FIG. 5), with 10 μM PD98059 completely blocking the HRGβ1-induced differentiation. Based on these observations Lessor, et al., concluded that sustained activation of the MEK/ERK pathway is both essential and sufficient for HRGβ1-induced differentiation of AU565 cells. Exposure to low doses of HRGβ1 (0.01 ng/ml) induced a 7-fold transient 5 min peak in ERK activation which dropped to control levels by 90 min. This dose showed no sustained activation (FIG. 1). The 0.01 ng/ml HRGβ1 treatement results in cell proliferation.

Consider a study with phorbol ester (TPA). ML-1, human myeloblastic leukemic cells, were treated with 0.3 ng/ml TPA. As a result, ERK2 activity increased with a 6- and 4-fold induction at 1 and 3 h, respectively. Thereafter, the activity decreased to below basal levels (He 1999¹⁷⁸, FIG. 1A). The time-dependent ERK2 activation was further illustrated by a shift to a slower-migrating form of ERK2, representing the phosphorylated ERK2 (Ibid, FIG. 1B). ML-1 cells treated with 0.3 ng/ml TPA for 3 days, followed by and additional 3 days in culture after removal of TPA, ceased to proliferate and displayed morphological features typical of monocytes/macrophages (Ibid, FIG. 6c). Exposure to PD98059, the MEK inhibitor, led to a 2- and 10-fold reduction in TPA-activated ERK2 activity at 1 and 3 h, receptively (Ibid, FIG. 3). Cells treated simultaneously with 10 μM PD98059 and 0.3 ng/ml TPA continued to proliferate and exhibited morphology of undifferentiated cells (Ibid, FIGS. 6A, D). Based on these observations, He, et al., concluded that activation of the MEK/ERK signaling pathway is necessary for TPA-induced mononuclear cell differentiation.

Consider a study with transforming growth factor β1 (TGFβ1). An enriched epithelial cell population from 20-day fetal rat lungs was immortalized with a replication-defective retrovirus encoding a temperature-sensitive SV40 T antigen (T Ag). One cell line, designated 20-3, maintained a tight epithelial-like morphology. At the permissive temperature (33° C.), 20-3 cells grow with a doubling time of 21 h. At the non-permissive temperature (40° C.), doubling time increased to more than 80 h (Levine 1998¹⁷⁹, FIG. 4a). The labeling index is a function of [³H]thymidine incorporation in DNA, and therefore correlates with cell replication. Treatment of 20-3 cells with 5 ng/ml TFGβ1 for 72 h decreased the labeling index to 80% at the permissive temperature (33° C.) and to less than 5% at the non-permissive temperature (40° C.) (Ibid, FIG. 5c). Treated cells cultured at the non-permissive temperature for 72 h and then shifted to the permissive temperature for additional 24 h showed an index below 10%. The low labeling index reveals the extensive terminal growth arrest occurred during the non-permissive temperature period. Treatment with the GABP kinase agent TFGβ1 resulted in reduced replication of the epithelial cells in both permissive and non-permissive temperatures.

6. Viruses in Cancer

Many studies report detection of viral genomes in human tumors. The following table summarizes some of these reports.

| Virus | Cancer |
|---|---|
| Epstein-Bar virus (EBV) | Burkitt's lymphoma (BL) |
| | Nasopharyngeal carcinoma (NPC) |
| | Hodgkin's disease |
| | Some T-cell lyphomas |
| | Polymorphic B cell lymphomas |
| | B-cell lymphoproliferation in |
| | immunosuppressed individuals |
| | Breast cancer |
| SV40 | Brain tumors |
| | Osteosacromas |
| | Mesotheliomas |
| HIV | Brest cancer |
| Human T cell lymphotrophic | Adult T-cell leukemia |
| virus-I (HTLV-I) | |
| Human papilloma virus | Anogenital cancers |
| (HPV) | |
| | Skin cancers |
| | Oral cancers |
| Hepatitis B virus (HBV) | Hepatocellular carcinoma |
| Hepatitis C virus (HCV) | Hepatocellular carcinoma |
| Human herpes virus 8 | Kaposi's sarcoma, |
| (HHV8, KSHV) | |
| | Body cavity lymphoma |

See also recent reviews on human tumor viruses, Butel 2000¹⁸⁰, zur Hansen 1999¹⁸¹, Hoppe-Seyler 1999¹⁸². On EBV and breast cancer see Bonnet 1999¹⁸³, Labrecque 1995¹⁸⁵, and the editorial by Magrath and Bhatia 1999¹⁸⁵ . On HIV and breast cancer see Rakowicz-Szulczynska 1998¹⁸⁶.

EBV, SV40, HIV and HTLV-I are GABP viruses. Microcompetition between a GABP virus and cellular genes causes cancer. An interesting aspect of microcompetition is its ability to explain how viral infection can cause cancer independent of proto-oncogene expression or viral integration into host DNA.

D. Clinical effects of microcompetition—Atherosclerosis

1. Motility

a) Introduction

The extracellular matrix (ECM) comprises of a several proteins, including collagens, fibronectin, laminins and proteoglycans, assembled into a network structure. Cells bind to ECM proteins through transmembrane-surface receptors. The receptors include integrins, cadherins, immunoglobulins, selectins and proteoglycans. The cadherins and selectins are mostly involved in cell-cell adhesion. The integrins and proteoglycans are mostly involved in cell-ECM binding. Cell-adhesion molecules connect external ligands and the cell cytoskeleton and participate in signal-transaction.

Cell is said to show motility if it changes position over time. A change of position of the entire cell is called migration. A change in position of any part of the cell periphery is called projection. The two processes share common features, such as, polarization, cytoskeletal reorganization and formation of new cell-ECM adhesion points.

The first phase in cell migration is polarisation. During polarisation the cell creates clear “front-back” asymmetry in which actin and cell-surface receptors accumulate at the front of the cell. The second phase of migration is protrusion of the plasma membrane from the front of the cell in a form of fine, tubular structures called filapodia, or broad, flat membrane sheet called lamellipodium. The third phase is establishing new ECM-cell points of contact. The binding prevents retraction of the newly extended membrane and provides “grip” for the tractional force required for cell movement. The two final stages of cell migration are flux of intracellular organelles into newly extended sections of the cell, and retraction of, or breaking off, the trailing edge. The result of this process is directional movement of cell body (Sanserson 1999¹⁸⁷)

A simple characterization of direction of movement is change in distance relative to a reference point in space. Let circulation define such a reference point. Movement of cells out, or away from circulation, will be called forward motility. Diapedesis of monocytes to enter the intima (also called migration, emigration or transmigration) is an example of forward motility. Movement of macrophages deeper into the intima is another example of forward motility. Movement of cells toward, or into circulation, will be called backward motility. Reverse transendothelial migration is an example of backward motility.

b) P-selectin-, β₂ integrin-, α₄integrin-propelled Forward Motility

The first part discusses the relation between p-selectin, β₂ integrin and α₄-integrin and motility without reference to direction. The direction issue is covered further on.

Leukocytes migration from blood into tissue starts with crossing the endothelium. This phase is called transedothelial migration, transmigration or emigration. Transmigration involves multiple steps, including rolling of leukocytes along the endothelium, firm adhesion of leukocytes to endothelium called margination, and movement of leukocytes through endothelial intercellular junctions. In this process P-selectin mediates rolling of leukocytes on the endothelium (Dore 1993¹⁸⁸). An increase in endothelial surface expression of P-selectin increases leukocytes rolling and transmigration.

Many studies demonstrated the role of CD18 (CD11a/CD18, CD11b/CD18, CD11 c/CD18) and VLA-4 (α₄β₁, CD49d/CD29) in this process of transedothelial migration (Shang 1998A¹⁸⁹, Shang 1998B¹⁹⁰, Meerschaert 1995¹⁹¹, Meerschaert 1994¹⁹², Chuluyan 1993¹⁹³, Kavanaugh 1991¹⁹⁴). The two studies by Shang, et al., (1998A, 1998B) also showed that these molecules participate in forward motility through a barrier of human synovial fibroblasts (HSF).

CD18 and α₄ also participate in motility inside the intima. Consider the following studies.

To test the effect of α₄ expression on cell motility, α₄ was expressed in a Chinese hamster ovary (CHO) cell line deficient in α₅β₁ integrin (CHO B2). The parental as deficient CHO B2 cells were unable to adhere, spread or migrate on a surface coated with 10 μg/ml mouse cellular fibronectin. Expression of α₄β₁ integrin in the CHO B2 cells enabled the cells to adhere, spread and migrate on the fibronectin coated surface (Wu 1995¹⁹⁵).

To test the effect of CD18 on cell motility, neutrophils were stimulated with 0.5×10⁻⁸ M fMLP. The stimulation increased random motility through a three-dimensional collagen type I gels (0.1 to 1.0 mg/mL). In a 0.4 mg/mL collagen gel, antibodies against CD18 (anti-CD18) decreased motility of stimulated neutrophils by 70% (Saltzman 1999¹⁹⁶). Based on these observations Saltzman, et al., concluded that under conditions of high hydration, or when fiber density is relatively low, neutrophils migration through collagen gels is CD18-dependent.

To test the effect of CD18 on cell motility, another study stimulated neutrophils with 10⁻⁸ M fMLP for 10 min. On unstimulated cells, CD18 was randomly distributed on the nonvillous planar cell body. Stimulation of the round, smooth neutrophils induced a front-tail polarity, i.e., a ruffled frontal pole and contracted rear end with a distinct tail knob at the posterior pole. Moreover, immunogold-labeling and backscattered electron images detected an 4-fold increase in CD18 surface membrane concentration compared to unstimulated cells. The immonogold-labled CD18 accumulated mainly on ruffled plasma membrane at the frontal pole of polar neutrophils. The contracted rear end showed few colloidal gold particles (Fernandez-Segura 1996¹⁹⁷). Based on these observations, Fernandez-Segura, et al., concluded that CD18 may participate in the locomotion of neutrophils.

A third study stimulated rat mesentery with platelet-activating factor (PAF; 10⁻⁷ M). At 30-40 min of the chemotactic stimulation, numerous polymorphonuclear leukocytes (PMNs), predominantly neutrophils and monocytes/macrophages, were observed migrating further in the extravascular tissue. Immunofluorescence flow cytometry revealed a 3-fold increase in CD18 expression on extravasated PMNs compared with blood PMNs. Intravital time-lapse videomicroscopy was used to analyze migration velocity of activated PMNs. Median migration velocity in response to PAF stimulation was 15.5±4.5 μm/min (mean±SD). Treatment with two different antibodies against CD18 significantly reduced migration velocity by 17% (mAb CL26) and 22% (mAb WT.3) (Werr 1998¹⁹⁸). Based on these in vivo observations Werr, et al, concluded that CD18 participates in extravascular PMN locomotion.

Since the extracellular matrix (ECM) contains fibronection and collagen, the observations in Wu (1995¹⁹⁹) and Saltzman (1999²⁰⁰) above are consistent with intimal α₄ integrin- and CD18-propelled leukocyte motility. Moreover, the morphological changes reported in Femandez-Segura (1996) and the extravascular CD18-propelled leukocyte motility reported in Werr (1998) support such a mechanism.

The first segment of leukocytes forward motility, transedothelial transmigration, is α₄ integrin- and CD18-propelled. From the basal side of the endothelium, leukocytes continue their forward motility into the intima until they reach a certain depth. Werr, et al., (1998) showed that forward motility in the extravascular space is CD18-propelled. Since the intima is sandwiched between the endothelium and the extravascular space, forward motility in the intimal segment is, most likely, CD18-propelled.

(See more on direction control, or “cell turning,” below)

c) TF-propelled Backward Motility

As above, the first part discusses the relation between TF and motility without reference to direction and the direction issue is covered further on. TF expression induces cell spreading. Consider the following studies.

The human breast cancer cell line MCF-7 constitutively expresses TF on the cell surface. aMCF-7 is a subline of MCF-7. Muller, et al., (1999²⁰¹) show that adhesion of aMCF-7 cells to surfaces coated with FVIIa or inactivated FVIIa (DEGR-FVIIa) was significantly accelerated during the first 2 h after seeding compared to BSA. In addition, the number of cells adhering to anti-TF IgG was significantly higher than the number of cells adhering to anti-FVII or a control IgG (Ibid, FIG. 6A). Accelerated adhesion and spreading of cells on surfaces coated with anti-TF mAb VIC7 was blocked by recombinant TF variants (sTFI₁₋₂₁₉, sTF₉₇₋₂₁₉) covering the epitope of anti-TF mAb VIC7 (residues 181-214). No effect was seen with sTF₁₋₁₂₂. However, if anti-TF IIID8 (epitope area 1-25) was used for coating, sTF₁₋₁₂₂ blocked accelerated adhesion and spreading of cells. To conclude, Muller, et al, results demonstrate that in vitro-cultured cells, that constitutively express TF on the cell surface, adhere and spread on surfaces coated with both catalytically active and inactive immobilized ligands for TF. Ott, et al., (1998²⁰²) showed that J82 bladder carcinoma cells that constitutively express high levels of TF adhere and spread on surfaces coated with monoclonal antibodies specific for the extracellular domain of TF. The spontaneously transformed endothelial cell line ECV304 or human HUVEC-C endothelial cells also adhered and spread on TF ligand when stimulated with TNFα to induce TF.

In malignant and nonmalignant spreading epithelial cells, TF is localized at the cell surface in close proximity to, or in association with both actin and actin-binding proteins in lamellipodes and microspikes, at ruffled membrane areas and at leading edges. Cellular TF expression at highly dynamic membrane areas suggest an association between TF and elements of the cytoskeleton (Muller 1999²⁰³). Cunningham, et al., (1992²⁰⁴) showed that cells deficient in actin binding protein 280 (ABP-280) have impaired cell motility. Transfection of ABP-280 in these cells restored translocational motility. Ott, et al., (1998²⁰⁵) identified ABP-280 as a ligand for the TF cytoplasmic domain. The study showed that ligation of the TF extracellular domain by either FVIIa or anti-TF resulted in ligation of the TF cytoplasmic domain by ABP-280, reorganization of the subcortical actin network, and expression of specific adhesion contacts different from integrin mediated focal adhesions.

Randolph, et al., (1998²⁰⁶) used an in vitro model consisted of HUVEC grown on reconstituted bovine type I collagen. The reverse transmigration assays used freshly isolated or precultured peripheral blood monoculear cells (PBMC) incubated with endothelium for 1 or 2 hours to allow accumulation of monocytes in the subendothelial collagen. Following initial incubation, the nonmigrated cells were removed by rinsing the cultures. At given intervals a few cultures were processed to enable counting of the cells underneath the endothelium. The remaining cultures were rinsed to remove cells that may have accumulated in the apical compartment by reverse transmigration, and incubation was continued. Let percent reverse transmigration represent the percentage decrease in the number of cells beneath the endothelium, relative to the number of subendothelial cells at 2 hours.

The results showed that mononuclear phagocytes (MP) that enter the subendothelial collagen later exit the cultures by retransversing the endothelium with a t1/2 of 48 hours. The endothelial monolayer remained intact throughout the experiments.

Two MoAbs against TF, VIC7 and HTF-K108, strongly inhibited reverse transmigration for at least 48 hours (Ibid, FIG. 2A). In comparison, 55 other isotype-matched MoAbs tested had little or no effect, specifically, anti-factor VIIa MoAbs IVE4 or IIH2 did not inhibit reverse transmigration (Ibid, FIG. 2C). A direct comparison of the effect of VIC7 relative to IB4, a MoAb against β2 integrin, revealed 78±15% inhibition of reverse transendothelial migration by VIC7 relative to no inhibition by IB4 in the same three experiments (Ibid, FIG. 2B). None of the MoAbs affected total number of live cells in the cultures.

Studies of epitope mapping showed that the epitope for VIC7 included recognition of at least some amino acids between 181-214. Soluble TF inhibited reverse transmigration by 69±2% in eight independent experiments (Ibid, FIG. 4). Only fragment containing amino acid residues carboxyl to residue 202 blocked reverse transmigration effectively (Ibid, FIG. 4). This result agrees well with the location of the epitope for VIC7.

Experiments were conducted to explore the existence of a ligand to TF on the endothelium. Unstimulated HUVEC were added to wells coated with TF or control proteins in the presence or absence of anti-TF MoAb. After 2 hours incubation, endothelial cell adhesion to TF fragments containing amino acid residues 202-219 was greater than their binding to control surfaces or to TF fragments lacking these residues (Ibid, FIG. 8A). Spreading of HUVEC during the first 2 hours was observed on surfaces coated with TF fragments 97-219 or 1-219. Surfaces coated with TF fragment spanning amino acids 1-122 showed much less spreading. These results show that endothelial cells express binding sites for TF, and that the TF residues 202-219 participate in this adhesion.

LPS stimulation increases cell surface TF activity through increased concentration of cell surface TF molecules and increased conversion of TF dimers to monomers. Monocytes and HUVEC were stimulated with LPS. VIC7 recognized a single band of 47 kD in the LPS-stimulated cells, but not in the unstimulated cell extracts (Ibid, FIG. 3). In unstimulated cells TF is self-associated, most likely in the 181-219 region, and, therefore, unavailable for VIC7 binding. LPS stimulation converts the dimers to monomers and exposes the VIC7 binding site. The same region participates in binding to endothelial cells. Since VIC7 inhibits reverse transmigration by competitive binding to the 181-219 region, self-association also inhibits reverse transmigration.

d) Cell Turning

Let CD18, α₄ integrin and TF be called propulsion genes. Since leukocyte forward motility is α₄ integrin- and CD18-propelled, and backward motility is TF-propelled, a signaling system should exist that coordinates expression of the proplusion genes. This system should determine the direction of cell motility.

Forward and backward motility are propelled through mostly different molecules. Antibodies against many molecules participating in forward motility do not inhibit reverse transmigraion. Randolph, et al., (1998²⁰⁷) tested a variety of MoAbs against a list of molecules known to mediate binding between leukocytes and endothelium during apical-to-basal transmigration. Even though MoAbs were shown to access subendothelial antigens, neutralizing MoAbs to E-selectin, vascular cell adhesion molecule-1 (VCAM-1), and platelet/endothelial cell adhesion molecule-1 (PECAM-1) showed no effect on reverse transmigration. Ott, et al., (1998²⁰⁸) showed that a RGD peptide known to block several matrix-binding integrins does not abolish spreading on VIIa (Ibid, FIG. 2A).

On the other hand, antibodies against TF, which participates in backward motility, do not inhibit forward motility. Resting monocytes do not express TF. LPS stimulates the expression of TF on resting monocytes. Randolph, et al., (1998) showed that the TF MoAb VIC7 inhibits adhesion of LPS-stimulated, but not resting, monocytes to unstimulated or TNF-activated HUVEC by 35±7%. However, VIC7 did not inhibit migration of LPS-stimulated monocytes already bound to the apical side of the endothelium. Since circulating monocytes do not express TF, it is reasonable to conclude that TF does not participate in adhesion to the endothelium during forward motility (TF adhesion to the apical side of the endothelium is probably important in backward motility, see below). Since TF also does not participate in the subsequent steps in apical-to-basal transendothelial migration, TF has no role in forward motility.

Ott, et al., (1998²⁰⁹) also noted that J82 cells spreading on TF ligand have a different morphology compared to cells adherent to fibronectin through integrins (Ibid, FIGS. 2A and 2B), which suggests a qualitative differences in the two adhesive events.

Extracellular signal-regulated kinase (ERK) agents are extracellular molecules which transmit a signal resulting in phosphorylation of ERK. See chapter on ERK for examples. GABP kinase agent stimulates GABP•p300 binding. In leukocytes, this binding stimulates transcription of CD18 and α₄, which, in turn, stimulates forward motility. Moreover, the stimulated binding of GABP•p300 represses TF, and therefore, represses backward motility.

A molecules is regarded a chemoattractant if it stimulates leukocytes forward motility. Considering chemoattraction in the framework of propulsion yields an interesting insight. In leukocytes, chemoattraction is the result of ERK phosphorylation. In other words, if a molecule phosphorylates ERK, it should show chemoattraction. FMLP is an example for such a molecule. fMLP is a syntactic compound representing bacterial products. Several studies demonstrated that FMLP binding to its receptor results in phosphorylation of ERK1 and ERK2 (Chang 1999²¹⁰ in rat neutrophils, Yagisawa 1999²¹¹ in human monocytes, Coffer 1998²¹² in human neutrophils). As a GABP kinase agent, fMLP should demonstrate chemoattraction. As expected, Yamada, et al., (1992²¹³) showed that fMLP is a chemoattractant for blood mononuclear cells.

Mildly oxidized LDL (also termed “minimally modified” LDL, and therefore denoted mmLDL) and oxidized LDL (oxLDL) are also GABP kinase agent. Consider the following studies.

Rat vascular smooth muscle cells (VSMC) were exposed to 25 μg/ml of Cu⁺²-oxidized LDL (oxLDL). The results showed a rapid stimulation of both ERK1 and ERK2 with peak activity at 5 min and return to near baseline by 60 min (Kusuhara 1997²¹⁴, FIG. 1). 25 μg/mL of minimally oxidized LDL (mmLDL) caused a smaller increase in ERK activity with a similar time course (Kusuhara et al., call this type of LDL “native LDL.” However, they propose that this type of LDL is actually minimally oxidized. Therefore, we call it mmLDL). The increase in ERK activity relative to 200 nmol/L PMA treatment, was 54.3% for oxLDL and 35.2% for mmLDL. Both oxLDL and mmLDL stimulated ERK activity in a concentration-dependent manner (Ibid, FIG. 3). Human monocytes showed minimal ERK stimulation by either oxLDL or mmLDL (Ibid, FIG. 7A). In contrast, human monocyte-derived macrophages cultured for 7 days showed significant ERK activity in response to oxLDL (Ibid, FIG. 7B) but no response to mmLDL (Ibid, FIG. 7B). Bovine aortic endothelial cells showed no response to either oxLDL or mmLDL (Ibid, FIG. 7C). Based on these observations Kusuhara, et al., concluded ERK activation is cell dependent, degree of oxidation dependent, LDL receptor dependent and that the rapidity of the ERK response to LDL indicates that ERK activation is LDL internalization independent.

Deigner, et al., (1996²¹⁵) reported similar effects of mmLDL and oxLDL on ERKin U-937 macrophage-like cells, Balagopalakrishna, et al., (1997²¹⁶) in aortic smooth muscle cell, Kamanna, et al., (1999²¹⁷) and Bassa, et al., (1998²¹⁸) in mesangial cells.

Both mmLDL and oxLDL are GABP kinase agent, and therefore, chemoattractants. Quinn, et al., (1987²¹⁹) demonstrated that oxLDL is chemoattractant when bound to macrophages in the subendothelial space. However, in contrast to stimulated macrophages, circulating monocytes are not chemoattracted by oxLDL binding. To chemoattract monocytes, oxLDL uses an indirect approach. Subendothelial oxLDL stimulates endothelial cells to produce monocytes chemoattractant (chemotactic) protein-1 (MCP-1, also called RANTES) which is a GABP kinase agent. MCP-1 is released to circulation and binds monocytes. A monocyte bound MCP-1 stimulates CD18 and α₄ integrin, resulting in adhesion to endothelium and transmigration.

Another special example is baterial LPS, a known chemoattractant which is ERK. LPS is direct chemoattractant when bound to its receptor (before internalization), and indirect chemoattractant through stimulation of MCP-1 which is a strong GABP kinase agent.

Oxidative stress decreases the binding of GABP to the N-box and reduces transcription of GABP stimulate genes and increases transcription of GABP suppressed genes (see above).

Oxidative stress reduces the binding of GABPα to the N-bo. XAssume the propulsion genes, TF, CD18 and α₄ integrin, are responsive to oxidative stress exclusively through GABP. GABP stimulates CD18 and α₄ integrin transcription. Reduced binding of GABPα to DNA decreased CD18 and α₄ integrin transcription resulting is diminished forward motility. On the other hand, GABP represses TF transcription, oxidative stress increases TF transcription, stimulating backward motility.

oxLDL increases TF transcription. Consider the following studies.

Exposure of human monocytic THP-1 cells for 10 hours to concentration up to 20 μmol/L Cu⁺² had not effect on procoagulant activity. However, in the presence of 1 μmol/L 8-hydroxyquinoline, Cu⁺² produced a dose dependent expression of procoagulant activity (Crutchley 1995²²⁰, Table 1). The effect of Cu⁺² was replicated with the copper transporting protein ceruloplasmin. Cu⁺² is known to produce lipid peroxidation and free radical generation. Therefore, the study tested the possibility that the procoagulant activity results from oxidative stress. Several lipophilic antioxidants, including probucol (20 μmol/L), vitamin E (50 μmol/L), BHT (50 μmol/L), and a 21-aminosteroid antioxidant U74389G (20 μmol/L), inhibited the Cu⁺² induced procoagulant activity (Ibid, FIG. 4). The increased procoagulant activity was due to TF. Cu⁺² induced intracellular oxidative stress which increased TF transcription. The kinetics of the induction of Cu⁺² was compared to LPS. Exposure to LPS or Cu⁺² resulted in TF mRNA increase. Relative to basal levels, LPS increased mRNA 2.5-fold after 2 hours of exposure declining to basal levels by 6 hours. In contrast, at 2 hours, Cu⁺² reduced mRNA levels to 50% followed by a 3.5-fold increase at 6 hours (see following figure). The cu+2 and LPS induced TF expression also differed in the response to antioxidants. While all four antioxidant inhibited cu⁺² induced TF expression, only vitamin E inhibited the LPS induced expression.

The LPS effect on TF transcription is mostly mediated through the NF-κB site. Crutchley, et al., (1995) results indicate that oxidative stress increased TF transcription through a different site. This conclusion is also supported by the negative effect of oxLDL on NF-κB binding to its site demonstrated in human T-lymphocytes (Caspar 1999²²¹), Raw 264.7, a mouse macrophage cell line (Matsumura 1999²²²), peritoneal macrophages (Hamilton 1998²²³), macrophages (Schackelford 1995²²⁴), and human monocyte derived macrophage (Ohlsson 1996²²⁵). The results in these studies are consistent with reduced binding of GABP to the N-box in the (−363 to −343) region of the TF gene.

Another study tested the effect of oxLDL on TF transcription. The binding of advanced glycation end products (AGE) with their receptor (RAGE) result in intracellular oxidative stress indicated by reduced glutathione (GSH) (Yan 1994²²⁶). Monocytes were incubated with AGE-albumin (AGE-alb) for 24 hours. The results show an increase in TF mRNA (Khechai 1997²²⁷, FIG. 1B). Presence of the translational inhibitor cycloheximide completely suppressed the AGE-alb induced TF mRNA accumulation (Ibid, FIG. 1B). The antioxidant N-Acetylcyteine (NAC) increases the levels GSH. NAC is easily transported into the cell. Incubation of cells with AGE-alb in the presence of 30 mmol/L NAC resulted in a concentration dependent inhibition of TF activity (Ibid, FIG. 2A) and TF antigen expression. Moreover, TF mRNA was almost completely suppressed (Ibid, FIG. 2C). Based on these results Khechai, et al., concluded that oxidative stress is responsible for TF gene expression.

Crutchley, et al., (1995²²⁸) showed that although reduced oxidative stress decreases TF mRNA, the LPS induced increase in TF mRNA is insensitive to certain antioxidant. Brisseau, et al., (1995²²⁹) showed a similar insensitivity of the LPS induced increase in TF mRNA to the antioxidant NAC. Since Khechai, et al., (1997) reported that NAC increases TF mRNA, the combined results of Brisseau, et al, (1995) and Khechai, et al., (1997) are also consistent with reduced GABP binding to the N-box in the (−363 to −343) region resulting from oxidative stress.

See also Ichikawa, et al., (1998²³⁰) which reported simliar results in human macrophage-like U937 cells treated with the oxidant AGE and the antioxidants catalase and probucol.

The induced TF is localized to regions important in cell motility. Consider the following studies.

Endotoxin treatment of human glioblastoma cells (U87MG) resulted in preferential localization of TF antigen in membrane ruffles and peripheral pseudopods. Most prominent TF staining was observed along thin cytoplasmic extensions at the periphery of the cells. Moreover, membrane blebs, associated with cell migration, were also heavily stained (Carson 1993²³¹). Endotoxin treatment of macrophages also resulted in a high concentration of TF antigen in membrane ruffles and microvilli relative to smooth areas of the plasma membrane or endocytosis pits (Lewis 1995²³², FIG. 2). The membrane ruffles and microvilli contained a delicate, three dimensional network of short fibrin fibers and fibrin protofibrils decorated in a linear fashion with the anti fibrin(ogen) antibodies. oxLDL treatment of macrophages resulted in similar preferential localization of TF antigen in membrane ruffles and microvilli.

Although the two studies use different terms, “cytoplasmic extensions” and “blebbed” (Carson 1993), and “microvilli” and “membrane ruffles” (Lewis 1995), the terms, most likely, describe the same phenomenon.

oxLDL increases TF activity. Consider the following study.

Lewis, et al., (1995²³³) demonstrate the effect of oxLDL treatment on TF activity. In culture, monocytes, and monocyte-derived macrophage expressed little or no procoagulant activity. Endotoxin treatment induced TF activity, peaking at 4 to 6 hours and decreasing over the following 18 hours (Ibid, FIG. 1). Cells exposed to minimally oxidized LDL (oxLDL) showed similar TF activation. The endotoxin and oxLDL treatments resulted in 115- and 58-fold increase in TF activity, respectively (Ibid, Table 1).

oxLDL also increases TF mRNA in smooth muscle cells (SMC) and endothelial cells. Consider the following two studies.

Quiescent rat SMC contained low levels of TF mRNA. Treatment of SMC with LDL or oxLDL significantly increased TF mRNA (Cui 1999²³⁴, FIG. 1). Densitometric analysis showed that oxLDL increases TF mRNA 38% more than LDL. The accumulation of TF mRNA induced by LDL or oxLDL was transient. Maximum level of TF mRNA was observed 1.5-2 hours following LDL or oxLDL stimulation (Ibid, FIG. 2), declining significantly over the following 5 hours. TF mRNA response to stimulation in human aortic SMC was similar. Nuclear run-on assays and mRNA stability experiments indicated that the increase in TF mRNA resulted mainly from increased transcription.

Another study exposed human endothelial cells to minimally oxidized LDL (oxLDL) or endotoxin for varying times. Northern blot analysis of total RNA showed a sharp increase in TF mRNA at 1 hour, a peak at 2 to 3 hours, and a decline to basal levels at 6 to 8 hours after treatment. The half-life of TF mRNA in oxLDL and endotoxin exposed endothelial cells was approximately 45 and 40 minutes, respectively. The rate of TF mRNA degradation was similar at 1 and 4 hours post treatment. Nuclear runoff assays showed a significant increase in TF transcription rate following exposure of the cells to oxLDL or LPS (Fei 1993²³⁵).

In monocytes/macrophages, oxLDL treatment reduces the binding of NF-κB to its site (see above). Since NF-κB stimulates TF transcription, the decreased binding diminishes the oxLDL positive effect on TF transcription mediated through the GABP site. In endothelial cells (Li 2000²³⁶) and smooth muscle cells (Maziere 1996²³⁷), oxLDL treatment increases the binding of NF-κB. This increase adds to the positive GABP mediated effect.

Oxidative stress reduces CD18 transcription. Consider the following study.

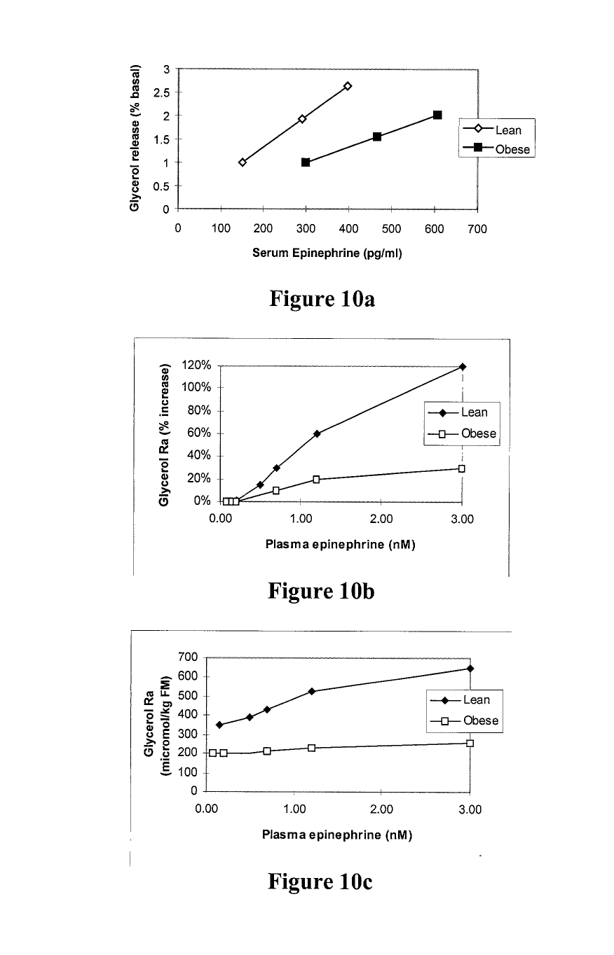

ICAM-1 is a ligand for CD18. Human polymorphonuclear leukocytes (PMN) were exposed to hypoxic condition. As a result, the adhesion of PMN to recombinant ICAM-1, but not BSA coated surfaces increased (Montoya 1997²³⁸, table 1). Anti-CD18 mAb abolished the increase adhesion (Ibid, FIG. 1). The antioxidant pyrrolidine dithiocarbamate (PDTC) reduced PMN intracellular oxidative stress (Ibid, FIG. 2). PDTC treatment of PMN increased PMN adhesion to tumor necrosis factor-α (TNFα) stimulated HUVEC monolayer (Ibid, FIG. 4). Pyrrolidine, which lacks antioxidant activity, failed to increase adhesion. Anti-CD18 abrogated the PDTC enhanced adhesion (Ibid, FIG. 5). Under flow conditions, a significant number of PMN were rolling at low velocities on the apical surface of the HUVEC monolayer. PDTC treatment reduced rolling distance and rolling velocities (Ibid, FIG. 10), increasing the number of stably adhered PMN. These observation indicate that reduced oxidative stress stimulates CD18 expression.

Hypoxia result in reduced oxidative stress, and therefore, stimulated GABP binding (Martin 1996²³⁹). Increased GABP binding stimulates CD18 transcription (Rosmarin 1998²⁴⁰), and therefore, CD18 adhesion. The observation in Montoya (1997, above) are consistent with such a mechanism.

Oxidative stress inducers of special importance (see below) are mmLDL and oxLDL.

mmLDL and oxLDL deplete intracellular GSH, and therefore induce oxidative stress. Consider the following studies.

GSH content was determined in cultured in human endothelial cells after 24 h incubation with native LDL or oxLDL at 30, 40 and 50 μg of protein/ml. The results showed that at 30 μg/mg, GSH content slightly but significantly increased (10%). In contrast, at 40 and 50 μg/ml, the GSH content decreased by 15 and 32%, respectively, (only significant at 50 μg/ml, P<0.05) (Therond 2000²⁴¹, FIG. 2B). Moreover, the results also showed that all oxLDL lipid fractions induced depletion of intracellular GSH (Ibid, FIG. 3B).

Another study tested the effect of a specific oxLDL fraction on intracellular GSH. Human promyelocytic leukemia cells U937 were treated with 7-ketocholesterol. U937 cells were used since they respond to oxysterols in concentrations similar to the concentrations observed in endothelial and smooth muscle cells, and since U937 are frequently used to model the response of macrophages to oxysterols in humans. The GSH content was measured by flow cytometry with monochlorobimane (Lizard 1998²⁴², FIG. 5A) At all time points, GSH content in the 7-ketocholesterol treated cells was lower compared to controls (P<0.05).

According to Gray and Shankar (1995²⁴³) “AthMØ (Atherosclerotic Macrophages) showed a substantial reduction in CD11b and CD18 cell surface expression. NMØ (Normal rabbit peripheral blood Monocytes), on the other hand, had a strong surface expression of both CD11b and CD18. . . . In comparison to NMØ that have been in cell culture for a short time, cell surface expression of the CD11b/CD18 integrin on AthMØ is strongly down-regulated. . . . Furthermore, these immunohistochemical studies provided evidence that the loss of CD11b/CD18 integrins is a function of the extent lipid loading and perhaps the stage of the foam cell formation. . . . It is our observation from looking at these cytologic preparations, that when stained for adhesion molecules, the smaller more normal appearing cells with very little lipid in them actually have the majority of staining, whereas the larger, more lipid laden cells have absolutely no staining in them.”

Mouse pertioneal macrophages were loaded with lipids by precincubation with acetylated LDL (acLDL) for various periods (100 μg/ml). The macrophages turned foam cells were used to fill the upper wells of a modified Boyden chamber. The lower wells contained Zymosan A activated mouse serum (ZAMS). Zymosan A is a cell-wall extract of Saccharomyces cerevisiae. ZAMS is a chemoattractant for macrophages. After 3 h, the membrane in the Boyden chamber was removed and the cells which did not migrate to the lower surface were wiped off. The migrated cells were fixed and counted. The results showed decreased macrophage migration with increased preincubation time with acLDL. Since, preincubation time correlated with lipid content, higher lipid content resulted in reduced migration (Trach 1996²⁴⁴, FIGS. 4a,b). (Similar results are reported in Pataki, et al., (1992²⁴⁵), an earlier study with H. Robenek as principle investigator.) Quinn, et al., (1985²⁴⁶) also reports reduced motility of resident macrophages with modified LDL as chemoattractant.

Bacterial particles are macrophage chemoattractants (for LPS, see above, for fMLP, see Yamada, et al., (1992²⁴⁷)). However, it seem likely that macrophage loading with one type of toxic substance (oxLDL, bacterial particle) reduces chemoattractance of the other. The results in the above studies are consistent with such a concept. In these studies, the zamosan chemoattractance was reduced with the increase in cell loading of modified LDL.

Bacterial particles, such as LPS or FMLP (a syntatic particle that represents bacterial products), are another important type of oxidative stress inducers (see below).

The products of the respiratory burst have low molecular weight, and therefore, diffuse out of the phagolysosome into cytoplasm and nucleus. The resulting oxidative stress effects TF transcription through the N-box and not the NF-κB site (see above). On the other hand, the bacterial particles, such as LPS, also increase TF transcritpion through the NF-κB site. These two effect act in synergy. Such a synergy is probably needed for quick removal of the relatively high toxic bacterial particles (compared to oxLDL toxicity) by faster clearance of bacterially loaded macrophages from infected tissues.

e) Net Propulsion

Consider a tissue resident molecule which is both an oxidant and a GABP kinase agent. As a GABP kinase agent the molecule chemoattracts circulating or resident leukocytes by increasing their expression of CD18 and α₄ integrin, inducing forward motility. The leukocyte migrate toward the molecule and phagocyte it. Once internalized, the molecule induces oxidative stress, i.e., depletes GSH, which, in turn, reduces binding of GABP to the N-boxes on TF, CD18 and α₄ integrin, resulting in increased expression of TF and reduces expression of CD18 and α₄ integrin. These changes reduces forward propulsion and increase backward propulsion, until backward propulsion is greater than forward propulsion. Since net force is the vector sum of all forces acting upon an object, the new net propulsion turns the leukocyte back toward circulation. The final step of this process is circulation reentry.

2. Atherosclerosis—Fibrous Cap Atheroma Formation

The first major class of atherosclerotic lesions is the fibrous cap atheroma. The fibrous cap is a distinct layer of connective tissue completely covering a lipid core. The fibrous cap consists of smooth muscle cells in a collagenous-proteoglycan matrix with a variable number of macrophages and lymphocytes (Virmnai 2000²⁴⁸). The following sections describe the mechanism of fibrous cap atheroma formation.

LDL Pollution

Plasma LDLs passively cross the endothelium (see below). Higher concentration of plasma LDL results in increased influx of LDL. Unlike other tissues, the intima lacks lymphatic vessels. Therefore, to reach the nearest lymphatic vessels, located in the medial layer, the LDL should pass through the intima. However, this passage is partly blocked by an elastic layer situated between the intima and the media (Pentikainen 2000²⁴⁹). According to Nordestgaard, et al., (1990²⁵⁰) “less than 15% of the LDL cholesteryl ester that entered the arterial intima penetrated beyond the internal elastic lamina.” A fraction of the influxed LDL is passively effluxed through the endothelium. Another fraction is hydrolyzed. The remaining intimal LDLs bind the extracelluar matrix (ECM). The ECM is composed of a tight negatively charged proteoglycan network. Certain sequences in the LDL apoB-100 contain clusters of positively charged amino acids lysine and arginine. These sequences, called heparin-binding domains, interact with the negatively charged sulphate groups of the glycosaminoglycan chains of the proteoglycans (Boren 1998²⁵¹, Pentikainen 2000²⁵²). Subendothelial agents modify (oxidize) the matrix bound LDL.

Passive Influx

Nordestgaard 1992²⁵³ reports a linear correlation between plasma concentration of cholesterol in LDL, IDL, VLDL and arterial influ. XMoreover, in cholesterol-fed rabbits, pigs and humans, arterial influx of lipoproteins depended on lipoprotein particle size. Other studies report that arterial influx of LDL in normal rabbits did not depend on endothelial LDL receptors. According to Nordestgaard, et al., these results indicate that the transfer of lipoprotein across endothelial cells and into the intima is a “nonspecific molecular sieving mechanism.” Schwenke (1997²⁵⁴) measured the intima-media permeability to LDL in different arterial regions in normal rabbits on a cholesterol-free chow diet. The results showed that the aortic arch is 2.5-fold more permeable to LDL compared to descending thoracic aorta (Ibid, Table 2). The concentration of undegraded LDL in the aortic arch was almost twice as great compared to the descending thoracic aorta (Ibid, Table 3). In cholesterol-fed rabbits, a result of hypercholesterolemia, the mass transport of LDL cholesterol into all arterial regions was greatly increased. However, hypercholesterolemia did not influence intima-media permeability of any arterial region (Ibid, Table 2). Kao, et al., (1994²⁵⁵), Kao, et al., (1995²⁵⁶) showed that open junctions with gap widths of 30-450 nm between adjacent endothelial cells were only observed in the breached regions of the aortic arch, and not in the unbranched regions of the thoracic aorta. Moreover, LDL labeled with colloidal gold were present within most of these open junctions, while no gold particles were found in the normal intercellular channels (i.e., 25 nm and less) of both regions. These results are consistent with a nonspecific molecular sieving mechanism.

Passive Efflux

Rabbits of the St Thomas's Hospital strain show elevated plasma levels of VLDL, IDL, and LDL. In both lesioned and nonlesioned aortic arches of these rabbits, the logarithms of the fractional loss of VLDL, IDL, LDL, HDL, were inversely and linearly correlated with the diameter of these macromolecules (Nordestgaard 1995²⁵⁷). This observation suggests that, similar to influx, the efflux of LDL through the endothelium can also be described as “nonspecific molecular sieving mechanism.”

a) LDL Clearance

Modified LDL is chemotactic to circulating monocytes (see above). As a result, endothelial cells increase the surface expression of P-selectin and circulating monocytes increase CD18 and α₄ integrin expression (other surface molecule also change their expression). The increased expression of forward propulsion genes increases adhesion of circulating monocyte to the endothelium (margination), and emigration (see forward motility above). Once in the intima, monocytes differentiate into macrophages and start to accumulate modified LDL turning into foam cells. The intracellular oxidative stress induced by the modified LDL particles decreases CD18 and α₄ integrin transcription and stimulates TF transcription. The decreased CD18 and α₄ integrin expression reduces forward propulsion. The transient increase in TF activity on the surface of foam cells induces backward propulsion. When backward propulsion surpasses forward propulsion the cell turns back. When the foam cells reach the endothelium, they first bind the basal surface and then the apical surface of the endothelium. When TF adhesion activity returns to basal level, the apical bound foam cells are released into circulation.

There is extensive research showing more adhesion and emigration of monoctyes in atherosclerosis.

The results of the following studies are consistent with clearance of foam cells:

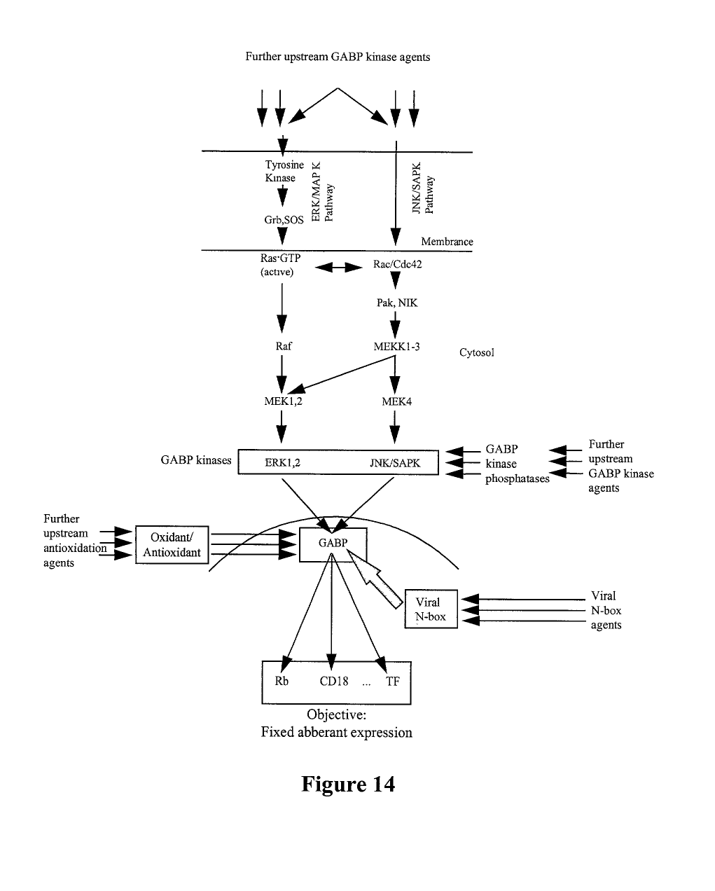

Twenty-two Yorkshire pigs were fed a high fat diet. The animals were killed 12, 15 and 30 weeks after diet initiation, and tissue samples were examined by light and electron microscopy. At 15 weeks, lesions were visible as raised ridges even at low magnification (Gerrity 1981²⁵⁸, FIG. 1). Large numbers of monocytes were adherent to the endothelium over lesions, generally in groups (Ibid, FIG. 5), unlike the diffused adhesion observed at prelesion areas. Foam cells overlaid lesions at all three stages, although more frequently at 12 and 15 weeks. The foam cells had numerous flaplike lamellipodia and globular substructure (Ibid, FIG. 6). Some foam cells were fixed while passing through the endothelium, trapped in endothelial junctions alone (Ibid, FIG. 8) or in pairs (Ibid, FIG. 9). In all cases, the attenuated endothelial cells were pushed luminally (Ibid, FIG. 14). The lumenal portion of the trapped foam cells had an irregular shape, with numerous cytoplasmic flaps (lamellipodia and veil structures), empty vacuoles and reduced lipid content compared to the intimal part of the cell (Ibid, FIG. 8 and 9). Foam cells were also infrequently found in buffy coat preparations from arterial blood samples (Ibid, FIG. 7), and rarely in venous blood. According to Gerrity, these findings are consistent with backward migration of foam cells and suggest that such a migration indicates the existence of a foam cell mediated lipid clearance system.

Another study fed 10 male pigtail monkeys an atherogenic diet and 4 monkeys a control diet. Twelve days after diet initiation, and at monthly intervals, up to 13 months, animals were killed and tissue samples were examined by light and electron microscopy. The endothelial surface of the aorta in control animals was covered with a smooth, structurally intact endothelium (Faggiotto 1984-I²⁵⁹, FIG. 4A). Occasionally, the surface showed small focal areas protruding into the lumen (Ibid, FIG. 4B). Cross sectional examination of the protrusions revealed foam cells underlying the intact endothelium (Ibid, FIG. 3A). During the first 3 months, the endothelium remained intact. However, on larger protrusions, the endothelium was extremely thin and highly deformed. At 3 months, the arterial surface contained focal sites of endothelial separation with a foam cell filling the gap (Ibid, FIG. 10A). The luminal section of the foam cell showed numerous lamellipodia. In addition, thin sections of endotheilium cells bridged over the exposed foam cell, deforming the surface of the foam cell (Ibid, FIG. 10B). Moreover, rare occasional foam cells were observed in blood smears of some controls. During the first 3 months, when the endothelium was intact, the number of circulating foam cells increased (Faggiotto 1984-II²⁶⁰, FIG. 10). Based on these observation Faggiotto, et al., concluded that foam cells egress from the artery wall into the blood stream, confirming Gerrity (1981) conclusions.

A third study fed 36 male New Zealand White rabbits a cholesterol-enriched diet and 37 rabbits a control diet. Both groups were exposed to electrical stimulation (ES) known to induce arteriosclerotic lesions. The stimulation program lasted 1, 2, 3, 7, 14, or 28 days. At these intervals, tissue samples were collected, processed, and examined by transmission electron microscopy (TEM). After 1 day of ES, intimal macrophages of hypercholesterolemic rabbits showed loading of lipids (Kling 1993²⁶¹, FIG. 3b). These cells were often responsible for markedly stretching the overlying endothelial cells. After 2 days, foam cells were fixed while passing through endothelial junctions (Ibid, FIG. 8a). Neighboring endothelial cells were often pushed luminally, indicating outward movement of the macrophage (Ibid, FIG. 8a). The outward movement of the cells was also supported by the finding that the intimal portion of the foam cells transmigrating the endothelium was intact, while the lumina portion was often ruptured and associated with platelets.(Ibid, FIGS. 8b,c). Under the prolonged influence of the atherogenic diet, emerging foam cells became more frequent. In all cases, the emerging foam cells migrated through endothelial junctions without damaging the endothelium. Based on these observations, Kling, et al., concluded that “similar to observations of Gerrity and Faggiotto, et al, we have electro microscopic evidence that the macrophages, loaded with lipid droplets, were capable of migrating back from the intima into the blood stream . . . thus ferrying lipid out of the vessel wall.”

The following studies show increased TF expression on foam cells:

Seven White Carneau pigeons were fed an atherogenic diet and three animals received a control diet. The diet regimen lasted 8-10 months shown to be sufficient to induce lesion in the thoracic aorta. The concentration of tissue factor (TF) antigen in circulating monocytes, cultured macrophages, and macrophages from atherosclerotic lesions was ultrastructurally analyzed using immunogold labeling. The plasma cholesterol of the cholesterol-fed animals was elevated compared to controls. Upon dissection, all cholesterol-fed animal revealed fatty streaks and atherosclerotic plaque at the celiac bifurcation of the thoracic aorta. Monocytes isolated from normocholesterolemic and hypercholesterolemic animals had approximately 1 immunogold particle per 2 μm of plasma membrane (Landers 1994²⁶², FIG. 2). The low level of TF antigen in the plasma membrane is consistent with the lack of TF procoagulant activity in freshly-isolated monocytes or monocyte-derived macrophages maintained in culture. Monocytes newly adherent to lesion surface also showed low level of TF antigen (0.3 particles/μm of plasma membrane). In contrast, lumenally exposed surface of foam cells projecting into the arterial lumen from subendothelial intima showed high level of TF antigen (7.3 particles/μm of plasma membrane). The distribution of TF concentrations on surface of macrophages was bimodal. Circulating and newly adherent macrophages had low level of TF antigen. Projecting foam cells had high level of TF antigen. (The immunogold labeling of the endothelial cells either underlying the adherent macrophages or flanking intimal foam cells protruding into the lumen was minimal.) According to Landers, et al., these observations are consistent with the egressing foam cells reported by Gerrity. Another unpublished observation reported in Landers, et al, (1994) is the association between short term lesions regression and the transient increase in clot formation on lesions.

Faggiotto 1984-I²⁶³ showed the existence of foam cells in peripheral blood smears from hyperlipidemic monkeys. Most of these cells showed no adherence to plastic cell culture dishes. However, TF induces such adherence. Since egressing foam cells show high concentration of TF antigen, either TF is removed from the cell surface while in circulation or, more likely, TF adhesion activity is reduced by encryption.

Lander, et al., (1994) and Faggiotto, et al., (1984) observations are consistent with the following model. Modified LDL increases TF transcription. The initial increase in TF concentration on surface of foam cells results in backward motility. The cells pass through gap junctions by first binding the basal and then apical side of the endothelium. Concurrently, the concentration of surface TF continues to increase. The additional surface TF deactivate many surface TF molecules through formation of TF dimers (encryption). The encrypted foam cells release from the endothelium surface and join circulation.

b) Atherogenesis

Let Trapped_{FC}, EgreSS_{FC} and Total_{FC} denote the number foam cells trapped in the intima, the number of foam cells in the process of egressing from the subendothelial space and the total number of intimal foam cells, respectively. Trapped_{FC}+Egress_{FC}=Total_{FC}. Denote the fraction of foam cells trapped in the intima with %_{Trapped}. Assume that inefficiencies in foam cell backward motility, denoted I, increase %_{Trapped}, which is the percentage of trapped foam cells. Also assume that %_{Trapped} is independent of Total_{FC}, the total number of intimal foam cells.

(1) Trapped_{Fc}=%_{Trapped}(I)×Total_{FC}.

Let Rateₗₑₛᵢₒₙₛ denote the rate of athersclerotic lesion formation.

(2) Rateₗₑₛᵢₒₙₛ=f(Trapped_{Fc})=f(%_{Trapped}(I)×Total_{FC}).

The following derivatives summarize the relation between changes in Total_{FC} or I and Rateₗₑₛᵢₒₙₛ.

$$\begin{matrix}\frac{\partial {Rate}_{lesions}}{\partial {Total}_{FC}} = \frac{\partial {Rate}_{lesions}}{\partial {Trapped}_{FC}} \cdot {\%}_{Trapped} & ( 3 ) \\ \frac{\partial {Rate}_{lesions}}{\partial I} = \frac{\partial {Rate}_{lesions}}{\partial {Trapped}_{FC}} \cdot {Total}_{FC} \cdot \frac{\partial {\%}_{Trapped}}{\partial I} \\ Consider equation 3. \\ \frac{\partial {Rate}_{lesions}}{\partial {Trapped}_{FC}} > 0. {\%}_{Trapped} & ( 4 )\end{matrix}$$

is fixed. Therefore,

$$\frac{\partial {\%}_{Trapped}}{\partial {Total}_{FC}} > 0 ,$$

an increase in total number of intimal foam cells increases the rate of lesions formation. An increase in LDL pollution increases the entry of monocytes which increases the total number of intimal foam cells resulting in increased rate of lesions formation.

$$Consider equation 4$$

$$\frac{\partial {\%}_{lesions}}{\partial {Total}_{FC}} > 0.$$

$${Total}_{FC} > 0. \frac{\partial {\%}_{Trapped}}{\partial I} > 0.$$

Therefore,

$$\frac{\partial {Rate}_{lesions}}{\partial I} > 0 ,$$

an increase in backward motility inefficiencies increases the rate of lesion formation.

There are numerous observations consistent with such a model of atherogenesis. Most of these observations relate to the effect of the total number of intimal foam cells on rate of lesion formation (equation 3). For instance, diet or genetically induced hypercholesterolemia increases plasma concentration of LDL resulting in increased LDL pollution. The increased oxLDL bound to the ECM chemoattracts monocytes. As expected in equation 3, the increase in TotalFc results in increased rate of lesion formation. Another example is LDL pollution of the edges of blood vessel bifurcations resulting from low shear stress (Malek 1999²⁶⁴). As expected, these areas show higher propensity to develop atherosclerotic lesions.

The opposite direction also holds. A reduction in LDL pollution reduces the rate of atherosclerotic formation. For instance, studies showed that in a animal, several months of a lipid-reduced diet resulted in a decreased number of foam cells and regression of fatty streaks (Trach 1996²⁶⁵, Pataki 1992²⁶⁶, Wissler 1990²⁶⁷, Dudrick 1987²⁶⁸, Tucker 1971²⁶⁹). Other studies showed that a genetic deficiency in ICAM-1, P-selectin or E-selectin (Collins 2000²⁷⁰), a genetic double deficiency in P-selectin and E-selectin (Dong 1998²⁷¹) or treatment with monoclonal antibodies against VAL4 or ICAM-1 (Patel 1997²⁷²) reduced monocyte recruitment resulting in diminished rate of atherosclerotic lesions formation. More studies showed that a mutation in all basic amino acids in the proteoglycan-binding region of apoB-100, which prevents binding of the heparin proteoglycans in ECM, resulted in only mild atherosclerosis despite strong hypercholesterolemia (Pentikainen 2000²⁷³). The diminished concentration of ECM bound oxLDL attracted less monocytes resulting in reduced Total_{FC}.

For a review of the different theories of atherosclerosis, see Stary, et al., (1994²⁷⁴).

c) Microcompetition

Latent infection of endothelial cells increases P-selectin expression inducing increased transmigration of monocytes. According to equation (3) above, the increased number of foam cells increases the rate of lesion formation.

The subendothelial environment transactivates latent viral infection in monocytes turned macrophages. Consider the following studies.

Cytomegalovirus (CMV) is a GABP virus. Circulating monocytes are nonpermissive for CMV replication. Monocytes show no expression of viral gene products even when cells harbor a viral genome (Taylor-Wiedeman 1994²⁷⁵). In monocytes the virus is in a latent state. Viral replication is dependent on expression of viral immediate-early (IE) gene products controlled by the major immediate-early promoter (MIEP). HL-60, promyelocytic cells that can differentiate into macrophages, were transfected with MIEP-CAT, a reporter-plasmid construct controlled by the CMV MIEP. Coculture of MIEP-CAT-transfected cells with endothelial cells (ECs) increased MIEP-CAT activity 1.7 fold over baseline activity in noncocultured HL-60 cells (Guetta 1997²⁷⁶, FIG. 1A). Coculture of MIEP-CAT-transfected cells with smooth muscle cells (SMCs) increased MIEP-CAT activity 4.5-fold over baseline (Ibid, FIG. 1B). Treatment with 50 to 200 μg/mL oxLDL activated MIEP, in a concentration dependent manner (Ibid, FIG. 2.). A 2.0-fold increase was the largest observed effect of oxLDL (Ibid, FIG. 1C). Coculture with ECs plus oxLDL led to a 7.1-fold increase over baseline, larger than the two separate effects. Based on these results Guetta, et al., concluded that exposure of monocytes turned macrophages to ECs, SMCs, and oxLDL in the subendothelial space favors transactivation of latent CMV.

Moreover, when cerulenin, an inhibitor of fatty acid biosynthesis, was added to mouse fibroblasts infected with Moloney murine leukemia virus (MMuLV), virus production was drastically reduced (Ikuta 1986B²⁷⁷, Katoh 1986²⁷⁸). Cerulenin also inhibited Rous sarcoma virus (RSV) production in chick embryo fibroblasts (Goldfine 1978²⁷⁹)

Following entry to the subendothelial space, monocytes differentiate into macrophages. Monocytes differentiation transactivated the HCMV IE gene (Taylor-Wiedeman 1994²⁸⁰), and, in some cases, produced productive HCMV infection (Ibanez 1991²⁸¹, Lathey 1991²⁸²). Similarly, differentiation of THP-1 premonocytes (Weinshenker 1988²⁸³), and T2 teratocarcinoma cells (Gonczol 1984²⁸⁴), also produced HCMV replication.

Subendothelial monocyte-derived macrophages are exposed to ECs, SMCs and oxLDL. If a macrophage harbors a GABP viral genome, the subendothelial environment stimulates viral replication. The increase in viral DNA intensifies microcompetition.

The increased viral replication in the subendothelial space intensifies microcompetition. The reduced expression of CD18 and α4 integrin stops the macrophage at a reduced intimal depth. The oxLDL deep in the intima is not cleared and remains ECM bound. While trapped foam cells form fatty streaks, the ECM bound oxLDLs form the lipid core of the atherosclerotic plaque. The following observations are consistent with such a mechanism.

The core of atherosclerotic plaque actually forms concurrently with fatty streaks. The core has a tendency to extend from a position initially deep in the intima toward the lumen of the artery with increasing age. The lipid in the core region seem to originate directly from plasma lipoproteins and not from foam cell necrosis. Foam cells are usually seen in superficial intima in the region between the core and the endothelial surface (Guyton 1995²⁸⁵)

The studies in Randolph, et al., (1996²⁸⁶) and Randolph, et al., (1998²⁸⁷) (see above) have a similar experimental setting. However, Randolph, et al., (1996) tested the effect of mAb against ICAM-1 and mAb against CD18 on reverse transmigration. The results showed that Fab fragments of nAb against ICAM-1 (R6.5) completely blocked egression of mononuclear phagocytes (MP) from IL-1-treated HUVEC/amnion cultures for a total of 5 h (Ibid, FIG. 9A). When incubation of MP-HUVEC cocultures (IL-1-pretreated HUVEC) was extended to 12 h, anti-ICAM-1 Fab fragments inhibited reverse transmigration of monocytes by 53% (Ibid, FIG. 9b). Anti CD18 Fab fragments (TS1/18) suppressed reverse transmigration by an average of 71% at 5 h of incubation (Ibid, FIG. 9a). Based on these observations Randolph, et al., concluded that one role of CD18 and ICAM-1 in reverse transmigation is to accelerate initial kinetics.

These results indicate the existence of an intial delay in the activation of TF propelled backward motility. This delay might be necessary for allowing other cell changes required for TF propelled motility such as cell skeleton modifications. During this delay, other molecules, such as CD18, propel backward motility.

Many studies measured the effect of certain agents on TF activity over the first few hours following treatment. For instance, Key, et al., (1993²⁸⁸) infected HUVEC with herpes simplex virus-1 (HSV-1), or exposed the cells to LPS and measured TF PCA activity. Schecter, et al., (1997²⁸⁹) measured the effect of platelet-derived growth factor (PDGF) stimulation on TF activity on surface of human aortic smooth muscle cell (SMC). Lewis, et al., (1995²⁹⁰) reported stimulated monocytes, and monocyte-derived macrophages with oxLDL or LPS (see above) and measured TF activity. The results showed that both agents had similar effects.

Combining the observations in Randolph, et al., (1996²⁹¹) with these observations suggests that TF driven backward motility starts around the time when TF activity is maximized. Moreover, TF propelled reverse transmigration occurs while TF activity is declining. This observation is called “soft landing.” It is proposed that soft landing might reduce the probability of a undesired coagulation reaction on the surface of egressed foam cells or might increase the probability of foam cells release from apical surface of endothelium.

FIG. 8 shows change in TF activity as a function of time for a control cell and a cell harboring a GABP viral genome. In general, ₐTF denotes TF activity and _{c}TF denotes TF surface concentration on cell surface. Let ₐTFₛₜₒₚ denote TF activity that cannot support reverse transmigration. If ₐTFₛₜₒₚ is reached before a foam cell has reached the apical surface of the endothelium, the cell is trapped. Let Δ_{c}TF_{oxLDL}, Δ_{c}TF_{V} denote an increase is TF membrane concentration resulting from stimulation with oxLDL and from microcompetition with a GABP virus, respectively. Let ₐTF_{basal} denote basal TF activity prior to stimulation.

Consider a control cell, denoted “cc,” and a cell harboring a GABP viral genome, denoted “vc.” Microcompetition between the TF promoter and the GABP virus stimulates TF transcription (see the section on TF gene, above). Let t−0 mark the time of monocyte completed differentiation into macrophage following entry to subendothelial space. For every t>0, microcompetition results in Δ_{c}TF_{V}(t)>0.

In both cells, for every t>0, _{c}TF(t)=_{c}TF_{basal}+Δ_{c}TF(t). However, for viral cell _{c}ΔTF(t)=Δ_{c}TF_{oxLDL}(t)+Δ_{c}TF_{V}(t) (we assume an additive effect for the oxLDL and V combination). Since Δ_{c}TF_{V}(t)>0 for viral cells, at any time t, TF concentration on surface of viral cell is greater than TF concentration on surface of control cell. Referring to FIG. 8, “cc, _{c}TF” and “vc, _{c}TF” lines represent the increase in TF surface concentration as a function of time for a control cell and viral harboring cell, respectively. The “cc, ₐTF” and “vc, ₐTF” curves represent the change in TF activity as a function of time for these cells. The vertical distance between “vc, _{c}TF” and “cc, _{c}TF” represents the effect of microcompetition on the surface concentration of TF. The increase in surface TF concentration shifts the “vc, ₐTF” curve to the left. As a rule, in both cells the same TF surface concentration generates the same TF activity. For instance, points 7 and 8 represent the same surface concentration and therefore produce the same activity, represented by points 5 and 9, the points of maximum activity. Points 1 and 3 also represent the same surface concentration. These points produce activity 2 and 4, the activity associated with cells at rest, or “stopped” cells.

For every delay≧0, tₛₜₒₚcc-tₛₜₐᵣₜ>tₛₜₒₚvc-tₛₜₐᵣₜ (see FIG. 8). The time the viral cell is actually moving towards circulation is shorter compared to control. Assume the probability of reaching the endothelium apical surface increases with movement time. Since the viral cell movement time is shorter, its probability of being trapped is higher.

Another observation relates to cell velocity. Assume the delay is the same for both cells. The shift of the “vc, _{c}TF” curve results in lower TF activity on the viral cell for every t of actual movement (every t>tₛₜₐᵣₜ in the figure). Assume that cell velocity depends on TF activity. Then, at any time, the viral cell is slower than control cell. The reduced velocity also increases the probability of being trapped.

Microcompetition between a GABP virus and TF increases the probability of being trapped in the subendothelial space. Denote the number of viral N-boxes with V_{Nbo}. XV_{Nbox} increases the inefficiencies in foam cell backward motility, denoted I in above clearance model.

Modify equation (2).

(5) Rateₗₑₛᵢₒₙₛ=f(%_{Trapped}(I(V_{Nbox}))×Total_{FC}

The following derivative represents the effect of V_{Nbox} on Rateₗₑₛᵢₒₙₛ, the rate of lesion formation.

$$\frac{\partial {Rate}_{lesions}}{\partial {V}_{Nbox}} = \frac{\partial {Rate}_{lesions}}{\partial {Trapped}_{FC}} \cdot {Total}_{FC} \cdot \frac{\partial {\%}_{Trapped}}{\partial I} \cdot \frac{\partial I}{\partial {V}_{Nbox}} \qquad ( 6 )$$

$$Consider equation ( 6 ) . \\ \frac{\partial {Rate}_{lesions}}{\partial {Trapped}_{FC}} > 0 , {Total}_{FC} > 0 , \frac{\partial {\%}_{Trapped}}{\partial I} > 0 ( see above ) .$$

$$\frac{\partial {\%}_{Trapped}}{\partial {V}_{Nbox}} > 0.$$

Therefore,

$$\frac{\partial {Rate}_{lesions}}{\partial {V}_{Nbox}} > 0.$$

Microcompetition increases the rate of lesion formation. Moreover, the larger the number of viral N-boxes in the infected cells, the higher the rate of lesion formation.

In addition, CD18 is also a GABP stimulated gene (see above). Therefore, microcompetition between the GABP virus and CD18 gene results in reduced expression of the cellular gene. According to Randolph, et al., (1996), the role of CD18 is to accelerate the initial kinetics of reverse transmigration (see above). A decrease in CD18 expression might further reduce foam cell velocity, increasing the probability of being trapped in the subendothelial space.

3. Atherosclerosis-intimal Thickening

A second major class of atherosclerotic lesions is pathological intimal thickening. Intimal thickening consists mainly of smooth muscle cells in a proteoglycan-rich matrix. Pathological intimal thickening class should be considered as a class independent of fibrous cap atheroma since the majority of lesion erosion occur over areas of intimal thickening with minimal or no evidence of a lipid core (Virmnai 2000²⁹²). Smooth muscle cell (SMC) proliferation, which results in neointima formation and intimal thickening, accounts for a significant rate of restenosis after percutaneous transluminal coronary angioplasty, a widespread treatment for coronary artery disease. The following identifies the cause of SMC proliferation, neointima formation and intimal thickening in atherosclerosis.

SMCs are permissive to HCMV (Zhou 1996²⁹³) and HSV (Benditt 1983²⁹⁴). Rb is a GABP stimulated gene. Microcompetition with viral DNA decreases Rb transcription in SMCs (see the section on cancer).

Rb mRNA is reduced in atherosclerotic plaque. Rabbits were fed a high cholesterol diet for six months. The results showed that the atherosclerotic plaques, covering 91% of the intimal aortic surface of aorta thoracalis, contained less Rb mRNA (P<0.05) compared to normal aortic arteries (Wang 1996²⁹⁵). Based on this result, Wang, et al., suggested that “the abnormal expression of . . . Rb antioncogene may play an important role in arterial SMC proliferation and pathogenesis of atherosclerosis.”

Rb is important in SMC arrest and differentiation. Increased Rb transcription (Claudio 1999²⁹⁶, Schwartz 1999²⁹⁷, Smith 1997²⁹⁸), or reduced pRb phosphorylation (Gallo 1999²⁹⁹) decreased SMC proliferation and neointima formation. Since microcompetition reduces Rb transcription, an infection with a GABP virus results in SMC proliferation, neointima formation and pathological intimal thickening.

4. Viruses in Atherosclerosis

The idea of infection as risk factor for atherosclerosis and related cardiovascular diseases is more than 100 years old. However, it was not until the 1970s that experimental data was published supporting the role of viruses in atherosclerosis. The mounting evidence linking infectious agents and atherosclerosis prompted the scientific community to organize the International Symposium of Infection and Atherosclerosis, held in Annecy, France, Dec. 6-9, 1998. The main objective of the symposium was to evaluate the role of infection in the induction/promotion of atherosclerosis on the basis of evidence from recent data on pathogenesis, epidemiologic and experimental studies and to define prevention strategies and promote further research. Consider the following studies presented at the symposium. The studies were published in a special issue of the American Heart Journal (see American Heart Journal, November 1999).

Chiu presenetd a study which found positive immunostainings for C pneumoniae (63.6%), cytomegalovirus (CMV) (42%), herpes simplex virus-1 (HSV-1) (9%), P gingivalis (42%), and S sanguis (12%) in carotid plaques. The study found 1 to 4 organisms in the same specimen (30%, 24%, 21%, and 6%, respectively). The micro-organisms were inmunolocalized mostly in macrophages (Chiu 1999³⁰⁰).

In a critical review of the epidemiologic evidence, Nieto suggested that “most epidemiologic studies to date (Nieto 1999³⁰¹, Table I and II) have used serum antibodies as surrogate of chromic viral infection. However, there is evidence suggesting that serum antibodies may not be a valid or reliable indicator of chromic or latent infections by certain viruses. In a pathology study of patients undergoing vascular surgery for atherosclerosis serology, for example, for the presence of serum cytomegalovirus antibodies was not related to the presence of cytomegalovirus DNA in atheroma specimens.” However, according to Nieto, four studies, Adam, et al., (1987³⁰²), Li, et al, (1996³⁰³), Liuzzo, et al., (1997³⁰⁴) and Blum, et al., (1998³⁰⁵) showed strong positive associations between CMV and clinical atherosclerosis. A strong association was also found in a 1974 survey of the participants in the Atherosclerosis Risk in Communities (ARIC) study between levels of cytomegalovirus antibodies and the presence of subclinical atherosclerosis, namely carotid intimal-medial thickness measured by B-mode ultrasound (Nieto 1999³⁰⁶)

Nieto also reported results of a prospective study of clinical incident coronary heart disease (CHD). The study was a nested case-control study from the Cardiovascular Health Study (CHS) conducted in an elderly cohort. Preliminary results from this study found no association between cytomegalovirus antibodies at baseline and incident CHD over a 5 year period. However, HSV-1 was strongly associated with incident CHD, particularly among smokers (odds ration [OR] 4.2). It should be noted that a more recent prospective study of CMV, HSV-1 in CHD found that participants in the Atherosclerosis Risk in Communities Study (ARIC) study with highest CMV antibody levels at base line (approximately upper 20%) showed increased relative risk (RR, 1.76, 95% confidence interval, 1.00-3.11) of CHD incident over a 5 year period, adjuste for age, sex and race. After adjustment for additional covariates of hypertension, diabetes, years of education, cigarette smoking, low-density lipoprotein and high-density lipoprotein cholesterol levels, and fibrinogen level, the RR increased slightly. The study found no association between CHD and the highest HSV-1 antibody levels (adjusted RR, 0.77; 95% confidence interval, 0.36-1.62) (Sorlie 2000³⁰⁷).

Nieto (1999) also mentioned some recent studies which documented increased risk of restenosis after angioplasty in patients with serologic evidence of cytomegalovirus infection. For instance, Nieto reported a study by Zhou and colleagues which included 75 consecutive patients undergoing directional coronary atherectomy for symptomatic coronary artery disease. Six months after atherectomy, the cytomegalovirus-seropositive patients showed significantly greater reduction in luminal diameter and significantly higher rate of restenosis compared to controls (43% vs 8% OR 8.7). These results were independent of known cardiovascular disease (CVD) risk factors.

Finally, Nieto mentioned that cytomegalovirus infection has been associated with another form of atherosclerotic disease: accelerated atherosclerosis in the coronaries after heart transplantation. In the first study showing this associations cytomegalovirus serology after transplantation seemed to be one of the most significant predictors of graft atherosclerosis and survival in general. This difference was independent of serologic status before transplantation and presence of symptomatic infection. Similar results have been replicated in subsequent studies.

Based on these studies Nieto concludes that “despite its limitations, the epidemiologic evidence reviewed above is consistent with a broad range of experimental and laboratory evidence linking viral (and other) infections and atherosclerosis disease.”

In a review of animal studies Fabricant, et al., (1999³⁰⁸) described their experiments with Marek's disease herpesvirus (MDV). The initial experiment used 4 groups of specific pathogen-free (SPF) whit leghorn chickens, P-line cockerels of the same hatch, genetically selected for susceptibility to MDV infection. Groups 1 and 2 were inoculated intratracheally at 2 days of age with 100 plaque forming units of clone-purified, cell free, CU-2 strain of low-virulence MDV. Groups 3 and 4 were controls. For the first 15 weeks, all birds in the 4 groups were fed the same commercial low cholesterol diet (LCD). Beginning with the 16th and ending with the 30th week, MDV-infected group 2 and uninfected group 4 were placed on a high cholesterol diet (HCD). The other two groups remained on LCD. Atherosclerotic lesions visible at gross inspection were only observed in MDV-infected birds of groups 1 (LCD) and 2 (HCD). These arterial lesions were found in coronary arteries, aortas, and major arterial branches. In some instances, the marked atherosclerotic changes involved entire segments of the major arteries practically occluding the arterial lumen. Other arterial lesions visible at gross inspection were observed as discrete plaques of 1 to 2 mm. These arterial lesions were not found in any of the uninfected birds of group 3 (LCD) or the uninfected hypercholestrolemic birds of group 4. Many proliferative arterial lesions with intimal and medial foam cells, cholesterol clefts, and extracellular lipid and calcium deposits had marked resemblance to chronic human atherosclerotic lesions. Moreover, immunization prevented the MDV-induced atherosclerotic lesions.

The main conclusion of the symposium was that “although studies are accumulating that indicate a possible relation between infection and atherosclerosis, none of them has yet provided definite evidence of a causal relations. . . . Moreover, the demonstration of a causative role of infectious agents in atherosclerosis would have an enormous impact on public health” (Dodet 1999³⁰⁹) (A similar view is expressed in a review published recently, see Fong 2000³¹⁰).

What is “definitive evidence?” What evidence will convince Dodet, and others, that viruses are not merely associated with atherosclerosis but actually cause the disease?

The research on viruses in cancer provides an answer. According to zur Hausen (1999³¹¹) “The mere presence of viral DNA within a human tumor represents a hint but clearly not proof for an aetiological relationship. The same accounts for seroepidemiological studies revealing elevated antibody titres against the respective infection.” What constitute a proof is evidence that meets the following four criteria, specifically the fourth one. According to zur Hausen “the fourth point could be taken as the most stringent criterion to pinpoint a causal role of an infection.”

TABLE 1
zur Hausen's criteria for defining a causal
role for an infection in cancer

1. Epidemiological plausibility and evidence that a virus
infection represents a risk factor for the development of a
specific tumor.
2. Regular presence and persistence of the nucleic acid of
the respective agent in cells of the specific tumor.
3. Stimulation of cell proliferation upon tranfection of the
respective genome or parts therefrom in corresponding
tissue culture cells.
4. Demonstration that the induction of proliferation and
the malignant phenotype of specific tumor cells depends
on functions exerted by the persisting nucleic acid of the
respective agent.

The fourth point requires an understanding of the “mechanisms of virus mediated cell transformation.” Crawford (1986³¹²) and Butel (2000³¹³) also emphasize the significance of understanding the mechanism in attributing a causality role to infection. According to Crawford: “one alternative approach to understudying the role of the papillomaviruses in cervical carcinoma is to identify the mechanisms by which this group of viruses may induce the malignant transformation of normal cells.” According to Butel: “molecular studies detected viral markers in tumors, but the mechanism of HBV involvement in liver carcinogenesis remains the subject of investigation today.” When the other kind of evidence is in place, understanding the mechanism turns a mere association into a causal relation.

The discovery of microcompetition and its effect on macrophage propulsion and SMC replication provides the mechanism that produces atherosclerosis. This discovery seem to supply the missing “definitive evidence” for a causal relationship between viruses and atherosclerosis.

5. Metastasis

The expression of TF is increased in various metastatic tumors, such as non-small-cell lung cancers (Sawada 1999³¹⁴), colorectal cancer (Shigemori 1998³¹⁵), melanoma (Meuller 1992³¹⁶), prostate cancer (Adamson 1993³¹⁷), colorectal carcinoma cell lines and metastatic liver sublines (Kataoka 1997³¹⁸), breast cancer (Sturm 1992³¹⁹), and in variety of cancer cell lines (Hu 1994³²⁰) Moreover, TF expression directly correlates with tumor aggressiveness (see above studies and also following reviews, Ruf 2000³²¹, Schwartz 1998³²²).

In an intervention study generated two matched sets of cloned human melanoma lines, one expressing a high level and the other a low level of normal human TF molecule, by retroviral-mediated transfections of a nonmetastatic parental line. The tumor cells were injected into the tail vein of severe combined immunodeficiency (SCID) mice. The results showed that metastatic tumors in 86% of the mice injected with the high-TF lines and in 5% of the mice injected with the low-TF lines (Bromberg 1995³²³). Based on these results, Bromberg, et al., concluded that “high TF level promotes metastasis of human melanoma in the SCID mouse model.”

TF is a GABP suppressed gene. Microcompetition increases TF transcription (see above). Therefore, an infection with a GABP virus promotes metastasis.

### E. Clinical Effect of Microcompetition—Osteoarthritis

1. Collagen type I α2 chain (COL1A2)

COL1A2 is a microcompetition-repressed gene, moreover, the COL1A2 is ERK responsive. ERK stimulates COL1A2 transcription. One study examined the influence of hypergravity on collagen synthesis in human osteoblast-like cells (hOB), as well as the involvement of the MAP kinase signaling cascade. They found that hypergravity led to significantly increased phosphorylation of ERK 1/2. When the MAPK kinase pathway was inhibited by PD98059, hypergravity-induced stimulation of both collagen synthesis as well as COL1A2 mRNA expression decreased by about 50% (Gebken 1999³²⁴).

2. COL1A2 Deficiency

COL1A2 causes EDS. A latent infection by a GABP virus results in microcompetition between viral DNA and the COL1A2 gene which decreases the expression of the cellular gene (see above). A heterozygous mutation of the COL1A2 gene causes the Ehlers-Danlos syndrome type-VII. EDS patients suffer from COL1A2 protein deficiency. Therefore, research on EDS type-VII can be used to gain insights on the effects of a GABP viral infection on animal and human health.

The COL1A2 deficieny in EDS type-VII causes hypermobility of joints (Byers 1997³²⁵, Giunta 1999³²⁶). A hypermobile joint is defined as a joint whose range of movement exceeds the norm for that individual, taking into consideration age, sex, and ethnic background. The primary cause of hypermobility is ligamentous laxity, which is determined by each person's fibrous protein genes (Grahame 1999³²⁷).

A high concentration of collagen type I, 55-65% of dry weight, is found in the matrix components of interarticular fibrocartilages (menisci) tissues. Meniscus tissues are found in the temporomandibular, temoclavicular, acromiocalvicular, wrist and knee joints. High concentration of collagen type I is also found in connecting fibrocartilages, such as vertebrae discs. As a result of COL1A2 deficiency, these joints show a higher degree of hypermobility compared to other joints. We call the temporomandibular, ternoclavicular, acromiocalvicular, wrist, knee and lumber joints the “Vulnerable Joints.”

A latent infection by a GABP virus results in microcompetition between viral DNA and the COL1A2 gene which decreases the expression of COL1A2. A COL1A2 deficiency causes hypermobility in vulnerable joints, specifically, in the lumbar joints. A infection also results in decreased expression of the hMT-II_{A} gene and obesity (see above). Therefore, obese people should show hypermobility in their lumbar joints.

A modified Schober test was used to examine lumbar mobility. To perform the test, the subjects were first asked to stand erect. While erect, three marks were placed on the subject's skin overlaying the lumbosacral spine. The first mark was placed at the lumbosacral junction, the second mark was placed 5 cm below the first, and the third mark was placed 10 cm above the junction. The subject was then asked to bend forward as far as possible, as though to touch the toes. The new distance between the second and third mark was measured. Lumbar mobility is defined as the difference between this measurement and the initial distance of 15 cm. The study group included 2,350 men and 670 women between the ages of 21 and 67 years.

Obesity (defined as weight/height) markedly affected the flexibility measurements. For every increase in obesity by one standard deviation, an increase of 0.4 cm was measured in the modified Schober measurement. The results showed that younger subjects are more mobile in their lumbar joints. Female subjects in their 20's showed an increase of 0.42 cm in the modified Schober measurement compared to female in their 60's. Man showed a 1.04 cm increase over the same age difference. The increased flexibility demonstrated by the most obese subjects (top 16%, or 1 SD of weight/height subjects) is equal to the increase in flexibility associated with 40 year age difference in female (0.4 cm compared to 0.42 cm), and is almost half the increase associated with that age difference in men (0.4 cm compared to 1.04 cm) (Batti'e 1987³²⁸).

A study with EDS patients found that 16 out of 22 over the age of 40 have osteoarthritis of one or more joints (referenced in Grahame 1989³²⁹). In the general population, evidence is more circumstantial. However, the Leeds groups produced evidence of a likely association between joint laxity and osteoarthritis (OA). The study compared 50 women with symptomatic OA to age matched controls. The study found a direct correlation between developing OA and the degree of hypermobility (Scoott 1979³³⁰)

The association between hypermobility and osteoarthritis was studied in specific joints. Sharma, et al., (1999³³¹) report that laxity is greater in the uninvolved knees of OA patients compared to knees of older controls. The authors concluded that at least some of the increased laxity of OA may predate the disease. Jonsson, et al., (1996³³²) compared 50 female patients with clinical thumb base (first carpometacarpal joint) OA to age matched controls. The results showed that hypermobility features were much more prevalent in the 50 patients compared to controls. The authors also report another study with 100 patients (including both males and females) that found a direct correlation between hypernobility and clinical severity of thumb base OA. They concluded that a causal relationship existes between articular hypermobility and thumb base OA.

Microcompetition causes hypermobility which causes osteoarthritis in vulnerable joints. Microcompetition also causes obesity. Therefore, obese people should show osteoarthritis in vulnerable joints.

A study compared the OA disease traits in different joints of female twins aged 48-70. The results showed that, in twins, an increase in the body weight increased the likelihood of developing osteoarthritis in the knee in both the tibiofemoral joint (TFJ) and patellofemoral joint (PFJ) and in the hand in the first carpometacarpal joint (CMC I). Specifically, after adjustment for other potential risk factors, for every 1 kg increase in body weight a twin had a 14% increased risk of developing TFJ osteophytes, a 32% increased risk of developing PFJ osteophytes, and a 10% increased risk of developing CMC osteophytes compared to their co-twin. Moreover, the weight difference was also observed in asymptomatic woman, which indicates that weight gain predates OA and, therefore, is not a result of OA (Cicuttini 1996³³³).

Note that this twin study demostrates an association betweeen obesity and OA independent of genetic factors, and is, therefore, inconsistent with the genetic mutation explantion of obesity (see above).

A longitudinal study began in 1962 with baseline examinations of clinical, biochemical, and radiologic characteristics. In 1985 follow-up examination characterized osteoarthritis in 1,276 participants, 588 males and 688 females, ages 50-74. Baseline obesity was measured by an index or relative weight. The results showed that the likelihood of developing osteoarthritis of the hand over the 23 year period increased with an increase in the index measuring baseline relative weight. Higher baseline relative weight was also associated with greater subsequent severity of the disease. Moreover, during the 23 year period, most subjects gained weight. However, after adjustment for baseline weight, the increase in body weight was not associated with either the likelihood of developing osteoarthritis of the hand or the severity of the disease, which indicates that OA is not a result of weight gain (Carman 1994³³⁴).

In obesity some joints seem to be susceptible to osteoarthritis while other are protected. The knees and the thumb base, for intance, are often damaged while the hips are disease free. Since both are weight bearing joints, the difference in susceptibility to osteoarthritis indicates a cause other than mechanical wear-and-tear. The pattern of OA in obesity also does not correspond to a general metabolic cause for the disease. A metabolically induced deterioration of cartilage should result in small differences in the severity of OA between joints, unlike the differences observed in joints of obese people. van Sasse, et al., call the pattern of OA in obesity “strange,” and claims that “whatever the final explanation for the etiology of OA, we believe that it will have to take into account the strange pattern of the association between OA and obesity” (van Saase 1988³³⁵).

These studies suggest three insights. First, obesity is associated with osteoarthritis in only specific joints—van Saase's “strange” list of susceptible joints. Second, obesity and osteoarthritis do not a result from of each other. Third, the association between obesity and osteoarthritis is independent of genetic factors. Obesity and OA resulting from microcompetition between viral and cellular DNA is consistent with all three insights. First, van Saase's “strange” list of susceptible joints coincides with the list of vulnerable joints. Second, both obesity and OA result from microcompetition and not from each other. Last, microcompetition results from a viral infection and not from a genetic mutation.

3. Collagen Type I α2 Chain (COL1A2), Obesity and Obstructive Sleep Apnea (OSA)

Obesity is associated with hypermobility of vulnerable joints. The temporomandibular joint belongs to the list of vulnerable joints. Therefore, in obesity the temporomandibular joint is hypermobile.

The mandible and tongue protrusion of obese patients was compared to controls. The subject was asked to protrude the mandible or tongue as far forward as possible (MAX), and 50% was measured as the midpoint between maximum protrusion and the position were the tongue tip is resting between the incisors (50%). The difference between resting position R and MAX and between R and 50%, is denoted R-MAX and R-50%, respectively. The results showed that obese subjects differed from controls in the degree of change in cross-sectional area (CSA) in the oropharyn. XThe 50% mandibular protrusion (R-50%) and the maximum tongue protrusion (R-MAX) produced greater relative increases in oropharyngeal cross-sectional area in obese subjects compared to controls (Ferguson 1997³³⁶). Increased oropharyngeal cross-sectional area indicates an increased capacity for mandibular protrusion. Such increased capacity indicates hypermobility of the temporomandibular joint.

During sleep, the tonic activity of the masseter decreases. In a supine position the mandible drops and the mouth opens. A hypermobile temporomandibular joint lets the mandibular drop further and the mouth open wider than a normal joint.

A study compared the time spent with mandibular opening in OSA patients and healthy controls. In controls, 88.9% of total sleep time was spent with narrow mandibular opening (less than 5 mm). In contrast, in OSA patients, 69.3% of the total sleep time was spent with wide mandibular opening (more than 5 mm). Moreover, in healthy adults, there was no difference in mandibular posture between the supine and lateral recumbent positions, while in OSA patients, sleep stage affects the mandibular opening during sleep in the supine position only (Miyamoto 1999³³⁷).

The abnormal low position of the hypermobile mandibular causes the upper airway disturbances during sleep. Therefore, hypermobility of the temporomandibular joint causes OSA.

Without reference to hypermobility of the temporomandibular joint, Miyamoto, et al., (1999) proposes a similar description of the events leading to apnoeic episodes.

Microcompetition causes obesity. Microcompetition also causes hypermobility of the temporomandibular joint which, in turn, causes OSA. Therefore, obesity is associated with OSA (note that the OSA patients in Ferguson, et al, (1997³³⁸) and Miyamoto, et al., (1999) studies above are obese).

### F. Clinical Effect of Microcompetition—Obesity

1. Background

a) The Obesity Epidemic

“The prevalence of obesity (defined as body mass index≧30 kg/m²) increased from 12.0% in 1991 to 17.9% in 1998. A steady increase was observed in all states; in both sexes; across age groups, races, education levels; and occurred regardless of smoking status” (Mokdad 1999³³⁹).

As proposed throughout the scientific community, the three “classical” causes of the obesity epidemic are increased energy intake, reduced energy expenditure, and genetic mutation.

b) Increased Energy Intake (“Too Much Food”)

Many large scale studies refute the idea that increased energy intake is the cause of obesity. The USDA Nationwide Food Consumption Survey 1977-1988 collected data from over 10,000 individuals. The analysis found that the average fat intake in the United States decreased from 41% to 37% of calorie intake between 1977 and 1988 and the average total energy intake decreased, by 3% in women and by 6% in men. “The reductions in average fat and energy intake were associated with a progressive increase in the prevalence of obesity in the US adult population” (Weinsier 1998³⁴⁰).

An even larger study reported similar results based on pooled data from NHANES II and III, USDA Nationwide Food Consumption Survey, Behavioral Risk Factor Survey System, and Calorie Control Council Report (Heini 1997³⁴¹). “In the adult US population the prevalence of overweight rose from 25.4% from 1976 to 1980 to 33.3% from 1988 to 1991, a 31% increase. During the same period, average fat intake, adjusted for total calories, dropped from 41.0% to 36.6%, an 11% decrease. Average total daily caloric intake also tended to decrease, from 1,854 kcal to 1,785 kcal (−4%). Men and women had similar trends. Concurrently, there was a dramatic rise in the percentage of the US population consuming low-calorie products, from 19% of the population in 1978 to 76% in 1991” (Ibid). The authors conclude that “reduced fat and calorie intake and frequent use of low-calorie food products have been associated with a paradoxical increase in the prevalence of obesity” (Ibid). Similar surveys conducted in Great Britain corroborate these studies.

c) Reduced Energy Expenditure (“Too Little Exercise”)

Many have turned their attention to reduced physical activity as an alternative explanation for the obesity epidemic. “The only available explanation for the paradoxical increase in body weight with a decrease in fat and energy intake is that physical activity declined” (Ibid). The data disprove this explanation as well.

In recent years several population surveys have shown unchanging levels of physical activity among Americans. For example, in the Behavioral Risk Factor Survey which included 30,000 to 80,000 individuals annually, the prevalence of obesity increased from 12% to 17.9% between 1991 and 1998 but physical inactivity did not change substantially (Ibid).

d) Genetic Mutation

“The fact that the increased rates of obesity have been observed within the last two decades has been viewed as evidence that genetic factors cannot be held responsible. Indeed, systematic changes of the population-based frequencies of specific alleles predisposing to obesity cannot possibly have occurred within this short time span.” (Hebebrand 2000³⁴²) A significant change in the human gene pool requires many generations. A genetic mutation explanation for the increase in obesity implies that the human gene pool has changed over a single generation. “Although research advances have highlighted the importance of molecular genetic factors in determining individual susceptibility to obesity, the landmark discoveries of leptin, uncoupling proteins and neuropeptides involved in body weight regulation, cannot explain the obesity epidemic” (Hill 1998³⁴³). “Genes related to obesity are clearly not responsible for the epidemic of obesity because the gene pool in the United States did not change significantly between 1980 and 1994” (Koplan 1999³⁴⁴).

2. Knockout Studies

a) Human Metallothionein-II_{A} (hMT-II_{A})

hMT-II_{A} is a microcompetition-suppressed gene. A latent infection by a GABP virus results in microcompetition between the viral DNA and the hMT-II_{A} gene which decreases the expression of the cellular gene (see above). A disruption of the metallothionein gene in transgenic mice also reduces the expression of the cellular gene. Therefore, research with MT-null mice can produce insights on the effects of a GABP viral infection on animal and human health.

MT-I and MT-II null mice are obese. Mice with disrupted MT-I and MT-II genes are apparently phenotypically normal. The disruption shows no adverse effect on the ability to reproduce and rear offspring. However, after weaning, MT-null mice consume more food and gain more weight at a more rapid rate than control mice. The majority of the adult male mice in the MT-null colony show moderate obesity (Beattie 1998³⁴⁵).

b) Integrin (β₂ leukocyte, CD18)

Notations and terminology:

β₂=CD18

α_{L}=CD11a (L for Leukocytes) expressed in all leukocytes

α_{M}=CD11b (M for Monocytes/Macrophage) expressed in monocytes/macrophages, granulocytes, natural killer cells, a sub population of T cells

LFA-1=Lymphocyte-Function-associated Antigens 1

MAC-1=Macrophage 1

CR3=Complement Receptor type 3

α_{L}β₂=CD11a/CD18=LFA-1 (LFA-1 binds ICAM-1 and ICAM-2)

α_{M}β₂=CD11b/CD18 MAC-1=CR3=Mo-1 (MAC-1 binds ICAM-1, C3b, fibrinogen and factor X)

CD18 is a microcompetition-suppressed gene. CD18 is a leukocyte-specific adhesion molecule. GABP binds three N-boxes in the CD18 promoter and transactivates the gene (Rosmarin 1995³⁴⁶, Rosmarin 1998³⁴⁷). Since CD18 is a GABP stimulated gene, latent infection by a GABP virus results in microcompetition between the viral DNA and the CD18 promoter which decreases the expression of CD18 (see Le Naour 1997³⁴⁸, Tanaka 1995³⁴⁹, Patarroyo 1988³⁵⁰ above). Moreover, the higher the concentration of viral DNA, the greater the decrease in CD18 expression.

ICAM-1 or MAC-1 null mice are obese. CD18 participates in forming the CD11a/CD18 molecule. CD11a/CD18 binds ICAM-1. ICAM-1 null mice (ICAM-1 −/−) gain more weight than control mice after 16 weeks of age, and eventually became obese despite no obvious increase in food intake. Under a high fat diet, ICAM-1 −/− mice show an increase susceptibility to obesity. CD18 also participates in forming the CD11b/CD18 molecule. CD11b/CD18 binds MAC-1. MAC-1 null mice (MAC-1 −/−) are also susceptible to diet-induced obesity and exhibited a strong similarity in weight gain with sex-matched ICAM-1 −/− mice (Dong 1997³⁵¹).

3. Pathogenesis

a) Hormone Sensitive Lipase (HSL) Gene

HSL is a microcompetition-suppressed gene. HSL mRNA, protein expression, and enzyme activity was measured in abdominal subcutaneous adipocytes from 34 obese drug-free and otherwise healthy males and females and 14 non-obese control subjects. The results showed reduced HSL mRNA, protein expression and enzyme activity (Large 1999³⁵², Table 3). The findings were age and gender independent. Based on these results Large, et al., conclude that “a decrease synthesis of the HSL protein at the transcriptional level is a likely factor behind the findings of decreased HSL expression in adipocytes from obese subjects. . . . Decreased HSL expression may at least in part explain the well-documented resistance to the lipolytic effect of catecholamines in obesity.”

In line with these results, a subsequent study by the same laboratory showed a 73% reduction in HSL protein levels in obesity (Elizalde 2000³⁵³, FIG. 4C and Table 1).

Catecholamines bind β₁-, β₂- and β₃-adrenergic receptors (β₁AR, β₂AR and β₃AR, respectively) and α₂ adrenergic receptors (α₂AR).

Activation of β₂AR (Maudsley 2000³⁵⁴, Pierce 2000³⁵⁵, Elorza 2000³⁵⁶, Luttrell 1999³⁵⁷, Daaka 1998³⁵⁸) or β₃AR (Cao 2000³⁵⁹, Gerhardt 1999³⁶⁰, Soeder 1999³⁶¹) activates ERK. ERK phosphorylates GABP. Phosphorylated GABP binds p300, resulting in increased HSL transcription.

Activation of β₁AR, β₂AR, β₃AR activates a cAMP dependent protein kinase A. The protein kinase phosphorylates HSL, resulting in increased hydrolytic activity against triacylglycersol and cholesteryl ester substrates. Insulin deactivates HSL via protein phosphatases or inhibition of protein kinase.

Microcompetition reduces HSL expression. Since HSL is rate limiting in triacylglycerol and diacylglycerol hydrolysis, microcompetition reduces steady state lipolysis. Moreover, as GABP kinase agent, β₂AR and β₃AR agonist, specifically, catecholamines, stimulate HSL transcription. Microcompetition also lessen the increase in HSL transcription, resulting in impaired stimulated lipolysis. FIG. 9 illustrates how microcompetition reduces lipolysis per adipocyte. At steady state, microcompetition reduces lipolysis per adipocyte. Microcompetition also reduces the slope of the lipolysis line. That is, with increased stimulation, the relative lipolysis deficiency (the vertical difference between the two lines) increases.

A number of in vivo and in vitro studies demonstrated reduced ability of catecholamines to stimulate lipids mobilization from subcutaneous adipose tissue.

Hellstrom, et al., (1996³⁶²) treated abdominal subcutaneous adipocytes from 13 non-obese subjects with at least one first-degree relative with body mass index of 27 kg/m2 or more (Hob) and 14 controls (Hnorm), with norepinephrine, a major endogenous lipolytic agent, isoprenaline, a non-selective beta-adrenoceptor agonist, forskolin, a direct activator of adenylyl cyclase, and dibutyryl cyclic AMP, activator of protein kinase and thereby HSL. The effect of these treatments on the glycerol release (pmol•cell•2h⁻¹) from adipocytes was examined. The average rate of lipolysis induced by all four treatments was reduced by about 50% (p from 0.001 to 0.01) in subjects with a family trait of obesity relative to controls.

Isoprenaline (Shimizu 1997³⁶³), dibutyryl cAMP (Shimizu 1997) and forskolin (Yarwood 1996³⁶⁴) activated ERK in adipocytes. Isoprenaline also activated ERK in CHO/K1 cells expressing the human β₃AR (Gerhardt 1999³⁶⁵). As GABP kinase agent the agonists phosphorylate GABP. Microcompetition in obese adipocytes reduces the maximum number of GABP molecules available for HSL promoter binding. Hence, the observed resistance for these agonists stimulation. Moreover, as expected, an increase in the agonist concentration increased the relative lipolysis deficiency.

Hellstrom, et al., (1996) also measured the HSL maximum activity and HSL mRNA at steady state. The maximum activiy was reduced 50% in Hob (p<0.05). mRNA (amol HSL/μg total nucleic acids) was reduced 20% (p>0.05, not significant). The study did not measure HSL mRNA after stimulation.

The following studies use the concept of maximum adipocyte lipolysis capacity in response to stimulation by various agonists. The studies compared glycerol release in adipocytes from obese male and female and controls. In all studies the adipocyte incubation in the presence of the agonist lasted 2 h.

Large, et al., (1999³⁶⁶) treated abdominal subcutaneous adipocytes from 34 obese drug-free and otherwise healthy males or females and 14 non-obese controls, with isoprenaline, a non-selective β-adrenergic receptor agonist, or dibutyryl cAMP, a phosphodiesterase resistant cAMP analogue. The results showed reduced maximum values for isoprenaline- and dibutyryl cAMP induced glycerol release by 40-50% in the obese group, when expressed per g lipid.

Hellstrom, et al., (2000³⁶⁷) treated abdominal subcutaneous adipocytes from 60 obese and 67 non obese subjects, age 19-60 y, with isoprenaline, dibutyryl cyclic AMP, and forskolin, an activator of the adenylyl cyclase. The results showed reduced maximum values for isoprenaline-, dibutyryl cAMP-, and forskolin induced glycerol release by 50% in the obese group. Moreover, 42 of the 67 lean subjects had at least one obese member among first degree relatives, but not all family members, and not both parents. The non-obese subject with the family trait for obesity showed a similar reduction in maximum glycerol release compare to lean subjects without the family trait.

Consider Bougneres 1997³⁶⁸. To study the effect of epinephrine on lipolysis in obesity, epinephrine was infused stepwise at fixed doses of 0.75 and then 1.50 μg/min to 9 obese children (160+/−5% ideal body weight) aged 12.1 +/−0.1 yr during the dynamic phase of fat deposition, and in 6 age-matched non-obese children. As an in vivo lipolysis index, the study used glycerol flu. XIn the basal state, obese children had a 30% lower rate of glycerol release per unit fat mass than lean children. FIG. 10a represents the measured relationship between epinephrine infusion and glycerol release.

Consider Horowitz (2000³⁶⁹). Lipolytic sensitivity to epinephrine was measured in 8 lean [body mass index (BMI): 21±1 kg/m(2)] and 10 upper body obese (UBO) women (BMI: 38±1 kg/m(2); waist circumference>100 cm). All subjects underwent a four-stage epinephrine infusion (0.00125, 0.005, 0.0125, and 0.025 microgram•kg fat-free mass⁻¹•min⁻¹) plus pancreatic hormonal clamp. Glycerol rates of appearance (Rₐ) in plasma were determined by stable isotope tracer methodology. FIG. 10b represents the measured percent change in glycerol release as a function of plasma epinephrine concentration. FIG. 10c represents the same results in terms of total glycerol release per fat mass (FM). Both Bougneres 1997 and Horowitz 2000 results are consistent with microcompetition as the underlying cause of catecholamine resistance in obesity.

HSL is a GABP gene. Microcompetition reduces HSL expression. Reduced HSL expression result in adipocyte hypertrophy. Consider the following study.

HSL knockout mice were generated by homologous recombination in embryonic stem cells. Cholesterol ester hydrolase (NCEH) activities were completely absent from both brown adipose tissue (BAT) and white adipose tissue (WAT) in mice homozygous for the mutant HSL allele (HSL−/−). The cytoplasmic area of BAT adipocytes was increased 5-fold in HSL−/− mice (Osuga 2000³⁷⁰, FIG. 3a). The median cytoplasmic areas in WAT was enlarged 2-fold (Ibid, FIG. 3b). The HSL knockout mice showed adipocyte hypertrophy.

Obesity is characterized by adipocyte hypertrophy. Osuga 2000 results are consistent with microcompetition as the underlying cause of adipocyte hypertrophy in obesity.

It is interesting that body weight of the HSL−/− mice was not different, at least until 24 weeks of age, from wild-type. The reason was probably lack of adipocyte hyperplasia in HSL−/− mice. Consider the following section.

b) Retinoblastoma Susceptible Gene (Rb)

Rb is a microcompetition-suppressed gene. Rb-null (pRb(−/−)) preadipocytes show higher proliferation rate compared to wild type. A study measured the percentage of pRb(−/−) 3T3 cells in S phase following five different treatments, cells grown in DMEM (asynchronous cells, marked A), cells grown to confluence in DMEM containing 10% calf serum and than maintained for 6 days in same mixture (marked C), confluent cells split into subconfluent conditions (marked CR), confluent cells treated for 6 days with an adipocyte differentiating mixture (marked D), and differentiated cell split into subconfluent conditions (market DR) (Classon 2000³⁷¹, FIG. 3A).

Asynchronous pRb(−/−) cells show a tendency for excessive cell replication. Moreover, pRb(−/−) differentiated cells show a higher probability for cell cycle re-entry. It should be emphasized that although pRb seem to affect the establishment of a permanent exit from cell cycle, pRb is not absolutely required since expression of C/EBPα and PPARγ bypasses the requirement for pRb and causes pRb(−/−) cells to differentiate into adipocytes (Classon 2000, FIG. 1B).

The transcription of the Rb gene increases with growth arrest and differentiation (see above). The relationship between pRb concentration and adipocyte differentiation was tested in a study that compared proliferative and differentiated brown (primary) and white (3T3-F442A) adipocytes in culture. The differentiation stage of the cells was determined following detection of lipid accumulation and expression of the specific differentiation markers aP2 and UCP-1. The results showed almost undetectable pRb levels in proliferative undifferentiated cells. On the other hand, pRb was clearly detected in nuclei of differentiated primary brown adipocytes (Puigserver 1998³⁷², FIG. 2A) with lipid accumulation in their cytoplasm and UCP-1 expression (Ibid, FIG. 3) and in 3T3-F442A cells with lipid accumulation and aP2 expression. Moreover, Puigserver, et al., note that “the pRb levels measured by immunoblotting clearly increased during differentiation of 3T3 F442A cells (Ibid, FIG. 2B)” and that “there was an apparent positive correlation between pRb expression and lipid accumulation, since nuclei from cells with more lipid droplets in their cytoplasm were more strongly immunostained for pRb than those of cells with less lipid droplets (Ibid, FIG. 2A).”

Richon, et al., (1992³⁷³) proposed the following model for the relationship between Rb and growth arrest and differentiation (see also above). An inducer increases Rb transcription resulting in higher hypo- and total-pRb concentration. The increase in hypo-pRb prolongs G1. However, the initial increase in hypo-pRb is most likely not sufficient for permanent G1 arrest. Therefore, cells reenter cell cycle for a few more generations. While cells continue to divide, the increased rate of transcription results in hypo-pRb accumulation. When a critical hypo-pRb concentration, or threshold, is reached, the cell irreversibly commit to terminal differentiation. This model describe the determination of the commitment to differentiate as a stochastic process with progressive increases in the probability of G1/G0 arrest and differentiation established through successive cell divisions. Such a model would predict an increase in the number of cell cycle generations required for producing the threshold Rb concentration, under conditions of suppressed Rb transcription. FIG. 11 illustrates how microcompetition reduces Rb transcription. Therefore, the number of generations required to reach the required Rb concentration ([Rb]₀) under microcompetition (N_{M}) is greater than the number in controls (N_{C}). In obesity, therefore, one should observe excessive replication in vitro (Roncari 1986³⁷⁴, Roncari 1981³⁷⁵) and hyperplasia in vivo.

Returning to the non-obese HSL−/− mice (Osuga 2000, see above). Both HSL and Rb are microcompetition-suppressed genes. Therefore, both genes show reduced expression in obesity, resulting in adipocyte hypertrophy and hyperplasia. Since Rb transcription is most likely independent of HSL expression, pRb in HSL−/− mice is not under expressed and adipocytes in HSL−/− mice are not hyperplastic.

4. Studies in Signaling

a) Resistant GABP Kinase Agent in Obesity

The following are GABP kinase agent showing cellular level or patient level resistance in obesity (for definition of cellular and patient level resistance and its relationship to microcompetition, see above).

Oxytocin: The oxytocin receptor (OTR) is a GABP gene (see above). Stock, et al., (1989³⁷⁶) tested whether the plasma level of oxytocin was elevated in obese subjects and if so, whether it was affected by weight reduction following gastric banding. Plasma levels of oxytocin were 4-fold higher in the obese subjects than in the control subjects. After the operation, oxytocin levels dropped dramatically, but were still markedly higher than control. Moreover, obese pregnant women need more oxytocin stimulation of labor. Johnson, et al., found that, compared to a control group matched for age and parity, there was a significantly increased need for oxytocin stimulation of labor in obese patients weighing at least 113.6 kg (250 pounds) during pregnancy (Johnson 1987³⁷⁷).

Zinc and Copper: Serum zinc, copper and magnesium levels were measured in healthy and obese children using atomic absorption spectrophotometry. Serum zinc and copper levels of obese children (mean value 102.40±2.78 micrograms/dL mean value 132.34±1.79 micrograms/dL, respectively) were markedly higher than control (mean value 80.49±2.98 micrograms/dL, and mean value 107.58±1.62 micrograms/dL, respectively). Serum copper concentrations were also significantly higher in obese children compared to healthy controls (Yakinci 1997³⁷⁸). Serum zinc and copper levels were also determined in 140 diabetic patients and 162 healthy controls. A sub group of patients were classified as overweight (greater than 15% relative body weight). Obese patients showed a statistically singnificant increase in zinc levels. The copper level positively correlated with the zinc level (D'Ocon 1987³⁷⁹). Taneja, et al., (1996³⁸⁰) measured the concentration of zinc in hair of obese man and woman. The results showed a positive linear correlation between body weight, or body weight/height ratio, and hair zinc concentration. The correlation was stronger in man.

The following hormones and cytokines, which are all GABP kinase agents, also show resistance in obesity.

Insulin: Patients with non-insulin-dependent diabetes mellitus (NIDDM) and/or obesity generally suffer from insulin resistance (IR). Interestingly, most NIDDM patients are obese. Ludvik, et al., studied the effect of obesity and NIDDM on insulin resistance. Both lean NIDDM subjects and obese normal subjects were significantly insulin resistant compared to lean normal subjects (Ludvik 1995³⁸¹). Another study observed kinetic defects in insulin action in insulin resistant nondiabetic obese subjects. Insulin-stimulated glucose disposal was slower to activate and more rapidly deactivated in obese than in normal subjects. Oral glucose tolerance tests (OGTTs) were done in five controls and five obese subjects. While each of the control subjects had normal glucose tolerance, only two obese subjects tested normal for glucose tolerance. The remaining three obese subjects had impaired glucose tolerance. During the OGTT, both glucose and insulin levels were significantly higher in the obese subjects than the controls (Prager 1987³⁸²).

Leptin: The levels of leptin in plasma increases with body weight (body mass index, BMI kg/m2). Plasma leptin levels are higher in females compared to males (Tasaka 1997³⁸³). The ob/ob mouse has a mutated ob gene. The deficiency of leptin in the ob/ob mouse produces severe obesity. Contrary to the ob/ob mouse (and the db/db mouse with the mutated leptin receptor), in most obese humans the leptin and leptin receptors genes are normal. Moreover, except for some rare cases, the level of leptin in obese humans is elevated rather than reduced (Bjorbaek 1999³⁸⁴).

Estrone, estradiol: Urinary excretion of estrone (E1), estradiol (E2) and estriol (E3) was measured in obese post-menopausal women before and 6-12 months following participation in a weight loss program. Prior to the weight loss program, there was a significant correlation between estrone, weight and the Quetelet-index of obesity and between estriol and the Quetelet-index (de Waard 1982³⁸⁵).

Levels of serum sex hormones were studied in healthy, white postmenopausal women (mean age 58 years). Extraction, column chomatography, and radioimmunassay were used in combination to measure the serum concentrations of estrone, estradiol, testosterone, and androstenedione. Obesity was a major predictor of estrone and estradiol levels. Obese women had estrone levels 40% higher than nonobese women (Cauley 1989³⁸⁶). In a subsequent study, Cauley, et al., (1994³⁸⁷) compared the sex steroid hormone levels between white and black women 65 years of age or older. The researchers used the same techniques to measure the serum levels of estrone, androstenedione, and testosterone as in the 1989 study. The results showed that black women had significantly higher serum estrone concentrations and markedly lower androstenedione levels compared to white women. There was a corresponding difference in the degree of obesity between the two groups.

Interleukin 1 β (IL-1β): Human coronary artery specimens from patients suffering from either coronary atherosclerosis or cardiomyopathy were studied for levels of IL-1β (Galea 1996³⁸⁸). The presence of IL-1β correlated with disease severity. The study discovered that IL-1β protein is elevated in the adventitial vessel walls of atherosclerotic coronary arteries compared to coronary arteries from nonischemic cardiomyopathic hearts. Serum IL-1β levels were also determined in patients with ischaemic heart disease. The results showed that the mean serum IL-1β concentrations were higher in patients with ischaemic heart disease, in particular in those with minimal coronary artery disease and angina (Hasdai 1996³⁸⁹).

Interleukin 6 (IL-6): Type II diabetes mellitus (non-insulin-dependent diabetes mellitud, NIDDM) is assoicated with increased blood concentrations of markers of the acute-phase response, including interleukin-6. The combination of hypertriglyceridaemia, low serum HDL-cholesterol concentrations, hypertension, obesity and accelerated atherosclerosis, termed metabolic syndrome X, is often associated with NIDDM. To investigate thisassociation, two groups of Caucasian NIDDM patients were studied. The first group, with any 4 or 5 features of syndrome X, was compared with the second group, with 0 or 1 feature of syndrome. XThe groups were matched for age, sex, diabetes duration, glycaemic control and diabetes treatment. Age and sex matched healthy non-diabetic subjects were controls. The results showed a marked increase in serum IL-6 between the three groups. The lowest levels were found in non-diabetic subjects, intermediate levels in NIDDM patients with 0 or 1 feature of syndrome X and the highest levels in NIDDM patients with a 4 or 5 features (Pickup 1997³⁹⁰, Pickup 1998³⁹¹)

Tumor necrosis factor a (TNFα): Sixty five patients were tested for TNFα levels. The majority of the patients had android obesity, elevated leptin, insulin resistant, coronarographically confirmed microvascular angina pectoris or IHD. Most of the patients suffered from a myocardial infarction with one or more significant stenoses on the epicardial coronary arteries. Fifty percent of the patients had elevated TNFα, and 28% elevated IL-6 (Hmciar 1999³⁹²).

b) Non Resistant GABP Kinase Agent in Obesity

Some GABP kinase agents show no resistance. Consider the following cases.

Interleukin 2 β (IL-2β): IL-2β is a GABP kinase agent with the receptors, interleukin 2 receptor β chain (IL-2Rβ) and IL-2 receptor γ-chain (γc). Both receptors are stimulated by GABP (Markiewicz 1996, Lin 1993). Microcompetition for GABP reduces the transcription of the receptors. Since any control in this pathway has to be downstream from the receptors, microcompeition for GABP diminshes the expression of the control. The reduced expression of the control reduces its repressive effect on IL-2β, which elevates the concentration of IL-2β. However, IL-2β itself is a GABP stimulated gene (Avots 1997³⁹³). Therefore, microcompetition also reduces the transcription of IL-2β. The combined effect of diminished repression on transcription and diminished transactivation of transcription can results in a decline, increase, or no change in the concentration of IL-2β in obesity.

GM-CSF: Granulocyte-macrophage colony stimulating factor (GM-CSF) is a GABP kinase agent. One study showed that GM-CSF (20 ng/ml) significantly inhibited neutrophil apoptosis. The inhibition of apoptosis was significantly attenuated by PD98059, an MEK1 specific inhibitor (Klein 2000³⁹⁴). Another study showed that bone marrow-derived macrophages proliferate in response to GM-CSF. The MEK1 specific inhibitor, PD98059, blocked the GM-CSF stimulated cell proliferation. Moreover, this study showed the time-course of ERK activation by GM-CSF, where maximal activation occurred 5 min after stimulation (Valledor 2000³⁹⁵). As a GABP kinase agent, one would expect to observe resistance in obesity and obesity related disease. However, the GM-CSF gene is transactivated by ets1 (Thomas 1997³⁹⁶). Therefore, microcompetition for ets1 can result in either a decline, increase or no change in GM-CSF concentration in obesity and obesity related diseases.

5. Studies with Viruses

Until recently, the relationship between viral infection and human obesity has been completely ignored.

a) Human Adenovirus 36 (Ad-36)

A recent study inoculated chickens and mice with human adenovirus Ad-36. Weight matched groups were inoculated with tissue culture media as non-infected controls. Ad-36 inoculated and uninfected control groups were housed in separate rooms under biosafety level 2 or better containment. The chickens study was repeated three times. The first chickens experiment included an additional weight matched group of chickens that was inoculated with CELO (chick embryo lethal orphan virus), an avian adenovirus. Food intakes and body weights were measured weekly. At the time of sacrifice blood was drawn and visceral fat was separated and weighed. Total body fat was determined by chemical extraction of carcass fat. In experiment 1, the results showed that the visceral fat of the Ad-36 chickens was 100% greater that controls (Dhurandhar 2000³⁹⁷, Table 1), in experiment 2, visceral fat was 128% greater than controls (Ibid, Table 3), in experiment 3, visceral fat was 74% greater than control (Ibid, Table 4). In all three experiments there was no difference in food intake or body weight between Ad-36 chickens and controls. Chickens inoculated with CELO virus showed no change in visceral fat. The Ad-36 mice visceral fat was 67% greater than controls and mean body weight was 9% greater. There was no difference in food intake. Sections of the brain and hypothalamus of Ad-36 inoculated animals showed no overt histopathological changes. Ad-36 DNA could be detected in adipose tissue, but not skeletal muscles of randomly selected animals for as long as 16 weeks after Ad-36 inoculation. Based on these results Dhurandhar concluded that “the role of viral disease in the etiology of human obesity must be considered.”

b) HIV

Recently, several studies documented a new syndrome associated with HIV infection termed “lipodystophy,” or “fat redistribution syndrome” (FRS). The symptoms typical of FRS, such as peripheral lipodystrophy, cenrtral adiposity, hyperlipidemia and insulin resistance (for a recent review see Behrens 2000³⁹⁸), are similar to syndrome X symptoms (Engelson 1999³⁹⁹) (Syndrome X is also known as “insulin resistance” or plain “=obesity.”) The cause of FRS is unknown. The temporal association between the recognition of FRS and the application of protease inhibitor therapy has led several investigators to conclude that FRS is a result of protease inhibitor therapy. However, since FRS was also identified in HIV-infected patients who were not taking protease inhibitors, other researchers concluded that FRS might be a characteristic of the HIV infection, only unmaked by prolonged survival associated with protease inhibitors treatment.

HIV is a GABP virus. HIV infection results in microcompetition between virus and the host. The microcompetition leads to obesity. (Moreover, recent studies report that HIV infection is assoicated with a greater risk of developing atherosclerosis and diabetes mellitus. Atherosclerosis and diabetes mellitus are another two diseases caused by microcompetition.)

6. Microcompetition-like Obesity

A genetic mutation, injury or diet can result in a deficiency in a GABP kinase agent or ERK receptor. Such deficiency produces a weak ERK signal. A weak ERK signal induces microcompetition-like symptoms.

a) Leptin

Homozygous mutations in genes encoding leptin or the leptin receptor lead to early-onset obesity and hyperphagia (Clement 1998⁴⁰⁰). For instance, mutation in the ob (leptin) gene is associated with obesity in the ob/ob mouse.

Obesity in the db/db mouse is associated with mutations in the db (leptin receptor) gene. An alternatively spliced transcript of the leptin receptor encodes a form with a long intracellular domain. The db/db mouse produces this alternatively spliced transcript with a 106 nucleotide insertion that prematurely terminates the intracellular domain. Moreover, the db/db mouse also exhibit a point mutation (G→T) in the same gene. The long intracellular domain form of the receptor participates in signal transduction. The inability to produce the long form in db/db mice contributes to their extreme obese phenotype (Chen 1996⁴⁰¹).

Obesity in the Zucker (fa/fa) rat is associated with mutations in the fa gene which encodes a leptin recpetor. The fa mutation is a missense mutation (269 gln→pro) in the extracellular domain of the leptin receptor. This mutation causes a decrease in cell-surface expression, a decrease in leptin binding affinity, defective signaling to the JAK-STAT pathway and reduced ability to activate transcription of the egr1 promoter (de Silva 1998⁴⁰²). Yamashita, et al., found that by binding to the long form of its receptor, leptin increased the tyrosine phosphorylation of STAT3 and ERK in Chinese hamster ovary (CHO) cells. In CHO cells with a fa mutated receptor, the leptin induced phosphorylation of both STAT3 and ERK was lower (Yamashita 1998⁴⁰³).

ERK complements

Let A and B be two GABP kinase agent. Assume that A is not an ERK receptor for B. Administration of B can alleviate the symptoms associated with a deficiency in A or an ERK receptor for A.

If A is not an ERK receptor for B, B will be called an “ERK Complement” for A. Notice that the relation is asymmetric. If B is downstream from A, B is an ERK complement for A, while A is not an ERK complement for B.

IL-1β as ERK complement for leptin

A low dose injection of human recombinant IL1β to gentically obese ob/ob and db/db mice normalized glucose blood levels for several hours (del Rey 1989⁴⁰⁴). In another study, chronic intracerebroventricular (ICV) microinjection of IL1β to obese (fa/fa) Zucker rats caused a 66.1% decrease in nighttime food intake (Ilyin 1996⁴⁰⁵).

Luheshi, et al., (1999⁴⁰⁶) showed that IL-1β is an ERK receptor for leptin. However, IL-1β can still be as ERK complement for leptin if leptin is not a receptor for IL-1β (asymmetry of the complement condition).

TNFα as ERK complement for leptin

ICV microinjection of TNFα (50, 100 and 500 ng/rat) to obese (fa/fa) Zucker rats decreased short-term feeding (4 hours) by 17%, 20%, and 20%, nighttime feeding (12 hours) by 13%, 14% and 13% and total daily food intake by 11%,12% and 11%, respectively (Plata Salaman 1997⁴⁰⁷).

LPS as ERK complement for leptin

Administration of LPS (0.1, 1, 10, 100 μg) to db/db mice induced a significant decrease in food intake (25%, 40%, 60%, 85%, respectively, in the first 24 hours post injection). The effect on ob/ob mice was similar (Faggioni 1997⁴⁰⁸).

b) Insulin

A mutation in the insulin receptor substrate-1 (IRS-1) is a risk factor for coronary artery disease (CAD). Insulin resistance is correlated with a higher risk of atherosclerosis. Insulin receptor substrate-1 (IRS-1) is a key component of tissue insulin sensitivity. A mutation (G972R) of the IRS-1 gene, which reduces IRS-1 function and has been connected to decreased sensitivity to insulin, was studied to see if it had any role in predisposing individuals to coronary artery disease (CAD). In this study, CAD patients had a much higher incidence of the mutation than the control group (18.9% versus 6.8%, respectively). The relative risk of CAD associated with the mutation increased in the obese patients and patients with a cluster of abnormalities of insulin resistance syndrome. These results indicate that The G972R mutation in the IRS-1 gene is a strong independent predictor of CAD. In addition, this mutation significantly enhanced the risk of CAD in both obese patients and in patients with clinical features of the insulin resistance syndrome (Baroni 1999⁴⁰⁹).

c) Transforming Growth Factor-β (TGFβ)

Mutations in the TFGβ receptor type II gene are associated with various cancers. Several human gastric cancer cells were studied for genetic abnormalities in the TGFβtype II receptor gene. Deletion of the type II receptor gene in two of eight cell lines, and amplification of the gene in another two lines was detected in Southern blots. Other abnormalities in the gastric cancer cells resistant to the growth inhibitory effect of TGFβ included expression of either truncated or no detectable TGFβ type II receptor mRNAs. The one cell line not resistant to the growth inhibitory effect of TGFβ showed no abnormalities in type II receptor gene (Park 1994⁴¹⁰). Mutation of the TFGβ receptor type II gene is characteristic of colon cancers with microsatellite instability or replication errors (RER+). Specific mutations in a polyadenine repeat of the TFGβ type II receptor gene are common in both RER+colon cancers and RER+gastric cancers (Myeroff 1995⁴¹¹).

Mutations in the TFGβ receptor type II gene are also associated with atherosclerosis. High fidelity PCR and restriction analysis was adapted to analyze deletions in an A10 microsatellite within TFGβ receptor type II gene. DNA from human atherosclerotic_lesions, and cells grown from lesions, showed acquired 1 and 2 bp deletions in TFGβ receptor type II gene. The mutations could be identified within specific patches of the lesion, while surrounding tissue, or unaffected arteries, exhibited the wild-type genotype. This deletion causes loss of receptor function, and thus, resistance to the antiproliferative and apoptotic effects of TGFβ1 (McCaffrey 1997⁴¹²).

A deficiency in the TFGβ receptor type II gene causes osteoarthritis. An overexpressed TFGβ cytoplasmically truncated type II receptor competes with the cellular receptors for complex formation, thereby acting as a dominant-negative mutant receptor. Transgenic mice expressing the dominant-negative mutant receptor in skeletal tissue developed progressive skeletal degeneration. The pathology strongly resembled human osteoarthritis. This controled expriment in mice shows that a weak TFGβ signal leads to the development of degenerative joint disease similar to osteoarthritis in humans (Serra 1997⁴¹³).

d) Estrone and Estradiol

The ovaries in polycystic ovary syndrome (PCOS) produce less estradiol as a response to follicle-stimulating hormone (Caruso 1993⁴¹⁴). PCOS is associated with high blood pressure, hyperinsulimia, insulin resistance and obesity.

Ovariectomy reduces the concentration of estradiol, sometimes to undetectable levels (Wronski 1987⁴¹⁵). Ovariectomy is also associated with obesity.

e) Zinc and Copper

Singh, et al., (1998⁴¹⁶) surveyed 3,575 subjects, aged 25 to 64 years. The results showed that prevalence of coronary artery disease (CAD), diabetes and glucose intolerance is associated with lower intake of dietary zinc. In addition, hypertension, hypertriglyceridemia and low high-density lipoprotein cholesterol levels increased as zinc intake decreased.

f) Metallothionein-null

Metallothionein is a receptor of the GABP kinase agent zinc. After weaning, MT-null mice consumed more food and gained more weight at a more rapid rate than control mice. The majority of the adult male mice in the MT-null colony showed moderate obesity (Beattie 1998⁴¹⁷).

g) CD18-null

Chinese hamster ovary (CHO) fibroblast cell lines were engineered to express the CD11a/CD18 or CD11b/CD18 antigen. These cell lines were induced with LPS. Otherwise LPS-nonresponsive fibroblasts became responsive to LPS upon heterologous expression of CD11a/CD18 and CD11b/CD18 (Flaherty 1997⁴¹⁸). CD11c/CD18 also activated cells after binding to LPS (Ingalls 1995⁴¹⁹). In another study, both wild type CD11b/CD18 and mutant CD11b/CD18 lacking the cytoplasmic domains still transmitted a signal in response to LPS (Ingalls 1997⁴²⁰). Although full length CD11b/CD18 is needed for productive phagocytic signals, LPS activation does not require the cytoplasmic domains. Perhaps CD11b/CD18 activates cells by presenting LPS to a downstream signal transducer (Ingalls 1997). These studies indicate that CD11a/CD18 and CD11b/CD18 are receptors of the GABP kinase agent LPS.

CD11a/CD18 binds the intercellular adhesion molecule-1 (ICAM-1). ICAM-1 null mice (ICAM-1 −/−) gained more weight than control mice after 16 weeks of age, and eventually became obese despite no obvious increase in food intake. ICAM-1 −/− mice also showed an increase susceptibility to develope obesity under a high fat diet.

CD11b/CD18 binds macrophage 1 (MAC-1). MAC-1 null mice (MAC-1 −/−) were also susceptible to diet-induced obesity, and exhibited a strong similarity in weight gain with sex-matched ICAM-1 −/− mice (Dong 1997⁴²¹).

### G. Microcompetition—like Clinical Symptoms

1. Drug Induced Microcompetition—like Symptoms

Drugs can induce microcompetition-like clinical symptoms. Common side effects associated with certain drugs are weight gain, insulin resistance, and hypertension. The following sections present the mechanism of these side effects.

a) Cytochrome P450

Three distinct pathways of arachidonic acid (AA) oxidation have been described. The enzyme systems involved are regiospecific and stereospecific. Of the three pathways, the products of the cyclooxygenase and lipoxygenase pathways have been extensivly researched. Theresearch on the products of the “third pathway”, the cytochrome P450-dependent monooxygenases, is less extensive. The “third pathway”, mediated by CYP enzymes, uses NADPH and molecular oxygen in a 1:1 stoichiometry. Three types of oxidative reactions are known to occur. Olefin epoxidation (epoxgenases) produces 4 sets of regio-isomers, the epoxyeicosatrienoic acids (EETS), specifically, the (5,6-), (8,9-), (11,12-) and 14,15-EETs. Allylic oxidation produces hydroxyeicosatetraenoic acids (HETEs), specifically, (5-), (8-) (9-), (11-), (12-) and 15-HETEs. FIG. 12 shows how omega oxidation produces the 19- and 20-HETEs.

b) Arachidonic Acid Metabolites Activate ERK

Rabbit VSMCs were treated with the vehicle dimethyl sulfoxide (VEH) alone or 20 μM PD98059 (PD) for 4 h and then exposed to 0.25 μM 12(R)-, 12(S)-, 15, or 20-hydroxyeicosatetraenoic acid (HETE) for 10 min. The study showed that MAP kinase activity was increased by 100%, 60% and 300% respectively after 12(S)-HETE, 15-HETE, and 20-HETE respectively for cells treated with VEH and more modestly for cells treated with PD (Muthalif 1998⁴²², FIG. 3A). The study also showed that 20-HETE specifically activated ERK1 and ERK2 (Ibid, FIG. 3D). Similar activation of MAPK by 12-, and 15-HETE are reported in Wen 1996⁴²³ and Rao 1994⁴²⁴.

Another study tested the effect of 14,15-epoxyeicosatrienoic acid (EET) on ERK activation. LLCPKc14, an established proximal tubule epithelial cell line derived from pig kidney, were treated with 14,15-EET (20 μm) for 15 min, then tyrosine phosphorylated proteins in cell lysates were immunoprecipitated with anti-phosphotyrosine antibodies and immunoblots probed with an antibody which recognizes ERK1 and ERK2. The results showed that 14,15-EET stimulated ERK1 and ERK2 phosphorylation (Chen 1999⁴²⁵, FIG. 2D).

To summerize, 12(S)-, 15, or 20-HETE and 14,15-EET activate ERK. In other words, these arachidonic acid metabolites are GABP kinase agent.

c) 12(S)-, 15, or 20-HETE and 14,15-EET CYP Specific Enzymes

The following table lists a few cytochrome P450 enzymes which produce GABP kinase agent metabolites. We call these enzymes CYP-ERKs. When the study is tissue specific, the tissue type is mentioned in the reference column.

| | GABP kinase agent | |
|---|---|---|
| Enzyme | product | Reference* |
| CYP1A2 | 14,15-EET | Rifkind 1995 (human liver) |
| CYP2B4 | 14(R),15(S)-EET | Zeldin 1995 (lung) |
| CYP2C8 | 14,15-EET | Rifkind 1995 (human liver) |
| CYP2C9 | 15(R)-HETE | Bylund 1998, |
| | 12-HETE | Rifkind 1995 (human liver) |
| CYP2C19 | 14,15-EET | Bylund 1998, Keeney 1998 (14S |
| | | 15R, skin keratinocytes) |
| | 12R-HETE | Keeney 1998 (skin keratinocytes) |
| | 15R-HETE | Keeney 1998 (skin keratinocytes) |
| CYP2C23 | 14,15-EET | Imaoka 1993 (rat kidney) |
| CYP2C29 | 14,15-EET | Luo 1998 |
| CYP2C39 | 14,15-EET | Luo 1998 |
| CYP2C37 | 12-HETE | Luo 1998 |

*Bylund 1998⁴²⁶, Imaoka 1993⁴²⁷, Zeldin 1995⁴²⁸, Rifkind 1995⁴²⁹, Luo 1998⁴³⁰, Keeney 1998⁴³¹

d) Drug Inhibition of CYP-ERK and Microcompetition—like Diseases

Microcompetition reduces the expression of GABP stimulated genes and increases the expression of GABP suppressed genes. Inhibition of a GABP kinase agent produces the same effect. Consider a drug which only inhibits CYP-ERK. That is, the drug has no other chemical reactions, such as inhibition of another enzyme. Call such a drug an “empty” drug. An empty drug should produce the same clinical profile as microcompetition.

The following table lists drugs which inhibit CYP-ERKs and their microcompetition-like side effects (mostly weight gain, some insulin resistance and atherosclerosis).

| | | |
|---|---|---|
| | | Microcompetition-like |
| Drug | Cytochrome P450 (CYP type) | symptoms |

Cytochrome P450 inhibitors

| | | |
|---|---|---|
| Phenytoin | Kidd 1999⁴³² (CYP2C9) | Egger 1981⁴³⁵ |
| | Ring 1996⁴³³ (CYP2C9) | |
| | Miners 1998⁴³⁴ (CYP2C9) | |
| Glipizide | Kidd 1999⁴³⁶ (CYP2C9) | Campbell 1994⁴³⁷ |
| Carbamazepin | Petersen 1995⁴³⁸ (CYP2C9) | Hogan 2000⁴⁴⁰ |
| | Meyer 1996⁴³⁹ (through drug | Mattson 1992⁴⁴¹ |
| | interaction) | |
| Valproic | Sadeque 1997⁴⁴² (check) | Bruni 1979⁴⁴³ |
| Acid, sodium | (CYP2C9) | Egger 1981⁴⁴⁴ |
| valproate | | Zaccara 1987⁴⁴⁵ |
| | | Mattson 1992⁴⁴⁶ |
| | | Sharpe 1995⁴⁴⁷ |
| Losartan | Song 2000⁴⁴⁸ (CYP2C9) | Camargo 1991⁴⁵¹ |
| | Meadowcroft 1999⁴⁴⁹ (CYP2C9) | |
| | Miners 1998⁴⁵⁰ (CYP2C9) | |
| Simvastatin | Transon 1996⁴⁵² (CYP2C9) | Matthews 1993^{453,I} |
| Olanzapine | Ring 1996⁴⁵⁴ (CYP2C9) | Osser 1999⁴⁴⁵ |
| | | Koran 2000⁴⁵⁶ |
| Clozapine | Ring 1996⁴⁵⁷ (CYP2C9) | Osser 1999⁴⁶⁰ |
| | Fang 1998⁴⁵⁸ (CYP2C9) | |
| | Prior 1999⁴⁵⁹ (CYP1A2, | |
| | CYP2C19) | |
| Fluvoxamine | Olesen 2000⁴⁶¹ (CYP1A2, | Harvey 2000^{464,II} |
| Fluoxetine | CYP2C19) | Sansone 2000⁴⁶⁵ |
| (Prozac) | Miners 1998⁴⁶² (CYP2C9) | Michelson 1999^{466,II} |
| | Schmider 1997⁴⁶³ (CYP2C9) | Darga 1991^{467,II} |
| Tolbutamide | Ring 1996⁴⁶⁸ (CYP2C9) | Wissler 1975^{471,III} |
| | Miners 1998⁴⁶⁹ (CYP2C9) | Ballagi-Pordany |
| | Lasker 1998⁴⁷⁰ (CYP2C9, | 1991^{472,III} |
| | CYP2C19) | |
| Anastrozole | Grimm 1997⁴⁷³ (CYP1A2, | Wiseman 1998⁴⁷⁴ |
| | CYP2C9) | Lonning 1998⁴⁷⁵ |
| | | Buzdar 1998⁴⁷⁶ |
| | | Jonat 1997⁴⁷⁷ |
| | | Buzdar 1997⁴⁷⁸ |
| | | Hannaford 1997⁴⁷⁹ |
| | | Buzdar 1997⁴⁸⁰ |
| | | Buzdar 1996⁴⁸¹ |
| | | Jonat 1996⁴⁸² |
| Nelfinavir (PI) | Khaliq 2000⁴⁸³ (CYP2C19) | VI |
| | Lillibridge 1998⁴⁸⁴ (CYP2C19, | |
| | CYP1A2)^{,V} | |
| Ritonavir (PI) | Muirhead 2000⁴⁸⁵ (CYP2C9) | VI |
| | Kumar 1999⁴⁸⁶ (CYP2C9, | |
| | CYP2C19) | |
| | Kumar 1996⁴⁸⁷ (CYP2C9) | |
| | Eagling 1997⁴⁸⁸ (CYP2C9) | |
| Amprenavir | Fung 2000⁴⁸⁹ (CYP2C9) | VI |
| (PI) | | |
| Saquinavir | Eagling 1997⁴⁹⁰ (CYP2C9) | VI |
| (PI) | | |

Cytochrome P450 inducers

| | | |
|---|---|---|
| Nifedipine | Fisslthaler 2000⁴⁹¹ (CYP2C9) | Krakoff 1993⁴⁹² |
| | | Maccario⁴⁹³ |
| | | Andronico 1991^{494,IV} |

^{I}Increase in BMI was associated with smaller decrease in common femoral arterial stiffness.
^{II}Fluoxetine produces a transient weigh loss leading to gain in body weight in the long term.
^{III}Tolbutamide induced atherosclerosis.
^{IV}Nifedipine reduced insulin resistance.
^{V}Inhibition occurred at supratherapeutic concentrations.
VI Replacing, or not including a protease inhibitor in therapy was associated with attenuated fat distribution abnormalities and insulin resistance (Barreiro 2000⁴⁹⁵, Mulligan 2000⁴⁹⁶, Gervasoni 1999⁴⁹⁷, Carr 2000⁴⁹⁸, Martinez 2000⁴⁹⁹, see also review, Passalaris 2000⁵⁰⁰).

Drugs are not “empty.” Drugs have other chemical reactions except inhibition of CYP-ERK. Take a microcompetition induced clinical symptom, such as weight gain. There are three possible events. The other chemical reactions might increase, decrease of not change body weight. Take the combined effect of CYP-ERK inhibition and the other chemical reactions. The H₀ hypothesis assumes a uniform (random) distribution on these events, that is, the probability of every such event is ⅓, that is, the probability that a CYP-ERK inhibitor causes weight gain is ⅓. The probability that each of two CYP-ERK different inhibitors cause weight gain is (⅓)*(⅓). In the table above there are 16 drugs, 15 CYP-ERK inhibitors and 1 CYP-ERK inducer. The probability that the 15 inhibitors increase weight and the 1 inducer reduces weight, under the H₀ assumption, is (⅓)¹⁶<0.0001.

H. Treatment

A healthy system is in stable equilibrium. Microcompetition induces a new, stable equilibrium which reflects the modified availability of transcription resources. Assume that the two equilibria are points in a measure space, that is, a space with a unit and direction. In fact, almost all molecular and clinical measurements defme such a space. Assume that any point in this space indicates a disease, and that the severity of the disease increases with the distance from the healthy system equilibrium. In this space, the distance between the microcompetition equilibrium and the healthy system equilibrium is small. The small distance between equilibria results in slow progression of the microcompetition diseases. Atherosclerosis or cancer, for instance, may take years to become clinically evident.

FIG. 13 illustrates the difference between the microcompetition equilibrium and the healthy system equilibrium. Denoting the difference between equilibria with Δ, the difference between the microcompetition equilibrium (M_{E}) and the healthy system equilibrium (H_{E}) is A(M_{E}-H_{E}). Most successful treatments create a new equilibrium (T_{E}) somewhere between M_{E} and H_{E}. The small distance between the microcompetition equilibrium and the healthy system equilibrium poses a challenge in measuring the effectiveness of such treatments. Since T_{E} is between M_{E} and H_{E}, the distance between T_{E} and M_{E} is even smaller than the distance between H_{E} and M_{E}, Δ(T_{E}-H_{E})<Δ(M_{E}-H_{E}). We assumed that the rate of disease progression/regression, of the microcompetition diseases is a function of the distance between equilibria. Hence, the difference in rate of disease progression between the rate of progression after treatment and during microcompetition is even smaller. Since the clinical changes induced by the move from point H_{E} to M_{E} are usually difficult to measure, the clinical changes induced by the move from point M_{E} to T_{E} are also difficult to measure (most likely even more difficult).

To address this issue, the following sections report results of studies which meet two conditions. One, since treatment effectiveness is reflection of the distance between two states of system equilibrium, only in vivo studies are included. Second, since the effect of treatment is slow to occur, only results of clinical and animal studies conducted over at least a few weeks are included. In some cases, the included studies report results which were obtained after years of treatment.

The studies are divided into three sections. The first section includes studies with GABP kinase agents. These agents stimulate the phosphorylation of a GABP kinase, such as ERK or JNK. The second section inludes studies with antioxidation agents. These agents reduce oxidation stress in infected cells. The third section includes studies with viral N-box agents. These agents reduce the concentration of viral DNA in the host.

FIG. 14 illustrates how aberrant GABP expression and function can be restored. The targets of these treatments are marked with filled boxes. Microcompetition between viral N-box and cellular genes for GABP is marked with a thick arrow.

1. GABP Kinase Agents

A GABP kinase agent stimulates the phosphorylation of a GABP kinase, such as ERK or JNK. The increase in the GABP kinase phosphorylation increases transcription of GABP stimulated genes and decreases transcription of GABP suppressed genes (see above). Since, microcompetition has the opposite effect on these classes of genes, a GABP kinase agent leads to slower progression of the microcompetition diseases.

a) Dietary Fiber

Dietary fibers produce sodium butyrate, a short chanin fatty acid (SCFA), during anaerobic fermentation in the colon. Sodium butyrate is a GABP kinase agent (see above). As a result, sodium butyrate phosphorylates GABP, which, in turn, potentiates binding of p300.

Microcompetition with a GABP virus decreases expression of metallothionein (see above). Sodium butyrate activated the metallothionein (MT) gene in certain carcinoma cell lines. Consider the following studies.

Different embryonal carcinoma cell lines show different basal levels of MT mRNA. For instance, F9 cell line shows intermediate basal levels of MT expression, while PC13, a similar cell line, shows very high levels. Since OC15S1 stem cells usually have very low basal levels, these cell were chosen for testing the effect of sodium butyrate on MT mRNA. OC15 embryonal carcinoma (OC15 EC) cells differentiate during 4 days in culture in the presence of retinoic acid (OC15 END). OC15 EC and OC15 END cells were treated with sodium butyrate and the MT mRNA was analyzed by Northern blots and quantified by densitomerty (Andrews 1987⁵⁰¹, FIG. 1). The results show that sodium butyrate increases MT mRNA in both undifferentiated OC15 EC and differentiated OC15 END cells. F9 EC cells, although having higher MT mRNA basal level, responded similarly to sodium butyrate treatment. It should be noted that the effect of sodium butyrate was specific since sodium propionate and sodium acetate, the other two products of bacterial fermentation in the colon, had no effect on MT mRNA.

Another study used ROS 17/2.8, a cloned rat osteosarcoma cell line. In this study, sodium butyrate induced MT synthesis in a dose-dependent manner (Thomas 1991⁵⁰²).

A third study used rat primary, non-transformed hepatocytes. Sodium treatment of these cells produced a 2-4 fold increase in MT mRNA (Liu 1992⁵⁰³, FIG. 6).

It is interesting that in the non-transformed cells sodium butyrate increased MT mRNA 2-4 fold, while in some carcinoma cell lines the increase was 20 fold (see, for instance, the increase in MT mRNA in OC15 embryonal carcinoma cells above). A compelling explanation is that the relative low basal MT mRNA in OC15 cells result from microcompetition with viral DNA present in these cells. In such a case, sodium butyrate should show a larger effect in OC15 relative to the non-transformed cells.

The Coronary Artery Risk Development in Young Adults (CARDIA) Study, a multicenter population-based cohort study, tested the change in cardiovascular disease (CVD) risk factors over a 10 year period (1985-1986 to 1995-1996) in Birmingham, Ala.; Chicago, Ill.; Minneapolis, Minn.; and Oakland, Calif. A total of 2,909 healthy black and white adults, age 18 to 30 years at enrollment, were included in the study. The results showed that dietary fiber consumption was inversely associated with body weight in both blacks and whites. At all levels of fat intake, subjects consuming most fiber gained less weight than those consuming the least fiber. Moreover, fiber consumption was also inversely associated with fasting insulin levels and systolic and diastolic blood pressure in both black and white (Ludwig 1999⁵⁰⁴).

Fifty-two overweight patients, mean body mass index (BMI)=29.3, participated in a 6 months, randomized, double-blind, placebo-controlled, parallel group design, study. The treatments included an energy restricted diet plus dietary fiber supplement of 7 g/day, or the diet plus placebo. The results showed that the fiber treated patients lost significantly more weight relative to the placebo treated patients (5.5±0.7 kg, vs. 3.0±0.5 kg, P=0.005). Hunger feelings, measured using visual analogue scales (VAS), were significantly reduced in the fiber-treated group, whereas a significant increase was seen in the placebo group (P<0.02) (Rigaud 1990⁵⁰⁵).

In another study, ninety-seven mildly obese females participated in 52 week, randomized, double-blind, placebo-controlled trial, study. The treatment consisted of a restricted diet providing 1,200 kcal/day and a dietary fiber supplement of 7 g/day for 11 weeks, (part I), followed by a diet providing 1,600 kcal/day and a dietary fiber supplement of 6 g/day for 16 weeks (part II). Finally placebo was withdrawn and all still adhering subjects were given a dietary fiber supplement of 6 g/day and an ad libium diet for the rest of the period (part III). Initial body weights were comparable in the fiber group versus placebo group. The results showed that during part I, the weight reduction in the fiber supplemented group was significantly higher compared to the placebo group (4.9 kg and 3.3 kg, respectively, P=0.05). Accumulated weight reduction during part II was still significantly higher in the fiber supplemented group compared to the placebo group (3.8 kg and 2.8 kg, respectively, P<0.05). Total weight loss in the fiber group after 52 weeks was 6.7 kg. Probability of adherence to the treatment regimen was significantly higher in the fiber group from week 13 and onwards (P<0.01). Initial blood pressures were comparable. A significant reduction of systolic blood pressure was observed in both groups. However, a significant reduction of diastolic blood pressure was observed in the fiber group only (P<0.05) (Ryttig 1989⁵⁰⁶).

These studies show that dietary fiber consumption induces weight loss, reduces insulin resistance and attenuates hypertension.

Soybean hull is a rich source of dietary fiber. Therefore, a diet enriched with soybean hull should attenuate atherosclerosis. Consider the following study. Twenty five monkeys were divided into 5 groups, each subjected to a different diet. The T1 group received the basal diet; T2, the basal diet plus palm oil; T3, the basal diet plus palm oil plus soybean hull; T4, the basal diet plus cholesterol, and T5, the basal diet plus cholesterol plus soybean hull. The diets were given for a period of 8 months and water were given ad lib. At the end of the experiment thorax surgery was performed on the animals under general anesthesia. The aorta was removed surgically for histopathological observation stained with hematoxylin and eosine. Histopathological observation of the aorta showed that adding soybean hull to the basal diet+palm oil diet reduced formation of atherosclerotic lesions from 46.67 to 31.25%. Adding soybean hull to the basal diet+cholesterol reduced formation of lesion from 86.25 to 53.38% (Piliang 1996⁵⁰⁷). Based on these observations, Piliang, et al., concluded that “the soybean hull given in the diet has the ability to prevent the development of atherosclerosis in the aorta of the experimental animals.”

Consumption of dietary fiber is associated with reduced risk of several types of cancer (Kim 2000⁵⁰⁸, Madar 1999⁵⁰⁹, Camire 1999⁵¹⁰, Mohandas 1999⁵¹¹, Heaton 1999⁵¹², Cummings 1999⁵¹³, Ravin 1999⁵¹⁴, Reddy 1999A⁵¹⁵, Reddy 1999B⁵¹⁶, Earnest 1999⁵¹⁷, Kritchevsky 1999⁵¹⁸, Cohen 1999⁵¹⁹).

b) Acarbose

Acarbose is a α-glucosidase inhibitor, a new class of drugs used in the treatment of diabetes mellitus. α-glucosidases are enzymes present on the brush border of the small intestine. The enzymes hydrolyze di- and oligosaccharides, derived from diet and luminal digestion of starch by pancreatic amylase, into monosaccharides. Sine only monosaccharides are transported across intestinal cell membrane, α-glucosidases inhibition reduces carbohydrate absorption.

Acarbose inhibits starch digestion in the human small intestine, and therefore, increases the amount of starch available for microbial fermentation to acetate, propionate, and butyrate in the colon. A study examined fermentations by fecal suspensions obtained from subjects who participated in an acarbose-placebo crossover trial. The results showed that the concentrations of acetate, propionate, and butyrate were 57, 13, and 30% of the total final concentrations, respectively, for acarbose treated subjects and 57, 20, and 23% for untreated subjects (Wolin 1999⁵²⁰, Table 1, the statistical significance for the difference between acarbose and placebo was P<0.002 for propionate, and P<0.02 for butyrate). Based on these results, Wolin, et al., concluded that “our results show that acarbose treatment results in decreases in the activities of colonic bacteria . . . that form propionate and an increase in the activity of bacteria that produce butyrate.”

To determine the effects of acarbose on colonic fermentation, another study gave subjects 50-200 mg acarbose or placebo (cornstarch), three times per day, with meals in a double-blind crossover study. Fecal concentrations of starch and starch-fermenting bacteria were measured and fecal fermentation products were determined after incubation of fecal suspensions with and without added substrate for 6 and 24 h. Substrate additions were cornstarch, cornstarch plus acarbose and potato starch. Dietary starch consumption was similar during acarbose and placebo treatment periods. The result showed that butyrate in feces, measured either as concentration or percentage of total short-chain fatty acids, was significantly greater with acarbose treatment compare to placebo, while propionate was significantly smaller (Wolin 1999⁵²¹, Table 1. P<0.0001). Moreover, butyrate production was significantly greater in fermentations in samples collected during acarbose treatment, whereas production of acetate and propionate was significantly less. Based on their results, Wolin, et al., concluded that “acarbose effectively augmented colonic butyrate production by several mechanisms; it reduced starch absorption, expanded concentrations of starch-fermenting and butyrate-producing bacteria and inhibited starch use by acetate- and propionate-producing bacteria.”

Acarbose or placebo were administered to non-insulin dependent diabetes (NIDDM) patients for 1 year in a randomized, double-blind, placebo-controlled, parallel design study (Wolever 1997⁵²², FIG. 1). After one year, the 130 subjects treated with acarbose experienced an average weight loss of 0.46±0.28 kg. In contrast, the 149 subject treated with placebo experienced a 0.33±0.25 kg weight gain (P=0.027). Interestingly, acarbose had no effect on energy intake, nutrient intakes, or dietary patterns.

e) Vanadate

An ERK phosphatase is an enzyme that inactivates ERK by dephosphorylation of either Thy, Tyr, or both residues (see above). The class of all ERK phosphatases includes, for instance, PP2A, a type 1/2 serine/threonine phosphatase, PTP1B, a protein tyrosine phosphatase, and MKP-1, a dual specificity phosphatase. Inhibition of an ERK phosphatase stimulates ERK phosphorylation. The increase in ERK phosphorylation increases transcription of GABP stimulated genes and decreases transcription of GABP suppressed genes (see above). Since, microcompetition has the opposite effect on these classes of genes, inhibition of an ERK phosphatase leads to slower progression of the microcompetition diseases. Consider vanadate as an example.

Vanadate (VO₄⁻³) and vanadate derivatives are general protein tyrosine phosphatase (PTP) inhibitors. Specifically, vanadate, and pervanadate (a general term for the variety of complexes formed between vanadate and hydrogen peroxide) was shown to inhibit the protein-tyrosine phosphatase PTP1B buyer 1997⁵²³).

PTPs dephosphorylate and deactivate ERK (see above). As general PTP inhibitors, vanadate and vanadate derivatives are expected to activate ERK. Such activation has been reported in several studies (Wang 2000⁵²⁴, Zhao 1996⁵²⁵, Pandey 1995⁵²⁶, D'Onofrio 1994⁵²⁷).

The bifunctional enzyme 6-phosphofructo-2-kinase (EC 2.7.1.105, PFK-2)/fructose-2,6-bisphosphatase (EC 3.1.3.46 FBPase-2) catalyzes the synthesis and degradation of fructos-2,6-bisphosphate. The rat PFK-2/FBPase-2 gene (gene A) codes for the fetal (F) mRNA, the muscle (M) mRNA, and the liver (L) mRNA. Each of these mRNAs originates from a different promoter in the gene. The F-type promoter includes an enhancer in the (−1809, −1615) region with three N-boxes at (−1747, −1742), (−1716, −1710) and (−1693, 1688) (Darville 1992⁵²⁸, FIG. 4). The enhancer stimulated transcription, especially in FTO2B hepatoma cells (Ibid, Table 1). DNase I protection of the enhancer with extracts from FTO2B cell, from C2C12 myoblasts or myocytes, or from liver, but not from muscle, showed one specific footprint corresponding to the middle N-box (Ibid, FIG. 5). Gel retardation assays with extracts from FTO2B and HTC cells, L6 myoblasts and myocytes, and liver, but not muscle, showed a major complex (Ibid, FIG. 6A). When this enhancer fragment was methylated at single purines using dimethylsulfate and subsequently incubated with FTO2B extracts, three contact points were detected located within the N-box (Ibid, FIG. 4). The three points of methylation interference coincide with contact points identified by the same technique in the two N-boxes of the adenovirus E1A core enhancer which binds GABP. A subsequent study (Dupriez 1993⁵²⁹) showed that changing the GG, essential for ets DNA binding, to CC in both distal and proximal N-boxes decreased promoter activity by 15-20%. Changing GG to CC in the middle N-box decreased promoter activity by 75%. The study also showed that anti-GABPα and anti-GA-BPβ antibodies inhibited formation of complexes formed on the middle N-box by FTO2B proteins (Ibid, FIG. 4, lane 5 and 6). Transfection with recombinant GABPα and GABPβ produced shifts that comigrated with these complexes and were inhibited by anti-GABPα antibodies (Ibid, FIG. 4, lane 12-16). These observations suggest that the F-type PFK-2/FBPase-2 is a GABP stimulated gene.

GABP viruses microcompete with the F-type PFK-2/FBPase-2 enhancer for GABP. Therefore, viral infection of cells decreases F-type PFK-2/FBPase-2 expression. Moreover, the higher the concentration of viral DNA, the greater the decrease in F-type PFK-2/FBPase-2 expression.

ERK activation is expected to stimulate transcription of GABP stimulated genes. The rat F-type PFK-2/FBPase-2 gene is a GABP stimulated gene. Therefore, vanadate should stimulate the transcription of F-type PFK-2/FBPase-2. Consider the following studies.

The effect of sodium orthovanadate oral administration on liver PFK-2/FBPase-2 mRNA content was measured in rats with streptozotocin (STZ)-induced diabetes. The mRNA content was measured after 3, 5, 7 and 15 days treatment (Miralpeix 1992⁵³⁰, FIG. 3). Vanadate treatment of diabetic animals produced a progressive increase in liver PFK-2/FBPase-2 mRNA content, reaching a normal level after 15 days. Similar results are reported in Inoue (1994⁵³¹).

The F-type PFK-2/FBPase-2 is usually not expressed in liver cells. However, the F-type mRNA increases in proliferating cells. Dupriez, et al., (1993⁵³²) measured tissue expression of the gene. F-type PFK-2/FBPase-2 mRNA was present in hepatoma, fibroblast, and myoblasts cell lines. The mRNA was found in fetal liver and muscle, the two fetal tissues examined. In adult tissues the mRNA was found in the lung and thymus. In the other adult tissues tested the mRNA was present at much lower concentrations or was undetectable. The highest concentration was in preterm placenta, with a decrease at term. The concentration decreased upon differentiation of L6 myoblasts into myocytes (Ibid, FIG. 2) and in Rat-1 fibroblasts make quiescent by lowering serum concentration in culture from 10 to 0.1%. Moreover, F-type mRNA concentration increased in FTO2B cells upon dexamethasone treatment. Based on these observations, Dupreiz, et al., concluded that the “expression of the F-type mRNA appears to correlate with cell proliferation.”

Usually, liver tissue shows limited cell proliferation. However, in the Miralpeix 1992 study (see above), vanadate was administred to male Sprague-Dawley rats one week after the animals were treated with a single intravenous injection of streptozotocin (STZ). As it turns out, STZ injection to Sprague-Dawley rats induces high hepatocyte proliferation. Consider the following study.

Hepatocyte proliferation was measured in Sprague-Dawley rats made diabetic by iv injection of STZ. The results showed 12% increase in the ratio of liver weight to body weight in diabetic rats 8 days after injection compare to normal rats, and 44% increase at 30 days (Herrman 1999⁵³³). The results also showed an increase in hepatocyte mitosis to 300% of normal at 8 days, a return to normal at 30 days, and a decrease to 25% at 90 days (Ibid, FIG. 1). Based on these results Herrman, et al., concluded that “hepatomegaly observed in streptozotocin-induced experimental diabetes may be due primarily to early hyperplasia.”

The Miralpeix 1992 study used a “1.4 kilobase rat liver PFK-2/FBPase-2 cDNA probe which corresponds to the mRNA for liver PFK-2/FBPase-2 devoid of the 5′ end coding for amino acids 1-90.” This probe does not distinguish between F-type and L-type PFK-2/FBPase-2 mRNA. Therefore, the reported increase in PFK-2/FBPase-2 mRNA is, most likely, a result of the increase in F-type PFK-2/FBPase-2 mRNA in hepatocytes induced to proliferate by a streptozotocin injection.

Five week-old Zucker rats, an animal model of obesity and insulin resistance, were divided into three groups of 6 rats: lean (Fa/fa) control, obese (fa/fa) control and obese (fa/fa)-vanadate treated. The rats in the treated group received sodium orthovanadate through drinking water for four months. Obese rats had significantly higher body weight compared to lean. However, body weight of vanadate-treated obese decreased 43% to levels comparable to lean control (Pugazhenthi 1995⁵³⁴, Table 1).

Similar results are reported in McNeill and Orvig (1996⁵³⁵). Wistar rats were divided into two groups, control (8 animals) and treated (11 animals). Treated animals recieved between 0.3 and 0.5 mmol/kg of bis(maltolato)oxovanadium/day in drinking water over a 77 day period. Begining at day 56 the treated animals showed reduced weight gain compared to controls (Ibid, FIG. 1, group 2 vs. group 1). (See also Dai 1994⁵³⁶, and Bhanot 1994^{537.})

Cruz, et al., (1995)⁵³⁸ tested the antineoplastic effect of orthovanadate on a subcutaneous MDAY-D2 tumor mouse model. Ten week old DBA/2j female mice were injected sucutaneously in the posterior lateral side with 4×10⁵ cell in 100 μl of PBS. On day 5, the mice were divided into two groups. One group received subcutaneous injections of 100 μl of PBS and another group received 100 μl of PBS containing 500 μg of orthovanadate daily. The orthovanadate was administrated subcutaneously on the opposite, tumor-free, posterior lateral side. On day 14, the mice were sacrificed, weighted and tumors dissected and weighted. The results showed decreased tumor growth in treated mice compared to controls (Ibid, FIG. 6). In control mice, the tumor weights varied from 0.86-1.74 g, whereas in orthovanadate treated mice, four mice showed no detectable tumors and 11 mice showed tumors varying from 0.08-0.47 g. Orthovanadate treatment reduced tumor growth by more than 85%, sometimes completely inhibiting tumor formation.

Another study tested the chemoprotective effect of vanadium against chemically induced hepatocarcinogenesis in rats. Initiation was performed by a single intraperitoneal injection of diethylnitrosamine (DENA; 200 mg kg⁻¹) followed by promotion with phenobarbital (0.05%) in diet. Vanadium (0.5 ppm) was provided ad libitum throughout the experiment in drinking water. The results showed after 20 weeks, vanadium reduced the incidence (P<0.01), total number and multiplicity (P<0.001), and altered the size distribution of visible persistent nodules (PNs) as compared with DENA controls (Bishayee and Chatteijee 1995⁵³⁹). Mean nodular volume (P<0.05) and nodular volume as a percent of liver volume (P<0.01) were also attenuated. Vanadium also caused a large decrease in number (P<0.001) and surface area (P<0.01) of gamma-glutamyltranspeptidase (GGT)-positive hepatocyte foci and in labeling index (P<0.001) of focal cells, coupled with increased (P<0.01) remodeling. The activity of GGT, measured quantitatively, was found to be significantly less in PNs (P<0.001) and non-nodular surrounding parenchyma (P<0.01) of vanadium supplemented rats. Histopathological analysis of liver sections showed well-maintained hepatocellular architecture compared to DENA control. Based on these results, Bishayee and Chatteijee (1995) concluded that “our results, thus, strongly suggest that vanadium may have a unique anti-tumor potential.” See also Liasko 1998⁵⁴⁰.

Numerous in vivo studies demonstrated reduced blood glucose in insulin deficient diabetic animals, and improved glucose homeostasis in obese, insulin-resistant diabetic animals, following treatment with vanadate. In human studies, insulin sensitivity improved in NIDDM patients and in some IDDM patient after treatment with vanadate (see recent reviews Goldfine 1995⁵⁴¹, Brichard 1995⁵⁴²)

As an example consider the Pugazhenthi, et al, (1995, see above) study. This study also tested the effect of vanadate on diabetes. The obese Zucker rats showed elevated plasma levels of glucose and insulin. Vanadate treatment decreased glucose and insulin by 36 and 80%, respectively (Ibid, Table 1).

d) PTPIB Knockout

Gene knockout is a special case of intervention. The results of a PTP1B gene knockout is PTP1B enzyme deficiency. Vanadate inhibits PTP1B (Huyer 1997⁵⁴³). Therefore, both PTP1B gene knockout and adminstration of vanadate result in reduced activity of the PTP1B enzyme. Considering the discussion above, the PTP1B gene knochout effect on clinical symtoms should be similar to the effects of vanadate.

A targeting vector was designed to delete a segment of the mouse homolog of the PTP1B gene. This segment included exon 5 and the tyrosine phosphatase active site in exon 6. The deleted segments were replaced with the neomycin resistance gene. Tow separate embryonic stem cell clones that undergone homologous recombination and possessed a single integration event were used to microinject Balb/c blastocyts. Chimeric males were mated with wild-type Balb/c female, and heterozygotes from this cross were mated to product animals homozygous for the PTP1B mutation (Elchebly 1999⁵⁴⁴, FIG. 1A). The PTP1B protein was absent in PTP1B null mice (PTP1B(−/−)), and heterozygotes (PTP1B(+/−)) expressed about half the amount of PTP1B relative to wild type mice (Ibid, FIG. 1B). PTP1B null mice grew normally, on regular diet did not show any significant difference in weight gain compared to wild-type mice, lived longer than 1.5 years without any signs of abnormality and were fertile. To study the effect of PTP1B gene knockout on obesity, the PTP1B(−/−), PTP1B(+/−) and wild type mice were fed a high-fat diet normally resulting in obesity. As expected, the wild-type mice rapidly gained weight. In contrast, the PTP1B(−/−), PTP1B(+/−) mice were protected from the diet induced weight gain (Ibid, FIG. 5). Based on these results, Elchebly, et al., concluded that PTP1B deficiency results in obesity resistance.

Another study reported results of a PTP1B gene disruption. Klaman, et al., (2000⁵⁴⁵) generated PTP1B-null mice by targeted disruption of the ATG coding exon (exon 1). The PTP1B-deficient mice showed low adiposity and protection from diet-induced obesity. The decreased adiposity resulted from reduced fat cell mass without a decrease in adipocyte number. Leanness in PTP1B-deficient mice was associated with increased basal metabolic rate and total energy expenditure.

Elchebly, et al., (1999⁵⁴⁶) also tested the effect of PTP1B gene knockout on diabetes. In fed state, PTP(−/−) mice on regular diet showed a 13% reduction and PTP(+/−) a 8% reduction in blood glucose concentration relative to wild type mice (Ibid, FIG. 2A). Fed PTP1B(−/−) mice on regular diet had circulating insulin levels about half of wild type fed animals (Ibid, FIG. 2B). The enhanced insulin sensitivity of the PTP1B(−/−) mice was also observed in glucose and insulin tolerance tests (Ibid, FIG. 3A and 3B). The PTP1B(−/−), PTP1B(+/−) and wild type mice were also fed a high-fat diet normally resulting in insulin resistance. As expected, the wild-type mice became insulin resistance. In conctrast, on high-fat diet the PTP1B(−/−) mice showed glucose and insulin concentrations similar to animals on normal diet (Ibid, Table 1). PTP1B(−/−) mice also showed enhanced insulin sensitivity relative to wild type in both glucose and insulin tolerance tests (Ibid, FIGS. 6A, 6B). On high-fat diet, the PTP1B(+/−) mice showed increased fasting concentrations of circulating insulin but similar fasting glucose concentrations relative to animals on normal diet (Ibid, Table 1). Based on these results, Elchebly, et al., concluded that PTP1 B deficiency results in enhanced insulin sensitivity.

The PTP1B-deficient mice in Klaman, et al., (2000⁵⁴⁷) showed similar enhanced insulin-stimulated whole-body glucose disposal.

As expected, both a PTP1B deficiency and vanadate result in obesity resistance and enhanced insulin sensitivity. We speculate that PTP1B gene knockout also induces cancer resistance.

2. Antioxidants

Microcompetition decreases binding of GABP to the N-bo. XOxidative stress also decreases the binding of GABP to the N-bo. XTherefore, microcompetition can be viewed as “excessive oxidative stress.” Some antioxidants reduce intracellular oxidative stress. These antioxidants stimulate the binding of GABP to the N-box attenuating the effect of microcompetition on transcription, resulting in slower progression of the microcomeptition diseases.

a) Garlic

Garlic is a free radicals scavenger. A study investigated, using high pressure liquid chromatography, the ability of unheated or heated garlic extract to scavenge hydroxyl radical (•OH) generated by photolysis of H₂O₂ (1.2-10 μmoles/ml) with ultraviolet (UV) light and trapped with salicylic acid (500 nmoles/ml). H₂O₂ produced •OH in a concentration-dependent manner as estimated by •OH adduct products 2,3-dihydroxybenzoic acid (DHBA) and 2,5-DHBA. Garlic extract (5-100 μl/ml) inhibited (30-100%) of 2,3-DHBA and 2,5-DHBA in a concentration-dependent manner (Prasad 1996⁵⁴⁸, FIG. 3). Garlic activity was reduced by 10% approximately, when heated to 100 degrees C. for 20, 40 or 60 min. Garlic extract also prevented the •OH-induced formation of malondialdehyde in rabbit liver homogenate in a concentration-dependent manner (Ibid, FIG. 10). In the absence of •OH, gralic did not affect MDA levels. Based on these results, Pasas, et al., (1996) concluded that “garlic extract is a powerful scavenger of •OH.”

Another study examined the antioxidant effects of garlic extract in a cellular system using bovine pulmonary artery endothelial cells (PAEC) and murine macrophages (J774). The study used intracellular glutathione (GSH) depletion as an index of oxidative stress. Oxidized LDL (Ox-LDL) caused a depletion of GSH. Pretreatment with aged garlic extract inhibited Ox-LDL induced peroxides in PAEC and suppressed peroxides in macrophages dose-dependently (Ide 1999⁵⁴⁹). In a cell free system, the aged garlic extract was shown to scavenge H₂O₂ dose-dependently. These results show that aged garlic extract prevents the Ox-LDL-induced depletion of GSH in endothelial cells and macrophages.

Garlic attenuates the formation of atherosclerotic plaque. A study de-endothelialized the right carotid artery of 24 rabbits by balloon catheterisation in order to produce a myointimal thickening. After 2 weeks the rabbits were randomly assigned to four groups. Group I received a standard diet (standard); Group II received standard diet supplemented with 800 μl/kg body weight/day the aged garlic extract “Kyolic” (standard+Kyolic); Group III received a standard diet supplemented with 1% cholesterol (cholesterol-enriched); and Group IV received standard diet supplemented with 1% cholesterol and Kyolic (cholesterol-enriched+Kyolic). After 6 weeks, the cholesterol-enriched diet caused a 6-fold increase in serum cholesterol level (Group III) compared to standard diet (Group I) (P<0.05) (Efendy 1997⁵⁵⁰, FIG. 1). At 6 weeks, the choleserol-enriched diet (Group III) showed fatty streak lesions covering approximately 70±8% of the surface area of the thoracic aorta. The cholesterol-enriched+Kyolic group (Group IV) showed fatty lesions in only 25±3% of the same surfce area (Ibid, FIGS. 2A and 2B), which represents an about 64% reduction. No lesions were present in Group I and II. The cholesterol-enriched diet also caused an increase in aortic arch cholesterol (2.1±0.1 mg cholesterol/g tissue) which was significantly reduced by Kyolic (1.7±0.2 mg cholesterol/g tissue) (P<0.05). Kyolic significantly inhibited the development of thickened, lipid-filled lesions in the pre-formed neointimas produced by balloon-catheter injury of the right carotid artery in cholesterol-fed rabbits (intima as percent of artery wall, Group III 42.6±6.5% versus Group IV 23.8±2.3%, P<0.01). Kyolic had little effect in rabbits on a standard diet (Group II 18.4±5.0% versus Group I 16.7±2.0%). In vitro studies showed that Kyolic inhibited smooth muscle proliferation (Ibid, FIG. 5). Based on these results, Efendy, et al., (1997) concluded that “Kyolic treatment reduces fatty streak development, vessel wall cholesterol accumulation and the development of fibro fatty plaques in neointimas of cholesterol-fed rabbits, thus providing protection against the onset of atherosclerosis.”

Jain (1978⁵¹¹), Jain (1976 51) and Bordia (1975⁵⁵³) reported similar observations. Jain (1978) and Jain (1976) used rabbits fed a 16 week standard or cholesterol-enriched diet supplemented with or without garlic extract. In both studies the results showed marked atherosclerotic lesions in animals fed a cholesterol-enriched diet relative to standard diet. The animals fed cholesterol-enriched diet supplemented with garlic extract showed attenuated lesions formation. Jain (1978) also reported reduced aorta cholesterol content in garlic treated animals. Bordia (1975) used rabbits fed a 3 months on similar diets. The results showed that garlic attenuated the formation of atherosclerotic plaque and the increase in lipid content of aorta.

Garlic treatment resulted in other favorable effects associated with attenuated atherosclerosis. A study measured the the elastic properties of the aorta using pulse wave velocity (PWV) and pressure-standardized elastic vascular resistance (EVR). The subjects included healthy adults (n=101; age, 50 to 80 years) who were taking 300 mg/d or more of standardized garlic powder for at least 2 years and 101 age- and sex-matched controls. Blood pressures, heart rate, and plasma lipid levels were similar in the two groups. The results showed that PWV (8.3±1.46 versus 9.8±2.45 m/s; P<0.0001) and EVR (0.63±0.21 versus 0.9±0.44 m²•s⁻²•mm Hg⁻¹; P<0.0001) were lower in the garlic group than in the control group (Breithaupt-Grogler 1997⁵⁵⁴, Table 1, FIG. 1). PWV showed significant positive correlation with age (garlic group, r=0.44; control group, r=0.52, FIG. 3) and systolic blood pressure (SBP) (garlic group, r=0.48; control group, r=0.54, FIG. 4). With any degree of increase in age or SBP, PWV increased less in the garlic group than in the control group (P<0.0001, FIG. 3, FIG. 4). ANCOVA and multiple regression analyses demonstrated that age and SBP were the most important determinants of PWV and that the effect of garlic on PWV was independent of confounding factors. According to Breithaupt-Grogler, et al., (1997), “The data suggested that the elastic properties of the aorta were maintained better in the garlic group that in the control group.” In it interesting that in experimental animals, changes of ratio of intimal (plaque) area to medial area during progression and regression of atherosclerosis correlated with changes in indices of aortic elastic properties. Pregression of atherosclerosis resulted in higher PWV, and vice versa (Farrar 1991⁵⁵⁵).

See also studies in the special supplement of the British Journal of Clinical Practice (1990, Supplement 69) dedicated to the clinical effects of garlic in ischemic heart disease.

Microcompetition increases the transcription of P-selectin on endothelial cells, increases the transcription of tissue factor (TF) and decreases the transcription of β₂ integrin and α₄ integrin on macrophages and decreases the transcription of retinoblastoma susceptible gene (Rb) in smooth muscle cells (SMC). Garlic reduces oxidative stress in endothelial cells, macrophages and SMCs. The reduced oxidative stress stimulates the binding of GABP to these genes, decreasing the transcription of TF and P-selectin and increasing the transcription of β₂ integrin, α₄ integrin and Rb. The change in the transcription of these genes attenuates the formation of atherosclerotic plaque and the thickening of the intima.

The anticancer properties of garlic were recognized thousands of years ago. The ancient Egyptians used garlic externally for treatment of tumors, and Hippocrates and physicians in ancient India are reported to have used garlic externally for cancer treatment. Recent studies confirmed these properties. See, for instance, the section “Garlic, Onions and Cancer,” in the recent review by Ali, et al., (2000⁵⁵⁶), the meta-analysis of the epidemiologic literature on garlic consumption and the risk of stomach and colon cancer (Fleischauer 2000⁵⁵⁷), and specific animals studies demonstrating garlic suppression of chemically induced tumors (Singh 1998⁵⁵⁸, Singh 1996⁵⁵⁹).

3. Viral N-box Agents

A viral N-box agent reduces the number of active viral N-boxes in the host cell. The reduction can be accomplished by reducing the copy number of viral genome, by binding and inhibition of the viral N-boxes, etc. The reduced number of active viral N-boxes eases microcompetition and slows progression of the microcompetition diseases.

a) Ganciclovir

Ganciclovir (Cytovene, DHPG) is guanosine analogue. The prodrug is phosphorylated by thymidine kinase to the active triphosphate form after uptake into the infected cell. The triphosphate form inhibits the viral DNA polymerase by competing with cellular deoxyguanosine triphosphate for incorporation into viral DNA causing chain termination. Ganciclovir is effective against herpes simplex virus 1 and 2 (HSV-1, HSV-2), cytomegalovirus (CMV), Epstein-Barr virus (EBV), and varicella-zoster virus (Spector 1999⁵⁶⁰V).

Aciclovir (acyclovir) and its oral form valacyclovir, and penciclovir and it oral form famciclovir are guanosine analogues similar to ganciclovir. These drugs are also effective against HSV-1, HSV-2 and CMV. See, for instance, a recent meta-analysis of 30 aciclovir clinical trials in HSV infections (Leflore 2000⁵⁶¹), a review on aciclovir recommended treatments in HSV infections (Kesson 1998⁵⁶²), reviews on valaciclovir effectiveness in HSV and CMV infections (Ormord 2000⁵⁶³, Bell 1999⁵⁶⁴) and a review on famciclovir and penciclovir (Sacks 1999⁵⁶⁵).

The load of viral DNA during latent infection is directly correlated with the extent of viral replication during the preceding productive infection (Reddehase 1994⁵⁶⁶, Collins 1993⁵⁶⁷). Therefore, reduction of viral replication should reduce the load of viral DNA during a subsequent lantent infection. Consider the following studies.

Bone marrow transplantation (BMT) was performed as a syngeneic BMT with female BALB/c (H-2^{d}) mice used at the age of 8 weeks as bone marrow donors and recipients. Two hours after BMT, the mice were infected subcutaneously in the left hind footpad with murine CMV. The mice were than divided into four groups. Three groups received therapy with increasing doses of CD8 T cells. The forth groups served as controls. The results showed that increasing doses of CD8 T cells significantly reduced the extent and duration of virus replication in vital organs, such as lungs and adrenal glands (Steffens 1998⁵⁶⁸, FIG. 2). Moreover, 12 months after BMT, the viral DNA load was measured. The results showed that the amount of DNA was smaller in the groups given CD8 T cell therapy. The viral DNA load in the lungs of mice given no immunotherapy was 5,000 viral genomes per 10 lung cells. The load following 10⁵ and 10⁶ CD8 T cells was 3,000 and 1,000 per 10⁶ lung cells, respectively. Since there were no infectious virus present, the study shows that attenuated viral replication during the acute phase of infection reduces the load of viral DNA during the subsequent phase of latent infection.

The study also measured the recurrence of viral infectivity following therapy. Five latently infected mice with no therapy and five mice treated with the 10⁷ CD8 T cells were subjected to immunoablative γ-ray treatment with 6.5 Gy. Recurrence of viral infectivity was measured 14 days later in separate lobes of the lungs. The no therapy group showed a high latent DNA load and recurrence of infectivity in all five mice in all five lobes of the lungs (with some variance). In contrast, the therapy group showed low load and recurrence of infectivity in only two mice and only in a single lobe in each mouse (Steffens 1998, FIG. 7). These results show that a reduction in viral replication reduces latent viral DNA load and the probability viral disease.

Thackary and Field, in a series of studies, also tested the effect preemptive therapy against viral infection. However, intsead of CD8 T cells, the studies administered famciclovir (FCV), valaciclovir (VACV), or human immunoglobulin (IgG) to mice infected via the ear pinna or the left side of the neck with either HSV-1 or HSV-2 (Thackray 2000A⁵⁶⁹, Thackray 2000B⁵⁷⁰, Thackray 2000C⁵⁷¹, Field 2000⁵⁷², Thackray 1998⁵⁷³). The results showed that 9-10 days of FCV treatment early in infection was effective in limiting the establishement of viral latency several months after treatment. Based on their results, Field and Thackary conclude that “Thus, the implication of our results is that even intensive antiviral therapy starting within a few hour of exposure is unlikely to compeletly abrogate latency. However, our results also show a significant reduction in the number of foci that are established and imply that there may also be a quantitative reduction in the latent genomes.” (Field 2000).

Another study compared the effect of aciclovir (ACV) and immunoglobulin (IgG) preemptive therapy on mice infected via scarified corneas with HSV-1. Both therapies were administered for 7 days commencing on the first day post infection. The results showed that ACV treatment resulted in a reduced copy number of latent HSV-1 genome on day 44 post infection relative to IgG (LeBlanc 1999⁵⁷⁴, FIG. 5). Since no untreated mice survived the infection, the study could not compare ACV treatment to no treatment. However, if we assume that IgG treatment either reduced or did not change the copy number of latent viral genome, we can conclude that the ACV preemptive treatment resulted in a reduced load of latent viral DNA.

Ganciclovir is similar to aciclovir and penciclovir. Therefore, a reasonable conclusion from these studies is that preemptive treatment with ganciclovir also reduces the load of viral DNA.

Accelerated coronary atherosclerosis can be observed in the donor heart following heart transplantation (TxCAD). Transplanting a heart from a CMV seropositive donor to a seronegative recipient increases the probability of a primary infection in the recipient (Bowden 1991⁵⁷⁵, Chou 1988⁵⁷⁶, Chou 1987⁵⁷⁷, Chou 1986⁵⁷⁸, Grundy 1988⁵⁷⁹, Grundy 1987⁵⁸⁰, Grundy 1986⁵⁸¹). The Thackary and LeBlanc studies demonstrated that administration of aciclovir or penciclovir prophylaxis early in primary infection reduces the load of the subsequent latent viral DNA in the infected animals (see above). Since microcompetition between viral and cellular DNA results in atherosclerosis, prophylactic administration of ganciclovir, a drug similar to aciclovir and penciclovir, early after heart transplantation, should reduce atherosclerosis. Consider the following study.

One hundred and forty-nine consecutive patients (131 men and 18 women, aged 48±13 years) randomly received either ganciclovir or placebo. The study drug was commenced on the first postoperative day and was administered 28 days. In 22% of patients the drug administration was delayed by up to 6 days due to acute-care problems. Immunosuppression consisted of muromonab-CD3 (OKT-3) prophylaxis and maintenance with cyclosporine, prednisone, and azathioprine. Coronary angiography was performed annually after heart transplantation. Mean follow-up time was 4.7±1.3 years. TxCAD was defined as presence of any angiographic disease irrespective of severity because of the recognized underestimation of TxCAD by angiography. The actuarial incidence of TxCAD was determined from these annual agiograms and fro autopsy data. CMV infection was determined in recipient and donor. The results showed that actuarial incidence of TxCAD at follow-up was 43±8% in patients treated with ganciclovir compared with 60±11% in placebo group (P<0.1). Moreover, the protective effect of ganciclovir was even more evident when the population of CMV seronegative recipients was considered exclusively. Of the 14 CMV seronegative recipients randomized to prophylactic ganciclovir, 4 (28%), developed TxCAD compared with 9 (69%) of the seronegative patients randomized to placebo (Valantine 1999⁵⁸²). The effect of ganciclovir is less evident in the population as a whole since among seropositive recipients there was no difference between ganciclovir and placebo. TxCAD developed in 22 (47%) of 48 patients randomized to ganciclovir compared with 21 (47%) of 46 in the placebo group. Base on these results, Valantine, et al., concluded that “prophylactic treatment with ganciclovir initiated immediately after heart transplantation reduces the incidence of TxCAD.”

It is interesting to note that in a multivariate analysis, the study found that the variable “CMV illness” was not an independent predictor of TxCAD when “lack of ganciclovir” and “donor age” were included in the analysis. We suspect that high correlation (multicollinearity) between “lack of ganciclovir” and “CMV illness” produced this result. Such a correlation was demonstrated in numerous studies. See, for instance, table 5 in Sia (2000⁵⁸³), which lists 10 clinical studies showing that early administration of ganciclovir prophylaxis in solid-organ transplantation resulted in reduced CMV disease compared to no treatment, administration of placebo, treatment with immunoglobulin or treatment with acyclovir. From this correlation we deduce that Valantine (1999) also measured reduced CMV disease (the study is mute on this statistic). The key parameter that determines the overall and organ-specific risks of CMV disease is the copy number of latent viral genome in various tissues (Reddehase 1994⁵⁸⁴). Therefore, the reduced CMV disease indicates a reduction in the copy number of latent viral genome, which, again, explains the reduced observed atherosclerosis.

b) Zidovudine (AZT), didanosine (ddI), zalcitabine (ddC)

Didanosine (2′,3′-dideoxyinosine, ddI) is a synthetic purine nucleoside analogue used against HIV infection. After passive diffusion into the cell, the drug undergoes phosphorylation by cellular (rather than viral, see above) enzymes to dideoxyadenosine-5′-triphosphate (ddATP), the active moiety. ddATP competes with the natural substrate for HIV-1 reverse transcriptase (deoxyadenosine 5′-triphosphate) and cellular DNA polymerase. Because ddATP lacks the 3′-hydroxyl group present in the naturally occurring nucleoside, incorporation into viral DNA leads to termination of DNA chain elongation and inhibition of viral DNA growth (see a recent review on ddI in Perry 1999⁵⁸⁵)

Zidovudine (retrovir, ZDV, AZT) and zalcitabine (ddC) are a nucleosides similar to ddI.

A study measured the change in HIV-1 DNA and RNA load relative to baseline in 42 antiretroviral naive HIV-1 infected persons treated with either AZT monotherapy, a combination of AZT+ddC or a combination of AZT+ddI over a period of 80 weeks (Breisten 1998⁵⁸⁶, FIG. 1A). At week 80, AZT treatment was associated with an increase, ddC+AZT with a decrease and ddI+AZT with a larger decrease in viral DNA. To compare the results statistically, the mean log change from baseline over all time points was compared between ddI+AZT and ddC+AZT. The mean change was −0.3375 and −0.20458 for ddI+AZT and ddC+AZT, respectively (P=0.02). It is interesting that, although not significant statistically (P=0.29), rank order of the ddI+AZT and ddC+AZT effect on RNA is reversed, that is, the mean effect of ddC+AZT on viral RNA was larger than ddI+AZT. Since the combination therapy of AZT and ddC is additive (Magnani 1997⁵⁸⁷), the ddC monotherapy effect on viral DNA was calculated as the ddC+AZT effect minus the AZT monotherapy effect. The calculated effect of ddC monotherapy on viral DNA was compared to the effect of AZT monotherapy. The mean log change from baseline over all time points was −0.15458 and −0.05 for ddC and AZT, respectively (P=0.09). The statistical analysis suggests that the ranking of ddI>ddC>AZT in terms of their effect on viral DNA, is significant. Moreover, the results suggest that at later time points, AZT tend to be associated with increased Viral DNA.

This statistical analysis is different than the analysis reported in paper. To test whether an “early” response occurred Bruisten, et al., averaged the values of weeks 4, 8, and 12 and for a “late” response the values of weeks 32, 40 and 48. The test showed that only the ddI+AZT treatment decreased the HIV-1 viral DNA “early” and “late.” P value of “early” compared to baseline is 0.002, p value of “late” compare to baseline is 0.052. The same values for ddC+AZT are 0.191 and 0.08. These values also indicate that ddI is more effective than ddC in reducing viral DNA.

Another study (Pauza 1994⁵⁸⁸) measured the total viral DNA by polymerase chain reaction assays for viral LTR sequences in 51 HIV infected patients. This assay detects linear, circular, and integrated HIV-1 DNA and also includes preintegration complexes that completed the first translocation step. Twenty patients were treated with AZT, 4 patients with ddI and 7 patients with ddC. After Southern blotting and hybridization, fragments were excised from the membrane and bound radioactivity was determined by scintillation counting. The measured LTR DNA levels were expressed on a scale of 1 to 5 (1 is lowest). Negative samples were labeled zero. The average ranking of viral DNA load for patients treated with ddI, ddC and AZT, was 2.25, 2.71 and 2.74, respectively. The difference between ddC and AZT is small. However, the average CD4/μl count for ddC and AZT treated patients was 82 and 191.55, respectively (p<0.03 for the difference). Hence, the viral DNA load of the AZT group is most likely biased downward. Overall, this ranking of treatment effectiveness measured in terms reduced viral DNA load is identical to the ranking in Breisten 1998 above.

A third study (Chun 1997⁵⁸⁹) measured total HIV-1 DNA in 9 patients. Eight patients were on triple therapy including two nucleosides and one protease inhibitor. One patient on received two nucleosides and two protease inhibitors. Six patients had undetectable plasma HIV RNA. The other three patients had 814, 2,800 and 6,518 copies/ml. The study also reports the year of seroconversion.

The viral DNA load is measured in copies of HIV-1 DNA per 10⁶ resting CD4+ T cells. The p values for the intercept and coefficient are 1.31E-05 and 0.131481, respectively. Since the sample size is small, the p value for the coefficient is considered as borderline significant, which means that even with triple and quadruple therapies, and in patients with mostly undetectable plasma HIV RNA, viral DNA load increases with an increase in the number of years since seroconversion.

The difference between the expected and the observed number of viral DNA copies was calculated for each patient. The therapy of two patients included ddI. The average difference for these patients was −828 copies. The therapy of five patients included AZT. The average difference for these patients was +317 copies. These results suggest that ddI is associated with a decrease and AZT and increase in the number of viral DNA copies in this group of patients.

Under different conditions, with monotherapy, triple and quadruple therapy with a protease inhibitor, with detectable and undetectable RNA, the results are consistent. ddI is associated with a larger reduction in viral DNA load compared to ddC, and AZT is associated with an increase in viral DNA load.

A study observed 306 six HIV-infected women between December 1997 and February 1998 (Gervasoni 1999⁵⁹⁰). The women were treated with two or more antiretroviral drugs. One hundred and sixty two patients were treated with two nucleosides (double therapy) and 144 with three or more drugs including at least one protease inhibitor (PI) (triple therapy). Fat redistribution (FR) was confirmed by means of a physical examination and dual-energy X-ray absorptiometry (DEXA). FR was observed in 32 women (10.5%) (12 on double therapy, 20 on triple therapy). The body changes were reported to gradually emerge over a period of 12-72 weeks. A statistical analysis showed that a combination treatment which included ddI was significantly associated with absence of FR (P=0.019). A combination treatment which included ddC was also significantly associated with absence of FR (P=0.049). The p values indicate that ddI-including combination was more effective than a ddC-including therapy in preventing FR. Contrary to ddI and ddC, a combination therapy which included AZT was associated with a low risk of developing FR (OR 0.3).

The association between ddI-, ddC- and AZT-including combinations with fat redistribution is consistent with their effect on reducing or increasing viral DNA load.

Another interesting observation in this study was the longer median total duration of antiretroviral drug treatment in women with FR compared to those without FR (1,187 versus 395 days). Only one of the 32 women with FR received antiretroviral drug therapy for less than 1,000 days. The risk of FR for women under antiretorviral drug therapy for more than 1,000 days was 10 times greater than in those who received shorter drug therapy (OR 10.8, P=0.0207).

A statistical analysis of results in Chun 1997 (see above) showed that viral DNA load increases with an increase in the number of years since seroconversion. Since the duration of antiretroviral drug treatment most often increases with the number of years since seroconversion, longer duration correlates with higher viral DNA load. Higher viral DNA load results in more intense microcompetition, and therefore, fat redistribution.

c) Garlic

Garlic has antiviral activity. See for instance Guo, et al., (1993⁵⁹¹) and Weber, et al., (1992⁵⁹²).

### Table of Cited References

¹ Scholer H, Haslinger A, Heguy A, Holtgreve H, Karin M. In vivo Competition Between a Metallothionein Regulatory Element and the SV40 Enhancer. Science 1986 232: 76-80.

² Adam G I, Miller S J, Ulleras E, Franklin G C. Cell-type-specific modulation of PDGF-B regulatory elements via viral enhancer competition: a caveat for the use of reference plasmids in transient transfection assays. Gene. Oct. 31, 1996; 178(1-2):25-9.

³ Allebach E S, Boettiger D, Pacifici M, Adams S L. Control of types I and II collagen and fibronectin gene expression in chondrocytes delineated by viral transformation. Mol Cell Biol. 1985 May;5(5):1002-8.

⁴ Parker I M, Smith A A, Gevers W. Absence of α2(1) Procollagen Synthesis in a Clone of SV40-transformed WI-38 Human Fibroblasts. The Journal of Biological Chemistry, 1989, 264(13): 7147-7152.

⁵ Moro L, Smith B D. Identification of collagen alpha1 (I) trimer and normal type I collagen in a polyoma virus-induced mouse tumor. Arch Biochem Biophys 1977 July; 182(1):33-41.

⁶Parker I M, Smith A A, Mundell K, Collins M, Boast S, Ramirez F. The abolition of collagen gene expression in SV40-transformed fibroblasts is associated with trans-acting factor switching. Nucleic Acids Research, 1992, 20(21): 5825-5830.

⁷ Le Naour R, Lussiez C, Raoul H, Mabondzo A, Dormont D. Expression of cell adhesion molecules at the surface of in vitro human immunodeficiency virus type 1-infected human monocytes: relationships with tumor necrosis factor alpha, interleukin 1beta, and interleukin 6 syntheses. AIDS Res Hum Retroviruses Jul. 1, 1997;13(10):841-55.

⁸ Tanaka Y, Fukudome K, Hayashi M, Takagi S, Yoshie O. Induction of ICAM-1 and LFA-3 by Tax1 of human T-cell leukemia virus type 1 and mechanism of down-regulation of ICAM-1 or LFA-1 in adult-T-cell-leukemia cell lines. Int J Cancer Feb. 8, 1995; 60(4):554-61.

⁹ Patarroyo M, Prieto J, Emberg I, Gahmberg C G. Absence, or low expression, of leukocyte adhesion molecules CD11 and CD18 on Burkitt lymphoma cells. Int J Cancer Jun. 15, 1988; 41(6):901-7.

¹⁰ Nuclear Respiratory Factor 2 should not be confused with NF-E2 Related Factor 2 which is also abbriviated NRF2 or NRF-2.

¹¹ Watanabe H, Imai T, Sharp P A, Handa H. Identification of two transcription factors that bind to specific elements in the promoter of the adenovirus early-region 4. Mol Cell Biol 1988 8(3):1290-300. The transcription factor binds to the promoter of the adenovirus early-region 4 (E4). Hence the name E4 transcription factor 1.

¹²¹² Enhancer Factor 1A should not be confused with Elongation Factor 1A which is also abbriviated EF-1A.

¹³ Suzuki F, Goto M, Sawa C, Ito S, Watanabe H, Sawada J, Handa H. Functional interactions of transcription factor human GA-binding protein subunits. J Biol Chem. Nov. 6, 1998; 273(45):29302-8.

¹⁴ Bannert N, Avots A, Baier M, Serfling E, Kurth R. GA-binding protein factors, in concert with the coactivator CREB binding protein/p300, control the induction of the interleukin 16 promoter in T lymphocytes. Proc, Natl, Acad, Sci, USA 1999 96:1541-1546.

¹⁵Kamei Y, Xu L, Heinzel T, Torchia J, Kurokawa R, Gloss B, Lin S C, Heyman R A, Rose D W, Glass C K, Rosenfeld M G. A CBP integrator complex mediates transcriptional activation and AP-1 inhibition by nuclear receptors. Cell. May 3, 1996; 85(3):403-14.

¹⁶ Horvai A E, Xu L, Korzus E, Brard G, Kalafus D, Mullen T M, Rose D W, Rosenfeld M G, Glass C K. Nuclear integration of JAK/STAT and Ras/AP-1 signaling by CBP and p300. Proc Natl Acad Sci U S A. Feb. 18, 1997;94(4):1074-9.

¹⁷ Hottiger M O, Felzien L K, Nabel G J. Modulation of cytokine-induced HIV gene expression by competitive binding of transcription factors to the coactivator p300. EMBO J. Jun. 1, 1998;17(11):3124-34.

¹⁸ Asano M, Murakami Y, Furukawa K, Yamaguchi-Iwai Y, Stake M, Ito Y. A Polayomavirus Enhancers Binding Protein, PEBP5, Responsive to 12-O-Tetradecanoylphorbol-13-Acetate but Distinct From AP-1. Journal of Virology 1990 64(12):5927-5938.

¹⁹ Higashino F, Yoshida K, Fujinaga Y, Kamio K, Fujinaga K. Isolation fo a cDNA Encoding the Adenovirus E1A Enhancer Binding Protein: A New Human Member of the ets Oncogene Family. Nucleic Acids Research 1993 21(3):547-553.

²⁰ Laimins L A, Tsichlis P, Khoury G. Multiple Enhancer Domains in the 3′ Terminus of the Prague Strain of Rous Sarcoma Virus. Nucleic Acids Research 1984 12(16):6427-6442.

²¹ LaMarco K L, McKnight S L. Purification of a set of cellular polypeptides that bind to the purine-rich cis-regulatory element of herpes simplex virus immediate early genes. Genes Dev 1989 3(9): 1372-83.

²² Douville P, Hagmann M, Georgiev O, Schaffner W. Positive and negative regulation at the herpes simplex virus ICP4 and ICP0 TAATGARAT motifs. Virology. Feb. 20, 1995;207(1):107-16.

²³ Boshart M, Weber F, Jahn G, Dorsch-Hasler K, Fleckenstein B, Schaffier W. A very strong enhancer is located upstream of an immediate early gene of human cytomegalovirus. Cell 1985 June;41(2):521-30.

²⁴ Gunther C V, Graves B J. Identification of ETS domain proteins in murine T lymphocytes that interact with the Moloney murine leukemia virus enhancer. Mol Cell Biol 1994 14(11): 7569-80

²⁵ Flory E, Hoffmeyer A, Smola U, Rapp U R, Bruder J T. Raf-1 kinase targets GA-binding protein in transcriptional regulation of the human immunodeficiency virus type 1 promoter. J Virol 1996 April;70(4):2260-8.

²⁶ Rawlins D R, Milman G, Hayward S D, Hayward G S. Sequence-specific DNA binding of the Epstein-Barr virus nuclear antigen (EBNA-1) to clustered sites in the plasmid maintenance region. Cell 1985 October;42(3):859-68.

²⁷ Mauclere P, Mahieux R, Garcia-Calleja J M, Salla R, Tekaia F, Millan J, De The G, Gessain A. A new HTLV-II subtype A isolate in an HIV-1 infected prostitute from Cameroon, Central Africa. AIDS Res Hum Retroviruses. 1995 August;11(8):989-93.

²⁸ Kornfeld H, Riedel N, Viglianti G A, Hirsch V, Mullins J I. Cloning of HTLV-4 and its relation to simian and human immunodeficiency viruses. Nature 1987 326(6113);610-613.

²⁹ Bannert N, Avots A, Baier M, Serfling E, Kurth R. GA-binding protein factors, in concert with the coactivator CREB binding protein/p300, control the induction of the interleukin 16 promoter in T lymphocytes. Proc, Natl, Acad, Sci, USA 1999 96:1541-1546.

³⁰ Flory E, Hoffmneyer A, Smola U, Rapp U R, Bruder J T. Raf-1 kinase targets GA-binding protein in transcriptional regulation of the human immunodeficiency virus type 1 promoter. J Virol 1996 April;70(4):2260-8.

³¹ Douville P, Hagmann M, Georgiev O, Schaffner W. Positive and negative regulation at the herpes simplex virus ICP4 and ICP0 TAATGARAT motifs. Virology. Feb. 20, 1995;207(1):107-16.

³² Bruder J T, Hearing P. Cooperative binding of EF-LA to the E1A enhancer region mediates synergistic effects on E1A transcription during adenovirus infection. J Virol. 1991 September;65(9):5084-7.

³³ Bruder J T, Hearing P. Nuclear factor EF-1 A binds to the adenovirus E1A core enhancer element and to other transcriptional control regions. Mol Cell Biol. 1989 November;9(11):5143-53.

³⁴ Ostapchuk P, Diffley J F, Bruder J T, Stillman B, Levine A J, Hearing P. Interaction of a nuclear factor with the polyomavirus enhancer region. Proc Natl Acad Sci U S A. 1986 November;83(22):8550-4.

³⁵ Gunther C V, Graves B J. Identification of ETS domain proteins in murine T lymphocytes that interact with the Moloney murine leukemia virus enhancer. Mol Cell Biol 1994 14(11): 7569-80

³⁶ Scholer H R, Gruss P. Specific interaction between enhancer-containing molecules and cellular components. Cell. 1984 February;36(2):403-11.

³⁷ Bannert N, Avots A, Baier M, Serfling E, Kurth R. GA-binding protein factors, in concert with the coactivator CREB binding protein/p300, control the induction of the interleukin 16 promoter in T lymphocytes. Proc, Natl, Acad, Sci, USA 1999 96:1541-1546.

³⁸ Hipskind R A, Bilbe G. MAP kinase signaling cascades and gene expression in osteoblasts. Front Biosci. Aug. 1, 1998;3:D804-16.

³⁹ Camps M, Nichols A, Arkinstall S. Dual specificity phosphatases: a gene family for control of MAP kinase function. FASEB J. 2000 January;14(l):6-16.

⁴⁰ Saxena M, Mustelin T. Extracellular signals and scores of phosphatases: all roads lead to MAP kinase. Semin Immunol. 2000 August;12(4):387-96.

⁴¹ Keyse S M. Protein phosphatases and the regulation of MAP kinase activity. Semin Cell Dev Biol. 1998 April;9(2): 143-52.

⁴² Espinos E, Le Van Thai A, Pomies C, Weber M J. Cooperation between phosphorylation and acetylation processes in transcriptional control. Mol Cell Biol 1999 May;19(5):3474-84.

⁴³ Schaeffer L, Duclert N, Huchet-Dymanus M, Changeux J P. Implication of a multisubunit Ets-related transcription factor in synaptic expression of the nicotinic acetylcholine receptor. EMBO J. Jun. 1, 1998;17(11):3078-90.

⁴⁴ Shiraishi M, Hirasawa N, Kobayashi Y, Oikawa S, Murakami A, Ohuchi K. Participation of mitogen-activated protein kinase in thapsigargin- and TPA-induced histamine production in murine macrophage RAW 264.7 cells. Br J Pharmacol 2000 February;129(3):515-24.

⁴⁵ Herrera R, Hubbell S, Decker S, Petruzzelli L. A role for the MEK/MAPK pathway in PMA-induced cell cycle arrest: modulation of megakaryocytic differentiation of K562 cells. Exp Cell Res Feb. 1, 1998;238(2):407-14.

⁴⁶ Stadheim T A, Kucera G L. Extracellular signal-regulated kinase (ERK) activity is required for TPA-mediated inhibition of drug-induced apoptosis. Biochem Biophys Res Commun Apr. 1, 1998;245(1):266-71.

⁴⁷ Yen A, Roberson M S, Varvayanis S. Retinoic acid selectively activates the ERK2 but not JNK/SAPK or p38 MAP kinases when inducing myeloid differentiation. In vitro Cell Dev Biol Anim. 1999 October;35(9):527-32.

⁴⁸ Liu M K, Brownsey R W, Reiner N E. Γ interferon induces rapid and coordinate activation of mitogen-activated protein kinase (extracellular signal-regulated kinase) and calcium-independent protein kinase C in human monocytes. Infect Immun, July 1994, 2722-2731, Vol 62, No. 7.

⁴⁹ Nishiya T, Uehara T, Edamatsu H, Kaziro Y, Itoh H, Nomura Y. Activation of Statl and subsequent transcription of inducible nitric oxide synthase gene in C6 glioma cells is independent of interferon-γ-induced MAPK activation that is mediated by p21ras. FEBS Lett May 12, 1997;408(1):33-8.

⁵⁰ Lessor T, Yoo J Y, Davis M, Hamburger A W. Regulation of heregulin betal-induced differentiation in a human breast carcinoma cell line by the extracellular-regulated kinase (ERK) pathway. J Cell Biochem Sep. 15, 1998;70(4):587-95.

⁵¹ Marte B M, Graus-Porta D, Jeschke M, Fabbro D, Hynes N E, Tavema D. NDF/heregulin activates MAP kinase and p70/p85 S6 kinase during proliferation or differentiation of mammary epithelial cells. Oncogene Jan. 5, 1995;10(1): 167-75.

⁵² Sepp-Lorenzino L, Eberhard I, Ma Z, Cho C, Serve H, Liu F, Rosen N, Lupu R. Signal transduction pathways induced by heregulin in MDA-MB-453 breast cancer cells. Oncogene Apr. 18, 1996;12(8):1679-87.

⁵³ Fiddes R J, Janes P W, Sivertsen S P, Sutherland R L, Musgrove E A, Daly R J. Inhibition of the MAP kinase cascade blocks heregulin-induced cell cycle progression in T-47D human breast cancer cells. Oncogene May 28, 1998;16(21):2803-13.

⁵⁴ Park J A, Koh J Y. Induction of an immnediate early gene egr-1 by zinc through extracellular signal-regulated kinase activation in cortical culture: its role in zinc-induced neuronal death. J Neurochem. 1999 August;73(2):450-6.

⁵⁵ Kiss Z, Crilly K S, Tomono M. Bombesin and zinc enhance the synergistic mitogenic effects of insulin and phosphocholine by a MAP kinase-dependent mechanism in Swiss 3T3 cells. FEBS Lett. Sep, 22, 1997;415(1):71-4.

⁵⁶ Wu W, Graves L M, Jaspers I, Devlin R B, Reed W, Samet J M. Activation of the EGF receptor signaling pathway in human airway epithelial cells exposed to metals. Am J Physiol. 1999 November;277(5 Pt l):L924-31.

⁵⁷ Samet J M, Graves L M, Quay J, Dailey L A, Devlin R B, Ghio A J, Wu W, Bromberg P A, Reed W. Activation of MAPKs in human bronchial epithelial cells exposed to metals. Am J Physiol. 1998 September;275(3 Pt 1):L551-8.

⁵⁸ Migliaccio A, Di Domenico M, Castoria G, de Falco A, Bontempo P, Nola E, Auricchio F. Tyrosine kinase/p21ras/MAP-kinase pathway activation by estradiol-receptor complex in MCF-7 cells. EMBO J. Mar. 15, 1996;15(6):1292-300.

⁵⁹ Ruzycky A L. Effects of 17 beta-estradiol and progesterone on mitogen-activated protein kinase expression and activity in rat uterine smooth muscle. Eur J Pharmacol. Apr. 11, 1996;300(3):247-54.

⁶⁰ Nuedling S, Kahlert S, Loebbert K, Meyer R, Vetter H, Grohe C. Differential effects of 17beta-estradiol on mitogen-activated protein kinase pathways in rat cardiomyocytes. FEBS Lett. Jul. 9, 1999;454(3):271-6.

⁶¹ Laporte J D, Moore P E, Abraham J H, Maksym G N, Fabry B, Panettieri R A Jr, Shore S A. Role of ERK MAP kinases in responses of cultured human airway smooth muscle cells to IL-lbeta. Am J Physiol. 1999 November;277(5 Pt l):L943-51.

⁶² Kumar A, Middleton A, Chambers T C, Mehta K D. Differential roles of extracellular signal-regulated kinase-1/2 and p38(MAPK) in interleukin-1beta- and tumor necrosis factor-alpha-induced low density lipoprotein receptor expression in HepG2 cells. J Biol Chem. Jun. 19, 1998;273(25):15742-8.

⁶³ Larsen C M, Wadt K A, Juhl L F, Andersen H U, Karlsen A E, Su M S, Seedorf K, Shapiro L, Dinarello C A, Mandrup-Poulsen T. Interleukin-1beta-induced rat pancreatic islet nitric oxide synthesis requires both the p38 and extracellular signal-regulated kinase 1/2 mitogen-activated protein kinases. J Biol Chem. Jun. 12, 1998;273(24):15294-300.

⁶⁴ Daeipour M, Kumar G, Amaral M C, Nel A E. Recombinant IL-6 activates p42 and p44 mitogen-activated protein kinases in the IL-6 responsive B cell line, AF-10. J Immunol. Jun. 1,1993;150(11):4743-53.

⁶⁵ Leonard M, Ryan M P, Watson A J, Schramek H, Healy E. Role of MAP kinase pathways in mediating IL-6 production in human primary mesangial and proximal tubular cells. Kidney Int. 1999 October;56(4): 1366-77.

⁶⁶ Hartsough M T, Mulder K M. Transforming growth factor beta activation of p44mapk in proliferating cultures of epithelial cells. J Biol Chem. Mar. 31, 1995;270(13):7117-24.

⁶⁷ Yonekura A, Osaki M, Hirota Y, Tsukazaki T, Miyazali Y, Matsumoto T, Ohtsuru A, Namba H, Shindo H, Yamashita S. Transforming growth factor-beta stimulates articular chondrocyte cell growth through p44/42 MAP kinase (ERK) activation. Endocr J. 1999 August;46(4):545-53.

⁶⁸ Strakova Z, Copland J A, Lolait S J, Soloff M S. ERK2 mediates oxytocin-stimulated PGE2 synthesis. Am J Physiol. 1998 April;274(4 Pt 1):E634-41.

⁶⁹ Copland J A, Jeng Y J, Strakova Z, Ives K L, Hellmich M R, Soloff M S. Demonstration of functional oxytocin receptors in human breast Hs578T cells and their up-regulation through a protein kinase C-dependent pathway. Endocrinology. 1999 May;140(5):2258-67.

⁷⁰ Hoare S, Copland J A, Wood T G, Jeng Y J, Izban M G, Soloff M S. Identification of a GABP alpha/beta binding site involved in the induction of oxytocin receptor gene expression in human breast cells, potentiation by c-Fos/c-Jun. Endocrinology. 1999 May;140(5):2268-79.

⁷¹ Flory E, Hoffmeyer A, Smola U, Rapp U R, Bruder J T. Raf-1 kinase targets GA-binding protein in transcriptional regulation of the human immunodeficiency virus type 1 promoter. J Virol 1996 April;70(4):2260-8.

⁷²Avots A, Hoffmneyer A, Flory E, Cimanis A, Rapp U R, Serfling E. GABP factors bind to a distal interleukin 2 (IL-2) enhancer and contribute to c-Raf-mediated increase in IL-2 induction. Molecular and Cellular Biology 1997 17(8):4381-4389.

⁷³ Hoffmeyer A, Avots A, Flory E, Weber C K, Serfling E, Rapp U R. The GABP-responsive element of the interleukin-2 enhancer is regulated by JNK/SAPK-activating pathways in T lymphocytes. J Biol Chem. Apr. 24, 1998;273(17):10112-9.

⁷⁴ Tomaras G D, Foster D A, Burrer C M, Taffet S M. ETS transcription factors regulate an enhancer activity in the third intron of TNF-alpha. J Leukoc Biol 1999 July;66(1):183-93.

⁷⁵ Zhong H, Voll R E, Ghosh S. Phosphorylation of NF-kappa B p65 by PKA stimulates transcriptional activity by promoting a novel bivalent interaction with the coactivator CBP/p300. Mol Cell. 1998 April;1(5):661-71.

⁷⁶ Bevilacqua M A, Faniello M C, Cimino F, Costanzo F. Okadaic acid stimulates H ferritin transcription in HeLa cells by increasing the interaction between the p300 CO-activator molecule and the transcription factor Bbf. Biochem Biophys Res Commun. Nov. 7, 1997;240(1): 179-82.

⁷⁷ Kuo M H, Allis C D. Roles of histone acetyltransferases and deacetylases in gene regulation. BioEassays 1998 20:615-626.

⁷⁸ Hebbes T R, Clayton A L, Thome A W, Crane-Robinson C. Core histone hyperacetylation co-maps with generalized DNase I sensitivity in the chicken beta-globin chromosomal domain. EMBO J. Apr. 15, 1994;13(8):1823-30.

⁷⁹ Kuhnert P, Peterhans E, Pauli U. Chromatin structure and DNase I hypersensitivity in the transcriptionally active and inactive porcine tumor necrosis factor gene locus. Nucleic Acids Res. Apr. 25, 1992;20(8): 1943-8.

⁸⁰ Tomaras G D, Foster D A, Burrer C M, Taffet S M. ETS transcription factors regulate an enhancer activity in the third intron of TNF-alpha. J Leukoc Biol 1999 July;66(1):183-93.

⁸¹ Espinos E, Le Van Thai A, Pomies C, Weber M J. Cooperation between phosphorylation and acetylation processes in transcriptional control. Mol Cell Biol 1999 May;19(5):3474-84.

⁸² Herschlag D., Johnson F. B., 1993. Synergism in transcriptional activation: a kinetic view. Genes and Development, 7:173-179.

⁸³ Espinos E, Le Van Thai A, Poniies C, Weber M J. Cooperation between phosphorylation and acetylation processes in transcriptional control. Mol Cell Biol 1999 May;19(5):3474-84.

⁸⁴ Bannert N, Avots A, Baier M, Serfling E, Kurth R. GA-binding protein factors, in concert with the coactivator CREB binding protein/p300, control the induction of the interleukin 16 promoter in T lymphocytes. Proc, Natl, Acad, Sci, USA 1999 96:1541-1546.

⁸⁵ Eckner R, Ewen M E, Newsome D, Gerdes M, DeCaprio J A, Lawrence J B, Livingston D M. Molecular cloning and functional analysis of the adenovirus E1A-associated 300-kD protein (p300) reveals a protein with properties of a transcriptional adaptor. Genes Dev. Apr. 15, 1994;8(8):869-84.

⁸⁶ Flory E, Hoffmeyer A, Smola U, Rapp U R, Bruder J T. Raf-1 kinase targets GA-binding protein in transcriptional regulation of the human imrnunodeficiency virus type 1 promoter. J Virol 1996 April;70(4):2260-8.

⁸⁷ Avots A, Hoffmeyer A, Flory E, Cimanis A, Rapp U R, Serfling E. GABP factors bind to a distal interleukin 2 (IL-2) enhancer and contribute to c-Raf-mediated increase in IL-2 induction. Molecular and Cellular Biology 17(8):4381-4389.

⁸⁸ Hoffmeyer A, Avots A, Flory E, Weber C K, Serfling E, Rapp U R. The GABP-responsive element of the interleukin-2 enhancer is regulated by JNK/SAPK-activating pathways in T lymphocytes. J Biol 89 Chem. Apr. 24, 1998;273(17):10112-9.

⁸⁹ Martin M E, Chinenov Y, Yu M, Schmidt T K, Yang X Y. Redox regulation of GA-binding protein-alpha DNA binding activity. J Biol Chem. Oct. 11, 1996;271(41):25617-23.

⁹⁰ Suzuki F, Goto M, Sawa C, Ito S, Watanabe H, Sawada J, Handa H. Functional interactions of transcription factor human GA-binding protein subunits. J Biol Chem. Nov. 6, 1998;273(45):29302-8.

⁹¹ Hoare S, Copland J A, Wood T G, Jeng Y J, Izban M G, Soloff M S. Identification of a GABP alpha/beta binding site involved in the induction of oxytocin receptor gene expression in human breast cells, potentiation by c-Fos/c-Jun. Endocrinology. 1999 May; 140(5):2268-79.

⁹² Rosmarin A G, Luo M, Caprio D G, Shang J, Simkevich C P. Sp1 cooperates with the ets transcription factor, GABP, to activate the CD18 (beta2 leukocyte integrin) promoter. J Biol Chem May 22, 1998;273(21):13097-103.

⁹³ Tomaras G D, Foster D A, Burrer C M, Taffet S M. ETS transcription factors regulate an enhancer activity in the third intron of TNF-alpha. J Leukoc Biol 1999 July;66(1):183-93.

⁹⁴ Bottinger E P, Shelley C S, Farokhzad O C, Arnaout M A. The human beta 2 integrin CD18 promoter consists of two inverted Ets cis elements. Mol Cell Biol. 1994 April;14(4):2604-15.

⁹⁵ Sowa Y, Shiio Y, Fujita T, Matsumoto T, Okuyama Y, Kato D, Inoue J, Sawada J, Goto M, Watanabe H, Handa H, Sakai T. Retinoblastoma binding factor 1 site in the core promoter region of the human RB gene is activated by hGABP/E4TF1. Cancer Res. Aug. 1, 1997;57(15):3145-8.

⁹⁶ Atlas E, Stramwasser M, Whiskin K, Mueller C R. GA-binding protein alpha/beta is a critical regulator of the BRCA1 promoter. Oncogene Apr. 6, 2000; 19(15):1933-40.

⁹⁷ Li X R, Chong A S, Wu J, Roebuck K A, Kumar A, Parrillo J E, Rapp U R, Kimberly R P, Williams J W, Xu. XTranscriptional regulation of Fas gene expression by GA-binding protein and AP-1 in T cell antigen receptor.CD3 complex-stimulated T cells. J Biol Chem. Dec. 3, 1999;274(49):35203-10.

⁹⁸ Donovan-Peluso M, George L D, Hassett A C. Lipopolysaccharide induction of tissue factor expression in THP-1 monocytic cells. Protein-DNA interactions with the promoter. J Biol Chem. Jan. 14, 1994;269(2): 1361-9.

⁹⁹ Groupp E R, Donovan-Peluso M. Lipopolysaccharide induction of THP-1 cells activates binding of c-Jun, Ets, and Egr-1 to the tissue factor promoter. J Biol Chem. May 24, 1996;271(21):12423-30.

¹⁰⁰ Hall A J, Vos H L, Bertina R M. Lipopolysaccharide induction of tissue factor in THP-1 cells involves Jun protein phosphorylation and nuclear factor kappaB nuclear translocation. J Biol Chem. Jan. 1, 1999;274(1):376-83.

¹⁰¹ Holzmuller H, Moll T, Hofer-Warbinek R, Mechtcheriakova D, Binder B R, Hofer E. A transcriptional repressor of the tissue factor gene in endothelial cells. Arterioscler Thromb Vasc Biol. 1999 July; 19(7):1804-11.

¹⁰² Donovan-Peluso M, George L D, Hassett A C. Lipopolysaccharide induction of tissue factor expression in THP-1 monocytic cells. Protein-DNA interactions with the promoter. J Biol Chem. Jan. 14, 1994;269(2): 1361-9.

¹⁰³ Holzmuller H, Moll T, Hofer-Warbinek R, Mechtcheriakova D, Binder B R, Hofer E. A transcriptional repressor of the tissue factor gene in endothelial cells. Arterioscler Thromb Vasc Biol. 1999 July;19(7):1804-11.

¹⁰⁴ Moll T, Czyz M, Holzmuller H, Hofer-Warbinek R, Wagner E, Winkler H, Bach F H, Hofer E. Regulation of the tissue factor promoter in endothelial cells. Binding of NF kappa B-, AP-1-, and Sp1-like transcription factors. J Biol Chem. 1995 Feb 24;270(8):3849-57.

¹⁰⁵Nathwani A C, Gale K M, Pemberton K D, Crossman D C, Tuddenham E G, McVey J H. Efficient gene transfer into human umbilical vein endothelial cells allows functional analysis of the human tissue factor gene promoter. Br J Haematol. 1994 September;88(1):122-8.

¹⁰⁶ Mackman N, Fowler B J, Edgington T S, Morrissey J H. Functional analysis of the human tissue factor promoter and induction by serum. Proc Natl Acad Sci U S A. 1990 March;87(6):2254-8.

¹⁰⁷ Key N S, Bach R R, Vercellotti G M, Moldow C F. Herpes simplex virus type I does not require productive infection to induce tissue factor in human umbilical vein endothelial cells. Lab Invest. 1993 June;68(6):645-51.

¹⁰⁸ Oeth P, Yao J, Fan S T, Mackman N. Retinoic acid selectively inhibits lipopolysaccharide induction of tissue factor gene expression in human monocytes. Blood. Apr. 15, 1998;91(8):2857-65.

¹⁰⁹Pendurthi U R, Williams J T, Rao L V. Resveratrol, a polyphenolic compound found in wine, inhibits tissue factor expression in vascular cells: A possible mechanism for the cardiovascular benefits associated with moderate consumption of wine. Arterioscler Thromb Vasc Biol. 1999 Feb;19(2):419-26.

¹¹⁰ Bach R R, Moldow C F. Mechanism of tissue factor activation on HL-60 cells. Blood. May 1, 1997;89(9):3270-6.

¹¹¹ Nemerson Y, Giesen P L. Some thoughts about localization and expression of tissue factor. Blood Coagul Fibrinolysis. 1998 March;9 Suppl 1:S45-7.

¹¹² Wolberg A S, Kon R H, Monroe D M, Ezban M, Roberts H R, Hoffman M. Deencryption of Cellular Tissue Factor Is Independent of Its Cytoplasmic Domain. Biochem Biophys Res Commun. Jun. 7, 2000;272(2):332-336.

¹¹³ Schecter A D, Giesen P L, Taby O, Rosenfield C L, Rossikhina M, Fyfe B S, Kohtz D S, Fallon J T, Nemerson Y, Taubman M B. Tissue factor expression in human arterial smooth muscle cells. TF is present in three cellular pools after growth factor stimulation. J Clin Invest. Nov. 1, 1997; 100(9):2276-85.

¹¹⁴ Schecter A D, Giesen P L, Taby O, Rosenfield C L, Rossikhina M, Fyfe B S, Kohtz D S, Fallon J T, Nemerson Y, Taubman M B. Tissue factor expression in human arterial smooth muscle cells. TF is present in three cellular pools after growth factor stimulation. J Clin Invest. Nov. 1, 1997; 100(9):2276-85.

¹¹⁵ Schecter A D, Giesen P L, Taby O, Rosenfield C L, Rossikhina M, Fyfe B S, Kohtz D S, Fallon J T, Nemerson Y, Taubman M B. Tissue factor expression in human arterial smooth muscle cells. TF is present in three cellular pools after growth factor stimulation. J Clin Invest. Nov. 1, 1997;100(9):2276-85.

¹¹⁶ Pan J, Xia L, McEver R P. Comparison of promoters for the murine and human P-selectin genes suggests species-specific and conserved mechanisms for transcriptional regulation in endothelial cells. J Biol Chem. Apr. 17, 1998;273(16):10058-67.

¹¹⁷ Rosmarin A G, Caprio D G, Kirsch D G, Handa H, Simkevich C P. GABP and PU. 1 compete for binding, yet cooperate to increase CD18 (beta 2 leukocyte integrin) transcription. J Biol Chem Oct. 6, 1995;270(40):23627-33.

¹¹⁸ Rosmarin A G, Luo M, Caprio D G, Shang J, Sirnkevich C P. Sp1 cooperates with the ets transcription factor, GABP, to activate the CD18 (beta2 leukocyte integrin) promoter. J Biol Chem May 22, 1998;273(21): 13097-103.

¹¹⁹ Le Naour R, Lussiez C, Raoul H, Mabondzo A, Dormont D. Expression of cell adhesion molecules at the surface of in vitro human immunodeficiency virus type 1-infected human monocytes: relationships with tumor necrosis factor alpha, interleukin 1beta, and interleukin 6 syntheses. AIDS Res Hum Retroviruses Jul. 1, 1997;13(10):841-55.

¹²⁰ Tanaka Y, Fukudome K, Hayashi M, Takagi S, Yoshie O. Induction of ICAM-1 and LFA-3 by Tax1 of human T-cell leukemia virus type 1 and mechanism of down-regulation of ICAM-1 or LFA-1 in adult-T-cell-leukemia cell lines. Int J Cancer Feb 8, 1995 60(4):554-61.

¹²¹ Patarroyo M, Prieto J, Ernberg I, Gahmberg C G. Absence, or low expression, of leukocyte adhesion molecules CD11 and CD18 on Burkitt lymphoma cells. Int J Cancer Jun. 15, 1988;41(6):901-7.

¹²² Rosen G D, Barks J L, Iademarco M F, Fisher R J, Dean D C. An intricate arrangement of binding sites for the Ets family of transcription factors regulates activity of the alpha 4 integrin gene promoter. J Biol Chem. Jun 3, 1994;269(22):15652-60.

¹²³ Talmud P J, Palmen J, Walker M. Identification of genetic variation in the human hormone-sensitive lipase gene and 5′ sequences: homology of 5′ sequences with mouse promoter and identification of potential regulatory elements. Biochem Biophys Res Commun. Nov. 27, 1998;252(3):661-8.

¹²⁴ Grober J, Laurell H, Blaise R, Fabry B, Schaak S, Holm C, Langin D. Characterization of the promoter of human adipocyte hormone-sensitive lipase. Biochem J. Dec. 1, 1997;328 (Pt 2):453-61.

¹²⁵ YU M, Yang X Y, Schmidt T, Chinenov Y, Wang R, Martin M E. GA-binding protein-dependent transcription initiator elements. Effect of helical spacing between polyomavirus enhancer a factor 3(PEA3)/Ets-binding sites on initiator activity. J Biol Chem Nov. 14, 1997;272(46):29060-7.

¹²⁶ Blaise R, Grober J, Rouet P, Tavernier G, Daegelen D, Langin D. Testis expression of hormone-sensitive lipase is conferred by a specific promoter that contains four regions binding testicular nuclear proteins. J Biol Chem. Apr. 2, 1999;274(14):9327-34.

¹²⁷ Talmud P J, Palmen J, Walker M. Identification of genetic variation in the human hormone-sensitive lipase gene and 5′ sequences: homology of 5′ sequences with mouse promoter and identification of potential regulatory elements. Biochem Biophys Res Commun. Nov. 27, 1998;252(3):661-8.

¹²⁸ Kawamura M, Jensen D F, Wancewicz E V, Joy L L, Khoo J C, Steinberg D. Hormone-sensitive lipase in differentiated 3T3-L1 cells and its activation by cyclic AMP-dependent protein kinase. Proc Natl Acad Sci U S A. 1981 February;78(2):732-6.

¹²⁹ Gordeladze J O, Hovik K E, Merendino J J, Hermouet S, Gutkind S, Accili D. Effect of activating and inactivating mutations of Gs- and Gi2-alpha protein subunits on growth and differentiation of 3T3-L1 preadipocytes. J Cell Biochem. 1997 February;64(2):242-57.

¹³⁰ Armelin H A, Armelin M C, Kelly K, Stewart T, Leder P, Cochran B H, Stiles C D. Functional role for c-myc in mitogenic response to platelet-derived growth factor. Nature. Aug. 23-29, 1984;310(5979):655-60.

¹³¹ Cepko C L, Roberts B E, Mulligan R C. Construction and applications of a highly transmissible murine retrovirus shuttle vector. Cell. 1984 July;37(3):1053-62.

¹³² Higgins C, Chatterjee S, Cherington V. The block of adipocyte differentiation by a C-terminally truncated, but not by full-length, simian virus 40 large tumor antigen is dependent on an intact retinoblastoma susceptibility protein family binding domain. J Virol. 1996 February;70(2):745-52.

¹³³ Higgins C, Chatterjee S, Cherington V. The block of adipocyte differentiation by a C-terminally truncated, but not by full-length, simian virus 40 large tumor antigen is dependent on an intact retinoblastoma susceptibility protein family binding domain. J Virol. 1996 February;70(2):745-52.

¹³⁴ Cherington V, Brown M, Paucha E, St Louis J, Spiegelman B M, Roberts T M. Separation of simian virus 40 large-T-antigen-transforming and origin-binding functions from the ability to block differentiation. Mol Cell Biol. 1988 March;8(3):1380-4.

¹³⁵ Sellers W R, Kaelin W G Jr. Role of the retinoblastoma protein in the pathogenesis of human cancer. J Clin Oncol 1997 November;15(11):3301-12.

¹³⁶ Dou Q P, An B. RB and apoptotic cell death. Front Biosci. Apr. 6, 1998;3:d419-30.

¹³⁷ Coppola J A, Lewis B A, Cole M D. Increased retinoblastoma gene expression is associated with late stages of differentiation in many different cell types. Oncogene 1990 November;5(11):1731-3.

¹³⁸ Levine R A, Hopman T, Guo L, Chang M J, Johnson N. Induction of retinoblastoma gene expression during terminal growth arrest of a conditionally immortalized fetal rat lung epithelial cell line and during fetal lung maturation. Exp Cell Res. Mar. 15, 1998;239(2):264-76.

¹³⁹ Slack R S, Hamel P A, Bladon T S, Gill R M, McBurney M W. Regulated expression of the retinoblastoma gene in differentiating embryonal carcinoma cells. Oncogene. 1993 June;8(6):1585-91.

¹⁴⁰ Richon V M, Rifkind R A, Marks P A. Expression and phosphorylation of the retinoblastoma protein during induced differentiation of murine erythroleukemia cells. Cell Growth Differ. 1992 July;3(7):413-20.

¹⁴¹ Schwartz B, Avivi-Green C, Polak-Charcon S. Sodium butyrate induces retinoblastoma protein dephosphorylation, p16 expression and growth arrest of colon cancer cells. Mol Cell Biochem. 1998 November;188(1-2):21-30.

¹⁴² Thompson M E, Jensen R A, Obermiller P S, Page D L, Holt J T. Decreased expression of BRCA1 accelerates growth and is often present during sporadic breast cancer progression. Nat Genet 1995 April ;9(4):444-50.

¹⁴³ Rao V N, Shao N, Ahmad M, Reddy E S. Antisense RNA to the putative tumor suppressor gene BRCA1 transforms mouse fibroblasts. Oncogene. Feb. 1, 1996;12(3):523-8.

¹⁴⁴ Holt J T, Thompson M E, Szabo C, Robinson-Benion C, Arteaga C L, King M C, Jensen R A. Growth retardation and tumour inhibition by BRCA1. Nat Genet. 1996 March; 12(3):298-302.

¹⁴⁵ Tait D L, Obetmiller P S, Redlin-Frazier S, Jensen R A, Welcsh P, Dann J, King M C, Johnson D H, Holt J T. A phase I trial of retroviral BRCA1sv gene therapy in ovarian cancer. Clin Cancer Res. 1997 November;3(11): 1959-68.

¹⁴⁶ Russell P A, Pharoah P D, De Foy K, Ramus S J, Symmonds I, Wilson A, Scott I, Ponder B A, Gayther S A. Frequent loss of BRCA1 mRNA and protein expression in sporadic ovarian cancers. Int J Cancer. 2000 August;87(3):317-321.

¹⁴⁷ Rio P G, Maurizis J C, Peffault de Latour M, Bignon Y J, Bernard-Gallon D J. Quantification of BRCA1 protein in sporadic breast carcinoma with or without loss of heterozygosity of the BRCA1 gene. Int J Cancer Mar. 15, 1999;80(6):823-6.

¹⁴⁸ Rice J C, Massey-Brown K S, Futscher B W. Aberrant methylation of the BRCA1 CpG island promoter is associated with decreased BRCA1 mRNA in sporadic breast cancer cells. Oncogene. Oct. 8, 1998;17(14):1807-12.

¹⁴⁹ Magdinier F, Ribieras S, Lenoir G M, Frappart L, Dante R. Down-regulation of BRCA1 in human sporadic breast cancer; analysis of DNA methylation patterns of the putative promoter region. Oncogene. Dec. 17, 1998;17(24):3169-76.

¹⁵⁰ Ozcelik H, To M D, Couture J, Bull S B, Andrulis I L. Preferential allelic expression can lead to reduced expression of BRCA1 in sporadic breast cancers. Int J Cancer. Jul. 3, 1998;77(1):1-6.

¹⁵¹ Thompson M E, Jensen R A, Obermiller P S, Page D L, Holt J T. Decreased expression of BRCA1 accelerates growth and is often present during sporadic breast cancer progression. Nat Genet 1995 April;9(4):444-50.

¹⁵² Russell P A, Pharoah P D, De Foy K, Ramus S J, Symmonds I, Wilson A, Scott I, Ponder B A, Gayther S A. Frequent loss of BRCA1 mRNA and protein expression in sporadic ovarian cancers. Int J Cancer. 2000 August;87(3):317-321.

¹⁵³ Rio P G, Maurizis J C, Peffault de Latour M, Bignon Y J, Bernard-Gallon D J. Quantification of BRCA1 protein in sporadic breast carcinoma with or without loss of heterozygosity of the BRCA1 gene. Int J Cancer Mar. 15, 1999;80(6):823-6.

¹⁵⁴Futreal P A, Liu Q, Shattuck-Eidens D, Cochran C, Harshman K, Tavtigian S, Bennett L M, Haugen-Strano A, Swensen J, Miki Y, et al. BRCA1 mutations in primary breast and ovarian carcinomas. Science Oct. 7, 1994;266(5182): 120-2.

¹⁵⁵ Merajver S D, Pham T M, Caduff R F, Chen M, Poy E L, Cooney K A, Weber B L, Collins F S, Johnston C, Frank T S. Somatic mutations in the BRCA1 gene in sporadic ovarian tumours. Nat Genet. 1995 April;9(4):439-43.

¹⁵⁶ Catteau A, Harris W H, Xu C F, Solomon E. Methylation of the BRCA1 promoter region in sporadic breast and ovarian cancer: correlation with disease characteristics. Oncogene. Mar. 18, 1999;18(11): 1957-65.

¹⁵⁷ Magdinier F, Ribieras S, Lenoir G M, Frappart L, Dante R. Down-regulation of BRCA1 in human sporadic breast cancer; analysis of DNA methylation patterns of the putative promoter region. Oncogene. Dec. 17, 1998;17(24):3169-76.

¹⁵⁸ Rice J C, Massey-Brown K S, Futscher B W. Aberrant methylation of the BRCA1 CpG island promoter is associated with decreased BRCA1 mRNA in sporadic breast cancer cells. Oncogene. Oct. 8, 1998;17(14): 1807-12.

¹⁵⁹ Dobrovic A, Simnpfendorfer D. Methylation of the BRCA1 gene in sporadic breast cancer. Cancer Res. Aug. 15, 1997;57(16):3347-50.

¹⁶⁰ Davidson W F, Giese T, Fredrickson T N. Spontaneous development of plasmacytoid tumors in mice with defective Fas-Fas ligand interactions. J Exp Med. Jun. 1, 1998;187(11):1825-38.

¹⁶¹ Drappa J, Vaishnaw A K, Sullivan K E, Chu J L, Elkon K B. Fas gene mutations in the Canale-Smith syndrome, an inherited lymphoproliferative disorder associated with autoimmunity. N Engl J Med. Nov. 28, 1996;335(22):1643-9.

¹⁶² Keane M M, Ettenberg S A, Lowrey G A, Russell E K, Lipkowitz S. Fas expression and function in normal and malignant breast cell lines. Cancer Res. Oct. 15, 1996;56(20):4791-8.

¹⁶³ Gratas C, Tohma Y, Barnas C, Taniere P, Hainaut P, Ohgaki H. Up-regulation of Fas (APO-1/CD95) ligand and down-regulation of Fas expression in human esophageal cancer. Cancer Res. May 15, 1998;58(10):2057-62.

¹⁶⁴ Strand S, Hofmann W J, Hug H, Muller M, Otto G, Strand D, Mariani S M, Stremmel W, Krammer P H, Galle P R. Lymphocyte apoptosis induced by CD95 (APO-1/Fas) ligand-expressing tumor cells—a mechanism of immune evasion? Nat Med. 1996 December;2(12):1361-6.

¹⁶⁵ Moller P, Koretz K, Leithauser F, Bruderlein S, Henne C, Quentmeier A, Krammer P H. Expression of APO-1 (CD95), a member of the NGF/TNF receptor superfamily, in normal and neoplastic colon epithelium. Int J Cancer. May 1, 1994;57(3):371-7.

¹⁶⁶ Leithauser F, Dhein J, Mechtersheimer G, Koretz K, Bruderlein S, Henne C, Schmidt A, Debatin K M, Krammer P H, Moller P. Constitutive and induced expression of APO-1, a new member of the nerve growth factor/tumor necrosis factor receptor superfamily, in normal and neoplastic cells. Lab Invest. 1993 October;69(4):415-29.

¹⁶⁷ Das H, Koizumi T, Sugimoto T, Chakraborty S, Ichimura T, Hasegawa K, Nishimura R. Quantitation of Fas and Fas ligand gene expression in human ovarian, cervical and endometrial carcinomas using real-time quantitative RT-PCR. Br J Cancer. 2000 May;82(10):1682-8.

¹⁶⁸ Butler L M, Hewett P J, Butler W J, Cowled P A. Down-regulation of Fas gene expression in colon cancer is not a result of allelic loss or gene rearrangement. Br J Cancer. 1998 May;77(9):1454-9.

¹⁶⁹ Keane M M, Ettenberg S A, Lowrey G A, Russell E K, Lipkowitz S. Fas expression and function in normal and malignant breast cell lines. Cancer Res. Oct. 15, 1996;56(20):4791-8.

¹⁷⁰ Bertoni F, Conconi A, Carobbio S, Realini C, Codegoni A M, Zucca E, Cavalli F. Analysis of Fas/CD95 gene somatic mutations in ovarian cancer cell lines. Int J Cancer. May 1, 2000;86(3):450.

¹⁷¹ Lee S H, Shin M S, Park W S, Kim S Y, Kim H S, Han J Y, Park G S, Dong S M, Pi J H, Kim CS, Kim S H, Lee J Y, Yoo N J. Alterations of Fas (Apo-1/CD95) gene in non-small cell lung cancer. Oncogene. Jun. 24, 1999;18(25):3754-60.

¹⁷² Lee S H, Shin M S, Park W S, Kim S Y, Dong S M, Pi J H, Lee H K, Kim H S, Jang J J, Kim CS, Kim S H, Lee J Y, Yoo N J. Alterations of Fas (APO-1/CD95) gene in transitional cell carcinomas of urinary bladder. Cancer Res Jul. 1, 1999;59(13):3068-72.

¹⁷³ Shin M S, Park W S, Kim S Y, Kim H S, Kang S J, Song K Y, Park J Y, Dong S M, Pi J H, Oh R R, Lee J Y, Yoo N J, Lee S H. Alterations of Fas (Apo-1/CD95) gene in cutaneous malignant melanoma. Am J Pathol 1999 June;154(6):1785-91.

¹⁷⁴ Butler L M, Hewett P J, Butler W J, Cowled P A. Down-regulation of Fas gene expression in colon cancer is not a result of allelic loss or gene rearrangement. Br J Cancer. 1998 May;77(9):1454-9.

¹⁷⁵ Butler L M, Dobrovic A, Bianco T, Cowled P A. Promoter region methylation does not account for the frequent loss of expression of the Fas gene in colorectal carcinoma. Br J Cancer. 2000 January;82(1):131-5.

¹⁷⁶ Lessor T, Yoo J Y, Davis M, Hamburger A W. Regulation of heregulin betal-induced differentiation in a human breast carcinoma cell line by the extracellular-regulated kinase (ERK) pathway. J Cell Biochem Sep. 15, 1998;70(4):587-95.

¹⁷⁷ Lessor T, Yoo J Y, Davis M, Hamburger A W. Regulation of heregulin betal-induced differentiation in a human breast carcinoma cell line by the extracellular-regulated kinase (ERK) pathway. J Cell Biochem Sep. 15, 1998;70(4):587-95.

¹⁷⁸ He H. Wang X, Gorospe M, Holbrook N J, Trush M A. Phorbol ester-induced mononuclear cell differentiation is blocked by the mitogen-activated protein kinase kinase (MEK) inhibitor PD98059. Cell Growth Differ 1999 May;10(5):307-15.

¹⁷⁹ Levine R A, Hopman T, Guo L, Chang M J, Johnson N. Induction of retinoblastoma gene expression during terminal growth arrest of a conditionally immortalized fetal rat lung epithelial cell line and during fetal lung maturation. Exp Cell Res. Mar. 15, 1998;239(2):264-76.

¹⁸⁰ Butel J S. Viral carcinogenesis: revelation of molecular mechanisms and etiology of human disease. Carcinogenesis. 2000 March;21(3):405-26.

¹⁸¹ zur Hausen H. Viruses in human cancers. Eur J Cancer. 1999 August;35(8):1174-81.

¹⁸² Hoppe-Seyler F, Butz K. Human tumor viruses. Anticancer Res. 1999 November-December; 19(6A):4747-58.

¹⁸³ Bonnet M, Guinebretiere J M, Kremm E, Grunewald V, Benhamou E, Contesso G, Joab I. Detection of Epstein-Barr virus in invasive breast cancers. J Natl Cancer Inst. Aug. 18, 1999;91(16): 1376-81.

¹⁸⁴ Labrecque L G, Barnes D M, Fentiman I S, Griffin B E. Epstein-Barr virus in epithelial cell tumors: a breast cancer study. Cancer Res. Jan. 1, 1995;55(1):39-45.

¹⁸⁵ Magrath I, Bhatia K. Breast cancer: a new Epstein-Barr virus-associated disease? J Natl Cancer Inst. Aug. 18, 1999;91(16):1349-50.

¹⁸⁶ Rakowicz-Szulczynska E M, Jackson B, Szulczynska A M, Smith M. Human immunodeficiency virus type 1-like DNA sequences and immunoreactive viral particles with unique association with breast cancer. Clin Diagn Lab Immunol. 1998 September;5(5):645-53.

¹⁸⁷ Sanserson C M, Smith G L. Cell motility and cell morphology: how some viruses take control. May 4, 1999, http://www-ermm.cbcu.cam.ac.uk/99000629h.htm

¹⁸⁸ Dore M, Korthuis R J, Granger D N, Entman M L, Smith C W. P-selectin mediates spontaneous leukocyte rolling in vivo. Blood Aug. 15, 1993;82(4):1308-16.

¹⁸⁹ Shang X Z, Issekutz A C. Contribution of CD11a/CD18, CD11b/CD18, ICAM-1 (CD54) and -2 (CD102) to human monocyte migration through endothelium and connective tissue fibroblast barriers. Eur J Immunol. 1998 June;28(6):1970-9.

¹⁹⁰ Shang X Z, Lang B J, Issekutz A C. Adhesion molecule mechanisms mediating monocyte migration through synovial fibroblast and endothelium barriers: role for CD11/CD18, very late antigen-4 (CD49d/CD29), very late antigen-5 (CD49e/CD29), and vascular cell adhesion molecule-1 (CD106). J Immunol. Jan. 1, 1998;160(1):467-74.

¹⁹¹ Meerschaert J, Furie M B. The adhesion molecules used by monocytes for migration across endothelium include CD11a/CD18, CD11b/CD18, and VLA-4 on monocytes and ICAM-1, VCAM-1, and other ligands on endothelium. J Immunol. Apr. 15, 1995;154(8):4099-112.

¹⁹² Meerschaert J, Furie M B. Monocytes use either CD11/CD18 or VLA-4 to migrate across human endothelium in vitro. J Immunol. Feb. 15, 1994; 152(4):1915-26.

¹⁹³ Chuluyan H E, Issekutz A C. VLA-4 integrin can mediate CD11/CD18-independent transendothelial migration of human monocytes. J Clin Invest. 1993 December;92(6):2768-77.

¹⁹⁴ Kavanaugh A F, Lightfoot E, Lipsky P E, Oppenheimer-Marks N. Role of CD11/CD18 in adhesion and transendothelial migration of T cells. Analysis utilizing CD18-deficient T cell clones. J Immunol. Jun. 15, 1991;146(12):4149-56.

¹⁹⁵ Wu C, Fields A J, Kapteijn B A, McDonald J A. The role of alpha 4 beta 1 integrin in cell motility and fibronectin matrix assembly. J Cell Sci. 1995 February;108 (Pt 2):821-9.

¹⁹⁶ Saltzman W M, Livingston T L, Parkhurst M R. Antibodies to CD18 influence neutrophil migration through extracellular matri. XJ Leukoc Biol. 1999 March;65(3):356-63.

¹⁹⁷Fernandez-Segura E, Garcia J M, Campos A. Topographic distribution of CD18 integrin on human neutrophils as related to shape changes and movement induced by chemotactic peptide and phorbol esters. Cell Immunol. Jul. 10, 1996; 171(1):120-5.

¹⁹⁸ Werr J, Xie X, Hedqvist P, Ruoslahti E, Lindbom L. betal integrins are critically involved in neutrophil locomotion in extravascular tissue In vivo. J Exp Med. Jun. 15, 1998;187(12):2091-6.

¹⁹⁹ Wu C, Fields A J, Kapteijn B A, McDonald J A. The role of alpha 4 beta 1 integrin in cell motility and fibronectin matrix assembly. J Cell Sci. 1995 February; 108 (Pt 2):821-9.

²⁰⁰ Saltzman W M, Livingston T L, Parkhurst M R. Antibodies to CD18 influence neutrophil migration through extracellular matri. XJ Leukoc Biol. 1999 March;65(3):356-63.

²⁰¹ Muller M, Albrecht S, Golfert F, Hofer A, Funk R H, Magdolen V, Flossel C, Luther T. Localization of tissue factor in actin-filament-rich membrane areas of epithelial cells. Exp Cell Res. Apr. 10, 1999;248(1): 136-47.

²⁰² Ott I, Fischer E G, Miyagi Y, Mueller B M, Ruf W. A role for tissue factor in cell adhesion and migration mediated by interaction with actin-binding protein 280. J Cell Biol. Mar. 9, 1998;140(5):1241-53.

²⁰³ Muller M, Albrecht S, Golfert F, Hofer A, Funk R H, Magdolen V, Flossel C, Luther T. Localization of tissue factor in actin-filament-rich membrane areas of epithelial cells. Exp Cell Res. Apr. 10, 1999;248(1):136-47.

²⁰⁴ Cunningham C C, Gorlin J B, Kwiatkowski D J, Hartwig J H, Janmey P A, Byers H R, Stossel T P. Actin-binding protein requirement for cortical stability and efficient locomotion. Science. Jan. 17, 1992;255(5042):325-7.

²⁰⁵ Ott I, Fischer E G, Miyagi Y, Mueller B M, Ruf W. A role for tissue factor in cell adhesion and migration mediated by interaction with actin-binding protein 280. J Cell Biol. Mar. 9, 1998; 140(5):1241-53.

²⁰⁶ Randolph G J, Luther T, Albrecht S, Magdolen V, Muller W A. Role of tissue factor in adhesion of mononuclear phagocytes to and trafficking through endothelium in vitro. Blood. Dec. 1, 1998;92(11):4167-77.

²⁰⁷ Randolph G J, Luther T, Albrecht S, Magdolen V, Muller W A. Role of tissue factor in adhesion of mononuclear phagocytes to and trafficking through endothelium in vitro. Blood. Dec. 1, 1998;92(11):4167-77.

²⁰⁸ Ott I, Fischer E G, Miyagi Y, Mueller B M, Ruf W. A role for tissue factor in cell adhesion and migration mediated by interaction with actin-binding protein 280. J Cell Biol. Mar. 9, 1998;140(5):1241-53.

²⁰⁹ Ott I, Fischer E G, Miyagi Y, Mueller B M, Ruf W. A role for tissue factor in cell adhesion and migration mediated by interaction with actin-binding protein 280. J Cell Biol. Mar. 9, 1998;140(5):1241-53.

²¹⁰ Chang L C, Wang J P. Examination of the signal transduction pathways leading to activation of extracellular signal-regulated kinase by formyl-methionyl-leucyl-phenylalanine in rat neutrophils. FEBS Lett. Jul. 2, 1999;454(1-2):165-8.

²¹¹ Yagisawa M, Saeki K, Okuma E, Kitamura T, Kitagawa S, Hirai H, Yazaki Y, Takaku F, Yuo A. Signal transduction pathways in normal human monocytes stimulated by cytokines and mediators: comparative study with normal human neutrophils or transformed cells and the putative roles in functionality and cell biology. Exp Hematol. 1999 June;27(6):1063-76.

²¹² Coffer P J, Geijsen N, M'rabet L, Schweizer R C, Maikoe T, Raaijmakers J A, Lammers J W, Koenderman L. Comparison of the roles of mitogen-activated protein kinase kinase and phosphatidylinositol 3-kinase signal transduction in neutrophil effector function. Biochem J Jan. 1, 1998;329 (Pt 1): 121-30.

²¹³ Yamada T, Fan J, Shimokama T, Tokunaga O, Watanabe T. Induction of fatty streak-like lesions in vitro using a culture model system simulating arterial intima. Am J Pathol. 1992 December; 141(6): 1435-44.

²¹⁴Kusuhara M, Chait A, Cader A, Berk B C. Oxidized LDL stimulates mitogen-activated protein kinases in smooth muscle cells and macrophages. Arterioscler Thromb Vasc Biol. 1997 January;17(1):141-8.

²¹⁵ Deigner H P, Claus R. Stimulation of mitogen activated protein kinase by LDL and oxLDL in human U-937 macrophage-like cells. FEBS Lett. May 6, 1996;385(3):149-53.

²¹⁶Balagopalakrishna C, Bhunia A K, Rifkind J M, Chatterjee S. Minimally modified low density lipoproteins induce aortic smooth muscle cell proliferation via the activation of mitogen activated protein kinase. Mol Cell Biochem. 1997 May;170(1-2):85-9.

²¹⁷ Kamanna V S, Bassa B V, Vaziri N D, Roh D D. Atherogenic lipoproteins and tyrosine kinase mitogenic signaling in mesangial cells. Kidney Int Suppl 1999 July;71 :S70-5.

²¹⁸ Bassa B V, Roh D D, Kirschenbaum M A, Kamanna VSAtherogenic lipoproteins stimulate mesangial cell p42 mitogen-activated protein kinase. J Am Soc Nephrol. 1998 March;9(3):488-96.

²¹⁹ Quinn M T, Parthasarathy S, Fong L G, Steinberg D. Oxidatively modified low density lipoproteins: a potential role in recruitment and retention of monocyte/macrophages during atherogenesis. Proc Natl Acad Sci U S A. 1987 May;84(9):2995-8.

²²⁰ Crutchley D J, Que B G. Copper-induced tissue factor expression in human monocytic THP-1 cells and its inhibition by antioxidants. Circulation. Jul. 15, 1995;92(2):238-43.

²²¹ Caspar-Bauguil S, Tkaczuk J, Haure M J, Durand M, Alcouffe J, Thomsen M, Salvayre R, Benoist H. Mildly oxidized low-density lipoproteins decrease early production of interleukin 2 and nuclear factor kappaB binding to DNA in activated T-lymphocytes. Biochem J. Jan. 15, 1999;337 (Pt 2):269-74.

²²² Matsumura T, Sakai M, Matsuda K, Furukawa N, Kaneko K, Shichiri M. Cis-acting DNA elements of mouse granulocyte/macrophage colony-stimulating factor gene responsive to oxidized low density lipoprotein. J Biol Chem. Dec. 31, 1999;274(53):37665-72.

²²³ Hamilton T A, Major J A, Armstrong D, Tebo J M. Oxidized LDL modulates activation of NFkappaB in mononuclear phagocytes by altering the degradation if IkappaBs. J Leukoc Biol. 1998 November;64(5):667-74.

²²⁴ Schackelford R E, Misra U K, Florine-Casteel K, Thai S F, Pizzo S V, Adams D O. Oxidized low density lipoprotein suppresses activation of NF kappa B in macrophages via a pertussis toxin-sensitive signaling mechanism. J Biol Chem. Feb. 24, 1995;270(8):3475-8.

²²⁵ Ohlsson B G, Englund M C, Karlsson A L, Knutsen E, Erixon C, Skribeck H, Liu Y, ondjers G, Wiklund O. Oxidized low density lipoprotein inhibits lipopolysaccharide-induced binding of nuclear factor-kappaB to DNA and the subsequent expression of tumor necrosis factor-alpha and interleukin-1beta in macrophages. J Clin Invest Jul. 1, 1996;98(1):78-89.

²²⁶ Yan S D, Schmidt A M, Anderson G M, Zhang J, Brett J, Zou Y S, Pinsky D, Stem D. Enhanced cellular oxidant stress by the interaction of advanced glycation end products with their receptors/binding proteins. J Biol Chem. Apr. 1, 1994;269(13):9889-97.

²²⁷ Khechai F, Ollivier V, Bridey F, Amar M, Hakim J, de Prost D. Effect of advanced glycation end product-modified albumin on tissue factor expression by monocytes. Role of oxidant stress and protein tyrosine Icinase activation. Arterioscler Thromb Vasc Biol. 1997 November; 17(11):2885-90.

²²⁸ Crutchley D J, Que B G. Copper-induced tissue factor expression in human monocytic THP-1 cells and its inhibition by antioxidants. Circulation. Jul. 15, 1995;92(2):238-43.

²²⁹ Brisseau G F, Dackiw A P, Cheung P Y, Christie N, Rotstein O D. Posttranscriptional regulation of macrophage tissue factor expression by antioxidants. Blood Feb. 15, 1995;85(4):1025-35.

²³⁰ Ichikawa K, Yoshinari M, Iwase M, Wakisaka M, Doi Y, Iino K, Yamamoto M, Fujishima M. Advanced glycosylation end products induced tissue factor expression in human monocyte-like U937 cells and increased tissue factor expression in monocytes from diabetic patients. Atherosclerosis. 1998 February;136(2):281-7.

²³¹ Carson S D, Pirruccello S J. Immunofluorescent studies of tissue factor on U87MG cells: evidence for non-uniform distribution. Blood Coagul Fibrinolysis. 1993 December;4(6):911-20.

²³² Lewis J C, Bennett-Cain A L, DeMars C S, Doellgast G J, Grant K W, Jones N L, Gupta M. Procoagulant activity after exposure of monocyte-derived muacrophages to minimally oxidized low density lipoprotein. Co-localization of tissue factor antigen and nascent fibrin fibers at the cell surface. Am J Pathol. 1995 October;147(4):1029-40.

²³³ Lewis J C, Bennett-Cain A L, DeMars C S, Doellgast G J, Grant K W, Jones N L, Gupta M. Procoagulant activity after exposure of monocyte-derived macrophages to minimally oxidized low density lipoprotein. Co-localization of tissue factor antigen and nascent fibrin fibers at the cell surface. Am J Pathol. 1995 October;147(4):1029-40.

²³⁴ Cui M Z, Penn M S, Chisolm G M. Native and oxidized low density lipoprotein induction of tissue factor gene expression in smooth muscle cells is mediated by both Egr-1 and Sp1. J Biol Chem. Nov. 12, 1999;274(46):32795-802.

²³⁵ Fei H, Berliner J A, Parhami F, Drake T A. Regulation of endothelial cell tissue factor expression by minimally oxidized LDL and lipopolysaccharide. Arterioscler Thromb. 1993 November; 13(11): 1711-7.

²³⁶ Li D, Mehta J L. Upregulation of endothelial receptor for oxidized LDL (LOX-1) by oxidized LDL and implications in apoptosis of human coronary artery endothelial cells: evidence from use of antisense LOX-1 mRNA and chemical inhibitors. Arterioscler Thromb Vasc Biol. 2000 April;20(4): 1116-22.

²³⁷ Maziere C, Auclair M, Djavaheri-Mergny M, Packer L, Maziere J C. Oxidized low density lipoprotein induces activation of the transcription factor NF kappa B in fibroblasts, endothelial and smooth muscle cells. Biochem Mol Biol Int. 1996 August;39(6): 1201-7.

²³⁸ Montoya M C, Luscinskas F W, del Pozo M A, Aragones J, de Landazuri M O. Reduced intracellular oxidative metabolism promotes firm adhesion of human polymorphonuclear leukocytes to vascular endothelium under flow conditions. Eur J Immunol. 1997 August;27(8):1942-51.

²³⁹ Martin M E, Chinenov Y, Yu M, Schmidt T K, Yang X Y. Redox regulation of GA-binding protein-alpha DNA binding activity. J Biol Chem. Oct. 11, 1996;271(41):25617-23.

²⁴⁰ Rosmarin A G, Luo M, Caprio D G, Shang J, Simkevich C P. Sp1 cooperates with the ets transcription factor, GABP, to activate the CD18 (beta2 leukocyte integrin) promoter. J Biol Chem May 22, 1998;273(21):13097-103.

²⁴¹ Therond P, Abella A, Laurent D, Couturier M, Chalas J, Legrand A, Lindenbaum A. In vitro study of the cytotoxicity of isolated oxidized lipid low-density lipoproteins fractions in human endothelial cells: relationship with the glutathione status and cell morphology. Free Radic Biol Med. Feb. 15, 2000;28(4):585-96.

²⁴² Lizard G, Gueldry S, Sordet O, Monier S, Athias A, Miguet C, Bessede G, Lemaire S, Solary E, Gambert P. Glutathione is implied in the control of 7-ketocholesterol-induced apoptosis, which is associated with radical oxygen species production. FASEB J. 1998 December;12(15): 1651-63.

²⁴³ Gray J L, Shankar R. Down regulation of CD11b and CD18 expression in atherosclerotic lesion-derived macrophages. Am Surg 1995 August;61(8):674-9; discussion 679-80.

²⁴⁴ Trach C C, Wulfroth P M, Severs N J, Robenek H. Influence of native and modified lipoproteins on migration of mouse peritoneal macrophages and the effect of the antioxidants vitamin E and Probucol. Eur J Cell Biol. 1996 October;71(2):199-205.

²⁴⁵ Pataki M, Lusztig G, Robenek H. Endocytosis of oxidized LDL and reversibility of migration inhibition in macrophage-derived foam cells in vitro. A mechanism for atherosclerosis regression? Arterioscler Thromb. 1992 August;12(8):936-44.

²⁴⁶ Quinn M T, Parthasarathy S. Steinberg D. Endothelial cell-derived chemotactic activity for mouse peritoneal macrophages and the effects of modified forms of low density lipoprotein. Proc Natl Acad Sci U S A. 1985 September;82(17):5949-53.

²⁴⁷ Yamada T, Fan J, Shimokama T, Tokunaga O, Watanabe T. Induction of fatty streak-like lesions in vitro using a culture model system simulating arterial intima. Am J Pathol. 1992 December;141(6):1435-44.

²⁴⁸ Virmani R, Kolodgie F D, Burke A P, Farb A, Schwartz S M. Lessons from sudden coronary death: a comprehensive morphological classification scheme for atherosclerotic lesions. Arterioscler Thromb Vasc Biol. 2000 May;20(5): 1262-75.

²⁴⁹ Pentikainen M O, Oomi K, Ala-Korpela M, Kovanen P T. Modified LDL—trigger of atherosclerosis and inflammaation in the arterial intima. J Intern Med. 2000 March;247(3):359-70.

²⁵⁰ Nordestgaard B G, Hjelms E, Stender S, Kjeldsen K. Different efflux pathways for high and low density lipoproteins from porcine aortic intima. Arteriosclerosis. 1990 May-June;10(3):477-85.

²⁵¹ Boren J, Olin K, Lee I, Chait A, Wight T N, Innerarity T L. Identification of the principal proteoglycan-binding site in LDL. A single-point mutation in apo-B 100 severely affects proteoglycan interaction without affecting LDL receptor binding. J Clin Invest. Jun. 15, 1998;101(12):2658-64.

²⁵²Pentikainen M O, Oomi K, Ala-Korpela M, Kovanen P T. Modified LDL—trigger of atherosclerosis and inflammation in the arterial intima. J Intern Med. 2000 March;247(3):359-70.

²⁵³ Nordestgaard B G, Tybjaerg-Hansen A, Lewis B. Influx in vivo of low density, intermediate density, and very low density lipoproteins into aortic intimas of genetically hyperlipidemic rabbits. Roles of plasma concentrations, extent of aortic lesion, and lipoprotein particle size as determinants. Arterioscler Thromb. 1992 January;12(1):6-18.

²⁵⁴ Schwenke D C. Comparison of aorta and pulmonary artery: II. LDL transport and metabolism correlate with susceptibility to atherosclerosis. Circ Res. 1997 September;81(3):346-54.

²⁵⁵ Kao C H, Chen J K, Yang V C. Ultrastructure and permeability of endothelial cells in branched regions of rat arteries. Atherosclerosis. 1994 January;105(1):97-114.

²⁵⁶ Kao C H, Chen J K, Kuo J S, Yang V C. Visualization of the transport pathways of low density lipoproteins across the endothelial cells in the branched regions of rat arteries. Atherosclerosis. 1995 July;l 16(1):27-41.

²⁵⁷ Nordestgaard B G, Wootton R, Lewis B. Selective retention of VLDL, IDL, and LDL in the arterial intima of genetically hyperlipidemic rabbits in vivo. Molecular size as a determinant of fractional loss from the intima-inner media. Arterioscler Thromb Vasc Biol 1995 April; 15(4):534-42.

²⁵⁸ Gerrity R G. The role of the monocyte in atherogenesis: II. Migration of foam cells from atherosclerotic lesions. Am J Pathol. 1981 May; 103(2):191-200.

²⁵⁹ Faggiotto A, Ross R, Harker L. Studies of hypercholesterolemia in the nonhuman primate. I. Changes that lead to fatty streak formation. Arteriosclerosis. 1984 July-August;4(4):323-40.

²⁶⁰ Faggiotto A, Ross R. Studies of hypercholesterolemia in the nonhuman primate. II. Fatty streak conversion to fibrous plaque. Arteriosclerosis. 1984 July-August;4(4):341-56.

²⁶¹ Kling D, Holzschuh T, Betz E. Recruitment and dynamics of leukocytes in the formation of arterial intimal thickening—a comparative study with normo- and hypercholesterolemic rabbits. Atherosclerosis. 1993 June;101(1):79-96.

²⁶² Landers S C, Gupta M, Lewis J C. Ultrastructural localization of tissue factor on monocyte-derived macrophages and macrophage foam cells associated with atherosclerotic lesions. Virchows Arch. 1994;425(1):49-54.

²⁶³ Faggiotto A, Ross R, Harker L. Studies of hypercholesterolemia in the nonhuman primate. I. Changes that lead to fatty streak formation. Arteriosclerosis. 1984 July-August;4(4):323-40.

²⁶⁴ Malek A M, Alper S L, Izumo S. Hemodynamic shear stress and its role in atherosclerosis. JAMA. Dec. 1, 1999;282(21):2035-42.

²⁶⁵ Trach C C, Wulfroth P M, Severs N J, Robenek H. Influence of native and modified lipoproteins on migration of mouse peritoneal macrophages and the effect of the antioxidants vitamin E and Probucol. Eur J Cell Biol. 1996 October;71(2): 199-205.

²⁶⁶ Pataki M, Lusztig G, Robenek H. Endocytosis of oxidized LDL and reversibility of migration inhibition in macrophage-derived foam cells in vitro. A mechanism for atherosclerosis regression? Arterioscler Thromb. 1992 August;12(8):936-44.

²⁶⁷ Wissler R W, Vesselinovitch D. Can atherosclerotic plaques regress? Anatomic and biochemical evidence from nonhuman animal models. Am J Cardiol. Mar. 20, 1990;65(12):33F-40F.

²⁶⁸ Dudrick S J. Regression of atherosclerosis by the intravenous infusion of specific biochemical nutrient substrates in animals and humans. Ann Surg. 1987 September;206(3):296-315.

²⁶⁹ Tucker C F, Catsulis C, Strong J P, Eggen D A. Regression of early cholesterol-induced aortic lesions in rhesus monkeys. Am J Pathol. 1971 December;65(3):493-514.

²⁷⁰ Collins R G, Velji R, Guevara NV, Hicks M J, Chan L, Beaudet A L. P-Selectin or intercellular adhesion molecule (ICAM)-1 deficiency substantially protects against atherosclerosis in apolipoprotein E-deficient mice. J Exp Med. 2000 Jan 3; 191(1):189-94.

²⁷¹ Dong Z M, Chapman S M, Brown A A, Frenette P S, Hynes R O, Wagner D D. The combined role of P- and E-selectins in atherosclerosis. J Clin Invest. Jul. 1, 1998;102(1):145-52.

²⁷² Patel S S, Thiagarajan R, Willerson J T, Yeh E T. Inhibition of alpha4 integrin and ICAM-1 markedly attenuate macrophage homing to atherosclerotic plaques in ApoE-deficient mice. Circulation. Jan 6-13, 1998;97(1):75-81.

²⁷³ Pentikainen M O, Oorni K, Ala-Korpela M, Kovanen P T. Modified LDL—trigger of atherosclerosis and inflammation in the arterial intima. J Intem Med. 2000 March;247(3):359-70.

²⁷⁴ Stary H C, Chandler A B, Glagov S, Guyton J R, Insull Jr W, Rosenfeld M E, Schaffer S A, Schwartz C J, Wagner W D, Wissler R W. A Definition of Initial, Fatty Streak, and Intermediate Lesions of Atherosclerosis. A Report From the Commnittee on Vascular Lesions of the Council on Arteriosclerosis, American Heart Association. AHA Medical/Scientific Statement. 1994. http://www.americanheart.org/Scientific/statements/1994/059401.html

²⁷⁵ Taylor-Wiedeman J, Sissons P, Sinclair J. Induction of endogenous human cytomegalovirus gene expression after differentiation of onocytes from healthy carriers. J Virol. 1994 March;68(3):1597-604.

²⁷⁶ Guetta E, Guetta V, Shibutani T, Epstein S E. Monocytes harboring cytomegalovirus: interactions with endothelial cells, smooth muscle cells, and oxidized low-density lipoprotein. Possible mechanisms for activating virus delivered by monocytes to sites of vascular injury. Circ Res 1997 July;81(1):8-16.

²⁷⁷ Ikuta K, Luftig R B. Inhibition of cleavage of Moloney murine leukemia virus gag and env coded precursor polyproteins by cerulenin. Virology. Oct. 15, 1986;154(1):195-206.

²⁷⁸ Katoh I, Yoshinaka Y, Luftig R B. The effect of cerulenin on Moloney murine leukemia virus morphogenesis. Virus Res. 1986 August;5(2-3):265-76.

²⁷⁹ Goldfine H, Harley J B, Wyke J A. Effects of inhibitors of lipid synthesis on the replication of Rous sarcoma virus. A specific effect of cerulenin on the processing of major non-glycosylated viral structural proteins. Biochim Biophys Acta Sep. 22, 1978;512(2):229-40.

²⁸⁰ Taylor-Wiedeman J, Sissons P, Sinclair J. Induction of endogenous human cytomegalovirus gene expression after differentiation of onocytes from healthy carriers. J Virol. 1994 March;68(3):1597-604.

²⁸¹ Ibanez C E, Schrier R, Ghazal P, Wiley C, Nelson J A. Human cytomegalovirus productively infects primary differentiated macrophages. J Virol. 1991 December;65(12):6581-8.

²⁸² Lathey J L, Spector S A. Unrestricted replication of human cytomegalovirus in hydrocortisone-treated macrophages. J Virol. 1991 November;65(11):6371-5.

²⁸³ Weinshenker B G, Wilton S, Rice G P. Phorbol ester-induced differentiation permits productive human cytomegalovirus infection in a monocytic cell line. J Immunol. Mar. 1, 1988;140(5):1625-31.

²⁸⁴ Gonczol E, Andrews P W, Plotkin S A. Cytomegalovirus replicates in differentiated but not in undifferentiated human embryonal carcinoma cells. Science. Apr. 13, 1984;224(4645):159-61.

²⁸⁵ Guyton J R. The role of lipoproteins in atherogenesis. Adv Exp Med Biol. 1995;369:29-38.

²⁸⁶ Randolph G J, Furie M B. Mononuclear phagocytes egress from an in vitro model of the vascular wall by migrating across endothelium in the basal to apical direction: role of intercellular adhesion molecule 1 and the CD11/CD18 integrins. J Exp Med. Feb. 1, 1996;183(2):451-62.

²⁸⁷ Randolph G J, Luther T, Albrecht S, Magdolen V, Muller W A. Role of tissue factor in adhesion of mononuclear phagocytes to and trafficking through endothelium in vitro. Blood. Dec. 1, 1998;92(11):4167-77.

²⁸⁸ Key N S, Bach R R, Vercellotti G M, Moldow C F. Herpes simplex virus type I does not require productive infection to induce tissue factor in human umbilical vein endothelial cells. Lab Invest. 1993 June;68(6):645-51.

²⁸⁹ Schecter A D, Giesen P L, Taby O, Rosenfield C L, Rossikhina M, Fyfe B S, Kohtz D S, Fallon J T, Nemerson Y, Taubman M B. Tissue factor expression in human arterial smooth muscle cells. TF is present in three cellular pools after growth factor stimulation. J Clin Invest. Nov. 1, 1997; 100(9):2276-85.

²⁹⁰ Lewis J C, Bennett-Cain A L, DeMars C S, Doellgast G J, Grant K W, Jones N L, Gupta M. Procoagulant activity after exposure of monocyte-derived macrophages to minimally oxidized low density lipoprotein. Co-localization of tissue factor antigen and nascent fibrin fibers at the cell surface. Am J Pathol. 1995 October;147(4):1029-40.

²⁹¹ Randolph G J, Furie M B. Mononuclear phagocytes egress from an in vitro model of the vascular wall by migrating across endothelium in the basal to apical direction: role of intercellular adhesion molecule 1 and the CD11/CD18 integrins. J Exp Med. Feb. 1, 1996;183(2):451-62.

²⁹² Virmani R, Kolodgie F D, Burke A P, Farb A, Schwartz S M. Lessons from sudden coronary death: a comprehensive morphological classification scheme for atherosclerotic lesions. Arterioscler Thromb Vasc Biol. 2000 May;20(5):1262-75.

²⁹³ Zhou Y F, Guetta E, Yu Z X, Finkel T, Epstein S E. Human cytomegalovirus increases modified low density lipoprotein uptake and scavenger receptor mRNA expression in vascular smooth muscle cells. J Clin Invest Nov. 1, 1996;98(9):2129-38.

²⁹⁴ Benditt E P, Barrett T, McDougall J K. Viruses in the etiology of atherosclerosis. Proc Natl Acad Sci U S A 1983 October;80(20):6386-9.

²⁹⁵ Wang H, Liu B, Fu M, Zeng C. [Transcriptional expression of oncogenes and Rb antioncogene in experimental atherosclerotic lesions]. Hua Hsi I Ko Ta Hsueh Hsueh Pao. 1996 June;27(2): 117-21. Chinese.

²⁹⁶ Claudio P P, Fratta L, Farina F, Howard C M, Stassi G, Numata S, Pacilio C, Davis A, Lavitrano M, Volpe M, Wilson J M, Trimarco B, Giordano A, Condorelli G. Adenoviral RB2/p130 gene transfer inhibits smooth muscle cell proliferation and prevents restenosis after angioplasty. Circ Res. Nov. 26, 1999;85(11):1032-9.

²⁹⁷ Schwartz L B, Moawad J, Svensson EC, Tufts R L, Meyerson S L, Baunoch D, Leiden J M. Adenoviral-mediated gene transfer of a constitutively active form of the retinoblastoma gene product attenuates neointimal thickening in experimental vein grafts. J Vasc Surg. 1999 May;29(5):874-81; discussion 882-3.

²⁹⁸ Smith R C, Wills K N, Antelman D, Perlman H, Truong L N, Krasinski K, Walsh K. Adenoviral constructs encoding phosphorylation-competent full-length and truncated forms of the human retinoblastoma protein inhibit myocyte proliferation and neointima formation. Circulation. Sep. 16, 1997;96(6): 1899-905.

²⁹⁹ Gallo R, Padurean A, Jayaraman T, Marx S, Roque M, Adeiman S, Chesebro J, Fallon J, Fuster V, Marks A, Badimon J J. Inhibition of intimal thickening after balloon angioplasty in porcine coronary arteries by targeting regulators of the cell cycle. Circulation. Apr. 27, 1999;99(16):2164-70.

³⁰⁰ Chiu B. Multiple infections in carotid atherosclerotic plaques. Am Heart J. 1999 November;138(5 Pt 2):534-536.

³⁰¹ Nieto F J. Viruses and atherosclerosis: A critical review of the epidemiologic evidence. Am Heart J 1999 November; 138(5 Pt 2):453-460.

³⁰² Adam E, Melnick J L, Probtsfield J L, Petrie B L, Burek J, Bailey K R, McCollum C H, DeBakey M E. High levels of cytomegalovirus antibody in patients requiring vascular surgery for atherosclerosis. Lancet. Aug. 8, 1987;2(8554):291-3.

³⁰³ Li B, Xu C, Wang Q. The detection of the antibodies of human cytomegalovirus in the sera of patients with coronary heart disease. Chung Hua Nei Ko Tsa Chih. 1996 November;35(11):741-3 (in Chinese).

³⁰⁴ Liuzzo G, Caligiuri G, Grillo R L, et al. Helicobacter pylori and cytomegalovirus infectoins are strongly associated with atherosclerosis, but are not responsible for the instability of angina [abstract]. J Am Coll Cardiol 1997;29(suppl A):217A.

³⁰⁵ Blum A, Giladi M, Weinberg M, Kaplan G, Pasternack H, Laniado S, Miller H. High anti-cytomegalovirus (CMV) IgG antibody titer is associated with coronary artery disease and may predict post-coronary balloon angioplasty restenosis. Am J Cardiol. Apr. 1, 1998;81(7):866-8.

³⁰⁶ Nieto F J. Viruses and atherosclerosis: A critical review of the epidemiologic evidence. Am Heart J 1999 November;138(5 Pt 2):453-460.

³⁰⁷ Sorlie P D, Nieto F J, Adam E, Folsom A R, Shahar E, Massing M. A prospective study of cytomegalovirus, herpes simplex virus 1, and coronary heart disease: the atherosclerosis risk in communities (ARIC) study. Arch Intem Med. Jul. 10, 2000;160(13):2027-32.

³⁰⁸ Fabricant C G, Fabricant J. Atherosclerosis induced by infection with Marek's disease herpesvirus in chickens. Am Heart J 1999 November; 138(5 Pt 2):S465-8.

³⁰⁹ Dodet B, Plotkin S A. Infection and atherosclerosis. Am Heart J 1999 November;138(5 Pt 2):417-418.

³¹⁰ Fong I W. Emerging relations between infectious diseases and coronary artery disease and atherosclerosis. CMAJ. Jul. 11, 2000;163(1):49-56.

³¹¹ zur Hausen H. Viruses in human cancers. Eur J Cancer. 1999 December;35(14):1878-85.

³¹² Crawford L. Criteria for establishing that a virus is oncogenic. Ciba Found Symp. 1986;120:104-16.

³¹³ Butel J S. Viral carcinogenesis: revelation of molecular mechanisms and etiology of human disease. Carcinogenesis. 2000 March;21(3):405-26.

³¹⁴ Sawada M, Miyake S, Ohdama S, Matsubara O, Masuda S, Yakumaru K, Yoshizawa Y. Expression of tissue factor in non-small-cell lung cancers and its relationship to metastasis. Br J Cancer. 1999 February;79(3-4):472-7.

³¹⁵ Shigemori C, Wada H, Matsumoto K, Shiku H, Nakamura S, Suzuki H. Tissue factor expression and metastatic potential of colorectal cancer. Thromb Haemost. 1998 December;80(6):894-8.

³¹⁶ Mueller B M, Reisfeld R A, Edgington T S, Ruf W. Expression of tissue factor by melanoma cells promotes efficient hematogenous metastasis. Proc Natl Acad Sci U S A. Dec. 15, 1992;89(24):11832-6.

³¹⁷ Adamson A S, Francis J L, Witherow R O, Snell M E. Urinary tissue factor levels in prostatic carcinoma: a potential marker of metastatic spread? Br J Urol. 1993 May;71(5):587-92.

³¹⁸ Kataoka H, Uchino H, Asada Y, Hatakeyama K, Nabeshima K, Sumiyoshi A, Koono M. Analysis of tissue factor and tissue factor pathway inhibitor expression in human colorectal carcinoma cell lines and metastatic sublines to the liver. Int J Cancer. Sep. 4, 1997;72(5):878-84.

³¹⁹ Sturm U, Luther T, Albrecht S, Flossel C, Grossmann H, Muller M. Immunohistological detection of tissue factor in normal and abnormal human mammary glands using monoclonal antibodies. Virchows Arch A Pathol Anat Histopathol. 1992;421(2):79-86.

³²⁰ Hu T, Bach R R, Horton R, Konigsberg W H, Todd M B. Procoagulant activity in cancer cells is dependent on tissue factor expression. Oncol Res. 1994;6(7):321-7.

³²¹ Ruf W, Fischer E G, Huang H Y, Miyagi Y, Ott I, Riewald M, Mueller B M. Diverse functions of protease receptor tissue factor in inflammation and metastasis. Immunol Res. 2000;21(2-3):289-92.

³²² Schwartz J D, Simantov R. Thrombosis and malignancy: pathogenesis and prevention. In vivo. 1998 November-December;12(6):619-24.

³²³ Bromberg M E, Konigsberg W H, Madison J F, Pawashe A, Garen A. Tissue factor promotes melanoma metastasis by a pathway independent of blood coagulation. Proc Natl Acad Sci U S A. Aug. 29, 1995;92(18):8205-9.

³²⁴ Gebken J, Luders B, Notbohm H, Klein H H, Brinckmann J, Muller P K, Batge B. Hypergravity stimulates collagen synthesis in human osteoblast-like cells: evidence for the involvement of p44/42 MAP-kinases (ERK 1/2). J Biochem (Tokyo). 1999 October;126(4):676-82.

³²⁵Byers P H, Duvic M, Atkinson M, Robinow M, Smith L T, Krane S M, Greally M T, Ludman M, Matalon R, Pauker S, Quanbeck D, Schwarze U. Ehlers-Danlos syndrome type VIIA and VIIB result from splice-junction mutations or genomic deletions that involve exon 6 in the COL1A1 and COL1A2 genes of type I collagen. Am J Med Genet Oct. 3, 1997;72(1):94-105.

³²⁶ Giunta C, Superti-Furga A, Spranger S, Cole W G, Steinmann B. Ehlers-Danlos syndrome type VII: clinical features and molecular defects. J Bone Joint Surg Am 1999 February;81(2):225-38.

³²⁷ Grahame R. Joint hypermobility and genetic collagen disorders: are they related? Arch Dis Child, 1999, 80:188-191.

³²⁸ Batti'e M C, Bigos S J, Sheehy A, Wortley M D. Spinal flexibility and individual factos that influence it. Physical Therapy 1987 67(5):653-658.

329 Grahame R. How often, when and how does joint hypermobility lead to osteoarthritis? Br J Rheumatol 1989 August;28(4):320.

³³⁰ Scott D, Bird H, Wright V. Joint laxity leading to osteoarthrosis. Rheumatol Rehabil. 1979 August; 18(3): 167-9.

³³¹ Sharma L, Lou C, Felson D T, Dunlop D D, Kirwan-Mellis G, Hayes K W, Weinrach D, Buchanan T S. Laxity in healthy and osteoarthritic knees. Arthritis Rheum 1999 May;42(5):861-70.

³³² Jonsson H, Valtysdottir S T, Kjartansson O, Brekkan A. Hypermobility associated with osteoarthritis of the thumb base: a clinical and radiological subset of hand osteoarthritis. Ann Rheum Dis 1996 August;55(8):540-3.

³³³ Cicuttini F M., Baker J R., Spector T D. The Association of Obesity with Osteoarthritis of the Hand and Knee in Women: A Twin Study. The Journal of Rheumatology, 1996, 23:7, 1221-1226.

³³⁴ Carman W J, Sowers M, Hawthorne V M, Weissfeld L A. Obesity as a risk factor for osteoarthritis of the hand and wrist: a prospective study. Am J Epidemiol Jan. 15, 1994; 139(2):119-29.

³³⁵ Van Saase J L C M, Vandenbroucke M P, van Romunde L K J, Valkenburg H A. Osteoarthritis and Obesity in the General Population. A Relationship Calling for and Explanation. The Journal of Rheumatology, 1988, 15(7): 1152-1158.

³³⁶ Ferguson K A, Love L L, Ryan C F. Effect of mandibular and tongue protrusion on upper airway size during wakefulness. Am J Respir Crit Care Med 1997 May;155(5):1748-54.

³³⁷ Miyamoto K, Ozbek M M, Lowe A A, Sjoholm T T, Love L L, Fleetham J A, Ryan C F. Mandibular posture during sleep in patients with obstructive sleep apnoea. Arch Oral Biol 1999 August;44(8):657-64.

³³⁸ Ferguson K A, Love L L, Ryan C F. Effect of mandibular and tongue protrusion on upper airway size during wakefulness. Am J Respir Crit Care Med 1997 May;155(5):1748-54.

³³⁹ Mokdad A H, Serdula M K, Dietz W H, Bowman B A, Marks J S, Koplan J P. The spread of the obesity epidemic in the United States, 1991-1998. JAMA. Oct. 27, 1999;282(16): 1519-22.

³⁴⁰ Weinsier R L, Hunter G R, Heini A F, Goran M I, Sell S M. The etiology of obesity: relative contribution of metabolic factors, diet, and physical activity. Am J Med. 1998 August; 105(2):145-50.

³⁴¹ Heini A F, Weinsier R L. Divergent trends in obesity and fat intake patterns: the American parado. XAm J Med. 1997 March;102(3):259-64.

³⁴² Hebebrand J, Wulftange H, Goerg T, Ziegler A, Hinney A, Barth N, Mayer H, Remschmidt H. Epidemic obesity: are genetic factors involved via increased rates of assortative mating? Int J Obes Relat Metab Disord. 2000 March;24(3):345-53.

³⁴³ Hill J O, Peters J C. Environmental contributions to the obesity epidemic. Science. May 29, 1998;280(5368):1371-4.

³⁴⁴ Koplan J P, Dietz W H. Caloric imbalance and public health policy. JAMA. Oct. 27, 1999;282(16):1579-81.

³⁴⁵ Beattie J H, Wood A M, Newman A M, Bremner I, Choo K H A, Michalska A E, Duncan J S, Trayhurn P. Obesity and hyperleptinemia in metallothionein (-I and-II) null mice. Proc. Natl. Acad. Sci. USA 1998 95(1): 358-363.

³⁴⁶ Rosmarin A G, Caprio D G, Kirsch D G, Handa H, Simkevich C P. GABP and PU. 1 compete for binding, yet cooperate to increase CD18 (beta 2 leukocyte integrin) transcription. J Biol Chem Oct, 6, 1995;270(40):23627-33.

³⁴⁷ Rosmarin A G, Luo M, Caprio D G, Shang J, Simkevich C P. Sp1 cooperates with the ets transcription factor, GABP, to activate the CD18 (beta2 leukocyte integrin) promoter. J Biol Chem May 22, 1998;273(21): 13097-103.

³⁴⁸ Le Naour R, Lussiez C, Raoul H, Mabondzo A, Dormont D. Expression of cell adhesion molecules at the surface of in vitro human immunodeficiency virus type 1-infected human monocytes: relationships with tumor necrosis factor alpha, interleukin 1beta, and interleukin 6 syntheses. AIDS Res Hum Retroviruses Jul. 1, 1997;13(10):841-55.

³⁴⁹ Tanaka Y, Fukudome K, Hayashi M, Takagi S, Yoshie O. Induction of ICAM-1 and LFA-3 by Tax1 of human T-cell leukemia virus type 1 and mechanism of down-regulation of ICAM-1 or LFA-1 in adult-T-cell-leukemia cell lines. Int J Cancer Feb. 8, 1995;60(4):554-61.

³⁵⁰ Patarroyo M, Prieto J, Ernberg I, Gahmberg C G. Absence, or low expression, of leukocyte adhesion molecules CD11 and CD18 on Burkitt lymphoma cells. Int J Cancer Jun. 15, 1988;41(6):901-7.

³⁵¹ Dong Z M, Gutierrez-Ramos J C, Coxon A, Mayadas T N, Wagner D D. A new class of obesity genes encodes leukocyte adhesion receptors. Proc. Natl. Acad. Sci. USA 1997 (94):7526-7530.

³⁵² Large V, Reynisdottir S, Langin D, Fredby K, Klannemark M, Holm C, Arner P. Decreased expression and function of adipocyte hormone-sensitive lipase in subcutaneous fat cells of obese subjects. J Lipid Res. 1999 November;40(11):2059-66.

³⁵³ Elizalde M, Ryden M, van Harmelen V, Eneroth P, Gyllenhanmmar H, Holm C, Ramel S, Olund A, Amer P, Andersson K. Expression of nitric oxide synthases in subcutaneous adipose tissue of nonobese and obese humans. J Lipid Res. 2000 August;41(8):1244-51.

³⁵⁴ Maudsley S, Pierce K L, Zamah A M, Miller W E, Ahn S, Daaka Y, Lefkowitz R J, Luttrell L M. The beta(2)-adrenergic receptor mediates extracellular signal-regulated kinase activation via assembly of a multi-receptor complex with the epidermal growth factor receptor. J Biol Chem. Mar. 31, 2000;275(13):9572-80.

³⁵⁵ Pierce K L, Maudsley S, Daaka Y, Luttrell L M, Lefkowitz R J. Role of endocytosis in the activation of the extracellular signal-regulated kinase cascade by sequestering and nonsequestering G protein-coupled receptors. Proc Natl Acad Sci U S A. Feb. 15, 2000;97(4):1489-94.

³⁵⁶ Elorza A, Samago S, Mayor F Jr. Agonist-dependent modulation of G protein-coupled receptor kinase 2 by mitogen-activated protein kinases. Mol Pharmacol. 2000 April;57(4):778-83.

³⁵⁷ Luttrell L M, Ferguson S S, Daaka Y, Miller W E, Maudsley S, Della Rocca G J, Lin F, Kawakatsu H, Owada K, Luttrell D K, Caron M G, Lefkowitz R J. Beta-arrestin-dependent formation of beta2 adrenergic receptor-Src protein kinase complexes. Science. Jan. 29, 1999;283(5402):655-61.

³⁵⁸ Daaka Y, Luttrell L M, Ahn S, Della Rocca G J, Ferguson S S, Caron M G, Lefkowitz R J. Essential role for G protein-coupled receptor endocytosis in the activation of mitogen-activated protein kinase. J Biol Chem. Jan. 9, 1998;273(2):685-8.

³⁵⁹ Cao W, Luttrell L M, Medvedev A V, Pierce K L, Daniel K W, Dixon T M, Lefkowitz R J, Collins S. Direct Binding of Activated c-Src to the Beta3-Adrenergic Receptor is Required for MAP Kinase Activation. J Biol Chem. Sep. 29, 2000.

³⁶⁰ Gerhardt C C, Gros J, Strosberg A D, Issad T. Stimulation of the extracellular signal-regulated kinase 1/2 pathway by human beta-3 adrenergic receptor: new pharmacological profile and mechanism of activation. Mol Pharmacol. 1999 February;55(2):255-62.

³⁶¹ Soeder K J, Snedden S K, Cao W, Della Rocca G J, Daniel K W, Luttrell L M, Collins S. The beta3-adrenergic receptor activates mitogen-activated protein kinase in adipocytes through a Gi-dependent mechanism. J Biol Chem. Apr. 23, 1999;274(17): 12017-22.

³⁶² Hellstrom L, Langin D, Reynisdottir S, Dauzats M, Amer P. Adipocyte lipolysis in normal weight subjects with obesity among first-degree relatives. Diabetologia. 1996 August;39(8):921-8.

³⁶³ Shimizu Y, Tanishita T, Minokoshi Y, Shimazu T. Activation of mitogen-activated protein kinase by norepinephrine in brown adipocytes from rats. Endocrinology. 1997 January;138(1):248-53.

³⁶⁴ Yarwood S J, Kilgour E, Anderson N G. Cyclic AMP stimulates the phosphorylation and activation of p42 and p44 muitogen-activated protein kinases in 3T3-F442A preadipocytes. Biochem Biophys Res Commun. Jul. 25, 1996;224(3):734-9.

³⁶⁵ Gerhardt C C, Gros J, Strosberg A D, Issad T. Stimulation of the extracellular signal-regulated kinase 1/2 pathway by human beta-3 adrenergic receptor: new pharmacological profile and mechanism of activation. Mol Pharmacol. 1999 February;55(2):255-62.

³⁶⁶ Large V, Reynisdottir S, Langin D, Fredby K, Klannemark M, Holm C, Arner P. Decreased expression and function of adipocyte hormone-sensitive lipase in subcutaneous fat cells of obese subjects. J Lipid Res. 1999 November;40(11):2059-66.

³⁶⁷ Hellstrom L, Reynisdottir S. Influence of heredity for obesity on adipocyte lipolysis in lean and obese subjects. Int J Obes Relat Metab Disord. 2000 March;24(3):340-4.

³⁶⁸ Bougneres P, Stunff C L, Pecqueur C, Pinglier E, Adnot P, Ricquier D. In vivo resistance of lipolysis to epinephrine. A new feature of childhood onset obesity. J Clin Invest. Jun. 1, 1997;99(11):2568-73.

³⁶⁹ Horowitz J F, Klein S. Whole body and abdominal lipolytic sensitivity to epinephrine is suppressed in upper body obese women. Am J Physiol Endocrinol Metab. 2000 June;278(6):E1144-52.

³⁷⁰ Osuga J, Ishibashi S, Oka T, Yagyu H, Tozawa R, Fujimoto A, Shionoiri F, Yahagi N, Kraemer F B, Tsutsumi O, Yamada N. Targeted disruption of hormone-sensitive lipase results in male sterility and adipocyte hypertrophy, but not in obesity. Proc Natl Acad Sci U S A. Jan. 18, 2000;97(2):787-92.

³⁷¹ Classon M, Kennedy B K, Mulloy R, Harlow E. Opposing roles of pRB and p107 in adipocyte differentiation. Proc Natl Acad Sci U S A. Sep. 26, 2000;97(20):10826-31.

³⁷² Puigserver P, Ribot J, Serra F, Gianotti M, Bonet M L, Nadal-Ginard B, Palou A. Involvement of the retinoblastoma protein in brown and white adipocyte cell differentiation: functional and physical association with the adipogenic transcription factor C/EBPalpha. Eur J Cell Biol. 1998 October;77(2):117-23.

³⁷³ Richon V M, Rifkind R A, Marks P A. Expression and phosphorylation of the retinoblastoma protein during induced differentiation of murine erythroleukemia cells. Cell Growth Differ. 1992 July;3(7):413-20.

³⁷⁴ Roncari D A, Kindler S, Hollenberg C H. Excessive proliferation in culture of reverted adipocytes from massively obese persons. Metabolism. 1986 January;35(1):1-4.

³⁷⁵ Roncari D A, Lau D C, Kindler S. Exaggerated replication in culture of adipocyte precursors from massively obese persons. Metabolism. 1981 May;30(5):425-7.

³⁷⁶ Stock S, Granstrom L, Backman L, Matthiesen A S, Uvnas-Moberg K. Elevated plasma levels of oxytocin in obese subjects before and after gastric banding. Int J Obes. 1989;13(2):213-22.

³⁷⁷ Johnson S R, Kolberg B H, Vamer M W, Railsback L D. Maternal obesity and pregnancy. Surg Gynecol Obstet. 1987 May;164(5):431-7.

³⁷⁸ Yakinci C, Pac A, Kucukbay F Z, Tayfun M, Gul A. Serum zinc, copper, and magnesium levels in obese children. Acta Paediatr Jpn. 1997 June;39(3):339-41.

³⁷⁹ D'Ocon C, Alonso de Armino V, Frasquet I. Levels of Zn and Cu in the serum of a diabetic population. Rev Esp Fisiol. 1987 September;43(3):335-8. Spanish.

³⁸⁰ Taneja S K, Mahajan M, Arya P. Excess bioavailability of zinc may cause obesity in humans. Experientia. 1996 Jan 16;52(1):31-3.

³⁸¹ Ludvik B, Nolan J J, Baloga J, Sacks D, Olefsky J. Effect of obesity on insulin resistance in normal subjects and patients with NIDDM. Diabetes. 1995 September;44(9):1121-5.

³⁸² Prager R, Wallace P, Olefsky J M. Hyperinsulinemia does not compensate for peripheral insulin resistance in obesity. Diabetes. 1987 March;36(3):327-34.

³⁸³ Tasaka Y, Yanagisawa K, Iwamoto Y. Human plasma leptin in obese subjects and diabetics. Endocr J. 1997 October;44(5):671-6.

³⁸⁴ Bjorbaek C, El-Haschimi K, Frantz J D, Flier J S. The role of SOCS-3 in leptin signaling and leptin resistance. J Biol Chem. Oct. 15, 1999;274(42):30059-65.

³⁸⁵ de Waard F, Poortman J, de Pedro-Alvarez Ferrero M, Baanders-van Halewijn E A. Weight reduction and oestrogen excretion in obese post-menopausal women. Maturitas. 1982 August;4(2): 155-62.

³⁸⁶ Cauley J A, Gutai J P, Kuller L H, LeDonne D, Powell J G. The epidemiology of serum sex hormones in postmenopausal women. Am J Epidemiol. 1989 June; 129(6):1120-31.

³⁸⁷ Cauley J A, Gutai J P, Kuller L H, Scoff J, Nevitt M C. Black-white differences in serum sex hormones and bone mineral density. Am J Epidemiol. May 15, 1994;139(10):1035-46.

³⁸⁸ Galea J, Armstrong J, Gadsdon P, Holden H, Francis S E, Holt C M. Interleukin-1 beta in coronary arteries of patients with ischemic heart disease. Arterioscler Thromb Vasc Biol. 1996 August;16(8):1000-6.

³⁸⁹ Hasdai D, Scheinowitz M, Leibovitz E, Sclarovsky S, Eldar M, Barak V. Increased serum concentrations of interleukin-1 beta in patients with coronary artery disease. Heart. 1996 July;76(1):24-8.

³⁹⁰ Pickup J C, Mattock M B, Chusney G D, Burt D. NIDDM as a disease of the innate immune system: association of acute-phase reactants and interleukin-6 with metabolic syndrome . XDiabetologia. 1997 November;40(11): 1286-92.

³⁹¹ Pickup J C, Crook M A. Is type II diabetes mellitus a disease of the innate immune system? Diabetologia. 1998 October;41(10):1241-8.

³⁹² Hmciar J, Gabor D, Hrnciarova M, Okapcova J, Szentivanyi M, Kurray P. [Relation between cytokines (TNF-alpha, IL-1 and 6) and homocysteine in android obesity and the phenomenon of insulin resistance syndromes]. Vnitr Lek. 1999 January;45(1): 11-6. Slovak.

³⁹³ Avots A, Hoffmeyer A, Flory E, Cimanis A, Rapp L R, Serfling E. GABP factors bind to a distal interleukin 2 (IL-2) enhancer and contribute to c-Raf-mediated increase in IL-2 induction. Molecular and Cellular Biology 1997 17(8):4381-4389.

³⁹⁴ Klein J B, Rane M J, Scherzer J A, Coxon P Y, Kettritz R, Mathiesen J M, Buridi A, McLeish K R. Granulocyte-macrophage colony-stimulating factor delays neutrophil constitutive apoptosis through phosphoinositide 3-kinase and extracellular signal-regulated kinase pathways. J Immunol Apr. 15, 2000;164(8):4286-91.

³⁹⁵ Valledor A F, Comalada M, Xaus J, Celada A. The differential time-course of extracellular-regulated kinase activity correlates with the macrophage response toward proliferation or activation. J Biol Chem Mar. 10, 2000;275(10):7403-9.

³⁹⁶ Thomas R S, Tymris M J, McKinlay L H, Shannon M F, Seth A, Kola I. ETS 1, NFkappaB and AP1 synergistically transactivate the human GM-CSF promoter. Oncogene. Jun. 12, 1997;14(23):2845-55.

³⁹⁷ Dhurandhar N V, Israel B A, Kolesar J M, Mayhew G F, Cook M E, Atkinson R L. Increased adiposity in animals due to a human virus. Int J Obes Relat Metab Disord. 2000 August;24(8):989-96.

³⁹⁸ Behrens G M, Stoll M, Schmidt R E. Lipodystrophy syndrome in HIV infection: what is it, what causes it and how can it be managed? Drug Saf. 2000 July;23(1):57-76.

³⁹⁹ Engelson E S, Kotler D P, Tan Y, Agin D, Wang J, Pierson R N Jr, Heyrusfield S B. Fat distribution in HIV-infected patients reporting truncal enlargement quantified by whole-body magnetic resonance imaging. Am J Clin Nutr. 1999 June;69(6):1162-9.

⁴⁰⁰ Clement K, Vaisse C, Lahlou N, Cabrol S, Pelloux V, Cassuto D, Gourmelen M, Dina C, Chambaz J, Lacorte J M, Basdevant A, Bougneres P, Lebouc Y, Froguel P, Guy-Grand B. A mutation in the human leptin receptor gene causes obesity and pituitary dysfunction. Nature. Mar. 26, 1998;392(6674):398-401.

⁴⁰¹ Chen H, Charlat O, Tartaglia L A, Woolf E A, Weng X, Ellis S J, Lakey N D, Culpepper J, Moore K J, Breitbart R E, Duyk G M, Tepper R I, Morgenstern J P. Evidence that the diabetes gene encodes the leptin receptor: identification of a mutation in the leptin receptor gene in db/db nice. Cell Feb. 9, 1996;84(3):491-5.

⁴⁰² da Silva B A, Bjorbaek C, Uotani S, Flier J S. Functional properties of leptin receptor isoforms containing the gln→pro extracellular domain mutation of the fatty rat. Endocrinology. 1998 September; 139(9):3681-90.

⁴⁰³ Yamashita T, Murakami T, Otani S, Kuwajima M, Shima K. Leptin receptor signal transduction: OBRa and OBRb of fa type. Biochem Biophys Res Commun. May 29, 1998;246(3):752-9.

⁴⁰⁴ del Rey A, Besedovsky H. Antidiabetic effects of interleukin 1. Proc Natl Acad Sci U S A 1989 August;86(15):5943-7.

⁴⁰⁵ Ilyin S E, Plata-Salaman C R. Molecular regulation of the brain interleukin-1 beta system in obese (fa/fa) and lean (Fa/Fa) Zucker rats. Brain Res Mol Brain Res. Dec. 31, 1996;43(1-2):209-18.

⁴⁰⁶ Luheshi G N, Gardner J D, Rushforth D A, Loudon A S, Rothwell N J. Leptin actions on food intake and body temperature are mediated by IL-1. Proc Natl Acad Sci U S A Jun. 8, 1999;96(12):7047-52.

⁴⁰⁷ Plata-Salaman C R, Vasselli J R, Sonti G. Differential responsiveness of obese (fa/fa) and lean (Fa/Fa) Zucker rats to cytokine-induced anorexia. Obes Res 1997 January;5(1):36-42.

⁴⁰⁸ Faggioni R, Fuller J, Moser A, Feingold K R, Grunfeld C. LPS-induced anorexia in leptin-deficient (ob/ob) and leptin receptor-deficient (db/db) mice. Am J Physiol. 1997 July;273(1 Pt 2):R181-6.

⁴⁰⁹ Baroni M G, D'Andrea M P, Montali A, Pannitteri G, Barilla F, Campagna F, Mazzei E, Lovari S, Seccareccia F, Campa P P, Ricci G, Pozzilli P, Urbinati G, Arca M. A common mutation of the insulin receptor substrate-1 gene is a risk factor for coronary artery disease. Arterioscler Thromb Vasc Biol. 1999 December;19(12):2975-80.

⁴¹⁰ Park K, Kim S J, Bang Y J, Park J G, Kim N K, Roberts A B, Spom M B. Genetic changes in the transforming growth factor beta (TGF-beta) type II receptor gene in human gastric cancer cells: correlation with sensitivity to growth inhibition by TGF-beta. Proc Natl Acad Sci U S A. Sep. 13, 1994;91(19):8772-6.

⁴¹¹ Myeroff L L, Parsons R, Kim S J, Hedrick L, Cho K R, Orth K, Mathis M, Kinzler K W, Lutterbaugh J, Park K, et al. A transforming growth factor beta receptor type II gene mutation common in colon and gastric but rare in endometrial cancers with microsatellite instability. Cancer Res. Dec. 1, 1995;55(23):5545-7.

⁴¹² McCaffrey T A, Du B, Consigli S, Szabo P, Bray P J, Harmer L, Weksler B B, Sanborn T A, Bergman G, Bush H L Jr. Genomic instability in the type II TGF-betal receptor gene in atherosclerotic and restenotic vascular cells. J Clin Invest. Nov. 1, 1997;100(9):2182-8.

⁴¹³ Serra R, Johnson M, Filvaroff E H, LaBorde J, Sheehan D M, Derynck R, Moses H L. Expression of a truncated, kinase-defective TGF-beta type II receptor in mouse skeletal tissue promotes terminal chondrocyte differentiation and osteoarthritis. J Cell Biol. Oct. 20, 1997;139(2):541-52.

⁴¹⁴ Caruso A, Fortini A, Fulghesu A M, Pistilli E, Cucinelli F, Lanzone A, Mancuso S. Ovarian sensitivity to follicle-stimulating hormone during the follicular phase of the human menstrual cycle and in patients with polycystic ovarian syndrome. Fertil Steril. 1993 January;59(1): 115-20.

⁴¹⁵ Wronski T J, Schenck P A, Cintron M, Walsh C C. Effect of body weight on osteopenia in ovariectomized rats. Calcif Tissue Int. 1987 March;40(3): 155-9.

⁴¹⁶ Singh R B, Niaz M A, Rastogi S S, Bajaj S, Gaoli Z, Shoumnin Z. Current zinc intake and risk of diabetes and coronary artery disease and factors associated with insulin resistance in rural and urban populations of North India. J Am Coll Nutr. 1998 December;17(6):564-70.

⁴¹⁷ Beattie J H, Wood A M, Newman A M, Bremner I, Choo K H, Michalska A E, Duncan J S, Trayhurn P. Obesity and hyperleptinemia in metallothionein (-I and -II) null mice. Proc Natl Acad Sci U S A Jan. 6, 1998;95(1):358-63.

⁴¹⁸ Flaherty S F, Golenbock D T, Milham F H, Ingalls R R. CD11/CD18 leukocyte integrins: new signaling receptors for bacterial endotoxin. J Surg Res. 1997 November;73(1):85-9.

⁴¹⁹ Ingalls R R, Golenbock D T. CD11 c/CD18, a transmembrane signaling receptor for lipopolysaccharide. J Exp Med. Apr. 1, 1995;181(4):1473-9.

⁴²⁰ Ingalls R R, Arnaout M A, Golenbock D T. Outside-in signaling by lipopolysaccharide through a tailless integrin. J limmunol. Jul. 1, 1997;159(1):433-8.

⁴²¹ Dong Z M, Gutierrez-Ramos J C, Coxon A, Mayadas T N, Wagner D D. A new class of obesity genes encodes leukocyte adhesion receptors. Proc. Natl. Acad. Sci. USA 1997 (94):7526-7530.

⁴²² Muthalif M M, Benter IF, Karzoun N, Fatima S, Harper J, Uddin M R, Malik K U. 20-Hydroxyeicosatetraenoic acid mediates calcium/calmodulin-dependent protein kinase II-induced mitogen-activated protein kinase activation in vascular smooth muscle cells. Proc Natd Acad Sci U S A. Oct. 13, 1998;95(21):12701-6.

⁴²³ Wen Y, Nadler J L, Gonzales N, Scott S, Clauser E, Natarajan R. Mechanisms of ANG II-induced mitogenic responses: role of 12-lipoxygenase and biphasic MAP kinase. Am J Physiol. 1996 October;271(4 Pt 1):C1212-20.

⁴²⁴Rao G N, Baas A S, Glasgow W C, Eling T E, Runge M S, Alexander R W. Activation of mitogen-activated protein kinases by arachidonic acid and its metabolites in vascular smooth muscle cells. J Biol Chem. Dec. 23, 1994;269(51):32586-91.

⁴²⁵ Chen J K, Wang D W, Falck J R, Capdevila J, Harris R C. Transfection of an active cytochrome P450 arachidonic acid epoxygenase indicates that 14,15-epoxyeicosatrienoic acid functions as an intracellular second messenger in response to epidermal growth factor. J Biol Chem. Feb. 19, 1999;274(8):4764-9.

⁴²⁶ Bylund J, Ericsson J, Oliw E H. Analysis of cytochrome P450 metabolites of arachidonic and linoleic acids by liquid chromatography-mass spectrometry with ion trap MS. Anal Biochem. Dec. 1, 1998;265(1):55-68.

⁴²⁷ Imaoka S, Wedlund P J, Ogawa H, Kimura S, Gonzalez F J, Kim H Y. Identification of CYP2C23 expressed in rat kidney as an arachidonic acid epoxygenase. J Pharmacol Exp Ther. 1993 November;267(2): 1012-6.

⁴²⁸ Zeldin D C, Plitman J D, Kobayashi J, Miller R F, Snapper J R, Falck J R, Szarek J L, Philpot R M, Capdevila J H. The rabbit pulmonary cytochrome P450 arachidonic acid metabolic pathway: characterization and significance. J Clin Invest. 1995 May;95(5):2150-60.

⁴²⁹ Rifkind A B, Lee C, Chang T K, Waxman D J. Arachidonic acid metabolism by human cytochrome P450s 2C8, 2C9, 2E1, and 1A2: regioselective oxygenation and evidence for a role for CYP2C enzymes in arachidonic acid epoxygenation in human liver microsomes. Arch Biochem Biophys. Jul. 10, 1995;320(2):380-9.

⁴³⁰ Luo G, Zeldin D C, Blaisdell J A, Hodgson E, Goldstein J A. Cloning and expression of murine CYP2Cs and their ability to metabolize arachidonic acid. Arch Biochem Biophys. Sep. 1, 1998;357(1):45-57.

⁴³¹ Keeney D S, Skinner C, Travers J B, Capdevila J H, Nanney L B, King L E Jr, Waterman M R. Differentiating keratinocytes express a novel cytochrome P450 enzyme, CYP2B 19, having arachidonate monooxygenase activity. J Biol Chem. Nov. 27, 1998;273(48):32071-9.

⁴³² Kidd R S, Straughn A B, Meyer M C, Blaisdell J, Goldstein J A, Dalton J T. Pharmacokinetics of chlorpheniramine, phenytoin, glipizide and nifedipine in an individual homozygous for the CYP2C9*3 allele. Pharmacogenetics. 1999 February;9(1):71-80.

⁴³³ Ring B J, Binkley S N, Vandenbranden M, Wrighton S A. In vitro interaction of the antipsychotic agent olanzapine with human cytochromes P450 CYP2C9, CYP2Cl9, CYP2D6 and CYP3A. Br J Clin Pharmacol. 1996 March;41(3):181-6.

⁴³⁴ Miners J O, Birkett D J. Cytochrome P4502C9: an enzyme of major importance in human drug metabolism. Br J Clin Pharmacol. 1998 June;45(6):525-38. Review.

⁴³⁵ Egger J, Brett E M. Effects of sodium valproate in 100 children with special reference to weight. Br Med J (Clin Res Ed). Aug. 29, 1981;283(6291):577-81.

⁴³⁶ Kidd R S, Straughn A B, Meyer M C, Blaisdell J, Goldstein J A, Dalton J T. Pharmacokinetics of chlorpheniramine, phenytoin, glipizide and nifedipine in an individual homozygous for the CYP2C9*3 allele. Pharmacogenetics. 1999 February;9(1):71-80.

⁴³⁷ Campbell I W, Menzies D G, Chalmers J, McBain A M, Brown I R. One year comparative trial of metformin and glipizide in type 2 diabetes mellitus. Diabete Metab 1994 July-August;20(4):394-400.

⁴³⁸ Petersen K U. Review article: omeprazole and the cytochrome P450 system. Aliment Pharmacol Ther 1995 February;9(1):l-9.

⁴³⁹ Meyer U A. Interaction of proton pump inhibitors with cytochromes P450: consequences for drug interactions. Yale J Biol Med 1996 May-June;69(3):203-9.

⁴⁴⁰ Hogan R E, Bertrand M E, Deaton R L, Sommerville K W. Total percentage body weight changes during add-on therapy with tiagabine, carbamazepine and phenytoin. Epilepsy Res. 2000 August;41(1):23-8.

⁴⁴¹ Mattson R H, Cramer J A, Collins J F. A comparison of valproate with carbamazepine for the treatment of complex partial seizures and secondarily generalized tonic-clonic seizures in adults. The Department of Veterans Affairs Epilepsy Cooperative Study No. 264 Group. N Engl J Med. Sep. 10, 1992;327(11):765-71.

⁴⁴² Sadeque A J M, Fisher M B, Korzekwa K R, Gonzalez F J, Rettie A E. Human CYP2C9 and CYP2A6 mediate formation of the hepatotoxin 4-ene-valproic acid. J Pharmacol Exp Ther. 1997 November;283(2):698-703.

⁴⁴³ Bruni J, Wilder B J. Valproic acid. Review of a new antiepileptic drug. Arch Neurol 1979 July;36(7):393 8.

⁴⁴⁴ Egger J, Brett E M. Effects of sodium valproate in 100 children with special reference to weight. Br Med J (Clin Res Ed). Aug. 29, 1981;283(6291):577-81.

⁴⁴⁵ Zaccara G, Campostrini R, Paganini M, Messori A, Valenza T, Arnetoli G, Zappoli R. Long-term treatment with sodium valproate: monitoring of venous ammonia concentrations and adverse effects. Ther Drug Monit. 1987;9(1):34-40.

⁴⁴⁶ Mattson R H, Cramer J A, Collins J F. A comparison of valproate with carbamazepine for the treatment of complex partial seizures and secondarily generalized tonic-clonic seizures in adults. The Department of Veterans Affairs Epilepsy Cooperative Study No. 264 Group. N Engl J Med. Sep. 10, 1992;327(11):765-71.

⁴⁴⁷ Sharpe C, Buchanan N. Juvenile myoclonic epilepsy: diagnosis, management and outcome. Med J Aust. Feb. 6, 1995;162(3):133-4.

⁴⁴⁸ Song J C, White C M. Pharmacologic, pharmacokinetic, and therapeutic differences among angiotensin II receptor antagonists. Pharmacotherapy. 2000 February;20(2):130-9. Review

⁴⁴⁹ Meadowcroft A M, Williamson K M, Patterson J H, Hinderliter A L, Pieper J A. The effects of fluvastatin, a CYP2C9 inhibitor, on losartan pharmacokinetics in healthy volunteers. J Clin Pharmacol. 1999 April;39(4):418-24.

⁴⁵⁰ Miners J O, Birkett D J. Cytochrome P4502C9: an enzyme of major importance in human drug metabolism. Br J Clin Pharmacol. 1998 June;45(6):525-38. Review.

⁴⁵¹ Camargo M J, von Lutterotti N, Pecker M S, James G D, Timmermans P B, Laragh J H. DuP 753 increases survival in spontaneously hypertensive stroke-prone rats fed a high sodium diet. Am J Hypertens. 1991 April;4(4 Pt 2):341S-345S.

⁴⁵² Transon C, Leemann T, Dayer P. In vitro comparative inhibition profiles of major human drug metabolising cytochrome P450 isozymes (CYP2C9, CYP2D6 and CYP3A4) by HMG-CoA reductase inhibitors. Eur J Clin Pharmacol. 1996;50(3):209-15.

⁴⁵³ Matthews P G, Wahlqvist M L, Marks S J, Myers K A, Hodgson J M. Improvement in arterial stiffness during hypolipidaemnic therapy is offset by weight gain. Int J Obes Relat Metab Disord. 1993 October;17(10):579-83.

⁴⁵⁴ Ring B J, Binkley S N, Vandenbranden M, Wrighton S A. In vitro interaction of the antipsychotic agent olanzapine with human cytochromes P450 CYP2C9, CYP2Cl9, CYP2D6 and CYP3A. Br J Clin Pharmacol. 1996 March;41(3):181-6.

⁴⁵⁵ Osser D N, Najarian D M, Dufresne R L. Olanzapine increases weight and serum triglyceride levels. J Clin Psychiatry. 1999 November;60(11):767-70.

⁴⁵⁶ Koran L M, Ringold A L, Elliott M A. Olanzapine augmentation for treatment-resistant obsessive-compulsive disorder. J Clin Psychiatry. 2000 July;61(7):514-7.

⁴⁵⁷ Ring B J, Binkley S N, Vandenbranden M, Wrighton S A. In vitro interaction of the antipsychotic agent olanzapine with human cytochromes P450 CYP2C9, CYP2Cl9, CYP2D6 and CYP3A. Br J Clin Pharmacol. 1996 March;41(3):181-6.

⁴⁵⁸ Fang J, Coutts R T, McKenna K F, Baker G B. Elucidation of individual cytochrome P450 enzymes involved in the metabolism of clozapine. Naunyn Schmiedebergs Arch Pharmacol. 1998 November;358(5):592-9.

⁴⁵⁹ Prior T I, Chue P S, Tibbo P, Baker G B. Drug metabolism and atypical antipsychotics. Eur Neuropsychopharmacol. 1999 June;9(4):301-9. Review.

⁴⁶⁰ Osser D N, Najarian D M, Dufresne R L. Olanzapine increases weight and serum triglyceride levels. J Clin Psychiatry. 1999 November;60(11):767-70.

⁴⁶¹ Olesen O V, Linnet K. Fluvoxamine-Clozapine drug interaction: inhibition in vitro of five cytochrome P450 isoforms involved in clozapine metabolism. J Clin Psychopharmacol. 2000 February;20(1):35-42.

⁴⁶² Miners J O, Birkett D J. Cytochrome P4502C9: an enzyme of major importance in human drug metabolism. Br J Clin Pharmacol. 1998 June;45(6):525-38. Review.

⁴⁶³ Schmider J, Greenblatt D J, von Moltke L L, Karsov D, Shader R I. Inhibition of CYP2C9 by selective serotonin reuptake inhibitors in vitro: studies of phenytoin p-hydroxylation. Br J Clin Pharmacol. 1997 November;44(5):495-8.

⁴⁶⁴ Harvey B H, Bouwer C D. Neuropharmacology of paradoxic weight gain with selective serotonin reuptake inhibitors. Clin Neuropharmacol. 2000 March-April;23(2):90-7. Review.

⁴⁶⁵ Sansone R A, Wiederman M W, Shrader J A. Naturalistic study of the weight effects of amitriptyline, fluoxetine, and sertraline in an outpatient medical setting. J Clin Psychopharmacol. 2000 April;20(2):272-4.

⁴⁶⁶ Michelson D, Amsterdam J D, Quitkin F M, Reirnherr F W, Rosenbaum J F, Zajecka J, Sundell K L, Kim Y, Beasley C M Jr. Changes in weight during a 1-year trial of fluoxetine. Am J Psychiatry. 1999 August; 156(8):1170-6.

⁴⁶⁷ Darga L L, Carroll-Michals L, Botsford S J, Lucas C P. Fluoxetine's effect on weight loss in obese subjects. Am J Clin Nutr. 1991 August;54(2):321-5.

⁴⁶⁵ Ring B J, Binkley S N, Vandenbranden M, Wrighton S A. In vitro interaction of the antipsychotic agent olanzapine with human cytochromes P450 CYP2C9, CYP2Cl9, CYP2D6 and CYP3A. Br J Clin Pharmacol. 1996 March;41(3):181-6.

⁴⁶⁹ Miners J O, Birkett D J. Cytochrome P4502C9: an enzyme of major importance in human drug metabolism. Br J Clin Pharmacol. 1998 June;45(6):525-38. Review.

⁴⁷⁰ Lasker J M, Wester M R, Aramsombatdee E, Raucy J L. Characterization of CYP2C19 and CYP2C9 from human liver: respective roles in microsomal tolbutamide, S-mephenytoin, and omeprazole hydroxylations. Arch Biochem Biophys. May 1, 1998;353(1): 16-28.

⁴⁷¹ Wissler R W, Borensztajn J, Rubenstein A, Getz G, Vesselinovitch D The effects of tolbutamide on the development of atherosclerosis in rhesus monkeys fed an average American table-prepared diet. Adv Exp Med Biol. 1975;63:379-80.

⁴⁷² Ballagi-Pordany G, Koltai M Z, Aranyi Z, Pogatsa G. Direct effect of hypoglycemic sulphonylureas on the cardiovascular system of dogs. Diabetes Res Clin Pract. 1991 January; 11(1):47-52.

⁴⁷³ Grimm S W, Dyroff M C. Inhibition of human drug metabolizing cytochromes P450 by anastrozole, a potent and selective inhibitor of aromatase. Drug Metab Dispos. 1997 May;25(5):598-602.

⁴⁷⁴ Wiseman L R, Adkins J C. Anastrozole. A review of its use in the management of postmenopausal women with advanced breast cancer. Drugs Aging. 1998 October; 13(4):321-32. Review.

⁴⁷⁵ Lonning P E. Aromatase inhibitors and their future role in post-menopausal women with early breast cancer. Br J Cancer. 1998 September;78 Suppl 4:12-5.

⁴⁷⁶ Buzdar A U. Anastrozole: a new addition to the armamentarium against advanced breast cancer. Am J Clin Oncol. 1998 April;21(2):161-6. Review.

⁴⁷⁷ Jonat W. Clinical overview of anastrozole—a new selective oral aromatase inhibitor. Oncology. 1997;54 Suppl 2:15-8. Review.

⁴⁷⁸ Buzdar A U, Jonat W, Howell A, Plourde P V. ARIMIDEX: a potent and selective aromatase inhibitor for the treatment of advanced breast cancer. J Steroid Biochem Mol Biol. 1997 April;61(3-6):145-9. Review.

⁴⁷⁹ Hannaford M. Role of new selective aromatase inhibitor in therapy for metastatic breast cancer in postmenopausal women. Nurse Pract. 1997 March;22(3):195-6, 201-2.

⁴⁸⁰ Buzdar A U, Jones S E, Vogel C L, Wolter J, Plourde P, Webster A. A phase III trial comparing anastrozole (1 and 10 milligrams), a potent and selective aromatase inhibitor, with megestrol acetate in postmenopausal women with advanced breast carcinoma. Arimidex Study Group. Cancer. Feb. 15, 1997;79(4):730-9.

⁴⁸¹ Buzdar A, Jonat W, Howell A, Jones S E, Blomqvist C, Vogel C L, Eiermann W, Wolter J M, Azab M, Webster A, Plourde P V. Anastrozole, a potent and selective aromatase inhibitor, versus megestrol acetate in postmenopausal women with advanced breast cancer: results of overview analysis of two phase III trials. Arimidex Study Group. J Clin Oncol. 1996 July; 14(7):2000-11.

⁴⁸² Jonat W, Howell A, Blomqvist C, Eiermann W, Winblad G, Tyrrell C, Mauriac L, Roche H, Lundgren S, Hellmund R, Azab M. A randomised trial comparing two doses of the new selective aromatase inhibitor anastrozole (Arimidex) with megestrol acetate in postmenopausal patients with advanced breast cancer. Eur J Cancer. 1996 March;32A(3):404-12.

⁴⁸³ Khaliq Y, Gallicano K, Seguin I, Fyke K, Carignan G, Bulman D, Badley A, Cameron D W. Single and multiple dose pharrnacokinetics of nelfmavir and CYP2C 19 activity in human immunodeficiency virus-infected patients with chronic liver disease. Br J Clin Pharmacol. 2000 August;50(2):108-15.

⁴⁸⁴ Lillibridge J H, Liang B H, Kerr B M, Webber S, Quart B, Shetty B V, Lee C A. Characterization of the selectivity and mechanism of human cytochrome P450 inhibition by the human immunodeficiency virus-protease inhibitor nelfinavir mesylate. Drug Metab Dispos. 1998 July;26(7):609-16.

⁴⁸⁵ Muirhead G J, Wulff M B, Fielding A, Kleinermans D, Buss N. Pharmacokinetic interactions between sildenafil and saquinavir/ritonavir. Br J Clin Pharmacol. 2000 August;50(2):99-107.

⁴⁸⁶Kumar G N, Dykstra J, Roberts E M, Jayanti V K, Hickman D, Uchic J, Yao Y, Surber B, Thomas S, Granneman GRPotent inhibition of the cytochrome P-450 3A-mediated human liver microsomal metabolism of a novel HIV protease inhibitor by ritonavir: A positive drug-drug interaction. Drug Metab Dispos. 1999 August;27(8):902-8.

⁴⁸⁷ Kumar G N, Rodrigues A D, Buko A M, Denissen J F. Cytochrome P450-mediated metabolism of the HIV-1 protease inhibitor ritonavir (ABT-538) in human liver microsomes. J Pharmacol Exp Ther. 1996 April;277(1):423-31.

⁴⁸⁸ Eagling V A, Back D J, Barry M G. Differential inhibition of cytochrome P450 isoforms by the protease inhibitors, ritonavir, saquinavir and indinavir. Br J Clin Pharmacol. 1997 August;44(2): 190-4.

⁴⁸⁹ Fung H B, Kirschenbaum H L, Hameed R. Amprenavir: a new human immunodeficiency virus type 1 protease inhibitor. Clin Ther. 2000 May;22(5):549-72. Review.

⁴⁹⁰ Eagling V A, Back D J, Barry M G. Differential inhibition of cytochrome P450 isoforms by the protease inhibitors, ritonavir, saquinavir and indinavir. Br J Clin Pharmacol. 1997 August;44(2): 190-4.

⁴⁹¹ Fisslthaler B, Hinsch N, Chataigneau T, Popp R, Kiss L, Busse R, Fleming I. Nifedipine increases cytochrome P4502C expression and endothelium-derived hyperpolarizing factor-mediated responses in coronary arteries. Hypertension. 2000 August;36(2):270-5.

⁴⁹² Krakoff LREffectiveness of nifedipine gastrointestinal therapeutic system for treatment of hypertension: results of the MATH Trial. J Cardiovasc Pharmacol. 1993;21 Suppl 2:S14-7.

⁴⁹³ Maccario M, Oleandri S E, Avogadri E, Rossetto R, Grottoli S, Procopio M, Camanni F, Ghigo E. Effects of 3-month nifedipine treatment on endocrine-metabolic parameters in patients with abdominal obesity and mild hypertension. J Endocrinol Invest. 1998 January;21(1):56-63.

⁴⁹⁴ Andronico G, Piazza G, Mangano M T, Mule G, Carone M B, Cerasola G. Nifedipine vs. enalapril in treatment of hypertensive patients with glucose intolerance. J Cardiovasc Pharmacol. 1991; 18 Suppl 10:S52-4.

⁴⁹⁵ Barreiro P, Soriano V, Blanco F, Casimiro C, de la Cruz J J, Gonzalez-Lahoz J. Risks and benefits of replacing protease inhibitors by nevirapine in HIV-infected subjects under long-term successful triple combination therapy. AIDS. May 5, 2000;14(7):807-12.

⁴⁹⁶ Mulligan K, Grunfeld C, Tai V W, Algren H, Pang M, Chemoff D N, Lo J C, Schambelan M. Hyperlipidemia and insulin resistance are induced by protease inhibitors independent of changes in body composition in patients with HIV infection. J Acquir Immune Defic Syndr. Jan. 1, 2000;23(1):35-43.

⁴⁹⁷ Gervasoni C, Ridolfo A L, Trifiro G, Santambrogio S, Norbiato G, Musicco M, Clerici M, Galli M, Moroni M. Redistribution of body fat in HIV-infected women undergoing combined antiretroviral therapy. AIDS. Mar. 11, 1999;13(4):465-71.

⁴⁹⁸ Carr A, Copper D A. A randomized, multicenter study of protease inhibitor (PI) substitution in avirernic patients with antiretroviral (ARV) lipodystrophy syndrome. 7th Conference on Retroviruses and Opportunistic Infections. 2000 (Abstract).

⁴⁹⁹ Martinez E, Blanco J L, Garcia M A, Buira E, Bianchi L, Conget I, Casamitjana R, Gatell J M. Impact of switching from HIV-1 protease Inhibitors (PI) to efavirenz (EFV) in patients with lipodystrophy. 7th Conference on Retroviruses and Opportunistic Infections. 2000 (Abstract).

⁵⁰⁰ Passalaris J D, Sepkowitz K A, Glesby M J. Coronary artery disease and human inununodeficiency virus infection. Clin Infect Dis. 2000 September;31(3):787-97.

⁵⁰¹ Andrews G K, Adamson E D. Butyrate selectively activates the metallothionein gene in teratocarcinoma cells and induces hypersensitivity to metal induction. Nuclein Acids Research. 1987 15(13): 5461-5475.

⁵⁰² Thomas D J, Angle C R, Swanson S A, Caffrey T C. Effect of sodium butyrate on metallothionein induction and cadmium cytotoxicity in ROS 17/2.8 cells. Toxicology Feb. 11, 1991;66(1):35-46.

⁵⁰³ Liu J, McKim J M Jr, Liu Y P, Klaassen C D. Effects of butyrate homologues on metallothionein induction in rat primary hepatocyte cultures. In vitro Cell Dev Biol 1992 May;28A(5):320-6.

⁵⁰⁴ Ludwig D S, Pereira M A, Kroenke C H, Hilner J E, Van Horn L, Slattery M L, Jacobs D R Jr. Dietary fiber, weight gain, and cardiovascular disease risk factors in young adults. JAMA. Oct. 27, 1999;282(16):1539-46.

⁵⁰⁵ Rigaud D, Ryttig K R, Angel L A, Apfelbaum M. Overweight treated with energy restriction and a dietary fibre supplement: a 6-month randomized, double-blind, placebo-controlled trial. Int J Obes. 1990 September;14(9):763-9.

⁵⁰⁶ Ryttig K R, Tellnes G, Haegh L, Boe E, Fagerthun H. A dietary fibre supplement and weight maintenance after weight reduction: a randomized, double-blind, placebo-controlled long-term trial. IntJ Obes. 1989;13(2):165-71.

⁵⁰⁷ Piliang W G, Djojosoebagio S, Suprayogi A. Soybean hull and its effect on atherosclerosis in non-human primates (Macaca fascicularis). Biomed Environ Sci 1996 September;9(2-3):137-43.

⁵⁰⁸ Kim Y I. AGA technical review: impact of dietary fiber on colon cancer occurrence. Gastroenterology. 2000 June;118(6):1235-57.

⁵⁰⁹ Madar Z, Stark A. Dietary fiber and colorectal cancer. N Engl J Med. Jun. 17, 1999;340(24): 1925-6; discussion 1926.

⁵¹⁰ Camire M E. Dietary fiber and colorectal cancer. N Engl J Med. Jun 17, 1999;340(24):1926.

⁵¹¹ Mohandas K M. Dietary fiber and colorectal cancer. N Engl J Med. Jun 17, 1999;340(24):1925; discussion 1926.

⁵¹² Heaton K W, Lewis S J. Dietary fiber and colorectal cancer. N Engl J Med. Jun 17, 1999;340(24): 1925; discussion 1926.

⁵¹³ Cumnlings J H, Southgate D A. Dietary fiber and colorectal cancer. N Engl J Med. Jun. 17, 1999;340(24):1925; discussion 1926.

⁵¹⁴ Ravin N D. Dietary fiber and colorectal cancer. N Engl J Med. Jun 17, 1999;340(24):1924-5; discussion 1926.

⁵¹⁵ Reddy B S. Role of dietary fiber in colon cancer: an overview. Am J Med. Jan. 25, 1999;106(1A): 16S-19S; discussion 50S-51S.

⁵¹⁶ Reddy B S. Prevention of colon carcinogenesis by components of dietary fiber. Anticancer Res. 1999 September-October; 19(5A):3681-3.

⁵¹⁷ Earnest D L, Einspahr J G, Alberts D S. Protective role of wheat bran fiber: data from marker trials. Am J Med. Jan. 25, 1999;106(1A):32S-37S.

⁵¹⁸ Kritchevsky D. Protective role of wheat bran fiber: preclinical data. Am J Med. Jan. 25, 1999;106(1A):28S-31S.

⁵¹⁹ Cohen L A. Dietary fiber and breast cancer. Anticancer Res. 1999 September-October;19(5A):3685-8.

⁵²⁰ Wolin M J, Miller T L, Yerry S, Zhang Y, Bank S, Weaver G A. Changes of fermentation pathways of fecal microbial comrnunities associated with a drug treatment that increases dietary starch in the human colon. Appl Environ Microbiol 1999 July;65(7):2807-12.

⁵²¹ Wolin M J, Miller T L, Yerry S, Zhang Y, Bank S, Weaver G A. Changes of fermentation pathways of fecal microbial communities associated with a drug treatment that increases dietary starch in the human colon. Appl Environ Microbiol 1999 July;65(7):2807-12.

⁵²² Wolever T M, Chiasson J L, Josse R G, Hunt J A, Palmason C, Rodger N W, Ross S A, Ryan E A, Tan M H. Small weight loss on long-term acarbose therapy with no change in dietary pattern or nutrient intake of individuals with non-insulin-dependent diabetes. Int J Obes Relat Metab Disord. 1997 September;21(9):756-63.

⁵²³ Huyer G, Liu S, Kelly J, Moffat J, Payette P, Kennedy B, Tsaprailis G, Gresser M J, Ramachandran C. Mechanism of inhibition of protein-tyrosine phosphatases by vanadate and pervanadate. J Biol Chem Jan. 10, 1997;272(2):843-51.

⁵²⁴ Wang Y Z, Bonner J C. Mechanism of extracellular signal-regulated kinase (ERK)-1 and ERK-2 activation by vanadium pentoxide in rat pulmonary myofibroblasts. Am J Respir Cell Mol Biol. 2000 May;22(5):590-6.

⁵²⁵ Zhao Z, Tan Z, Diltz C D, You M, Fischer E H. Activation of mitogen-activated protein (MAP) kinase pathway by pervanadate, a potent inhibitor of tyrosine phosphatases. J Biol Chem. Sep. 6, 1996;271(36):22251-5.

⁵²⁶ Pandey S K, Chiasson J L, Srivastava A K. Vanadium salts stimulate mitogen-activated protein (MAP) kinases and ribosomal S6 kinases. Mol Cell Biochem. Dec. 6-20, 1995;153(1-2):69-78.

⁵²⁷ D'Onofrio F, Le M Q, Chiasson J L, Srivastava A K. Activation of mitogen activated protein (MAP) kinases by vanadate is independent of insulin receptor autophosphorylation. FEBS Lett. Mar. 7, 1994;340(3):269-75.

⁵²⁸ Darville M I, Antoine I V, Rousseau G G. Characterization of an enhancer upstream from the muscle-type promoter of a gene encoding 6-phosphofructo-2-kinase/fructose-2,6-bisphosphatase. Nucleic Acids Res. Jul. 25, 1992;20(14):3575-83.

⁵²⁹ Dupriez V J, Darville M I, Antoine I V, Gegonne A, Ghysdael J, Rousseau G G. Characterization of a hepatoma mRNA transcribed from a third promoter of a 6-phosphofructo-2-kinase/fructose-2,6-bisphosphatase-encoding gene and controlled by ets oncogene-related products. Proc Natl Acad Sci U S A. Sep. 1, 1993;90(17):8224-8.

⁵³⁰ Miralpeix M, Carballo E, Bartrons R, Crepin K, Hue L, Rousseau G G. Oral administration of vanadate to diabetic rats restores liver 6-phosphofructo-2-kinase content and mRNA. Diabetologia. 1992 March;35(3):243-8.

⁵³¹ Inoue H, Kaku K, Matsutani A, Tao T, Ayame H, Kaneko T. Insulin-like effects of vanadate on rat liver 6-phosphofructo-2-kcinase/fructose-2,6-bisphosphatase mRNA and protein inductions in diabetic rats. Endocr J. 1994 February;41(1):75-82.

⁵³² Dupriez V J, Darville M I, Antoine I V, Gegonne A, Ghysdael J, Rousseau G G. Characterization of a hepatoma mRNA transcribed from a third promoter of a 6-phosphofructo-2-kinase/fructose-2,6-bisphosphatase-encoding gene and controlled by ets oncogene-related products. Proc Natl Acad Sci U S A. Sep. 1, 1993;90(17):8224-8.

⁵³³ Herrman C E, Sanders R A, Klaunig J E, Schwarz L R, Watkins J B 3rd. Decreased apoptosis as a mechanism for hepatomegaly in streptozotocin-induced diabetic rats. Toxicol Sci 1999 July;50(1): 146-51.

⁵³⁴ Pugazhenthi S, Tanha F, Dahl B, Khandelwal R L. Decrease in protein tyrosine phosphatase activities in vanadate-treated obese Zucker (fa/fa) rat liver. Mol Cell Biochem. Dec 6-20, 1995;153(1-2):125-9.

⁵³⁵ McNeill J H, Orvig C. Bis(maltolato)oxovanadium compositions for the treatment of elevated blood sugar. U.S. Pat. No. 5,527,790, Jun. 18, 1996.

⁵³⁶ Dai S, Thompson K H, McNeill J H. One-year treatment of streptozotocin-induced diabetic rats with vanadyl sulphate. Pharmacol Toxicol 1994 February;74(2):101-9.

⁵³⁷ Bhanot S, McNeill J H. Vanadyl sulfate lowers plasma insulin and blood pressure in spontaneously hypertensive rats. Hypertension. 1994 November;24(5):625-32.

⁵³⁸ Cruz T F, Morgan A, Min W. In vitro and in vivo antineoplastic effects of orthovanadate. Mol Cell Biochem. Dec. 6-20, 1995;153(1-2):161-6.

⁵³⁹ Bishayee A, Chatterjee M. Inhibitory effect of vanadium on rat liver carcinogenesis initiated with diethylnitrosamine and promoted by phenobarbital. Br J Cancer. 1995 June;71(6): 1214-20.

⁵⁴⁰ Liasko R, Kabanos T A, Karkabounas S, Malamas M, Tasiopoulos A J, Stefanou D, Collery P, Evangelou A. Beneficial effects of a vanadium complex with cysteine, administered at low doses on benzo(alpha)pyrene-induced leiomyosarcomas in Wistar rats. Anticancer Res. 1998 September-October; 18(5A):3609-13.

⁵⁴¹ Goldfme A B, Simonson D C, Folli F, Patti M E, Kahn C R. In vivo and in vitro studies of vanadate in human and rodent diabetes mellitus. Mol Cell Biochem. Dec 6-20, 1995;153(1-2):217-31.

⁵⁴² Brichard S M, Henquin J C. The role of vanadium in the management of diabetes. Trends Pharmacol Sci. 1995 August;16(8):265-70.

⁵⁴³ Huyer G, Liu S, Kelly J, Moffat J, Payette P, Kennedy B, Tsaprailis G, Gresser M J, Ramachandran C. Mechanism of inhibition of protein-tyrosine phosphatases by vanadate and pervanadate. J Biol Chem Jan 10, 1997;272(2):843-51.

⁵⁴⁴ Elchebly M, Payette P, Michaliszyn E, Cromlish W, Collins S, Loy A L, Normandin D, Cheng A, Himnns-Hagen J, Chan C C, Ramachandran C, Gresser M J, Tremblay M L, Kennedy B P. Increased insulin sensitivity and obesity resistance in mice lacking the protein tyrosine phosphatase-1B gene. Science. Mar. 5, 1999;283(5407):1544-8.

⁵⁴⁵ Klaman L D, Boss O, Peroni O D, Kim J K, Martino J L, Zabolotny J M, Moghal N, Lubkin M, Kim Y B, Sharpe A H, Stricker-Krongrad A, Shulman G I, Neel B G, Kahn B B. Increased energy expenditure, decreased adiposity, and tissue-specific insulin sensitivity in protein-tyrosine phosphatase 1B-deficient mice. Mol Cell Biol. 2000 August;20(15):5479-89.

⁵⁴⁶ Elchebly M, Payette P, Michaliszyn E, Cromlish W, Collins S, Loy A L, Normandin D, Cheng A, Hirruns-Hagen J, Chan C C, Ramachandran C, Gresser M J, Tremblay M L, Kennedy B P. Increased insulin sensitivity and obesity resistance in mice lacking the protein tyrosine phosphatase-1B gene. Science. Mar 5, 1999;283(5407):1544-8.

⁵⁴⁷ Klaman L D, Boss O, Peroni O D, Kim J K, Martino J L, Zabolotny J M, Moghal N, Lubkin M, Kim Y B, Sharpe A H, Stricker-Krongrad A, Shulman G I, Neel B G, Kahn B B. Increased energy expenditure, decreased adiposity, and tissue-specific insulin sensitivity in protein-tyrosine phosphatase 1B-deficient mice. Mol Cell Biol. 2000 August;20(15):5479-89.

⁵⁴⁸ Prasad K, Laxdal V A, Yu M, Raney B L. Evaluation of hydroxyl radical-scavenging property of garlic. Mol Cell Biochem. Jan. 12, 1996;154(1):55-63.

⁵⁴⁹ Ide N, Lau BH. Aged garlic extract attenuates intracellular oxidative stress. Phytomedicine 1999 May;6(2):125-31.

⁵⁵⁰ Efendy J L, Simmons D L, Campbell G R, Campbell J H. The effect of the aged garlic extract, ‘Kyolic’, on the development of experimental atherosclerosis. Atherosclerosis. Jul. 11, 1997;132(1):37-42.

⁵⁵¹ Jain R C, Konar DBEffect of garlic oil in experimental cholesterol atherosclerosis. Atherosclerosis. 1978 February;29(2): 125-9.

⁵⁵² Jain R C. Onion and garlic in experimental cholesterol induced atherosclerosis. Indian J Med Res. 1976 October;64(10):1509-15.

⁵⁵³ Bordia A, Arora S K, Kothari L K, Jain K C, Rathore B S, Rathore A S, Dube M K, Bhu N. The protective action of essential oils of onion and garlic in cholesterol-fed rabbits. Atherosclerosis. 1975 July-August;22(1): 103-9.

⁵⁵⁴ Breithaupt-Grogler K, Ling M, Boudoulas H, Belz G G. Protective effect of chronic garlic intake on elastic properties of aorta in the elderly. Circulation. Oct 21, 1997;96(8):2649-55.

⁵⁵⁵ Farrar D J, Bond M G, Riley W A, Sawyer J K. Anatomic correlates of aortic pulse wave velocity and carotid artery elasticity during atherosclerosis progression and regression in monkeys. Circulation. 1991 May;83(5):1754-63.

⁵⁵⁶ Ali M, Thomson M, Afzal M. Garlic and onions: their effect on eicosanoid metabolism and its clinical relevance. Prostaglandins Leukot Essent Fatty Acids. 2000 February;62(2):55-73.

⁵⁵⁷ Fleischauer A T, Poole C, Arab L. Garlic consumption and cancer prevention: meta-analyses of colorectal and stomach cancers. Am J Clin Nutr. 2000 October;72(4): 1047-52.

⁵⁵⁸ Singh A, Shukla Y. Antitumour activity of diallyl sulfide on polycyclic aromatic hydrocarbon-induced mouse skin carcinogenesis. Cancer Lett. Sep. 25, 1998;131(2):209-14.

⁵⁵⁹ Singh S V, Mohan R R, Agarwal R, Benson P J, Hu X, Rudy M A, Xia H, Katoh A, Srivastava S K, Mukhtar H, Gupta V, Zaren H A. Novel anti-carcinogenic activity of an organosulfide from garlic: inhibition of H-RAS oncogene transformed tumor growth in vivo by diallyl disulfide is associated with inhibition of p21^{H-ras} processing. Biochem Biophys Res Commun. Aug 14, 1996;225(2):660-5.

⁵⁶⁰ Spector S A. Oral ganciclovir. Adv Exp Med Biol 1999;458:121-7.

⁵⁶¹ Leflore S, Anderson P L, Fletcher C V. A risk-benefit evaluation of aciclovir for the treatment and prophylaxis of herpes simplex virus infections. Drug Saf 2000 August;23(2):131-42.

⁵⁶² Kesson A M. Use of aciclovir in herpes simplex virus infections. J Paediatr Child Health. 1998 February;34(1):9-13.

⁵⁶³ Ormrod D, Scott L J, Perry C M. Valaciclovir: a review of its long term utility in the management of genital herpes simplex virus and cytomegalovirus infections. Drugs 2000 April;59(4):839-63.

⁵⁶⁴ Bell A R. Valaciclovir update. Adv Exp Med Biol. 1999;458:149-57.

⁵⁶⁵ Sacks S L, Wilson B. Famciclovir/penciclovir. Adv Exp Med Biol. 1999;458:135-47.

⁵⁶⁶ Reddehase M J, Balthesen M, Rapp M, Jonjic S, Pavic I, Koszinowski UH. The conditions of primary infection define the load of latent viral genome in organs and the risk of recurrent cytomegalovirus disease. J Exp Med Jan. 1, 1994;179(1):185-93.

⁵⁶⁷ Collins T, Pomeroy C, Jordan M C. Detection of latent cytomegalovirus DNA in diverse organs of mice. J Infect Dis 1993 September; 168(3):725-9.

⁵⁶⁸ Steffens H P, Kurz S, Holtappels R, Reddehase M J. Preemptive CD8 T-cell immunotherapy of acute cytomegalovirus infection prevents lethal disease, limits the burden of latent viral genomes, and reduces the risk of virus recurrence. J Virol. 1998 March;72(3):1797-804.

⁵⁶⁹ Thackray A M, Field H J. The effects of antiviral therapy on the distribution of herpes simplex virus type 1 to ganglionic neurons and its consequences during, inunediately following and several months after treatment. J Gen Virol 2000 October;81 Pt 10:2385-96.

⁵⁷⁰ Thackray A M, Field H J. Further evidence from a murine infection model that famciclovir interferes with the establishment of HSV-1 latent infections. J Antimicrob Chemother. 2000 June;45(6):825-33.

⁵⁷¹ Thackray A M, Field H J. Effects of famciclovir and valacyclovir on herpes simplex virus type 1 infection, latency, and reactivation in mice: how dissimilar are study results? J Infect Dis 2000 April;181(4):1517-8.

⁵⁷² Field H J, Thackray A M. Early therapy with valaciclovir or famciclovir reduces but does not abrogate herpes simplex virus neuronal latency. Nucleosides Nucleotides Nucleic Acids. 2000 January-February;19(1-2):461-70.

⁵⁷³ Thackray A M, Field H J. Famciclovir and valaciclovir differ in the prevention of herpes simplex virus type 1 latency in mice: a quantitative study. Antimicrob Agents Chemother 1998 July;42(7):1555-62.

⁵⁷⁴ LeBlanc R A, Pesnicak L, Godleski M, Straus S E. Treatment of HSV-1 infection with immunoglobulin or acyclovir: comparison of their effects on viral spread, latency, and reactivation. Virology. Sep. 15, 1999;262(1):230-6.

⁵⁷⁵ Bowden R A. Cytomegalovirus infections in transplant patients: methods of prevention of primary cytomegalovirus. Transplant Proc 1991 June;23(3 Suppl 3):136-8, discussion 138.

⁵⁷⁶ Chou S W, Norman D J. The influence of donor factors other than serologic status on transmission of cytomegalovirus to transplant recipients. Transplantation 1988 July;46(1):89-93.

⁵⁷⁷ Chou S W. Cytomegalovirus infection and reinfection transmitted by heart transplantation. J Infect Dis. 1987 May;155(5):1054-6.

⁵⁷⁸ Chou S W. Acquisition of donor strains of cytomegalovirus by renal-transplant recipients. N Engl J Med. May 29, 1986;314(22):1418-23.

⁵⁷⁹ Grundy J E, Lui S F, Super M, Berry N J, Sweny P, Fernando O N, Moorhead J, Griffiths P D. Symptomatic cytomegalovirus infection in seropositive kidney recipients: reinfection with donor virus rather than reactivation of recipient virus. Lancet Jul. 16, 1988;2(8603):132-5.

⁵⁸⁰ Grundy J E, Super M, Lui S, Sweny P, Griffiths P D. The source of cytomegalovirus infection in seropositive renal allograft recipients is frequently the donor kidney. Transplant Proc. 1987 February;19(1 Pt 3):2126-8.

⁵⁸¹ Grundy J E, Super M, Lui S F, Griffiths P D. Donor strains of cytomegalovirus in renal-transplant recipients. N Engl J Med. Nov. 6, 1986;315(19):1229.

⁵⁸² Valantine H A, Gao S Z, Menon S G, Renlund D G, Hunt S A, Oyer P, Stinson E B, Brown B W Jr, Merigan T C, Schroeder J S. Impact of prophylactic immediate posttransplant ganciclovir on development of transplant atherosclerosis: a post hoc analysis of a randomized, placebo-controlled study. Circulation. Jul. 6, 1999;100(1):61-6.

⁵⁸³ Sia I G, Patel R. New strategies for prevention and therapy of cytomegalovirus infection and disease in solid-organ transplant recipients. Clin Microbiol Rev. 2000 January; 13(1):83-121.

⁵⁸⁴ Reddehase M J, Balthesen M, Rapp M, Jonjic S, Pavic I, Koszinowski U H. The conditions of prirnary infection defme the load of latent viral genome in organs and the risk of recurrent cytomegalovirus disease. J Exp Med Jan 1, 1994;179(1):185-93.

⁵⁸⁵ Perry C M, Noble S. Didanosine: an updated review of its use in HIV infection. Drugs. 1999 December;58(6): 1099-135.

⁵⁸⁶ Bruisten S M, Reiss P, Loeliger A E, van Swieten P, Schuurnan R, Boucher C A, Weverling G J, Huisman J G. Cellular proviral HIV type 1 DNA load persists after long-term RT-inhibitor therapy in HIV type 1 infected persons. AIDS Res Hum Retroviruses. Aug 10, 1998; 14(12):1053-8.

⁵⁸⁷ Magnani M, Rossi L, Fratemale A, Casabianca A, Brandi G, Benafti U, De Flora A. Targeting antiviral nucleotide analogues to macrophages. J Leukoc Biol 1997 July;62(1):133-7.

⁵⁸⁸ Pauza C D, Trivedi P, McKechnie T S, Richman D D, Graziano F M. 2-LTR circular viral DNA as a marker for human immnunodeficiency virus type 1 infection in vivo. Virology. 1994 December;205(2):470-8.

⁵⁸⁹ Chun T W, Stuyver L, Mizell S B, Ehler L A, Mican J A, Baseler M, Lloyd A L, Nowak M A, Fauci A S. Presence of an inducible HIV-1 latent reservoir during highly active antiretroviral therapy. Proc Natl Acad Sci U S A. Nov 25, 1997;94(24):13193-7.

⁵⁹⁰ Gervasoni C, Ridolfo A L, Trifiro G, Santambrogio S, Norbiato G, Musicco M, Clerici M, Galli M, Moroni M. Redistribution of body fat in HIV-infected women undergoing combined antiretroviral therapy. AIDS. Mar 11, 1999;13(4):465-71.

⁵⁹¹ Guo N L, Lu D P, Woods G L, Reed E, Zhou G Z, Zhang L B, Waldman R H. Demonstration of the anti-viral activity of garlic extract against human cytomegalovirus in vitro. Chin Med J (Engl) 1993 February;106(2):93-6.

⁵⁹² Weber N D, Andersen D O, North J A, Murray B K, Lawson L D, Hughes B G. In vitro virucidal effects of Allium sativum (garlic) extract and compounds. Planta Med. 1992 October;58(5):417-23.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of treating adverse affects associated with a disruption of GABP's metabolic pathway, comprising administering to a patient an effective amount of an agent selected from a group consisting of:
a) an agent that increases concentration of a GABP stimulator;
b) an agent that decreases concentration of a GABP suppressor;
c) an agent that increases effectiveness of a GABP stimulator; and
d) an agent that decreases effectiveness of a GABP suppressor.

2. The method of claim 1 wherein said adverse affect associated with microcompetition is cancer and said patient is being treated for cancer.

3. The method of claim 1 wherein said adverse affect associated with microcompetition is atherosclerosis and said patient is being treated for atherosclerosis.

4. The method of claim 1 wherein said adverse affect associated with microcompetition is osteoarthritis and said patient is being treated for osteoarthritis.

5. The method of claim 1 wherein said adverse affect associated with microcompetition is obesity and said patient is being treated for obesity.

6. A method of treating adverse affects associated with a disruption of GABP's metabolic pathway, comprising administering an effective amount of an agent selected from a group consisting of:
a) an agent that increases concentration of GABPα;
b) an agent that increases concentration of GABPβ;
c) an agent that decreases concentration of GABPγ;
d) an agent that increases phosphorylation of GABP;
e) an agent that increases affinity between GABP and p300/CBP;
f) an agent that stimulates binding of GABP to DNA; and
g) an agent that increases concentration of p300/CBP.

7. The method of claim 6 wherein said adverse affect associated with microcompetition is cancer and said patient is being treated for cancer.

8. The method of claim 6 wherein said adverse affect associated with microcompetition is atherosclerosis and said patient is being treated for atherosclerosis.

9. The method of claim 6 wherein said adverse affect associated with microcompetition is osteoarthritis and said patient is being treated for osteoarthritis.

10. The method of claim 6 wherein said adverse affect associated with microcompetition is obesity and said patient is being treated for obesity.

11. A method of treating adverse affects associated with a disruption of GABP's metabolic pathway, comprising administering an effective amount of an agent selected from a group consisting of:
a) an agent that increases concentration of a GABP kinase;
b) an agent that stimulates phosphorylation of a GABP kinase;
c) an agent that increases affinity between GABP and GABP kinase;
d) an agent that decreases oxidative effects on GABP; and
e) an agent that decreases foreign DNA N-boxes in cells.

12. The method of claim 11 wherein said adverse affect associated with microcompetition is cancer and said patient is being treated for cancer.

13. The method of claim 11 wherein said adverse affect associated with microcompetition is atherosclerosis and said patient is being treated for atherosclerosis.

14. The method of claim 11 wherein said adverse affect associated with microcompetition is osteoarthritis and said patient is being treated for osteoarthritis.

15. The method of claim 11 wherein said adverse affect associated with microcompetition is obesity and said patient is being treated for obesity.

16. A method of treating adverse affects associated with a disruption of GABP's metabolic pathway, comprising administering an effective amount of an agent that decreases oxidative effects on GABP.

17. The method of claim 16 wherein said adverse affect associated with microcompetition is cancer and said patient is being treated for cancer.

18. The method of claim 16 wherein said adverse affect associated with microcompetition is atherosclerosis and said patient is being treated for atherosclerosis.

19. The method of claim 16 wherein said adverse affect associated with microcompetition is osteoarthritis and said patient is being treated for osteoarthritis.

20. The method of claim 16 wherein said adverse affect associated with microcompetition is obesity and said patient is being treated for obesity.

21. A method of treating adverse affects associated with a disruption of GABP's metabolic pathway, comprising administering an effective amount of an agent that decreases foreign DNA N-boxes in cells.

22. The method of claim 20 wherein said adverse affect associated with microcompetition is cancer and said patient is being treated for cancer.

23. The method of claim 20 wherein said adverse affect associated with microcompetition is atherosclerosis and said patient is being treated for atherosclerosis.

24. The method of claim 20 wherein said adverse affect associated with microcompetition is osteoarthritis and said patient is being treated for osteoarthritis.

25. The method of claim 20 wherein said adverse affect associated with microcompetition is obesity and said patient is being treated for obesity.

26. A method of identifying compounds to be tested for treating adverse affects associated with a disruption of GABP's metabolic pathway, comprising:
a) providing an assay capable of determining compounds that perform a function selected from a group consisting of:
i) increases concentration of a GABP stimulator,
ii) decreases concentration of a GABP suppressor,
iii) increases effectiveness of a GABP stimulator, and
iv) an agent that decreases effectiveness of a GABP suppressor; and
b) running sample compounds through said assay and identifying any compound that performs one of said functions as a compound to be tested.

27. The method of claim 26 wherein said adverse affect associated with microcompetition is cancer and said patient is being treated for cancer.

28. The method of claim 26 wherein said adverse affect associated with microcompetition is atherosclerosis and said patient is being treated for atherosclerosis.

29. The method of claim 26 wherein said adverse affect associated with microcompetition is osteoarthritis and said patient is being treated for osteoarthritis.

30. The method of claim 26 wherein said adverse affect associated with microcompetition is obesity and said patient is being treated for obesity.

31. A method of identifying compounds to be tested for treating adverse affects associated with a disruption of GABP's metabolic pathway, comprising:
a) providing an assay capable of determining compounds that perform a function selected from a group consisting of:
i) increases concentration of GABPα,
ii) increases concentration of GABPβ,
iii) decreases concentration of GABPγ,
iv) increases phosphorylation of GABP,
v) increases affinity between GABP and p300/CBP,
vi) stimulates binding of GABP to DNA, and
vii) increases concentration of p300/CBP; and
b) running sample compounds through said assay and identifying any compound that performs one of said functions as a compound to be tested.

32. The method of claim 31 wherein said adverse affect associated with microcompetition is cancer and said patient is being treated for cancer.

33. The method of claim 31 wherein said adverse affect associated with microcompetition is atherosclerosis and said patient is being treated for atherosclerosis.

34. The method of claim 31 wherein said adverse affect associated with microcompetition is osteoarthritis and said patient is being treated for osteoarthritis.

35. The method of claim 31 wherein said adverse affect associated with microcompetition is obesity and said patient is being treated for obesity.

36. A method of identifying compounds to be tested for treating adverse affects associated with a disruption of GABP's metabolic pathway, comprising:
a) providing an assay capable of determining compounds that perform a function selected from a group consisting of:
i) increases concentration of a GABP kinase,
ii) stimulates phosphorylation of a GABP kinase, and
iii) increases affinity between GABP and GABP kinase; and
b) running sample compounds through said assay and identifying any compound that performs one of said functions as a compound to be tested.

37. The method of claim 36 wherein said adverse affect associated with microcompetition is cancer and said patient is being treated for cancer.

38. The method of claim 36 wherein said adverse affect associated with microcompetition is atherosclerosis and said patient is being treated for atherosclerosis.

39. The method of claim 36 wherein said adverse affect associated with microcompetition is osteoarthritis and said patient is being treated for osteoarthritis.

40. The method of claim 36 wherein said adverse affect associated with microcompetition is obesity and said patient is being treated for obesity.

41. A method of identifying compounds to be tested for treating adverse affects associated with a disruption of GABP's metabolic pathway, comprising:
a) providing an assay capable of determining compounds that decrease oxidative effect on GABP; and
b) running sample compounds through said assay and identifying any compound that performs one of said functions as a compound to be tested.

42. The method of claim 41 wherein said adverse affect associated with microcompetition is cancer and said patient is being treated for cancer.

43. The method of claim 41 wherein said adverse affect associated with microcompetition is atherosclerosis and said patient is being treated for atherosclerosis.

44. The method of claim 41 wherein said adverse affect associated with microcompetition is osteoarthritis and said patient is being treated for osteoarthritis.

45. The method of claim 41 wherein said adverse affect associated with microcompetition is obesity and said patient is being treated for obesity.

46. A method of identifying compounds to be tested for treating adverse affects associated with a disruption of GABP's metabolic pathway, comprising:
a) providing an assay capable of determining compounds decreases foreign DNA N-boxes in cells; and
b) running sample compounds through said assay and identifying any compound that performs one of said functions as a compound to be tested.

47. The method of claim 46 wherein said adverse affect associated with microcompetition is cancer and said patient is being treated for cancer.

48. The method of claim 46 wherein said adverse affect associated with microcompetition is atherosclerosis and said patient is being treated for atherosclerosis.

49. The method of claim 46 wherein said adverse affect associated with microcompetition is osteoarthritis and said patient is being treated for osteoarthritis.

50. The method of claim 46 wherein said adverse affect associated with microcompetition is obesity and said patient is being treated for obesity.
